SEQUENCE LISTING (584 sequences)

SEQ ID NO: 1 - protein, 1423 aa, Artificial Sequence
  PEPTIDE 1..23: 3X FLAG Peptide
  Mutant (variant) Cas9 protein of the invention
  SIGNAL 24..40: Nuclear localization signal
  SITE 1042: Corresponds to Lys1003 in the wt Cas9 sequence with N-terminal methionine
  SITE 1043: Corresponds to Leu1004 in the sequence with N-terminal methionine
  SITE 1046: Corresponds to Glu1007 in the wt Cas9 sequence with N-terminal methionine
  SITE 1052: Corresponds to Tyr1013 in the wt Cas9 sequence with N-terminal methionine
  SITE 1053: Corresponds to Lys1014 in the wt Cas9 sequence with N-terminal methionine
  SITE 1056: Corresponds to Asp1017 in the wt Cas9 sequence with N-terminal methionine
  SIGNAL 1408..1423: Nuclear localization signal
MDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 2 - protein, 1367 aa, Streptococcus pyogenes serotype M1
  SITE 1002: Corresponds to Lys1003 in the wt Cas9 sequence with N-terminal methionine
  SITE 1003: Corresponds to Leu1004 in the sequence with N-terminal methionine
  SITE 1006: Corresponds to Glu1007 in the wt Cas9 sequence with N-terminal methionine
  SITE 1012: Corresponds to Tyr1013 in the wt Cas9 sequence with N-terminal methionine
  SITE 1013: Corresponds to Lys1014 in the wt Cas9 sequence with N-terminal methionine
  SITE 1016: Corresponds to Asp1017 in the wt Cas9 sequence with N-terminal methionine
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 1370 aa, Streptococcus pyogenes serotype M4
  SITE 1001: Corresponds to Lys1003 in the wt Cas9 sequence with N-terminal methionine
  SITE 1002: Corresponds to Leu1004 in the sequence with N-terminal methionine
  SITE 1005: Corresponds to Glu1007 in the wt Cas9 sequence with N-terminal methionine
  SITE 1011: Corresponds to Tyr1013 in the wt Cas9 sequence with N-terminal methionine
  SITE 1012: Corresponds to Lys1014 in the wt Cas9 sequence with N-terminal methionine
  SITE 1015: Corresponds to Asp1017 in the wt Cas9 sequence with N-terminal methionine
DKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQTYNQLFEENPINASRVDAKAILSARLSKSRRLENLIAQLPGEKKNGLFGNLIALSLGLTPNFKSNFDLAEDAKLQLSKDTYDDDLDNLLAQIGDQYADLFLAAKNLSDATLLSDILRVNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDKEMIEERLKTYAHLFDDKVMKQLKRRHYTGWGRLSRKLINGIRDKQSGKTILDFLKSDGFANRNFIQLIHDDSLTFKEAIQKAQVSGQGHSLHEQIANLAGSPAIKKGILQSVKVVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDDVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKRFFYSNIMNFFKTEITLANGEIRKRPLIETNEETGEIVWDKGRDFATVRKVLSMPQVNIVKKTEVQTGALTNESIYARGSFDKLISRKHRFESSKYGGFGSPTVTYSVLVVAKSKVQDGKVKKIKTGKELIGITLLDKLVFEKNPLKFIEDKGYGNVQIDKCIKLPKYSLFEFENGTRRMLASVMANNNSRGDLQKANEMFLPAKLVTLLYHAHKIESSKELEHEAYILDHYNDLYQLLSYIERFASLYVDVEKNISKVKELFSNIESYSISEICSSVINLLTLTASGAPADFKFLGTTIPRKRYGSPQSILSSTLIHQSITGLYETRIDLSQLGSD

SEQ ID NO: 4 - protein, 15 aa, Streptococcus pyogenes
  Wild type loop sequence
KLESEFVYGDYKVYD

SEQ ID NO: 5 - protein, 15 aa, Artificial Sequence
  HF-B1 mutant loop sequence
KLESGFVYGDGKVYD

SEQ ID NO: 6 - protein, 6 aa, Artificial Sequence
  HF-B2 mutant loop sequence
KGKVYD

SEQ ID NO: 7 - protein, 15 aa, Artificial Sequence
  HF-B3 mutant loop sequence
  SITE 2: Xaa is deletion
  SITE 3: Xaa is deletion
  SITE 4: Xaa is deletion
  SITE 5: Xaa is deletion
  SITE 6: Xaa is deletion
  SITE 7: Xaa is deletion
  SITE 8: Xaa is deletion
  SITE 9: Xaa is deletion
  SITE 10: Xaa is deletion
  SITE 11: Xaa is deletion
  SITE 12: Xaa is deletion
  SITE 13: Xaa is deletion
  SITE 14: Xaa is deletion
KXXXXXXXXXXXXXD

SEQ ID NO: 8 - protein, 15 aa, Artificial Sequence
  HF-B4 mutant loop sequence
  SITE 2: Xaa is deletion
  SITE 3: Xaa is deletion
  SITE 4: Xaa is deletion
  SITE 5: Xaa is deletion
  SITE 6: Xaa is deletion
  SITE 8: Xaa is deletion
  SITE 9: Xaa is deletion
  SITE 10: Xaa is deletion
  SITE 11: Xaa is deletion
  SITE 12: Xaa is deletion
  SITE 13: Xaa is deletion
  SITE 14: Xaa is deletion
KXXXXXGXXXXXXXD

SEQ ID NO: 9 - protein, 4 aa, Artificial Sequence
  HF-B5 mutant loop sequence
KGKD

SEQ ID NO: 10 - protein, 6 aa, Artificial Sequence
  HF-B6 mutant loop sequence
KLKVYD

SEQ ID NO: 11 - protein, 7 aa, Artificial Sequence
  HF-B7 mutant loop sequence
KLGKVYD

SEQ ID NO: 12 - protein, 7 aa, Artificial Sequence
  HF-B8 mutant loop sequence
KLPKVYD

SEQ ID NO: 13 - protein, 8 aa, Artificial Sequence
  HF-B9 (B-SpCas9-HF1) mutant loop sequence
KLGGKVYD

SEQ ID NO: 14 - protein, 8 aa, Artificial Sequence
  HF-B10 mutant loop sequence
KLGPKVYD

SEQ ID NO: 15 - protein, 9 aa, Artificial Sequence
  HF-B11 mutant loop sequence
KLGGGKVYD

SEQ ID NO: 16 - protein, 10 aa, Artificial Sequence
  HF-B12 mutant loop sequence
KLGGGGKVYD

SEQ ID NO: 17 - protein, 7 aa, Artificial Sequence
  HF-B13 mutant loop sequence
KLGGVYD

SEQ ID NO: 18 - protein, 6 aa, Artificial Sequence
  HF-B14 mutant loop sequence
KLGGYD

SEQ ID NO: 19 - protein, 5 aa, Artificial Sequence
  HF-B15 mutant loop sequence
KLGGD

SEQ ID NO: 20 - protein, 8 aa, Artificial Sequence
  HF-B16 mutant loop sequence
KLSSKVYD

SEQ ID NO: 21 - protein, 5 aa, Artificial Sequence
  HF-B17 mutant loop sequence
KLSSD

SEQ ID NO: 22 - protein, 8 aa, Artificial Sequence
  HF-B18 mutant loop sequence
KLESKVYD

SEQ ID NO: 23 - protein, 9 aa, Artificial Sequence
  HF-B19 mutant loop sequence
KLESGKVYD

SEQ ID NO: 24 - DNA, 4101 bp, Streptococcus pyogenes
  Nucleotide sequence coding wild type cas9 protein
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcgtacctgaacgccgtcgtgggaaccgccctgatcaaaaagtaccctaagctggaaagcgagttcgtgtacggcgactacaaggtgtacgacgtacggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 25 - DNA, 53 bp, Artificial Sequence
  i5-FANCF2-ONtarget-FWD PCR primer
tcgtcggcagcgtcagatgtgtataagagacagggtgctgacgtaggtagtgc

SEQ ID NO: 26 - DNA, 55 bp, Artificial Sequence
  i7-FANCF2-ONtarget-REV PCR primer
gtctcgtgggctcggagatgtgtataagagacaggtcacagtatgtctctggcgt

SEQ ID NO: 27 - DNA, 53 bp, Artificial Sequence
  i5-FANCF2-ONtarget-FWD2 PCR primer
tcgtcggcagcgtcagatgtgtataagagacaggggccgggaaagagttgctg

SEQ ID NO: 28 - DNA, 56 bp, Artificial Sequence
  i7-FANCF2-ONtarget-REV2 PCR primer
gtctcgtgggctcggagatgtgtataagagacaggccctacatctgctctccctcc

SEQ ID NO: 29 - DNA, 53 bp, Artificial Sequence
  i5-VEGFA3-ONtarget-FWD2 PCR primer
tcgtcggcagcgtcagatgtgtataagagacagtccagatggcacattgtcag

SEQ ID NO: 30 - DNA, 54 bp, Artificial Sequence
  i7-FVEGFA3-ONtarget-REV2 PCR primer
gtctcgtgggctcggagatgtgtataagagacagagggagcaggaaagtgaggt

SEQ ID NO: 31 - DNA, 53 bp, Artificial Sequence
  i5-VEGFA3-ONtarget-REV2 PCR primer
tcgtcggcagcgtcagatgtgtataagagacagagggagcaggaaagtgaggt

SEQ ID NO: 32 - DNA, 54 bp, Artificial Sequence
  i7-FVEGFA3-ONtarget-FWD2 PCR primer
gtctcgtgggctcggagatgtgtataagagacagtccagatggcacattgtcag

SEQ ID NO: 33 - DNA, 44 bp, Artificial Sequence
  T7-+G-Site51 primer
aaaaaataatacgactcactatagcgatgcccttcagctcgatg

SEQ ID NO: 34 - DNA, 44 bp, Artificial Sequence
  T7-+G-Site52 primer
aaaaaataatacgactcactatagcaacatcctggggcacaagc

SEQ ID NO: 35 - DNA, 44 bp, Artificial Sequence
  T7-+G-Site53 primer
aaaaaataatacgactcactatagcagctcgatgcggttcacca

SEQ ID NO: 36 - DNA, 44 bp, Artificial Sequence
  T7-+G-Site54 primer
aaaaaataatacgactcactatagcaaggaggacggcaacatcc

SEQ ID NO: 37 - DNA, 44 bp, Artificial Sequence
  T7-+G-Site55 primer
aaaaaataatacgactcactatagtcagctcgatgcggttcacc

SEQ ID NO: 38 - DNA, 44 bp, Artificial Sequence
  T7-+G-Site56 primer
aaaaaataatacgactcactatagaaggaggacggcaacatcct

SEQ ID NO: 39 - DNA, 44 bp, Artificial Sequence
  T7-+G-Site57 primer
aaaaaataatacgactcactatagaggaggacggcaacatcctg

SEQ ID NO: 40 - DNA, 44 bp, Artificial Sequence
  T7-+G-Site58 primer
aaaaaataatacgactcactatagcatgcccgaaggctacgtcc

SEQ ID NO: 41 - DNA, 44 bp, Artificial Sequence
  T7-+G-Site59 primer
aaaaaataatacgactcactatagcgtgctgcttcatgtggtcg

SEQ ID NO: 42 - DNA, 45 bp, Artificial Sequence
  T7-+2G-Site51 primer
aaaaaataatacgactcactataggcgatgcccttcagctcgatg

SEQ ID NO: 43 - DNA, 45 bp, Artificial Sequence
  T7-+2G-Site52 primer
aaaaaataatacgactcactataggcaacatcctggggcacaagc

SEQ ID NO: 44 - DNA, 45 bp, Artificial Sequence
  T7-+2G-Site53 primer
aaaaaataatacgactcactataggcagctcgatgcggttcacca

SEQ ID NO: 45 - DNA, 45 bp, Artificial Sequence
  T7-+2G-Site54 primer
aaaaaataatacgactcactataggcaaggaggacggcaacatcc

SEQ ID NO: 46 - DNA, 45 bp, Artificial Sequence
  T7-+2G-Site55 primer
aaaaaataatacgactcactataggtcagctcgatgcggttcacc

SEQ ID NO: 47 - DNA, 45 bp, Artificial Sequence
  T7-+2G-Site56 primer
aaaaaataatacgactcactataggaaggaggacggcaacatcct

SEQ ID NO: 48 - DNA, 45 bp, Artificial Sequence
  T7-+2G-Site57 primer
aaaaaataatacgactcactataggaggaggacggcaacatcctg

SEQ ID NO: 49 - DNA, 45 bp, Artificial Sequence
  T7-+2G-Site58 primer
aaaaaataatacgactcactataggcatgcccgaaggctacgtcc

SEQ ID NO: 50 - DNA, 45 bp, Artificial Sequence
  T7-+2G-Site59 primer
aaaaaataatacgactcactataggcgtgctgcttcatgtggtcg

SEQ ID NO: 51 - DNA, 43 bp, Artificial Sequence
  T7-Site-1 primer
aaaaaataatacgactcactatagggcacgggcagcttgccgg

SEQ ID NO: 52 - DNA, 43 bp, Artificial Sequence
  T7-Site3 primer
aaaaaataatacgactcactataggtggtgcagatgaacttca

SEQ ID NO: 53 - DNA, 43 bp, Artificial Sequence
  T7-Site4 primer
aaaaaataatacgactcactataggagcgcaccatcttcttca

SEQ ID NO: 54 - DNA, 43 bp, Artificial Sequence
  T7-Site16 primer
aaaaaataatacgactcactataggcgagggcgatgccaccta

SEQ ID NO: 55 - DNA, 43 bp, Artificial Sequence
  T7-Site21 primer
aaaaaataatacgactcactataggcatcgacttcaaggagga

SEQ ID NO: 56 - DNA, 43 bp, Artificial Sequence
  T7-Site22 primer
aaaaaataatacgactcactataggtgttctgctggtagtggt

SEQ ID NO: 57 - DNA, 43 bp, Artificial Sequence
  T7-Site31 primer
aaaaaataatacgactcactatagggcgaggagctgttcaccg

SEQ ID NO: 58 - DNA, 43 bp, Artificial Sequence
  T7-Site33 primer
aaaaaataatacgactcactataggtgcccatcctggtcgagc

SEQ ID NO: 59 - DNA, 43 bp, Artificial Sequence
  T7-Site37 primer
aaaaaataatacgactcactataggtcagggtggtcacgaggg

SEQ ID NO: 60 - DNA, 43 bp, Artificial Sequence
  T7-Site46 primer
aaaaaataatacgactcactataggttgtcgggcagcagcacg

SEQ ID NO: 61 - DNA, 43 bp, Artificial Sequence
  T7-Site48 primer
aaaaaataatacgactcactataggtgctcaggtagtggttgt

SEQ ID NO: 62 - DNA, 44 bp, Artificial Sequence
  T7-+G-Site1 primer
aaaaaataatacgactcactataggggcacgggcagcttgccgg

SEQ ID NO: 63 - DNA, 44 bp, Artificial Sequence
  T7-+G-Site3 primer
aaaaaataatacgactcactatagggtggtgcagatgaacttca

SEQ ID NO: 64 - DNA, 44 bp, Artificial Sequence
  T7-+G-Site4 primer
aaaaaataatacgactcactatagggagcgcaccatcttcttca

SEQ ID NO: 65 - DNA, 44 bp, Artificial Sequence
  T7-+G-Site16 primer
aaaaaataatacgactcactatagggcgagggcgatgccaccta

SEQ ID NO: 66 - DNA, 44 bp, Artificial Sequence
  T7-+G-Site21 primer
aaaaaataatacgactcactatagggcatcgacttcaaggagga

SEQ ID NO: 67 - DNA, 44 bp, Artificial Sequence
  T7-+G-Site22 primer
aaaaaataatacgactcactatagggtgttctgctggtagtggt

SEQ ID NO: 68 - DNA, 44 bp, Artificial Sequence
  T7-+G-Site31 primer
aaaaaataatacgactcactataggggcgaggagctgttcaccg

SEQ ID NO: 69 - DNA, 44 bp, Artificial Sequence
  T7-+G-Site33 primer
aaaaaataatacgactcactatagggtgcccatcctggtcgagc

SEQ ID NO: 70 - DNA, 44 bp, Artificial Sequence
  T7-+G-Site37 primer
aaaaaataatacgactcactatagggtcagggtggtcacgaggg

SEQ ID NO: 71 - DNA, 44 bp, Artificial Sequence
  T7-+G-Site46 primer
aaaaaataatacgactcactatagggttgtcgggcagcagcacg

SEQ ID NO: 72 - DNA, 44 bp, Artificial Sequence
  T7-+G-Site48 primer
aaaaaataatacgactcactatagggtgctcaggtagtggttgt

SEQ ID NO: 73 - DNA, 43 bp, Artificial Sequence
  T7-Site5 primer
aaaaaataatacgactcactatagtcgccctcgaacttcacct

SEQ ID NO: 74 - DNA, 43 bp, Artificial Sequence
  T7-Site13 primer
aaaaaataatacgactcactatagctgaagggcatcgacttca

SEQ ID NO: 75 - DNA, 43 bp, Artificial Sequence
  T7-Site17 primer
aaaaaataatacgactcactatagctgaagcactgcacgccgt

SEQ ID NO: 76 - DNA, 43 bp, Artificial Sequence
  T7-Site18 primer
aaaaaataatacgactcactatagtgaaccgcatcgagctgaa

SEQ ID NO: 77 - DNA, 43 bp, Artificial Sequence
  T7-Site20 primer
aaaaaataatacgactcactatagacgtagccttcgggcatgg

SEQ ID NO: 78 - DNA, 43 bp, Artificial Sequence
  T7-Site23 primer
aaaaaataatacgactcactatagtggtcacgagggtgggcca

SEQ ID NO: 79 - DNA, 43 bp, Artificial Sequence
  T7-Site38 primer
aaaaaataatacgactcactatagtaggtcagggtggtcacga

SEQ ID NO: 80 - DNA, 43 bp, Artificial Sequence
  T7-Site39 primer
aaaaaataatacgactcactatagcactgcacgccgtaggtca

SEQ ID NO: 81 - DNA, 43 bp, Artificial Sequence
  T7-Site44 primer
aaaaaataatacgactcactatagatgccgttcttctgcttgt

SEQ ID NO: 82 - DNA, 43 bp, Artificial Sequence
  T7-Site45 primer
aaaaaataatacgactcactatagcacggggccgtcgccgatg

SEQ ID NO: 83 - DNA, 43 bp, Artificial Sequence
  T7-Site47 primer
aaaaaataatacgactcactatagtggttgtcgggcagcagca

SEQ ID NO: 84 - DNA, 44 bp, Artificial Sequence
  T7-+G-Site5 primer
aaaaaataatacgactcactataggtcgccctcgaacttcacct

SEQ ID NO: 85 - DNA, 44 bp, Artificial Sequence
  T7-+G-Site13 primer
aaaaaataatacgactcactataggctgaagggcatcgacttca

SEQ ID NO: 86 - DNA, 44 bp, Artificial Sequence
  T7-+G-Site17 primer
aaaaaataatacgactcactataggctgaagcactgcacgccgt

SEQ ID NO: 87 - DNA, 44 bp, Artificial Sequence
  T7-+G-Site18 primer
aaaaaataatacgactcactataggtgaaccgcatcgagctgaa

SEQ ID NO: 88 - DNA, 44 bp, Artificial Sequence
  T7-+G-Site20 primer
aaaaaataatacgactcactataggacgtagccttcgggcatgg

SEQ ID NO: 89 - DNA, 44 bp, Artificial Sequence
  T7-+G-Site23 primer
aaaaaataatacgactcactataggtggtcacgagggtgggcca

SEQ ID NO: 90 - DNA, 44 bp, Artificial Sequence
  T7-+G-Site38 primer
aaaaaataatacgactcactataggtaggtcagggtggtcacga

SEQ ID NO: 91 - DNA, 44 bp, Artificial Sequence
  T7-+G-Site39 primer
aaaaaataatacgactcactataggcactgcacgccgtaggtca

SEQ ID NO: 92 - DNA, 44 bp, Artificial Sequence
  T7-+G-Site44 primer
aaaaaataatacgactcactataggatgccgttcttctgcttgt

SEQ ID NO: 93 - DNA, 44 bp, Artificial Sequence
  T7-+G-Site45 primer
aaaaaataatacgactcactataggcacggggccgtcgccgatg

SEQ ID NO: 94 - DNA, 44 bp, Artificial Sequence
  T7-+G-Site47 primer
aaaaaataatacgactcactataggtggttgtcgggcagcagca

SEQ ID NO: 95 - DNA, 44 bp, Artificial Sequence
  T7-+G-Site43 primer
aaaaaataatacgactcactataggaagggcatcgacttcaagg

SEQ ID NO: 96 - DNA, 44 bp, Artificial Sequence
  T7-+G-VEGFAsite2 primer
aaaaaataatacgactcactataggaccccctccaccccgcctc

SEQ ID NO: 97 - DNA, 44 bp, Artificial Sequence
  T7-+G-FANCFsite2 primer
aaaaaataatacgactcactataggctgcagaagggattccatg

SEQ ID NO: 98 - DNA, 22 bp, Artificial Sequence
  T7-reverse primer
aaaaaagcaccgactcggtgcc

SEQ ID NO: 99 - DNA, 20 bp, Artificial Sequence
  FANCF site 2-for primer
gggccgggaaagagttgctg

SEQ ID NO: 100 - DNA, 22 bp, Artificial Sequence
  FANCF site 2-rev primer
gccctacatctgctctccctcc

SEQ ID NO: 101 - DNA, 22 bp, Artificial Sequence
  VEGFA site 2-for primer
cgaggaagagagagacggggtc

SEQ ID NO: 102 - DNA, 23 bp, Artificial Sequence
  VEGFA site 2-rev primer
ctccaatgcacccaagacagcag

SEQ ID NO: 103 - DNA, 24 bp, Artificial Sequence
  HEK site 4-for primer
agagaagttggagtgaaggcagag

SEQ ID NO: 104 - DNA, 24 bp, Artificial Sequence
  HEK site 4-rev primer
gtcagacgtccaaaaccagactcc

SEQ ID NO: 105 - DNA, 20 bp, Artificial Sequence
  ZSCAN2-for primer
agtgtggggtgtgtgggaag

SEQ ID NO: 106 - DNA, 22 bp, Artificial Sequence
  ZSCAN2-rev primer
acgggacttgactcagaccact

SEQ ID NO: 107 - DNA, 20 bp, Artificial Sequence
  EMX1 site 2-for primer
ggagcagctggtcagagggg

SEQ ID NO: 108 - DNA, 22 bp, Artificial Sequence
  EMX1 site 2-rev
ccatagggaagggggacactgg

SEQ ID NO: 109 - DNA, 23 bp, Artificial Sequence
  DNMT1 site 4-for primer
ccagaatgcacaaagtactgcac

SEQ ID NO: 110 - DNA, 21 bp, Artificial Sequence
  DNMT1 site 4-rev
gccaaagcccgagagagtgcc

SEQ ID NO: 111 - DNA, 20 bp, Artificial Sequence
  EGFP site 1 spacer sequence
gggcacgggcagcttgccgg

SEQ ID NO: 112 - DNA, 20 bp, Artificial Sequence
  EGFP site 2 spacer sequence
gagctggacggcgacgtaaa

SEQ ID NO: 113 - DNA, 20 bp, Artificial Sequence
  EGFP site 3 spacer sequence
ggtggtgcagatgaacttca

SEQ ID NO: 114 - DNA, 20 bp, Artificial Sequence
  EGFP site 4 spacer sequence
ggagcgcaccatcttcttca

SEQ ID NO: 115 - DNA, 20 bp, Artificial Sequence
  EGFP site 5 spacer sequence
gtcgccctcgaacttcacct

SEQ ID NO: 116 - DNA, 20 bp, Artificial Sequence
  EGFP site 6 spacer sequence
gcgcgatcacatggtcctgc

SEQ ID NO: 117 - DNA, 20 bp, Artificial Sequence
  EGFP site 7 spacer sequence
gtccatgccgagagtgatcc

SEQ ID NO: 118 - DNA, 20 bp, Artificial Sequence
  EGFP site 8 spacer sequence
gccgtcgtccttgaagaaga

SEQ ID NO: 119 - DNA, 20 bp, Artificial Sequence
  EGFP site 9 spacer sequence
gaagttcgagggcgacaccc

SEQ ID NO: 120 - DNA, 20 bp, Artificial Sequence
  EGFP site 10 spacer sequence
gtctttgctcagggcggact

SEQ ID NO: 121 - DNA, 20 bp, Artificial Sequence
  EGFP site 11 spacer sequence
ggtctttgctcagggcggac

SEQ ID NO: 122 - DNA, 20 bp, Artificial Sequence
  EGFP site 12 spacer sequence
gtgctcaggtagtggttgtc

SEQ ID NO: 123 - DNA, 20 bp, Artificial Sequence
  EGFP site 13 spacer sequence
gctgaagggcatcgacttca

SEQ ID NO: 124 - DNA, 20 bp, Artificial Sequence
  EGFP site 14 spacer sequence
gtaccagcactagcctcctg

SEQ ID NO: 125 - DNA, 20 bp, Artificial Sequence
  EGFP site 15 spacer sequence
ggcatcgccctcgccctcgc

SEQ ID NO: 126 - DNA, 20 bp, Artificial Sequence
  EGFP site 16 spacer sequence
ggcgagggcgatgccaccta

SEQ ID NO: 127 - DNA, 20 bp, Artificial Sequence
  EGFP site 17 spacer sequence
gctgaagcactgcacgccgt

SEQ ID NO: 128 - DNA, 20 bp, Artificial Sequence
  EGFP site 18 spacer sequence
gtgaaccgcatcgagctgaa

SEQ ID NO: 129 - DNA, 20 bp, Artificial Sequence
  EGFP site 19
gtcagggtggtcacgagggt

SEQ ID NO: 130 - DNA, 20 bp, Artificial Sequence
  EGFP site 20 spacer sequence
gacgtagccttcgggcatgg

SEQ ID NO: 131 - DNA, 20 bp, Artificial Sequence
  EGFP site 21 spacer sequence
ggcatcgacttcaaggagga

SEQ ID NO: 132 - DNA, 20 bp, Artificial Sequence
  EGFP site 22 spacer sequence
ggtgttctgctggtagtggt

SEQ ID NO: 133 - DNA, 20 bp, Artificial Sequence
  EGFP site 23 spacer sequence
gtggtcacgagggtgggcca

SEQ ID NO: 134 - DNA, 20 bp, Artificial Sequence
  EGFP site 24 spacer sequence
gtcgtgctgcttcatgtggt

SEQ ID NO: 135 - DNA, 20 bp, Artificial Sequence
  EGFP site 25 spacer sequence
ggtccgagagtctgtagcca

SEQ ID NO: 136 - DNA, 20 bp, Artificial Sequence
  EGFP site 26 spacer sequence
gctgacggtcaggagccagg

SEQ ID NO: 137 - DNA, 20 bp, Artificial Sequence
  EGFP site 27 spacer sequence
ggcagagcaggctgacggtc

SEQ ID NO: 138 - DNA, 20 bp, Artificial Sequence
  EGFP site 28 spacer sequence
gagcaggcagagcaggctga

SEQ ID NO: 139 - DNA, 20 bp, Artificial Sequence
  EGFP site 29 spacer sequence
gaggccagagcaggcagagc

SEQ ID NO: 140 - DNA, 20 bp, Artificial Sequence
  EGFP site 30 spacer sequence
gctctgcctgctctggcctc

SEQ ID NO: 141 - DNA, 20 bp, Artificial Sequence
  EGFP site 31 spacer sequence
gggcgaggagctgttcaccg

SEQ ID NO: 142 - DNA, 20 bp, Artificial Sequence
  EGFP site 32 spacer sequence
gaccaggatgggcaccaccc

SEQ ID NO: 143 - DNA, 20 bp, Artificial Sequence
  EGFP site 33 spacer sequence
ggtgcccatcctggtcgagc

SEQ ID NO: 144 - DNA, 20 bp, Artificial Sequence
  EGFP site 34 spacer sequence
gccgtccagctcgaccagga

SEQ ID NO: 145 - DNA, 20 bp, Artificial Sequence
  EGFP site 35 spacer sequence
ggccacaagttcagcgtgtc

SEQ ID NO: 146 - DNA, 20 bp, Artificial Sequence
  EGFP site 36 spacer sequence
ggtggtcacgagggtgggcc

SEQ ID NO: 147 - DNA, 20 bp, Artificial Sequence
  EGFP site 37 spacer sequence
ggtcagggtggtcacgaggg

SEQ ID NO: 148 - DNA, 20 bp, Artificial Sequence
  EGFP site 38 spacer sequence
gtaggtcagggtggtcacga

SEQ ID NO: 149 - DNA, 20 bp, Artificial Sequence
  EGFP site 39 spacer sequence
gcactgcacgccgtaggtca

SEQ ID NO: 150 - DNA, 20 bp, Artificial Sequence
  EGFP site 40 spacer sequence
gcttcatgtggtcggggtag

SEQ ID NO: 151 - DNA, 20 bp, Artificial Sequence
  EGFP site 41 spacer sequence
gcgctcctggacgtagcctt

SEQ ID NO: 152 - DNA, 20 bp, Artificial Sequence
  EGFP site 42 spacer sequence
ggtgaaccgcatcgagctga

SEQ ID NO: 153 - DNA, 20 bp, Artificial Sequence
  EGFP site 43 spacer sequence
gaagggcatcgacttcaagg

SEQ ID NO: 154 - DNA, 20 bp, Artificial Sequence
  EGFP site 44 spacer sequence
gatgccgttcttctgcttgt

SEQ ID NO: 155 - DNA, 20 bp, Artificial Sequence
  EGFP site 45 spacer sequence
gcacggggccgtcgccgatg

SEQ ID NO: 156 - DNA, 20 bp, Artificial Sequence
  EGFP site 46 spacer sequence
ggttgtcgggcagcagcacg

SEQ ID NO: 157 - DNA, 20 bp, Artificial Sequence
  EGFP site 47 spacer sequence
gtggttgtcgggcagcagca

SEQ ID NO: 158 - DNA, 20 bp, Artificial Sequence
  EGFP site 48 spacer sequence
ggtgctcaggtagtggttgt

SEQ ID NO: 159 - DNA, 20 bp, Artificial Sequence
  EGFP site 49 spacer sequence
gttggggtctttgctcaggg

SEQ ID NO: 160 - DNA, 20 bp, Artificial Sequence
  EGFP site 50 spacer sequence
gccgagagtgatcccggcgg

SEQ ID NO: 161 - DNA, 20 bp, Artificial Sequence
  EGFP site 51-no 5′ G spacer sequence
cgatgcccttcagctcgatg

SEQ ID NO: 162 - DNA, 20 bp, Artificial Sequence
  EGFP site 52-no 5′ G spacer sequence
caacatcctggggcacaagc

SEQ ID NO: 163 - DNA, 20 bp, Artificial Sequence
  EGFP site 53-no 5′ G spacer sequence
cagctcgatgcggttcacca

SEQ ID NO: 164 - DNA, 20 bp, Artificial Sequence
  EGFP site 54-no 5′ G spacer sequence
caaggaggacggcaacatcc

SEQ ID NO: 165 - DNA, 20 bp, Artificial Sequence
  EGFP site 55-no 5′ G spacer sequence
tcagctcgatgcggttcacc

SEQ ID NO: 166 - DNA, 20 bp, Artificial Sequence
  EGFP site 56-no 5′ G spacer sequence
aaggaggacggcaacatcct

SEQ ID NO: 167 - DNA, 20 bp, Artificial Sequence
  EGFP site 57-no 5′ G spacer sequence
aggaggacggcaacatcctg

SEQ ID NO: 168 - DNA, 20 bp, Artificial Sequence
  EGFP site 58-no 5′ G spacer sequence
catgcccgaaggctacgtcc

SEQ ID NO: 169 - DNA, 20 bp, Artificial Sequence
  EGFP site 59-no 5′ G spacer sequence
cgtgctgcttcatgtggtcg

SEQ ID NO: 170 - DNA, 21 bp, Artificial Sequence
  EGFP site 1B spacer sequence
ggggcacgggcagcttgccgg

SEQ ID NO: 171 - DNA, 21 bp, Artificial Sequence
  EGFP site 2B spacer sequence
ggagctggacggcgacgtaaa

SEQ ID NO: 172 - DNA, 21 bp, Artificial Sequence
  EGFP site 3B spacer sequence
gggtggtgcagatgaacttca

SEQ ID NO: 173 - DNA, 21 bp, Artificial Sequence
  EGFP site 4B spacer sequence
gggagcgcaccatcttcttca

SEQ ID NO: 174 - DNA, 21 bp, Artificial Sequence
  EGFP site 5B spacer sequence
ggtcgccctcgaacttcacct

SEQ ID NO: 175 - DNA, 21 bp, Artificial Sequence
  EGFP site 6B spacer sequence
ggcgcgatcacatggtcctgc

SEQ ID NO: 176 - DNA, 21 bp, Artificial Sequence
  EGFP site 7B spacer sequence
ggtccatgccgagagtgatcc

SEQ ID NO: 177 - DNA, 21 bp, Artificial Sequence
  EGFP site 8B spacer sequence
ggccgtcgtccttgaagaaga

SEQ ID NO: 178 - DNA, 21 bp, Artificial Sequence
  EGFP site 9B spacer sequence
ggaagttcgagggcgacaccc

SEQ ID NO: 179 - DNA, 21 bp, Artificial Sequence
  EGFP site 13B spacer sequence
ggctgaagggcatcgacttca

SEQ ID NO: 180 - DNA, 21 bp, Artificial Sequence
  EGFP site 14B spacer sequence
ggtaccagcactagcctcctg

SEQ ID NO: 181 - DNA, 21 bp, Artificial Sequence
  EGFP site 15B spacer sequence
gggcatcgccctcgccctcgc

SEQ ID NO: 182 - DNA, 21 bp, Artificial Sequence
  EGFP site 24B spacer sequence
ggtcgtgctgcttcatgtggt

SEQ ID NO: 183 - DNA, 21 bp, Artificial Sequence
  EGFP site 25B spacer sequence
gggtccgagagtctgtagcca

SEQ ID NO: 184 - DNA, 21 bp, Artificial Sequence
  EGFP site 27B spacer sequence
gggcagagcaggctgacggtc

SEQ ID NO: 185 - DNA, 21 bp, Artificial Sequence
  EGFP site 28B spacer sequence
ggagcaggcagagcaggctga

SEQ ID NO: 186 - DNA, 21 bp, Artificial Sequence
  EGFP site 29B spacer sequence
ggaggccagagcaggcagagc

SEQ ID NO: 187 - DNA, 21 bp, Artificial Sequence
  EGFP site 30B spacer sequence
ggctctgcctgctctggcctc

SEQ ID NO: 188 - DNA, 21 bp, Artificial Sequence
  EGFP site 31B spacer sequence
ggggcgaggagctgttcaccg

SEQ ID NO: 189 - DNA, 21 bp, Artificial Sequence
  EGFP site 32B spacer sequence
ggaccaggatgggcaccaccc

SEQ ID NO: 190 - DNA, 21 bp, Artificial Sequence
  EGFP site 33B spacer sequence
gggtgcccatcctggtcgagc

SEQ ID NO: 191 - DNA, 21 bp, Artificial Sequence
  EGFP site 34B spacer sequence
ggccgtccagctcgaccagga

SEQ ID NO: 192 - DNA, 21 bp, Artificial Sequence
  EGFP site 35B spacer sequence
gggccacaagttcagcgtgtc

SEQ ID NO: 193 - DNA, 21 bp, Artificial Sequence
  EGFP site 37B spacer sequence
gggtcagggtggtcacgaggg

SEQ ID NO: 194 - DNA, 21 bp, Artificial Sequence
  EGFP site 38B spacer sequence
ggtaggtcagggtggtcacga

SEQ ID NO: 195 - DNA, 21 bp, Artificial Sequence
  EGFP site 39B spacer sequence
ggcactgcacgccgtaggtca

SEQ ID NO: 196 - DNA, 21 bp, Artificial Sequence
  EGFP site 40B spacer sequence
ggcttcatgtggtcggggtag

SEQ ID NO: 197 - DNA, 21 bp, Artificial Sequence
  EGFP site 41B spacer sequence
ggcgctcctggacgtagcctt

SEQ ID NO: 198 - DNA, 21 bp, Artificial Sequence
  EGFP site 42B spacer sequence
gggtgaaccgcatcgagctga

SEQ ID NO: 199 - DNA, 21 bp, Artificial Sequence
  EGFP site 43B spacer sequence
ggaagggcatcgacttcaagg

SEQ ID NO: 200 - DNA, 21 bp, Artificial Sequence
  EGFP site 44B spacer sequence
ggatgccgttcttctgcttgt

SEQ ID NO: 201 - DNA, 21 bp, Artificial Sequence
  EGFP site 45B spacer sequence
ggcacggggccgtcgccgatg

SEQ ID NO: 202 - DNA, 21 bp, Artificial Sequence
  EGFP site 46B spacer sequence
gggttgtcgggcagcagcacg

SEQ ID NO: 203 - DNA, 21 bp, Artificial Sequence
  EGFP site 47B spacer sequence
ggtggttgtcgggcagcagca

SEQ ID NO: 204 - DNA, 21 bp, Artificial Sequence
  EGFP site 49B spacer sequence
ggttggggtctttgctcaggg

SEQ ID NO: 205 - DNA, 21 bp, Artificial Sequence
  EGFP site 50B spacer sequence
ggccgagagtgatcccggcgg

SEQ ID NO: 206 - DNA, 21 bp, Artificial Sequence
  EGFP site 10C spacer sequence
ggtctttgctcagggcggact

SEQ ID NO: 207 - DNA, 21 bp, Artificial Sequence
  EGFP site 11C spacer sequence
gggtctttgctcagggcggac

SEQ ID NO: 208 - DNA, 21 bp, Artificial Sequence
  EGFP site 12C spacer sequence
ggtgctcaggtagtggttgtc

SEQ ID NO: 209 - DNA, 21 bp, Artificial Sequence
  EGFP site 16C spacer sequence
gggcgagggcgatgccaccta

SEQ ID NO: 210 - DNA, 21 bp, Artificial Sequence
  EGFP site 17C spacer sequence
ggctgaagcactgcacgccgt

SEQ ID NO: 211 - DNA, 21 bp, Artificial Sequence
  EGFP site 18C spacer sequence
ggtgaaccgcatcgagctgaa

SEQ ID NO: 212 - DNA, 21 bp, Artificial Sequence
  EGFP site 19C spacer sequence
ggtcagggtggtcacgagggt

SEQ ID NO: 213 - DNA, 21 bp, Artificial Sequence
  EGFP site 20C spacer sequence
ggacgtagccttcgggcatgg

SEQ ID NO: 214 - DNA, 21 bp, Artificial Sequence
  EGFP site 21C spacer sequence
gggcatcgacttcaaggagga

SEQ ID NO: 215 - DNA, 21 bp, Artificial Sequence
  EGFP site 22C spacer sequence
gggtgttctgctggtagtggt

SEQ ID NO: 216 - DNA, 21 bp, Artificial Sequence
  EGFP site 23C spacer sequence
ggtggtcacgagggtgggcca

SEQ ID NO: 217 - DNA, 21 bp, Artificial Sequence
  EGFP site 26C spacer sequence
ggctgacggtcaggagccagg

SEQ ID NO: 218 - DNA, 21 bp, Artificial Sequence
  EGFP site 36C spacer sequence
gggtggtcacgagggtgggcc

SEQ ID NO: 219 - DNA, 21 bp, Artificial Sequence
  EGFP site 48C spacer sequence
gggtgctcaggtagtggttgt

SEQ ID NO: 220 - DNA, 23 bp, Artificial Sequence
  EGFP site 1 target sequence with PAM
gggcacgggcagcttgccggtgg

SEQ ID NO: 221 - DNA, 23 bp, Artificial Sequence
  EGFP site 2 target sequence with PAM
gagctggacggcgacgtaaacgg

SEQ ID NO: 222 - DNA, 23 bp, Artificial Sequence
  EGFP site 3 target sequence with PAM
ggtggtgcagatgaacttcaggg

SEQ ID NO: 223 - DNA, 23 bp, Artificial Sequence
  EGFP site 4 target sequence with PAM
ggagcgcaccatcttcttcaagg

SEQ ID NO: 224 - DNA, 23 bp, Artificial Sequence
  EGFP site 5 target sequence with PAM
gtcgccctcgaacttcacctcgg

SEQ ID NO: 225 - DNA, 23 bp, Artificial Sequence
  EGFP site 6 target sequence with PAM
gcgcgatcacatggtcctgctgg

SEQ ID NO: 226 - DNA, 23 bp, Artificial Sequence
  EGFP site 7 target sequence with PAM
gtccatgccgagagtgatcccgg

SEQ ID NO: 227 - DNA, 23 bp, Artificial Sequence
  EGFP site 8 target sequence with PAM
gccgtcgtccttgaagaagatgg

SEQ ID NO: 228 - DNA, 23 bp, Artificial Sequence
  EGFP site 9 target sequence with PAM
gaagttcgagggcgacaccctgg

SEQ ID NO: 229 - DNA, 23 bp, Artificial Sequence
  EGFP site 10 target sequence with PAM
gtctttgctcagggcggactggg

SEQ ID NO: 230 - DNA, 23 bp, Artificial Sequence
  EGFP site 11 target sequence with PAM
ggtctttgctcagggcggactgg

SEQ ID NO: 231 - DNA, 23 bp, Artificial Sequence
  EGFP site 12 target sequence with PAM
gtgctcaggtagtggttgtcggg

SEQ ID NO: 232 - DNA, 23 bp, Artificial Sequence
  EGFP site 13 target sequence with PAM
gctgaagggcatcgacttcaagg

SEQ ID NO: 233 - DNA, 23 bp, Artificial Sequence
  EGFP site 14 target sequence with PAM
gtaccagcactagcctcctgagg

SEQ ID NO: 234 - DNA, 23 bp, Artificial Sequence
  EGFP site 15 target sequence with PAM
ggcatcgccctcgccctcgccgg

SEQ ID NO: 235 - DNA, 23 bp, Artificial Sequence
  EGFP site 16 target sequence with PAM
ggcgagggcgatgccacctacgg

SEQ ID NO: 236 - DNA, 23 bp, Artificial Sequence
  EGFP site 17 target sequence with PAM
gctgaagcactgcacgccgtagg

SEQ ID NO: 237 - DNA, 23 bp, Artificial Sequence
  EGFP site 18 target sequence with PAM
gtgaaccgcatcgagctgaaggg

SEQ ID NO: 238 - DNA, 23 bp, Artificial Sequence
  EGFP site 19 target sequence with PAM
gtcagggtggtcacgagggtggg

SEQ ID NO: 239 - DNA, 23 bp, Artificial Sequence
  EGFP site 20 target sequence with PAM
gacgtagccttcgggcatggcgg

SEQ ID NO: 240 - DNA, 23 bp, Artificial Sequence
  EGFP site 21 target sequence with PAM
ggcatcgacttcaaggaggacgg

SEQ ID NO: 241 - DNA, 23 bp, Artificial Sequence
  EGFP site 22 target sequence with PAM
ggtgttctgctggtagtggtcgg

SEQ ID NO: 242 - DNA, 23 bp, Artificial Sequence
  EGFP site 23 target sequence with PAM
gtggtcacgagggtgggccaggg

SEQ ID NO: 243 - DNA, 23 bp, Artificial Sequence
  EGFP site 24 target sequence with PAM
gtcgtgctgcttcatgtggtcgg

SEQ ID NO: 244 - DNA, 23 bp, Artificial Sequence
  EGFP site 25 target sequence with PAM
ggtccgagagtctgtagccatgg

SEQ ID NO: 245 - DNA, 23 bp, Artificial Sequence
  EGFP site 26 target sequence with PAM
gctgacggtcaggagccaggagg

SEQ ID NO: 246 - DNA, 23 bp, Artificial Sequence
  EGFP site 27 target sequence with PAM
ggcagagcaggctgacggtcagg

SEQ ID NO: 247 - DNA, 23 bp, Artificial Sequence
  EGFP site 28 target sequence with PAM
gagcaggcagagcaggctgacgg

SEQ ID NO: 248 - DNA, 23 bp, Artificial Sequence
  EGFP site 29 target sequence with PAM
gaggccagagcaggcagagcagg

SEQ ID NO: 249 - DNA, 23 bp, Artificial Sequence
  EGFP site 30 target sequence with PAM
gctctgcctgctctggcctcagg

SEQ ID NO: 250 - DNA, 23 bp, Artificial Sequence
  EGFP site 31 target sequence with PAM
gggcgaggagctgttcaccgggg

SEQ ID NO: 251 - DNA, 23 bp, Artificial Sequence
  EGFP site 32 target sequence with PAM
gaccaggatgggcaccaccccgg

SEQ ID NO: 252 - DNA, 23 bp, Artificial Sequence
  EGFP site 33 target sequence with PAM
ggtgcccatcctggtcgagctgg

SEQ ID NO: 253 - DNA, 23 bp, Artificial Sequence
  EGFP site 34 target sequence with PAM
gccgtccagctcgaccaggatgg

SEQ ID NO: 254 - DNA, 23 bp, Artificial Sequence
  EGFP site 35 target sequence with PAM
ggccacaagttcagcgtgtccgg

SEQ ID NO: 255 - DNA, 23 bp, Artificial Sequence
  EGFP site 36 target sequence with PAM
ggtggtcacgagggtgggccagg

SEQ ID NO: 256 - DNA, 23 bp, Artificial Sequence
  EGFP site 37 target sequence with PAM
ggtcagggtggtcacgagggtgg

SEQ ID NO: 257 - DNA, 23 bp, Artificial Sequence
  EGFP site 38 target sequence with PAM
gtaggtcagggtggtcacgaggg

SEQ ID NO: 258 - DNA, 23 bp, Artificial Sequence
  EGFP site 39 target sequence with PAM
gcactgcacgccgtaggtcaggg

SEQ ID NO: 259 - DNA, 23 bp, Artificial Sequence
  EGFP site 40 target sequence with PAM
gcttcatgtggtcggggtagcgg

SEQ ID NO: 260 - DNA, 23 bp, Artificial Sequence
  EGFP site 41 target sequence with PAM
gcgctcctggacgtagccttcgg

SEQ ID NO: 261 - DNA, 23 bp, Artificial Sequence
  EGFP site 42 target sequence with PAM
ggtgaaccgcatcgagctgaagg

SEQ ID NO: 262 - DNA, 23 bp, Artificial Sequence
  EGFP site 43 target sequence with PAM
gaagggcatcgacttcaaggagg

SEQ ID NO: 263 - DNA, 23 bp, Artificial Sequence
  EGFP site 44 target sequence with PAM
gatgccgttcttctgcttgtcgg

SEQ ID NO: 264 - DNA, 23 bp, Artificial Sequence
  EGFP site 45 target sequence with PAM
gcacggggccgtcgccgatgggg

SEQ ID NO: 265 - DNA, 23 bp, Artificial Sequence
  EGFP site 46 target sequence with PAM
ggttgtcgggcagcagcacgggg

SEQ ID NO: 266 - DNA, 23 bp, Artificial Sequence
  EGFP site 47 target sequence with PAM
gtggttgtcgggcagcagcacgg

SEQ ID NO: 267 - DNA, 23 bp, Artificial Sequence
  EGFP site 48 target sequence with PAM
ggtgctcaggtagtggttgtcgg

SEQ ID NO: 268 - DNA, 23 bp, Artificial Sequence
  EGFP site 49 target sequence with PAM
gttggggtctttgctcagggcgg

SEQ ID NO: 269 - DNA, 23 bp, Artificial Sequence
  EGFP site 50 target sequence with PAM
gccgagagtgatcccggcggcgg

SEQ ID NO: 270 - DNA, 23 bp, Artificial Sequence
  EGFP site 51-no 5′ G target sequence with PAM
cgatgcccttcagctcgatgcgg

SEQ ID NO: 271 - DNA, 23 bp, Artificial Sequence
  EGFP site 52-no 5′ G target sequence with PAM
caacatcctggggcacaagctgg

SEQ ID NO: 272 - DNA, 23 bp, Artificial Sequence
  EGFP site 53-no 5′ G target sequence with PAM
cagctcgatgcggttcaccaggg

SEQ ID NO: 273 - DNA, 23 bp, Artificial Sequence
  EGFP site 54-no 5′ G target sequence with PAM
caaggaggacggcaacatcctgg

SEQ ID NO: 274 - DNA, 23 bp, Artificial Sequence
  EGFP site 55-no 5′ G target sequence with PAM
tcagctcgatgcggttcaccagg

SEQ ID NO: 275 - DNA, 23 bp, Artificial Sequence
  EGFP site 56-no 5′ G target sequence with PAM
aaggaggacggcaacatcctggg

SEQ ID NO: 276 - DNA, 23 bp, Artificial Sequence
  EGFP site 57-no 5′ G target sequence with PAM
aggaggacggcaacatcctgggg

SEQ ID NO: 277 - DNA, 23 bp, Artificial Sequence
  EGFP site 58-no 5′ G target sequence with PAM
catgcccgaaggctacgtccagg

SEQ ID NO: 278 - DNA, 23 bp, Artificial Sequence
  EGFP site 59-no 5′ G target sequence with PAM
cgtgctgcttcatgtggtcgggg

SEQ ID NO: 279 - DNA, 23 bp, Artificial Sequence
  EGFP site 1B target sequence with PAM
gggcacgggcagcttgccggtgg

SEQ ID NO: 280 - DNA, 23 bp, Artificial Sequence
  EGFP site 2B target sequence with PAM
gagctggacggcgacgtaaacgg

SEQ ID NO: 281 - DNA, 23 bp, Artificial Sequence
  EGFP site 3B target sequence with PAM
ggtggtgcagatgaacttcaggg

SEQ ID NO: 282 - DNA, 23 bp, Artificial Sequence
  EGFP site 4B target sequence with PAM
ggagcgcaccatcttcttcaagg

SEQ ID NO: 283 - DNA, 23 bp, Artificial Sequence
  EGFP site 5B target sequence with PAM
gtcgccctcgaacttcacctcgg

SEQ ID NO: 284 - DNA, 23 bp, Artificial Sequence
  EGFP site 6B target sequence with PAM
gcgcgatcacatggtcctgctgg

SEQ ID NO: 285 - DNA, 23 bp, Artificial Sequence
  EGFP site 7B target sequence with PAM
gtccatgccgagagtgatcccgg

SEQ ID NO: 286 - DNA, 23 bp, Artificial Sequence
  EGFP site 8B target sequence with PAM
gccgtcgtccttgaagaagatgg

SEQ ID NO: 287 - DNA, 23 bp, Artificial Sequence
  EGFP site 9B target sequence with PAM
gaagttcgagggcgacaccctgg

SEQ ID NO: 288 - DNA, 23 bp, Artificial Sequence
  EGFP site 13B target sequence with PAM
gctgaagggcatcgacttcaagg

SEQ ID NO: 289 - DNA, 23 bp, Artificial Sequence
  EGFP site 14B target sequence with PAM
gtaccagcactagcctcctgagg

SEQ ID NO: 290 - DNA, 23 bp, Artificial Sequence
  EGFP site 15B target sequence with PAM
ggcatcgccctcgccctcgccgg

SEQ ID NO: 291 - DNA, 23 bp, Artificial Sequence
  EGFP site 24B target sequence with PAM
gtcgtgctgcttcatgtggtcgg

SEQ ID NO: 292 - DNA, 23 bp, Artificial Sequence
  EGFP site 25B target sequence with PAM
ggtccgagagtctgtagccatgg

SEQ ID NO: 293 - DNA, 23 bp, Artificial Sequence
  EGFP site 27B target sequence with PAM
ggcagagcaggctgacggtcagg

SEQ ID NO: 294 - DNA, 23 bp, Artificial Sequence
  EGFP site 28B target sequence with PAM
gagcaggcagagcaggctgacgg

SEQ ID NO: 295 - DNA, 23 bp, Artificial Sequence
  EGFP site 29B target sequence with PAM
gaggccagagcaggcagagcagg

SEQ ID NO: 296 - DNA, 23 bp, Artificial Sequence
  EGFP site 30B target sequence with PAM
gctctgcctgctctggcctcagg

SEQ ID NO: 297 - DNA, 23 bp, Artificial Sequence
  EGFP site 31B target sequence with PAM
gggcgaggagctgttcaccgggg

SEQ ID NO: 298 - DNA, 23 bp, Artificial Sequence
  EGFP site 32B target sequence with PAM
gaccaggatgggcaccaccccgg

SEQ ID NO: 299 - DNA, 23 bp, Artificial Sequence
  EGFP site 33B target sequence with PAM
ggtgcccatcctggtcgagctgg

SEQ ID NO: 300 - DNA, 23 bp, Artificial Sequence
  EGFP site 34B target sequence with PAM
gccgtccagctcgaccaggatgg

SEQ ID NO: 301 - DNA, 23 bp, Artificial Sequence
  EGFP site 35B target sequence with PAM
ggccacaagttcagcgtgtccgg

SEQ ID NO: 302 - DNA, 23 bp, Artificial Sequence
  EGFP site 37B target sequence with PAM
ggtcagggtggtcacgagggtgg

SEQ ID NO: 303 - DNA, 23 bp, Artificial Sequence
  EGFP site 38B target sequence with PAM
gtaggtcagggtggtcacgaggg

SEQ ID NO: 304 - DNA, 23 bp, Artificial Sequence
  EGFP site 39B target sequence with PAM
gcactgcacgccgtaggtcaggg

SEQ ID NO: 305 - DNA, 23 bp, Artificial Sequence
  EGFP site 40B target sequence with PAM
gcttcatgtggtcggggtagcgg

SEQ ID NO: 306 - DNA, 23 bp, Artificial Sequence
  EGFP site 41B target sequence with PAM
gcgctcctggacgtagccttcgg

SEQ ID NO: 307 - DNA, 23 bp, Artificial Sequence
  EGFP site 42B target sequence with PAM
ggtgaaccgcatcgagctgaagg

SEQ ID NO: 308 - DNA, 23 bp, Artificial Sequence
  EGFP site 43B target sequence with PAM
gaagggcatcgacttcaaggagg

SEQ ID NO: 309 - DNA, 23 bp, Artificial Sequence
  EGFP site 44B target sequence with PAM
gatgccgttcttctgcttgtcgg

SEQ ID NO: 310 - DNA, 23 bp, Artificial Sequence
  EGFP site 45B target sequence with PAM
gcacggggccgtcgccgatgggg

SEQ ID NO: 311 - DNA, 23 bp, Artificial Sequence
  EGFP site 46B target sequence with PAM
ggttgtcgggcagcagcacgggg

SEQ ID NO: 312 - DNA, 23 bp, Artificial Sequence
  EGFP site 47B target sequence with PAM
gtggttgtcgggcagcagcacgg

SEQ ID NO: 313 - DNA, 23 bp, Artificial Sequence
  EGFP site 49B target sequence with PAM
gttggggtctttgctcagggcgg

SEQ ID NO: 314 - DNA, 23 bp, Artificial Sequence
  EGFP site 50B target sequence with PAM
gccgagagtgatcccggcggcgg

SEQ ID NO: 315 - DNA, 24 bp, Artificial Sequence
  EGFP site 10C target sequence with PAM
ggtctttgctcagggcggactggg

SEQ ID NO: 316 - DNA, 23 bp, Artificial Sequence
  EGFP site 11C target sequence with PAM
ggtctttgctcagggcggactgg

SEQ ID NO: 317 - DNA, 24 bp, Artificial Sequence
  EGFP site 12C target sequence with PAM
ggtgctcaggtagtggttgtcggg

SEQ ID NO: 318 - DNA, 24 bp, Artificial Sequence
  EGFP site 16C target sequence with PAM
gggcgagggcgatgccacctacgg

SEQ ID NO: 319 - DNA, 24 bp, Artificial Sequence
  EGFP site 17C target sequence with PAM
ggctgaagcactgcacgccgtagg

SEQ ID NO: 320 - DNA, 24 bp, Artificial Sequence
  EGFP site 18C target sequence with PAM
ggtgaaccgcatcgagctgaaggg

SEQ ID NO: 321 - DNA, 24 bp, Artificial Sequence
  EGFP site 19C target sequence with PAM
ggtcagggtggtcacgagggtggg

SEQ ID NO: 322 - DNA, 24 bp, Artificial Sequence
  EGFP site 20C target sequence with PAM
ggacgtagccttcgggcatggcgg

SEQ ID NO: 323 - DNA, 24 bp, Artificial Sequence
  EGFP site 21C target sequence with PAM
gggcatcgacttcaaggaggacgg

SEQ ID NO: 324 - DNA, 24 bp, Artificial Sequence
  EGFP site 22C target sequence with PAM
gggtgttctgctggtagtggtcgg

SEQ ID NO: 325 - DNA, 24 bp, Artificial Sequence
  EGFP site 23C target sequence with PAM
ggtggtcacgagggtgggccaggg

SEQ ID NO: 326 - DNA, 23 bp, Artificial Sequence
  EGFP site 26C target sequence with PAM
gctgacggtcaggagccaggagg

SEQ ID NO: 327 - DNA, 23 bp, Artificial Sequence
  EGFP site 36C target sequence with PAM
ggtggtcacgagggtgggccagg

SEQ ID NO: 328 - DNA, 23 bp, Artificial Sequence
  EGFP site 48C target sequence with PAM
ggtgctcaggtagtggttgtcgg

SEQ ID NO: 329 - skipped

SEQ ID NO: 330 - DNA, 23 bp, Artificial Sequence
  EGFP site 51 target sequence with PAM
cgatgcccttcagctcgatgcgg

SEQ ID NO: 331 - DNA, 23 bp, Artificial Sequence
  EGFP site 52 target sequence with PAM
caacatcctggggcacaagctgg

SEQ ID NO: 332 - DNA, 23 bp, Artificial Sequence
  EGFP site 53 target sequence with PAM
cagctcgatgcggttcaccaggg

SEQ ID NO: 333 - DNA, 23 bp, Artificial Sequence
  EGFP site 55 target sequence with PAM
tcagctcgatgcggttcaccagg

SEQ ID NO: 334 - DNA, 23 bp, Artificial Sequence
  EGFP site 59 target sequence with PAM
cgtgctgcttcatgtggtcgggg

SEQ ID NO: 335 - DNA, 23 bp, Artificial Sequence
  EGFP site 60 target sequence with PAM
ccgtccagctcgaccaggatggg

SEQ ID NO: 336 - DNA, 23 bp, Artificial Sequence
  EGFP site 61 target sequence with PAM
caactacaagacccgcgccgagg

SEQ ID NO: 337 - DNA, 23 bp, Artificial Sequence
  EGFP site 62 target sequence with PAM
aagggcgaggagctgttcaccgg

SEQ ID NO: 338 - DNA, 23 bp, Artificial Sequence
  EGFP site 63 target sequence with PAM
aagttcagcgtgtccggcgaggg

SEQ ID NO: 339 - DNA, 23 bp, Artificial Sequence
  EGFP site 64 target sequence with PAM
agcgtgtccggcgagggcgaggg

SEQ ID NO: 340 - DNA, 23 bp, Artificial Sequence
  EGFP site 65 target sequence with PAM
acgagggtgggccagggcacggg

SEQ ID NO: 341 - DNA, 23 bp, Artificial Sequence
  EGFP site 66 target sequence
agaagtcgtgctgcttcatgtgg

SEQ ID NO: 342 - DNA, 23 bp, Artificial Sequence
  EGFP site 67 target sequence with PAM
accatcttcttcaaggacgacgg

SEQ ID NO: 343 - DNA, 23 bp, Artificial Sequence
  EGFP site 68 target sequence with PAM
aaggaggacggcaacatcctggg

SEQ ID NO: 344 - DNA, 23 bp, Artificial Sequence
  EGFP site 69 target sequence with PAM
tcagctcgatgcggttcaccagg

SEQ ID NO: 345 - DNA, 23 bp, Artificial Sequence
  EGFP site 70 target sequence with PAM
tcgtgctgcttcatgtggtcggg

SEQ ID NO: 346 - DNA, 23 bp, Artificial Sequence
  EGFP site 71 target sequence with PAM
ttcaagtccgccatgcccgaagg

SEQ ID NO: 347 - DNA, 23 bp, Artificial Sequence
  EGFP site 72 target sequence with PAM
tgaagaagatggtgcgctcctgg

SEQ ID NO: 348 - DNA, 23 bp, Artificial Sequence
  EGFP site 73 target sequence with PAM
tgtactccagcttgtgccccagg

SEQ ID NO: 349 - DNA, 23 bp, Artificial Sequence
  EGFP site 74 target sequence with PAM
tgccgtcctcgatgttgtggcgg

SEQ ID NO: 350 - DNA, 23 bp, Artificial Sequence
  EGFP site 75 target sequence with PAM
ccgcgccgaggtgaagttcgagg

SEQ ID NO: 351 - DNA, 21 bp, Artificial Sequence
  EGFP site 51 21G-sgRNA (spacer sequence)
gcgatgcccttcagctcgatg

SEQ ID NO: 352 - DNA, 21 bp, Artificial Sequence
  EGFP site 52 21G-sgRNA (spacer sequence)
gcaacatcctggggcacaagc

SEQ ID NO: 353 - DNA, 21 bp, Artificial Sequence
  EGFP site 53 21G-sgRNA (spacer sequence)
gcagctcgatgcggttcacca

SEQ ID NO: 354 - DNA, 21 bp, Artificial Sequence
  EGFP site 55 21G-sgRNA (spacer sequence)
gtcagctcgatgcggttcacc

SEQ ID NO: 355 - DNA, 21 bp, Artificial Sequence
  EGFP site 59 21G-sgRNA (spacer sequence)
gcgtgctgcttcatgtggtcg

SEQ ID NO: 356 - DNA, 21 bp, Artificial Sequence
  EGFP site 60 21G-sgRNA (spacer sequence)
gccgtccagctcgaccaggat

SEQ ID NO: 357 - DNA, 21 bp, Artificial Sequence
  EGFP site 61 21G-sgRNA (spacer sequence)
gcaactacaagacccgcgccg

SEQ ID NO: 358 - DNA, 21 bp, Artificial Sequence
  EGFP site 62 21G-sgRNA (spacer sequence)
gaagggcgaggagctgttcac

SEQ ID NO: 359 - DNA, 21 bp, Artificial Sequence
  EGFP site 63 21G-sgRNA (spacer sequence)
gaagttcagcgtgtccggcga

SEQ ID NO: 360 - DNA, 21 bp, Artificial Sequence
  EGFP site 64 21G-sgRNA (spacer sequence)
gagcgtgtccggcgagggcga

SEQ ID NO: 361 - DNA, 21 bp, Artificial Sequence
  EGFP site 65 21G-sgRNA (spacer sequence)
gacgagggtgggccagggcac

SEQ ID NO: 362 - DNA, 21 bp, Artificial Sequence
  EGFP site 66 21G-sgRNA (spacer sequence)
gagaagtcgtgctgcttcatg

SEQ ID NO: 363 - DNA, 21 bp, Artificial Sequence
  EGFP site 67 21G-sgRNA (spacer sequence)
gaccatcttcttcaaggacga

SEQ ID NO: 364 - DNA, 21 bp, Artificial Sequence
  EGFP site 68 21G-sgRNA (spacer sequence)
gaaggaggacggcaacatcct

SEQ ID NO: 365 - DNA, 21 bp, Artificial Sequence
  EGFP site 69 21G-sgRNA (spacer sequence)
gtcagctcgatgcggttcacc

SEQ ID NO: 366 - DNA, 21 bp, Artificial Sequence
  EGFP site 70 21G-sgRNA (spacer sequence)
gtcgtgctgcttcatgtggtc

SEQ ID NO: 367 - DNA, 21 bp, Artificial Sequence
  EGFP site 71 21G-sgRNA (spacer sequence)
gttcaagtccgccatgcccga

SEQ ID NO: 368 - DNA, 21 bp, Artificial Sequence
  EGFP site 72 21G-sgRNA (spacer sequence)
gtgaagaagatggtgcgctcc

SEQ ID NO: 369 - DNA, 21 bp, Artificial Sequence
  EGFP site 73 21G-sgRNA (spacer sequence)
gtgtactccagcttgtgcccc

SEQ ID NO: 370 - DNA, 21 bp, Artificial Sequence
  EGFP site 74 21G-sgRNA (spacer sequence)
gtgccgtcctcgatgttgtgg

SEQ ID NO: 371 - DNA, 21 bp, Artificial Sequence
  EGFP site 75 21G-sgRNA (spacer sequence)
gccgcgccgaggtgaagttcg

SEQ ID NO: 372 - DNA, 63 bp, Artificial Sequence
  EGFP site 51 ribosyme flanked sgRNA (ribosyme- spacer sequence)
gcatcgctgatgagtccgtgaggacgaaacgagtaagctcgtccgatgcccttcagctcgatg

SEQ ID NO: 373 - DNA, 63 bp, Artificial Sequence
  EGFP site 52 ribosyme flanked sgRNA (ribosyme- spacer sequence)
atgttgctgatgagtccgtgaggacgaaacgagtaagctcgtccaacatcctggggcacaagc

SEQ ID NO: 374 - DNA, 63 bp, Artificial Sequence
  EGFP site 53 ribosyme flanked sgRNA (ribosyme- spacer sequence)
gagctgctgatgagtccgtgaggacgaaacgagtaagctcgtccagctcgatgcggttcacca

SEQ ID NO: 375 - DNA, 63 bp, Artificial Sequence
  EGFP site 55 ribosyme flanked sgRNA (ribosyme- spacer sequence)
agctgactgatgagtccgtgaggacgaaacgagtaagctcgtctcagctcgatgcggttcacc

SEQ ID NO: 376 - DNA, 63 bp, Artificial Sequence
  EGFP site 59 ribosyme flanked sgRNA (ribosyme- spacer sequence)
agcacgctgatgagtccgtgaggacgaaacgagtaagctcgtccgtgctgcttcatgtggtcg

SEQ ID NO: 377 - DNA, 63 bp, Artificial Sequence
  EGFP site 60 ribosyme flanked sgRNA (ribosyme- spacer sequence)
ggacggctgatgagtccgtgaggacgaaacgagtaagctcgtcccgtccagctcgaccaggat

SEQ ID NO: 378 - DNA, 63 bp, Artificial Sequence
  EGFP site 61 ribosyme flanked sgRNA (ribosyme- spacer sequence)
tagttgctgatgagtccgtgaggacgaaacgagtaagctcgtccaactacaagacccgcgccg

SEQ ID NO: 379 - DNA, 63 bp, Artificial Sequence
  EGFP site 62 ribosyme flanked sgRNA (ribosyme- spacer sequence)
gcccttctgatgagtccgtgaggacgaaacgagtaagctcgtcaagggcgaggagctgttcac

SEQ ID NO: 380 - DNA, 63 bp, Artificial Sequence
  EGFP site 63 ribosyme flanked sgRNA (ribosyme- spacer sequence)
gaacttctgatgagtccgtgaggacgaaacgagtaagctcgtcaagttcagcgtgtccggcga

SEQ ID NO: 381 - DNA, 63 bp, Artificial Sequence
  EGFP site 64 ribosyme flanked sgRNA (ribosyme- spacer sequence)
cacgctctgatgagtccgtgaggacgaaacgagtaagctcgtcagcgtgtccggcgagggcga

SEQ ID NO: 382 - DNA, 63 bp, Artificial Sequence
  EGFP site 65 ribosyme flanked sgRNA (ribosyme- spacer sequence)
cctcgtctgatgagtccgtgaggacgaaacgagtaagctcgtcacgagggtgggccagggcac

SEQ ID NO: 383 - DNA, 63 bp, Artificial Sequence
  EGFP site 66 ribosyme flanked sgRNA (ribosyme- spacer sequence)
acttctctgatgagtccgtgaggacgaaacgagtaagctcgtcagaagtcgtgctgcttcatg

SEQ ID NO: 384 - DNA, 63 bp, Artificial Sequence
  EGFP site 67 ribosyme flanked sgRNA (ribosyme- spacer sequence)
gatggtctgatgagtccgtgaggacgaaacgagtaagctcgtcaccatcttcttcaaggacga

SEQ ID NO: 385 - DNA, 63 bp, Artificial Sequence
  EGFP site 68 ribosyme flanked sgRNA (ribosyme- spacer sequence)
ctccttctgatgagtccgtgaggacgaaacgagtaagctcgtcaaggaggacggcaacatcct

SEQ ID NO: 386 - DNA, 63 bp, Artificial Sequence; identical to SEQ ID NO: 375
  EGFP site 69 ribosyme flanked sgRNA (ribosyme- spacer sequence)

SEQ ID NO: 387 - DNA, 63 bp, Artificial Sequence
  EGFP site 70 ribosyme flanked sgRNA (ribosyme- spacer sequence)
gcacgactgatgagtccgtgaggacgaaacgagtaagctcgtctcgtgctgcttcatgtggtc

SEQ ID NO: 388 - DNA, 63 bp, Artificial Sequence
  EGFP site 71 ribosyme flanked sgRNA (ribosyme- spacer sequence)
cttgaactgatgagtccgtgaggacgaaacgagtaagctcgtcttcaagtccgccatgcccga

SEQ ID NO: 389 - DNA, 63 bp, Artificial Sequence
  EGFP site 72 ribosyme flanked sgRNA (ribosyme- spacer sequence)
tcttcactgatgagtccgtgaggacgaaacgagtaagctcgtctgaagaagatggtgcgctcc

SEQ ID NO: 390 - DNA, 63 bp, Artificial Sequence
  EGFP site 73 ribosyme flanked sgRNA (ribosyme- spacer sequence)
agtacactgatgagtccgtgaggacgaaacgagtaagctcgtctgtactccagcttgtgcccc

SEQ ID NO: 391 - DNA, 63 bp, Artificial Sequence
  EGFP site 74 ribosyme flanked sgRNA (ribosyme- spacer sequence)
acggcactgatgagtccgtgaggacgaaacgagtaagctcgtctgccgtcctcgatgttgtgg

SEQ ID NO: 392 - DNA, 63 bp, Artificial Sequence
  EGFP site 75 ribosyme flanked sgRNA (ribosyme- spacer sequence)
gcgcggctgatgagtccgtgaggacgaaacgagtaagctcgtcccgcgccgaggtgaagttcg

SEQ ID NO: 393 - DNA, 20 bp, Artificial Sequence
  EGFP site 51 tRNA flanked sgRNA (spacer sequence)
cgatgcccttcagctcgatg

SEQ ID NO: 394 - DNA, 20 bp, Artificial Sequence
  EGFP site 52 tRNA flanked sgRNA (spacer sequence)
caacatcctggggcacaagc

SEQ ID NO: 395 - DNA, 20 bp, Artificial Sequence
  EGFP site 53 tRNA flanked sgRNA (spacer sequence)
cagctcgatgcggttcacca

SEQ ID NO: 396 - DNA, 20 bp, Artificial Sequence
  EGFP site 55 tRNA flanked sgRNA (spacer sequence)
tcagctcgatgcggttcacc

SEQ ID NO: 397 - DNA, 20 bp, Artificial Sequence
  EGFP site 59 tRNA flanked sgRNA (spacer sequence)
cgtgctgcttcatgtggtcg

SEQ ID NO: 398 - DNA, 20 bp, Artificial Sequence
  EGFP site 60 tRNA flanked sgRNA (spacer sequence)
ccgtccagctcgaccaggat

SEQ ID NO: 399 - DNA, 20 bp, Artificial Sequence
  EGFP site 61 tRNA flanked sgRNA (spacer sequence)
caactacaagacccgcgccg

SEQ ID NO: 400 - DNA, 20 bp, Artificial Sequence
  EGFP site 62 tRNA flanked sgRNA (spacer sequence)
aagggcgaggagctgttcac

SEQ ID NO: 401 - DNA, 20 bp, Artificial Sequence
  EGFP site 63 tRNA flanked sgRNA (spacer sequence)
aagttcagcgtgtccggcga

SEQ ID NO: 402 - DNA, 20 bp, Artificial Sequence
  EGFP site 64 tRNA flanked sgRNA (spacer sequence)
agcgtgtccggcgagggcga

SEQ ID NO: 403 - DNA, 20 bp, Artificial Sequence
  EGFP site 65 tRNA flanked sgRNA (spacer sequence)
acgagggtgggccagggcac

SEQ ID NO: 404 - DNA, 20 bp, Artificial Sequence
  EGFP site 66 tRNA flanked sgRNA (spacer sequence)
agaagtcgtgctgcttcatg

SEQ ID NO: 405 - DNA, 20 bp, Artificial Sequence
  EGFP site 67 tRNA flanked sgRNA (spacer sequence)
accatcttcttcaaggacga

SEQ ID NO: 406 - DNA, 20 bp, Artificial Sequence
  EGFP site 68 tRNA flanked sgRNA (spacer sequence)
aaggaggacggcaacatcct

SEQ ID NO: 407 - DNA, 20 bp, Artificial Sequence
  EGFP site 69 tRNA flanked sgRNA (spacer sequence)
tcagctcgatgcggttcacc

SEQ ID NO: 408 - DNA, 20 bp, Artificial Sequence
  EGFP site 70 tRNA flanked sgRNA (spacer sequence)
tcgtgctgcttcatgtggtc

SEQ ID NO: 409 - DNA, 20 bp, Artificial Sequence
  EGFP site 71 tRNA flanked sgRNA (spacer sequence)
ttcaagtccgccatgcccga

SEQ ID NO: 410 - DNA, 20 bp, Artificial Sequence
  EGFP site 72 tRNA flanked sgRNA (spacer sequence)
tgaagaagatggtgcgctcc

SEQ ID NO: 411 - DNA, 20 bp, Artificial Sequence
  EGFP site 73 tRNA flanked sgRNA (spacer sequence)
tgtactccagcttgtgcccc

SEQ ID NO: 412 - DNA, 20 bp, Artificial Sequence
  EGFP site 74 tRNA flanked sgRNA (spacer sequence)
tgccgtcctcgatgttgtgg

SEQ ID NO: 413 - DNA, 20 bp, Artificial Sequence
  EGFP site 75 tRNA flanked sgRNA (spacer sequence)
ccgcgccgaggtgaagttcg

SEQ ID NO: 414 - DNA, 20 bp, Artificial Sequence
  FANCF site 1 spacer sequence
gcgatccaggtgctgcagaa

SEQ ID NO: 415 - DNA, 20 bp, Artificial Sequence
  FANCF site 2 spacer sequence
gctgcagaagggattccatg

SEQ ID NO: 416 - DNA, 21 bp, Artificial Sequence
  FANCF site 3 spacer sequence
gtagggccttcgcgcacctca

SEQ ID NO: 417 - DNA, 21 bp, Artificial Sequence
  FANCF site 4 spacer sequence
gccaaggtgaaagcggaagta

SEQ ID NO: 418 - DNA, 21 bp, Artificial Sequence
  FANCF site 5 spacer sequence
gtccaaggtgaaagcggaagt

SEQ ID NO: 419 - DNA, 21 bp, Artificial Sequence
  FANCF site 6 spacer sequence
gcgccgtctccaaggtgaaag

SEQ ID NO: 420 - DNA, 21 bp, Artificial Sequence
  FANCF site 7 spacer sequence
gcctgctctctctgcgcctgc

SEQ ID NO: 421 - DNA, 21 bp, Artificial Sequence
  FANCF site 8 spacer sequence
gccagcaggcgcagagagagc

SEQ ID NO: 422 - DNA, 21 bp, Artificial Sequence
  FANCF site 9 spacer sequence
gcaggacgtcacagtgaccga

SEQ ID NO: 423 - DNA, 21 bp, Artificial Sequence
  FANCF site 10 spacer sequence
gttagcgaacttccaggccct

SEQ ID NO: 424 - DNA, 21 bp, Artificial Sequence
  FANCF site 11 spacer sequence
gcgtcacagtgaccgagggcc

SEQ ID NO: 425 - DNA, 21 bp, Artificial Sequence
  FANCF site 12 spacer sequence
gtccgggattagcgaacttcc

SEQ ID NO: 426 - DNA, 21 bp, Artificial Sequence
  VEGFA site 1 spacer sequence
gcttcatgtacagagagccca

SEQ ID NO: 427 - DNA, 21 bp, Artificial Sequence
  VEGFA site 3 spacer sequence
gaacagctacatatttgggac

SEQ ID NO: 428 - DNA, 20 bp, Artificial Sequence
  VEGFA site 4 spacer sequence
gtcccaaatatgtagctgtt

SEQ ID NO: 429 - DNA, 21 bp, Artificial Sequence
  VEGFA site 5 spacer sequence
gcaaatatgtagctgtttggg

SEQ ID NO: 430 - DNA, 21 bp, Artificial Sequence
  VEGFA site 6 spacer sequence
gtttgggaggtcagaaatagg

SEQ ID NO: 431 - DNA, 20 bp, Artificial Sequence
  VEGFA site 7 spacer sequence
gggtggggggagtttgctcc

SEQ ID NO: 432 - DNA, 21 bp, Artificial Sequence
  VEGFA site 8 spacer sequence
gctttgctaggaatattgaag

SEQ ID NO: 433 - DNA, 20 bp, Artificial Sequence
  VEGFA site 9 spacer sequence
ggcaggggaaggcggagagc

SEQ ID NO: 434 - DNA, 21 bp, Artificial Sequence
  VEGFA site 10 spacer sequence
gtctccccacccgtccctgtc

SEQ ID NO: 435 - DNA, 20 bp, Artificial Sequence
  VEGFA site 11 spacer sequence
gagagccggacagggacggg

SEQ ID NO: 436 - DNA, 20 bp, Artificial Sequence
  VEGFA site 12 spacer sequence
ggacagggacgggtggggag

SEQ ID NO: 437 - DNA, 21 bp, Artificial Sequence
  mouseSprn site 1B spacer sequence
gttctgcccagtaggatgaac

SEQ ID NO: 438 - DNA, 21 bp, Artificial Sequence
  mouseSprn site 2B spacer sequence
gactggactgctgccacgtgc

SEQ ID NO: 439 - DNA, 21 bp, Artificial Sequence
  mouseSprn site 3B spacer sequence
gctggactgctgccacgtgct

SEQ ID NO: 440 - DNA, 21 bp, Artificial Sequence
  mouseSprn site 4B spacers sequence
gcacgtgctgggctctgctgc

SEQ ID NO: 441 - DNA, 21 bp, Artificial Sequence
  mouseSprn site 5B spacer sequence
gcagcagcagagcccagcacg

SEQ ID NO: 442 - DNA, 20 bp, Artificial Sequence
  HEK site 4 spacer sequence
ggcactgcggctggaggtgg

SEQ ID NO: 443 - DNA, 20 bp, Artificial Sequence
  VEGFA site 2 spacer sequence
gaccccctccaccccgcctc

SEQ ID NO: 444 - DNA, 20 bp, Artificial Sequence
  ZSCAN2 spacer sequence
gtgcggcaagagcttcagcc

SEQ ID NO: 445 - DNA, 21 bp, Artificial Sequence
  ZSCAN2-21G spacer sequence
ggtgcggcaagagcttcagcc

SEQ ID NO: 446 - DNA, 20 bp, Artificial Sequence
  EMX1 site 2 spacer sequence
gtcacctccaatgactaggg

SEQ ID NO: 447 - DNA, 21 bp, Artificial Sequence
  EMX1 site 2-21G spacer sequence
ggtcacctccaatgactaggg

SEQ ID NO: 448 - DNA, 20 bp, Artificial Sequence
  DNMT1 site 4 spacer sequence
ggagtgagggaaacggcccc

SEQ ID NO: 449 - DNA, 21 bp, Artificial Sequence
  DNMT1 site 4-21G spacer sequence
gggagtgagggaaacggcccc

SEQ ID NO: 450 - DNA, 23 bp, Artificial Sequence
  FANCF site 1 sequence with PAM
gcgatccaggtgctgcagaaggg

SEQ ID NO: 451 - DNA, 23 bp, Artificial Sequence
  FANCF site 2 sequence with PAM
gctgcagaagggattccatgagg

SEQ ID NO: 452 - DNA, 23 bp, Artificial Sequence
  FANCF site 3 sequence with PAM
tagggccttcgcgcacctcatgg

SEQ ID NO: 453 - DNA, 23 bp, Artificial Sequence
  FANCF site 4 sequence with PAM
ccaaggtgaaagcggaagtaggg

SEQ ID NO: 454 - DNA, 23 bp, Artificial Sequence
  FANCF site 5 sequence with PAM
tccaaggtgaaagcggaagtagg

SEQ ID NO: 455 - DNA, 23 bp, Artificial Sequence
  FANCF site 6 sequence with PAM
cgccgtctccaaggtgaaagcgg

SEQ ID NO: 456 - DNA, 23 bp, Artificial Sequence
  FANCF site 7 sequence with PAM
cctgctctctctgcgcctgctgg

SEQ ID NO: 457 - DNA, 23 bp, Artificial Sequence
  FANCF site 8 sequence with PAM
ccagcaggcgcagagagagcagg

SEQ ID NO: 458 - DNA, 23 bp, Artificial Sequence
  FANCF site 9 sequence with PAM
caggacgtcacagtgaccgaggg

SEQ ID NO: 459 - DNA, 23 bp, Artificial Sequence
  FANCF site 10 sequence with PAM
ttagcgaacttccaggccctcgg

SEQ ID NO: 460 - DNA, 23 bp, Artificial Sequence
  FANCF site 11 sequence with PAM
cgtcacagtgaccgagggcctgg

SEQ ID NO: 461 - DNA, 23 bp, Artificial Sequence
  FANCF site 12 sequence with PAM
tccgggattagcgaacttccagg

SEQ ID NO: 462 - DNA, 23 bp, Artificial Sequence
  VEGFA site 1 sequence with PAM
cttcatgtacagagagcccaggg

SEQ ID NO: 463 - DNA, 23 bp, Artificial Sequence
  VEGFA site 3 sequence with PAM
aacagctacatatttgggactgg

SEQ ID NO: 464 - DNA, 23 bp, Artificial Sequence
  VEGFA site 4 sequence with PAM
gtcccaaatatgtagctgtttgg

SEQ ID NO: 465 - DNA, 23 bp, Artificial Sequence
  VEGFA site 5 sequence with PAM
caaatatgtagctgtttgggagg

SEQ ID NO: 466 - DNA, 23 bp, Artificial Sequence
  VEGFA site 6 sequence with PAM
tttgggaggtcagaaataggggg

SEQ ID NO: 467 - DNA, 23 bp, Artificial Sequence
  VEGFA site 7 sequence with PAM
gggtggggggagtttgctcctgg

SEQ ID NO: 468 - DNA, 23 bp, Artificial Sequence
  VEGFA site 8 sequence with PAM
ctttgctaggaatattgaagggg

SEQ ID NO: 469 - DNA, 23 bp, Artificial Sequence
  VEGFA site 9 sequence with PAM
ggcaggggaaggcggagagccgg

SEQ ID NO: 470 - DNA, 23 bp, Artificial Sequence
  VEGFA site 10 sequence with PAM
tctccccacccgtccctgtccgg

SEQ ID NO: 471 - DNA, 23 bp, Artificial Sequence
  VEGFA site 11 sequence with PAM
gagagccggacagggacgggtgg

SEQ ID NO: 472 - DNA, 23 bp, Artificial Sequence
  VEGFA site 12 sequence with PAM
ggacagggacgggtggggagagg

SEQ ID NO: 473 - DNA, 23 bp, Artificial Sequence
  mouseSprn site 1B sequence with PAM
ttctgcccagtaggatgaactgg

SEQ ID NO: 474 - DNA, 23 bp, Artificial Sequence
  mouseSprn site 2B sequence with PAM
actggactgctgccacgtgctgg

SEQ ID NO: 475 - DNA, 23 bp, Artificial Sequence
  mouseSprn site 3B sequence with PAM
ctggactgctgccacgtgctggg

SEQ ID NO: 476 - DNA, 23 bp, Artificial Sequence
  mouseSprn site 4B sequence with PAM
cacgtgctgggctctgctgctgg

SEQ ID NO: 477 - DNA, 23 bp, Artificial Sequence
  mouseSprn site 5B sequence with PAM
cagcagcagagcccagcacgtgg

SEQ ID NO: 478 - DNA, 23 bp, Artificial Sequence
  HEK site 4 sequence with PAM
ggcactgcggctggaggtggggg

SEQ ID NO: 479 - DNA, 23 bp, Artificial Sequence
  VEGFA site 2 sequence with PAM
gaccccctccaccccgcctccgg

SEQ ID NO: 480 - DNA, 23 bp, Artificial Sequence
  ZSCAN2 sequence with PAM
gtgcggcaagagcttcagccggg

SEQ ID NO: 481 - DNA, 23 bp, Artificial Sequence
  ZSCAN2-21G sequence with PAM
gtgcggcaagagcttcagccggg

SEQ ID NO: 482 - DNA, 23 bp, Artificial Sequence
  EMX1 site 2 sequence with PAM
gtcacctccaatgactagggtgg

SEQ ID NO: 483 - DNA, 23 bp, Artificial Sequence
  EMX1 site 2-21G sequence with PAM
gtcacctccaatgactagggtgg

SEQ ID NO: 484 - DNA, 23 bp, Artificial Sequence
  DNMT1 site 4 sequence with PAM
ggagtgagggaaacggccccagg

SEQ ID NO: 485 - DNA, 23 bp, Artificial Sequence
  DNMT1 site 4-21G sequence with PAM
ggagtgagggaaacggccccagg

SEQ ID NO: 486 - DNA, 20 bp, Artificial Sequence
  EGFP site 1 G19H mixed mismatched spacer sequence
ghgcacgggcagcttgccgg

SEQ ID NO: 487 - DNA, 20 bp, Artificial Sequence
  EGFP site 1 C17D mixed mismatched spacer sequence
gggdacgggcagcttgccgg

SEQ ID NO: 488 - DNA, 20 bp, Artificial Sequence
  EGFP site 1 C15D mixed mismatched spacer sequence
gggcadgggcagcttgccgg

SEQ ID NO: 489 - DNA, 20 bp, Artificial Sequence
  EGFP site 2 A19B mixed mismatched spacer sequence
gbgctggacggcgacgtaaa

SEQ ID NO: 490 - DNA, 20 bp, Artificial Sequence
  EGFP site 2 C17D mixed mismatched spacer sequence
gagdtggacggcgacgtaaa

SEQ ID NO: 491 - DNA, 20 bp, Artificial Sequence
  EGFP site 2 G15H mixed mismatched spacer sequence
gagcthgacggcgacgtaaa

SEQ ID NO: 492 - DNA, 20 bp, Artificial Sequence
  EGFP site 3 T18V mixed mismatched spacer sequence
ggvggtgcagatgaacttca

SEQ ID NO: 493 - DNA, 20 bp, Artificial Sequence
  EGFP site 3 G16H mixed mismatched spacer sequence
ggtghtgcagatgaacttca

SEQ ID NO: 494 - DNA, 20 bp, Artificial Sequence
  EGFP site 3 G14H mixed mismatched spacer sequence
ggtggthcagatgaacttca

SEQ ID NO: 495 - DNA, 20 bp, Artificial Sequence
  EGFP site 4 A18B mixed mismatched spacer sequence
ggbgcgcaccatcttcttca

SEQ ID NO: 496 - DNA, 20 bp, Artificial Sequence
  EGFP site 4 C16D mixed mismatched spacer sequence
ggagdgcaccatcttcttca

SEQ ID NO: 497 - DNA, 20 bp, Artificial Sequence
  EGFP site 4 C14D mixed mismatched spacer sequence
ggagcgdaccatcttcttca

SEQ ID NO: 498 - DNA, 20 bp, Artificial Sequence
  EGFP site 5 T19V mixed mismatched spacer sequence
gvcgccctcgaacttcacct

SEQ ID NO: 499 - DNA, 20 bp, Artificial Sequence
  EGFP site 5 G17H mixed mismatched spacer sequence
gtchccctcgaacttcacct

SEQ ID NO: 500 - DNA, 20 bp, Artificial Sequence
  EGFP site 5 C15D mixed mismatched spacer sequence
gtcgcdctcgaacttcacct

SEQ ID NO: 501 - skipped

SEQ ID NO: 502 - DNA, 20 bp, Artificial Sequence
  EGFP site 6 C17D mixed mismatched spacer sequence
gcgdgatcacatggtcctgc

SEQ ID NO: 503 - DNA, 20 bp, Artificial Sequence
  EGFP site 6 A15B mixed mismatched spacer sequence
gcgcgbtcacatggtcctgc

SEQ ID NO: 504 - DNA, 20 bp, Artificial Sequence
  EGFP site 7 T19V mixed mismatched spacer sequence
gvccatgccgagagtgatcc

SEQ ID NO: 505 - DNA, 20 bp, Artificial Sequence
  EGFP site 7 C17D mixed mismatched spacer sequence
gtcdatgccgagagtgatcc

SEQ ID NO: 506 - DNA, 20 bp, Artificial Sequence
  EGFP site 7 T15V mixed mismatched spacer sequence
gtccavgccgagagtgatcc

SEQ ID NO: 507 - DNA, 20 bp, Artificial Sequence
  EGFP site 8 C18D mixed mismatched spacer sequence
gcdgtcgtccttgaagaaga

SEQ ID NO: 508 - DNA, 20 bp, Artificial Sequence
  EGFP site 8 T16V mixed mismatched spacer sequence
gccgvcgtccttgaagaaga

SEQ ID NO: 509 - DNA, 20 bp, Artificial Sequence
  EGFP site 8 G14H mixed mismatched spacer sequence
gccgtchtccttgaagaaga

SEQ ID NO: 510 - DNA, 20 bp, Artificial Sequence
  EGFP site 9 A18B mixed mismatched spacer sequence
gabgttcgagggcgacaccc

SEQ ID NO: 511 - DNA, 20 bp, Artificial Sequence
  EGFP site 9 T16V mixed mismatched spacer sequence
gaagvtcgagggcgacaccc

SEQ ID NO: 512 - DNA, 20 bp, Artificial Sequence
  EGFP site 9 C14D mixed mismatched spacer sequence
gaagttdgagggcgacaccc

SEQ ID NO: 513 - DNA, 20 bp, Artificial Sequence
  EGFP site 10 T19V mixed mismatched spacer sequence
gvctttgctcagggcggact

SEQ ID NO: 514 - DNA, 20 bp, Artificial Sequence
  EGFP site 10 T17V mixed mismatched spacer sequence
gtcvttgctcagggcggact

SEQ ID NO: 515 - DNA, 20 bp, Artificial Sequence
  EGFP site 10 T15V mixed mismatched spacer sequence
gtcttvgctcagggcggact

SEQ ID NO: 516 - DNA, 20 bp, Artificial Sequence
  EGFP site 11 T18V mixed mismatched spacer sequence
ggvctttgctcagggcggac

SEQ ID NO: 517 - DNA, 20 bp, Artificial Sequence
  EGFP site 11 T16V mixed mismatched spacer sequence
ggtcvttgctcagggcggac

SEQ ID NO: 518 - DNA, 20 bp, Artificial Sequence
  EGFP site 11 T14V mixed mismatched spacer sequence
ggtcttvgctcagggcggac

SEQ ID NO: 519 - DNA, 20 bp, Artificial Sequence
  EGFP site 12 G18H mixed mismatched spacer sequence
gthctcaggtagtggttgtc

SEQ ID NO: 520 - DNA, 20 bp, Artificial Sequence
  EGFP site 12 T16V mixed mismatched spacer sequence
gtgcvcaggtagtggttgtc

SEQ ID NO: 521 - DNA, 20 bp, Artificial Sequence
  EGFP site 12 A14B mixed mismatched spacer sequence
gtgctcbggtagtggttgtc

SEQ ID NO: 522 - DNA, 20 bp, Artificial Sequence
  EGFP site 15 C18D mixed mismatched spacer sequence
ggdatcgccctcgccctcgc

SEQ ID NO: 523 - DNA, 20 bp, Artificial Sequence
  EGFP site 15 T16V mixed mismatched spacer sequence
ggcavcgccctcgccctcgc

SEQ ID NO: 524 - DNA, 20 bp, Artificial Sequence
  EGFP site 15 G14H mixed mismatched spacer sequence
ggcatchccctcgccctcgc

SEQ ID NO: 525 - DNA, 20 bp, Artificial Sequence
  EGFP site 16 C18D mixed mismatched spacer sequence
ggdgagggcgatgccaccta

SEQ ID NO: 526 - DNA, 20 bp, Artificial Sequence
  EGFP site 16 A16B mixed mismatched spacer sequence
ggcgbgggcgatgccaccta

SEQ ID NO: 527 - DNA, 20 bp, Artificial Sequence
  EGFP site 16 G14H mixed mismatched spacer sequence
ggcgaghgcgatgccaccta

SEQ ID NO: 528 - DNA, 20 bp, Artificial Sequence
  EGFP site 24 T19V mixed mismatched spacer sequence
gvcgtgctgcttcatgtggt

SEQ ID NO: 529 - DNA, 20 bp, Artificial Sequence
  EGFP site 24 G17H mixed mismatched spacer sequence
gtchtgctgcttcatgtggt

SEQ ID NO: 530 - DNA, 20 bp, Artificial Sequence
  EGFP site 24 G15H mixed mismatched spacer sequence
gtcgthctgcttcatgtggt

SEQ ID NO: 531 - DNA, 20 bp, Artificial Sequence
  EGFP site 43 A18B mixed mismatched spacer sequence
gabgggcatcgacttcaagg

SEQ ID NO: 532 - DNA, 20 bp, Artificial Sequence
  EGFP site 43 G16H mixed mismatched spacer sequence
gagghgcatcgacttcaagg

SEQ ID NO: 533 - DNA, 20 bp, Artificial Sequence
  EGFP site 43 C14D mixed mismatched spacer sequence
gaggggdatcgacttcaagg

SEQ ID NO: 534 - DNA, 8077 bp, Artificial Sequence
  pX330-Flag-WT SpCas9 (without sgRNA; with silent mutations)
  misc_feature 826..891: /note=“3xFLAG tag ”
  misc_feature 898..928: /note=“NLS”
  misc_feature 943..5043: /note=“Human codon optimized S. pyogenes Cas9 ”
ctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcgtacctgaacgccgtcgtgggaaccgccctgatcaaaaagtaccctaagctggaaagcgagttcgtgtacggcgactacaaggtgtacgacgtacggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 535 - DNA, 64 bp, Artificial Sequence
  9214-SpCas9_bact_exp-for forward primer
aaaaaaactagtgaaaacctgtatttccagggagcagcctcgatggactataaggaccacgacg

SEQ ID NO: 536 - DNA, 33 bp, Artificial Sequence
  9214-SpCas9_bact_exp-rev reverse primer
aaaaaagcggccgcaacagatggctggcaacta

SEQ ID NO: 537 - DNA, 94 bp, Artificial Sequence
  8717for forward primer
cgcgtacctgaacgccgtcgtgggaaccgccctgatcaaaaagtaccctaagctggaaagcgggttcgtgtacggcgacggcaaggtgtacgat

SEQ ID NO: 538 - DNA, 94 bp, Artificial Sequence
  8717rev reverse primer
gtacatcgtacaccttgccgtcgccgtacacgaacccgctttccagcttagggtactttttgatcagggcggttcccacgacggcgttcaggta

SEQ ID NO: 539 - DNA, 67 bp, Artificial Sequence
  8727for forward primer
cgcgtacctgaacgccgtcgtgggaaccgccctgatcaaaaagtaccctaagggtaaggtgtacgat

SEQ ID NO: 540 - DNA, 67 bp, Artificial Sequence
  8727rev reverse primer
gtacatcgtacaccttacccttagggtactttttgatcagggcggttcccacgacggcgttcaggta

SEQ ID NO: 541 - DNA, 55 bp, Artificial Sequence
  8724for forward primer
cgcgtacctgaacgccgtcgtgggaaccgccctgatcaaaaagtaccctaaggat

SEQ ID NO: 542 - DNA, 55 bp, Artificial Sequence
  8724rev reverse primer
gtacatccttagggtactttttgatcagggcggttcccacgacggcgttcaggta

SEQ ID NO: 543 - DNA, 58 bp, Artificial Sequence
  8725for forward primer
cgcgtacctgaacgccgtcgtgggaaccgccctgatcaaaaagtaccctaagggtgat

SEQ ID NO: 544 - DNA, 58 bp, Artificial Sequence
  8725rev reverse primer
gtacatcacccttagggtactttttgatcagggcggttcccacgacggcgttcaggta

SEQ ID NO: 545 - DNA, 61 bp, Artificial Sequence
  8726for forward primer
cgcgtacctgaacgccgtcgtgggaaccgccctgatcaaaaagtaccctaagggtaaagat

SEQ ID NO: 546 - DNA, 61 bp, Artificial Sequence
  8726rev reverse primer
gtacatctttacccttagggtactttttgatcagggcggttcccacgacggcgttcaggta

SEQ ID NO: 547 - DNA, 67 bp, Artificial Sequence
  8718for forward primer
cgcgtacctgaacgccgtcgtgggaaccgccctgatcaaaaagtaccctaagctgaaggtgtacgat

SEQ ID NO: 548 - DNA, 67 bp, Artificial Sequence
  8718rev reverse primer
gtacatcgtacaccttcagcttagggtactttttgatcagggcggttcccacgacggcgttcaggta

SEQ ID NO: 549 - DNA, 70 bp, Artificial Sequence
  8721for forward primer
cgcgtacctgaacgccgtcgtgggaaccgccctgatcaaaaagtaccctaagctgggtaaggtgtacgat

SEQ ID NO: 550 - DNA, 70 bp, Artificial Sequence
  8721rev reverse primer
gtacatcgtacaccttacccagcttagggtactttttgatcagggcggttcccacgacggcgttcaggta

SEQ ID NO: 551 - DNA, 70 bp, Artificial Sequence
  8730for forward primer
cgcgtacctgaacgccgtcgtgggaaccgccctgatcaaaaagtaccctaagctgcctaaggtgtacgat

SEQ ID NO: 552 - DNA, 70 bp, Artificial Sequence
  8730rev reverse primer
gtacatcgtacaccttaggcagcttagggtactttttgatcagggcggttcccacgacggcgttcaggta

SEQ ID NO: 553 - DNA, 73 bp, Artificial Sequence
  8719for forward primer
cgcgtacctgaacgccgtcgtgggaaccgccctgatcaaaaagtaccctaagctgggtggcaaggtgtacgat

SEQ ID NO: 554 - DNA, 73 bp, Artificial Sequence
  8719rev reverse primer
gtacatcgtacaccttgccacccagcttagggtactttttgatcagggcggttcccacgacggcgttcaggta

SEQ ID NO: 555 - DNA, 73 bp, Artificial Sequence
  8729for forward primer
cgcgtacctgaacgccgtcgtgggaaccgccctgatcaaaaagtaccctaagctgggtcctaaggtgtacgat

SEQ ID NO: 556 - DNA, 73 bp, Artificial Sequence
  8729rev reverse primer
gtacatcgtacaccttaggacccagcttagggtactttttgatcagggcggttcccacgacggcgttcaggta

SEQ ID NO: 557 - DNA, 76 bp, Artificial Sequence
  8741for forward primer
cgcgtacctgaacgccgtcgtgggaaccgccctgatcaaaaagtaccctaagctgggtggcggaaaggtgtacgat

SEQ ID NO: 558 - DNA, 76 bp, Artificial Sequence
  8741rev reverse primer
gtacatcgtacacctttccgccacccagcttagggtactttttgatcagggcggttcccacgacggcgttcaggta

SEQ ID NO: 559 - DNA, 79 bp, Artificial Sequence
  8742for forward primer
cgcgtacctgaacgccgtcgtgggaaccgccctgatcaaaaagtaccctaagctgggtggcggaggtaaggtgtacgat

SEQ ID NO: 560 - DNA, 79 bp, Artificial Sequence
  8742rev reverse primer
gtacatcgtacaccttacctccgccacccagcttagggtactttttgatcagggcggttcccacgacggcgttcaggta

SEQ ID NO: 561 - DNA, 70 bp, Artificial Sequence
  8738for forward primer
cgcgtacctgaacgccgtcgtgggaaccgccctgatcaaaaagtaccctaagctgggtggcgtgtacgat

SEQ ID NO: 562 - DNA, 70 bp, Artificial Sequence
  8738rev reverse primer
gtacatcgtacacgccacccagcttagggtactttttgatcagggcggttcccacgacggcgttcaggta

SEQ ID NO: 563 - DNA, 67 bp, Artificial Sequence
  8739for forward primer
cgcgtacctgaacgccgtcgtgggaaccgccctgatcaaaaagtaccctaagctgggtggctacgat

SEQ ID NO: 564 - DNA, 67 bp, Artificial Sequence
  8739rev reverse primer
gtacatcgtagccacccagcttagggtactttttgatcagggcggttcccacgacggcgttcaggta

SEQ ID NO: 565 - DNA, 64 bp, Artificial Sequence
  8740for forward primer
cgcgtacctgaacgccgtcgtgggaaccgccctgatcaaaaagtaccctaagctgggtggcgat

SEQ ID NO: 566 - DNA, 64 bp, Artificial Sequence
  8740rev reverse primer
gtacatcgccacccagcttagggtactttttgatcagggcggttcccacgacggcgttcaggta

SEQ ID NO: 567 - DNA, 73 bp, Artificial Sequence
  8756for forward primer
cgcgtacctgaacgccgtcgtgggaaccgccctgatcaaaaagtaccctaagctgtctagcaaggtgtacgat

SEQ ID NO: 568 - DNA, 73 bp, Artificial Sequence
  8756rev reverse primer
gtacatcgtacaccttgctagacagcttagggtactttttgatcagggcggttcccacgacggcgttcaggta

SEQ ID NO: 569 - DNA, 64 bp, Artificial Sequence
  8728for forward primer
cgcgtacctgaacgccgtcgtgggaaccgccctgatcaaaaagtaccctaagctgtctagcgat

SEQ ID NO: 570 - DNA, 64 bp, Artificial Sequence
  8728rev reverse primer
gtacatcgctagacagcttagggtactttttgatcagggcggttcccacgacggcgttcaggta

SEQ ID NO: 571 - DNA, 73 bp, Artificial Sequence
  8722for forward primer
cgcgtacctgaacgccgtcgtgggaaccgccctgatcaaaaagtaccctaagctggaaagcaaggtgtacgat

SEQ ID NO: 572 - DNA, 73 bp, Artificial Sequence
  8722rev reverse primmer
gtacatcgtacaccttgctttccagcttagggtactttttgatcagggcggttcccacgacggcgttcaggta

SEQ ID NO: 573 - DNA, 76 bp, Artificial Sequence
  8723for forward primer
cgcgtacctgaacgccgtcgtgggaaccgccctgatcaaaaagtaccctaagctggaaagcggtaaggtgtacgat

SEQ ID NO: 574 - DNA, 76 bp, Artificial Sequence
  8723rev reverse primer
gtacatcgtacaccttaccgctttccagcttagggtactttttgatcagggcggttcccacgacggcgttcaggta

SEQ ID NO: 575 - DNA, 73 bp, Artificial Sequence
  8752for forward primer
cgcgtacctgaacgccgtcgtgggaaccgccctgatcaaaaagtaccctgcgctgggtggcaaggtgtacgat

SEQ ID NO: 576 - DNA, 73 bp, Artificial Sequence
  8752rev reverse primer
gtacatcgtacaccttgccacccagcgcagggtactttttgatcagggcggttcccacgacggcgttcaggta

SEQ ID NO: 577 - DNA, 24 bp, Artificial Sequence
  HF+primer_rev reverse primer
gaagccgccgtacttcttaggatc

SEQ ID NO: 578 - DNA, 27 bp, Artificial Sequence
  HF+primer_for forward primer
gaggaaaacgaggacattctggaagat

SEQ ID NO: 579 - DNA, 33 bp, Artificial Sequence
  A661Rfor forward primer
cggctggggcaggctgagccggaagctgatcaa

SEQ ID NO: 580 - DNA, 28 bp, Artificial Sequence
  A661Rrev reverse primer
cggctcagcctgccccagccggtgtatc

SEQ ID NO: 581 - DNA, 35 bp, Artificial Sequence
  A695Qfor forward primer
aaacttcatgcagctgatccacgacgacagcctga

SEQ ID NO: 582 - DNA, 39 bp, Artificial Sequence
  A695Qrev reverse primer
cgtggatcagctgcatgaagtttctgttggcgaagccgt

SEQ ID NO: 583 - DNA, 32 bp, Artificial Sequence
  A926Qrev reverse primer
gctttgtgatctgccgggtttccaccagctgt

SEQ ID NO: 584 - DNA, 33 bp, Artificial Sequence
  A926Qfor forward primer
ggaaacccggcagatcacaaagcacgtggcaca

(19) United States
(12) Patent Application Publication
(10) Pub. No.: US 2023/0031899 A1
(43) Pub. Date: Feb. 2, 2023

(54) VARIANT CAS9

(71) Applicant: BIOSPIRÁL-2006 FEJLESZTÕ ÉS TANÁCSADÓ KFT., Szeged (HU)

(72) Inventors: Ervin WELKER, Budaörs (HU); Péter KULCSÁR, Pécs (HU); András TÁLAS, Pomáz (HU); Eszter TÓTH, Budapest (HU); Zoltán LIGETI, Budapest (HU); Antal NYESTE, Budapest (HU); Zsombor WELKER, Budaörs (HU)

(21) Appl. No.: 17/781,452

(22) PCT Filed: Feb. 25, 2021
(86) PCT No.: PCT/HU2021/050015

§ 371 Date: Jun. 1, 2022

(30) Foreign Application Priority Data
Feb. 25, 2020 (HU) P2000068
Feb. 26, 2020 (HU) P2000072
Mar. 6, 2020 (HU) P2000083
Dec. 23, 2020 (HU) PCT/HU2020/050063

(51) Int. Cl. C12N 9/22; C12N 15/90

(52) U.S. Cl. CPC C12N 9/22; C12N 15/907; C12N 2310/20; C12N 2800/80

(57) ABSTRACT

Some aspects of the present disclosure provide compositions, methods, and kits for improving the specificity and/or targetable sequence space of endonucleases programmable by RNA such as Cas9. Also provided herein are variants of Cas9 that have been engineered to have improved specificity for cleaving nucleic acid targets. Also provided herein are variants of Cas9 with increased fidelity that have been engineered to became compatible with 5′ extended sgRNAs such as 21G-sgRNAs. Such Cas9 variants are useful in clinical and research settings involving site-specific modification of DNA (e.g., genomic modification), epigenomic engineering, transcriptome regulation, genome targeting.

### TECHNICAL FIELD

The invention relates, at least in part, to engineered Clustered Regularly Interspaced Short Palindromic Repeats (CRISPRs)/CRISPR-associated protein 9 (Cas9) nucleases with altered and improved target specificity and/or altered or extended target space and their use in genomic engineering, epigenomic engineering, genome targeting, transcriptome regulation, genome editing, and in vitro diagnostics and in medical applications.

### BACKGROUND OF THE INVENTION

The introduction of the CRISPR (clustered regularly interspaced short palindromic repeats)/Cas9 (CRISPR associated protein 9) system as a genome engineering tool clearly revolutionized the field of biotechnology. CRISPR/Cas proteins are RNA-guided endonucleases (RGNs) that can be directed to cleave a chosen DNA or RNA sequence and they provide an adaptive immunity against mobile viruses, plasmids and transposons in archaea and bacteria. The CRISPR/Cas systems can be classified into two major classes according to our current knowledge. Class 1 encompasses the type I, type III and type IV groups which have multiple subunit effector complexes. Class 2 contains much simpler systems with single multifunctional and multidomain protein effector modules. Class 2 consist of type II (including the Cas9 proteins), type V and type VI groups. Nucleases of type II systems contain Cas proteins with similar domain architecture, including a RuvC-like and a HNH nuclease domains each cleaving one DNA strand. Type V (such as Cas12a proteins [former name: Cpf1]) systems contain effectors with only one active RuvC-like nuclease domain for cleaving both DNA strands, while in case of type VI subtypes two HEPN RNase domains are presented (Koonin et al., 2017; Makarova et al., 2015; Makarova et al., 2018).

Since the first published results, many different CRISPR nucleases have proved their values for genome engineering applications, but among them presently SpCas9 is the most commonly used genome engineering tool.

The ribonucleoprotein (RNP) complex of the Cas9 nucleases involve the Cas9 protein itself and two Cas9-associated RNAs [CRISPR RNA (crRNA) and the trans-activating crRNA (tracrRNA)] possessing sequences complementary to each other. Complementarity between the targeted DNA site and the spacer sequence of the crRNA and the presence of a short protospacer-adjacent motif (PAM) at the 3′-end of the target site are also required to the binding and cleavage to occur (Anders et al., 2016; Anders et al., 2014; Cong et al., 2013; Garneau et al., 2010; Jiang et al., 2015; Jinek et al., 2012; Jinek et al., 2014; Mali et al., 2013c; Nishimasu et al., 2014). The length of the required spacer sequence and the PAM motif varies depending on the species the Cas9 originates from. In case of SpCas9 a 20-nucleotide long spacer sequence and an NGG PAM motif downstream of the target sequence on the non-targeted DNA strand are needed (Mojica et al., 2009) (FIG. 1A). It has been shown that SpCas9 nucleases can be guided to the desired target site by a fused crRNA and tracrRNA, named single guide RNA (sgRNA, sometimes referred to as gRNA) (Jinek et al., 2012) (FIG. 1B).

The Streptococcus pyogenes Cas9 (SpCas9) nuclease, along with other RNA-guided nucleases of the type II CRISPR system, has proved its value for genome engineering applications (Cho et al., 2013; Cong et al., 2013; Hsu et al., 2014; Jinek et al., 2012; Jinek et al., 2013; Komor et al., 2017; Koonin et al., 2017; Mali et al., 2013b; Mali et al., 2013c; Sander and Joung, 2014; Tanenbaum et al., 2014; Vora et al., 2016; Wu et al., 2018; Zuris et al., 2015). Intensive research has been focused on increasing its potential by minimizing off-target activity which restricts its use in areas where high specificity is essential (Cho et al., 2014; Fu et al., 2014a; Fu et al., 2014b; Guilinger et al., 2014; Mali et al., 2013a; Ran et al., 2013; Tsai et al., 2014; Tsai et al., 2015; Wyvekens et al., 2015). The most promising approaches to decrease its off-target activity are the generation of increased fidelity mutant variants, such as eSpCas9, SpCas9-HF1 and HypaSpCas9 developed by rational design (Chen et al., 2017; Kleinstiver et al., 2016; Slaymaker et al., 2016), evoSpCas9 developed by exploiting a selection scheme (Casini et al., 2018) or the HeFSpCas9 variants developed by combining the mutations found in eSpCas9 and SpCas9-HF1 (Chen et al., 2017; Kulcsar et al., 2017). Limitations of this approach include increased target selectivity, meaning that these nucleases do not or limitedly cut at several target sites that are otherwise cleaved by the wild type (WT) SpCas9. Another limitation of using increased fidelity mutant variants is their reduced compatibility with 5′-altered sgRNAs. Indeed, most of the increased fidelity nucleases can routinely be used only with fully matching 20-nucleotide-long spacers (20G-sgRNAs) (Casini et al., 2018; Kim et al., 2017; Kleinstiver et al., 2016; Kulcsar et al., 2017; Slaymaker et al., 2016; Zhang et al., 2017). It is plausible that they do not work well with 5′ mismatching or truncated sgRNAs because, by design, they are inherently characterized by a lower spacer-target mismatch tolerance (i.e. they are sensitive to structural alterations within the DNA-RNA hybrid helix which is bundled up inside the protein structure). However, it is less obvious why they possess diminished activity with 5′-extended sgRNAs, given that the extension is supposed to protrude from the structure of the nuclease (Anders et al., 2014; Jinek et al., 2014). Some of the extensions were also shown to increase the fidelity of the nuclease action for which an explanation is still missing (Cho et al., 2014; Kocak et al., 2019). An understanding of this effect may lead to a better comprehension of the main factors that determine specificity and effectivity of the action of increased fidelity SpCas9 nucleases. This issue also has technical aspects: to comply with the sequence requirement of the promoters commonly used to transcribe the sgRNA [such as the human U6 promoter in mammalian cells (Goomer and Kunkel, 1992) or the T7 promoter in vitro (Beckert and Masquida, 2011; Milligan et al., 1987; Moreno-Mateos et al., 2015)], 5′ G-extended sgRNAs are frequently used with the WT SpCas9 when appropriate 20G-N19-NGG targets cannot be identified bioinformatically. Indeed, there are 27 knock-out pooled sgRNA libraries at Addgene (as of Jun. 24, 2019; https://www.addgene.org/pooled-library/) and none of them is restricted to 20G-N19-NGG target sequences. Such shortage of appropriate targets is also a general problem with applications where there is little room to maneuver, for example when a specific position needs to be targeted by exploiting single strand oligos, when using either dCas9-FokI nucleases or base editors or when tagging proteins. Although some methods have been adapted, there is no general approach to extend the target space available for increased fidelity SpCas9 variants beyond the 20G-N19-NGG target sequences (Gao and Zhao, 2014; Nissim et al., 2014; Nowak et al., 2016; Xie et al., 2015). The use of chemically synthesized sgRNAs in pre-assembled ribonucleoprotein (RNP) form circumvents this problem in certain cases; however, RNPs are not suitable for use in pooled library screens and are prohibitively expensive for large-scale or high-throughput studies. Furthermore, it is specifically reported that eSpCas9, SpCas9-HF1, Hypa- and evoSpCas9 have strongly reduced activities when they are applied by the RNP delivery method (Vakulskas et al., 2018). Other approaches exploiting ribozyme- or tRNA fusions have not been well characterized for the sequence dependence of sgRNA-processing. These systems have not been applied to any large-scale studies, and none of the pooled sgRNA libraries included in the 45 activation, repression or knock-out libraries currently available at Addgene (https://www.addgene.org/pooled-library/) is built on ribozyme- or tRNA-sgRNA fusion vectors.

Here we report a solution to the problem of reduced activity of increased fidelity mutants with extended 21G-sgRNA (“activity vs. fidelity problem” in short), applicable to WT SpCas9 and all of its variants including all increased fidelity SpCas9 nucleases, that results in high specificity editing of a considerably wider target range, even when applied as pre-assembled RNP complex, the form which can further increase editing specificity. The solution is applicable to Cas9 proteins, having in their wild type form a surface loop comprising amino acids being in contact with the 5′ end of a crRNA having a target specific spacer sequence of a Cas9 associated RNA.

The inventors have unexpectedly found that, in Cas9 proteins, by modifying a surface loop comprising amino acids being in contact with the 5′ end of the target specific spacer sequence of a crRNA (preferably sgRNA), the available target space for Cas9 variant with an 5′ extension of the spacer sequence in said crRNA or sgRNA can be increased. In other words, the room in said protein structure at the 5′ end of the cr- or sgRNA to accommodate the said 5′ G-extension can be increased or broadened or widened. The modification of said loop should normally reduce or disrupt the association between (i) the spacer sequence (preferably the 5′ end thereof) and (ii) the amino acids sterically proximal to or being in contact with (at least in the wild type sequence) the 5′ end of the spacer sequence. Thereby a longer spacer sequence e.g. with an 5′ G extension can be accommodated and used in said variant Cas9-protein. Thus, the mutations in the mutant (variant) Cas9 proteins allow using longer spacer sequences, e.g. sgRNAs with longer, e.g. 21 nucleotide long spacer sequences (21G-sgRNAs).

Moreover, it has been also surprisingly found that the fidelity of a wild type Cas9 protein can be increased, preferably without significant impairment of its activity. From another point of view, an increased fidelity mutant of the invention may have an increased activity with 21 nucleotide long spacer sequence, or may have a higher fidelity than the wild type Cas9 or in comparison with a reference Cas9 not having the same mutation according to the invention.

### BRIEF DESCRIPTION OF THE INVENTION

The invention relates to an isolated variant Cas9 protein, having, as compared to a (corresponding) wild type Cas9 sequence (e.g. a reference Cas9 sequence) a segment, preferably a surface loop comprising amino acids being in contact with the 5′ end of a target specific spacer sequence of a Cas9 associated RNA (crRNA), preferably of a single guide RNA (sgRNA), said variant Cas9 protein comprising a mutation in the loop which mutation

i) reduces or disrupts the association between said amino acids and the spacer sequence, preferably the 5′ end thereof, and/or
ii) increases the fidelity of said Cas9 protein, and/or
iii) broadens/widens the space in said protein structure at the 5′ end of the crRNA or sgRNA to accommodate the said 5′ G-extension, and/or
iv) increases the available target space for a spacer sequence in the cr- or sgRNAs with a 5′ extension of the spacer sequence, and/or
without disruption of the structural features of the folded polypeptide chain, whereas activity of the Cas9 is maintained on a target DNA complementary to said spacer sequence.

Preferably ii) an increase in the fidelity is due to i) reduction of the association between said amino acids and the spacer sequence.

Preferably iv) an increase in the available target space is due to i) reduction of the association between said amino acids and the spacer sequence.

In a preferred embodiment the invention relates to an isolated variant Cas9 protein which is a variant of a Streptococcus pyogenes Cas9 (SpCas9) protein according to claim 1, having a mutation in the segment, or preferably in the surface loop (preferably between the amino acids Leu1004 and Asp1017, preferably between Leu1004 and Lys1014), said loop comprising the following positions: Glu1007 and Tyr1013, wherein said mutation comprises mutation(s) of one or more amino acids,

wherein said mutation preferably disrupts the capping of the 5′ end of the single guide RNA or crRNA, preferably sgRNA, (said capping being) formed by said Glu1007 and Tyr1013.
In other Cas9 proteins having a corresponding loop with amino acids corresponding to said amino acids based on sequence alignment or 3D structural comparison the mutations are to be carried out in the corresponding positions.

In a preferred embodiment the invention the isolated variant Cas9 protein is a variant Cas9 protein which comprises a mutant (variant) segment (or sequence), wherein said mutant (variant) segment is present in the position of the wild type segment from Leu1004 to Asp1017 of SpCas9 or a corresponding segment of a wild type Cas9 protein comprising said surface loop as defined herein, and said mutant (variant) segment comprising mutations which are, independently from each other, selected from the group consisting of the following deletions and substitutions:

one or more deletion(s) in a segment from Leu1004 to Asp1017 of SpCas9 or a corresponding segment of a wild type Cas9, wherein the length of the deleted segment is e.g. 4 to 12 amino acids or 6 to 12 amino acids, preferably 7 to 11 amino acids or 8 to 10 amino acids or highly preferably 9 amino acids;

one or more substitution(s) to Pro (P), Val (V), Ile (I), Leu (L), Ser (S), Thr (T), Cys (C), Met (M), Lys (K), Gly (G), Ala (A), in particular substitutions to Leu (L), Thr (T), Cys (C), Lys (K), Ser (S), Gly (G), Ala (A), preferably substitutions to Gly (G) or Ala (A), in particular Gly (G), in an insertion comprising this substitution i.e. these amino acid(s), wherein the length of the insertion is 1, 2, 3, 4, 5 or 6 amino acids, preferably 1, 2, 3 or 4 amino acids, more preferably 2 or 3 amino acids, highly preferably 2 amino acids.

In a more preferred embodiment the invention the isolated variant Cas9 protein is a variant Cas9 protein which comprises a mutant (variant) segment, wherein said mutant (variant) segment is present in the position of the wild type segment from Leu1004 to Asp1017 of SpCas9 or a corresponding segment of a wild type Cas9 protein comprising said surface loop as defined herein (or wherein said mutant (variant) segment replaces the wild type segment KLESEFVYGDYKVYD (SEQ ID NO. 4) of SpCas9 or a corresponding segment of a wild type Cas9 protein), and said mutant (variant) segment comprising mutations which are, independently from each other, selected from the group consisting of the following deletions and insertions:

one or more deletion(s) in said segment, wherein the length of the deleted segment(s) altogether is 4 to 12 amino acids or 6 to 12 amino acids, preferably 7 to 11 amino acids or 8 to 10 amino acids or highly preferably 9 amino acids;

an insertion having the length of 1, 2, 3, 4, 5 or 6 amino acids, preferably 1, 2, 3 or 4 amino acids, more preferably 2 or 3 amino acids, highly preferably 2 amino acids, said amino acid(s) being selected from the group consisting of Pro (P), Val (V), Ile (I), Leu (L), Ser (S), Thr (T), Cys (C), Met (M), Lys (K), Gly (G), Ala (A), in particular substitutions to Leu (L), Thr (T), Cys (C), Lys (K), Ser (S), Gly (G), Ala (A), preferably substitutions to Gly (G) or Ala (A), in particular Gly (G).

In a particularly preferred embodiment said mutant (variant) segment comprises mutations which are, independently from each other, selected from the group consisting of the following deletions and insertions:

one or more deletion(s) in said segment, wherein the length of the deleted segment(s) altogether is 7 to 11 amino acids or 8 to 10 amino acids or highly preferably 9 amino acids;

an insertion having the length of 1, 2, 3 or 4 amino acids, said amino acid(s) being selected from the group consisting of Leu (L), Thr (T), Cys (C), Lys (K), Ser (S), Gly (G), Ala (A), preferably substitutions to Gly (G) or Ala (A), in particular Gly (G).

In a highly preferred embodiment the invention the isolated variant Cas9 protein is a variant Cas9 protein which comprises a sequence, wherein said sequence replaces the wild type segment KLESEFVYGDYKVYD (SEQ ID NO. 4) of SpCas9 or a corresponding sequence of a wild type Cas9 protein, and said segment having the sequence selected from the following group consisting of SEQ ID NOs 6 to 23 as listed below:

| 1003-------------1017 | |
|---|---|
| KLESEFVYGDYKVYD | SEQ ID NO: 4 |
| KGKVYD | SEQ ID NO: 6 |
| KD | SEQ ID NO: 7 |
| KGD | SEQ ID NO: 8 |
| KGKD | SEQ ID NO: 9 |
| KLKVYD | SEQ ID NO: 10 |
| KLGKVYD | SEQ ID NO: 11 |
| KLPKVYD | SEQ ID NO: 12 |
| KLGGKVYD | SEQ ID NO: 13 |
| KLGPKVYD | SEQ ID NO: 14 |
| KLGGGKVYD | SEQ ID NO: 15 |
| KLGGGGKVYD | SEQ ID NO: 16 |
| KLGGVYD | SEQ ID NO: 17 |
| KLGGYD | SEQ ID NO: 18 |
| KLGGD | SEQ ID NO: 19 |
| KLSSKVYD | SEQ ID NO: 20 |
| KLSSD | SEQ ID NO: 21 |
| KLESKVYD | SEQ ID NO: 22 |
| KLESGKVYD | SEQ ID NO: 23 |

The length of the insertion(s) and/or the substitution(s) is not calculated into the length of the deletion(s), i.e. the length of the deletion(s) is the difference between the number of amino acids of the wild type segment and the mutant (variant) segment.

In a preferred isolated variant of the SpCas9 protein the segment is a surface loop which comprises one or more mutations of the following amino acids of the wild type sequence: Glu1007 and Tyr1013, wherein said one or more mutation(s)

disrupt the association between said amino acids and the spacer sequence, preferably the 5′ end of the spacer sequence, and/or

broaden/widen the space in said protein structure at the 5′ end of the cr- or sgRNA to accommodate the said 5′ G-extension, and/or

increase the available target space for an 5′ extension of the spacer sequence, and/or

increases the fidelity of said Cas9 protein,

wherein preferably said mutations are, independently from each other, selected from the group consisting of deletions as well as substitutions, optionally substitutions to Pro (P), Val (V), Ile (I), Leu (L), Ser (S), Thr (T), Cys (C), Met (M), Lys (K), Gly (G), Ala (A), in particular substitutions to Leu (L), Thr (T), Cys (C), Lys (K), Ser (S), Gly (G), Ala (A), preferably substitutions to Gly (G) or Ala (A), in particular Gly (G).

In a preferred isolated variant Cas9 protein, preferably SpCas9 protein, said mutation comprising a deletion of a segment of said surface loop (i.e. a smaller segment within the surface loop), preferably wherein the amino acids which are associated with the 5′ end of the spacer sequence are included in the deleted segment; preferably wherein the length of the deleted segment is e.g. 4 to 12 amino acids or 6 to 12 amino acids, preferably 7 to 11 amino acids or 8 to 10 amino acids or highly preferably 9 amino acids; possibly there are two or more, e.g. two or three shorter segments deleted.

In a preferred isolated variant Cas9 protein, preferably SpCas9 protein, said deleted segment is between amino acids Leu1004 and Asp1017 or preferably between Leu1004 and Lys1014 of the SpCas9 or corresponding amino acids in other Cas9 protein, wherein said amino acids are preferably maintained or preserved, and wherein the length of the deleted segment is thus limited by these amino acids (i.e. at most 12 or preferably 9 in said Cas9 protein, preferably SpCas9 protein), whereas the mutant preferably also comprises insertion (thus substitution) having a length of 1 to 6 amino acids as defined herein.

Preferably, said mutation also comprises an insertion to replace the segment deleted, wherein said amino acids being in contact with the 5′ end of a crRNA or sgRNA are replaced or deleted, said insertion having a length of 1 to 6 amino acids and comprising amino acids which are different from acidic and aromatic amino acids, and the space filling/steric effect/volume of the amino acids is smaller than that of the wild type amino acids,

preferably amino acids selected from the group consisting of Pro (P), Val (V), Ile (I), Leu (L), Ser (S), Thr (T), Cys (C), Met (M), Lys (K), Gly (G), Ala (A), in particular Pro (P), Val (V), Ile (I), Leu (L), Lys (K), Ser (S), Gly (G), Ala (A), preferably Gly (G) and Ala (A).

Preferably, the length of the insertion is 1, 2, 3, 4, 5 or 6 amino acids, preferably 1, 2, 3 or 4 amino acids, more preferably 2 or 3 amino acids, highly preferably 2 amino acids.

i) In a preferred isolated variant Cas9 protein said insertion is selected from the following group of peptides and amino acids:

(Gly)ₘ, wherein m is an integer from 1 to 6,
(Ala)ₙ, wherein n is an integer from 1 to 6,
(Ser)ₒ, wherein o is an integer from 1 to 4,
(Gly)ₓ(Pro)_{y}, wherein x and y are, independently from each other, integers from 1 to 4 wherein the sum of x+y is not more than 6,
(Gly)ₓ(Ala)_{y}, wherein x and y are, independently from each other, integers from 1 to 4 wherein the sum of x+y is not more than 6,
(Gly)ₓ(Lys)_{y}, wherein x and y are, independently from each other, integers from 1 to 4 wherein the sum of x+y is not more than 6,
(Gly)ₓ(Ser)_{y}, wherein x and y are, independently from each other, integers from 1 to 4 wherein the sum of x+y is not more than 6.

ii) In a more preferred isolated variant Cas9 protein said insertion is selected from the following group of peptides and amino acids:

(Gly)ₘ, wherein m is an integer from 1 to 4, preferably 1, 2, 3 or 4,
(Ala)ₙ, wherein n is an integer from 1 to 4 preferably 1, 2, 3 or 4.

In a preferred isolated variant Cas9 protein the wild type Cas9 protein is an SpCas9, and the mutation comprises a deletion of one or more segment(s) of the surface loop comprising amino acids Glu1007 and/or Tyr1013; preferably wherein the length of the deleted segment is 6 to 12 amino acids, preferably 7 to 11 amino acids or 8 to 10 amino acids or highly preferably 9 amino acids, and optionally said mutation also comprises an insertion to replace the segment deleted, so that Glu1007 and/or Tyr1013 are deleted or replaced, and preferably the insertion is defined in item (i) above. In a preferred isolated variant the mutation comprises a deletion of one or more segment(s) of the surface loop comprising amino acids Glu1007 and/or Tyr1013 (including preferred options) and preferably the insertion is defined in item (ii) above; in a particularly preferred embodiment the insertion comprises or consists of Gly amino acids.

In a preferred isolated variant Cas9 protein said mutation comprises an insertion, said insertion having a length of 1 to 6 amino acids and comprising amino acids selected from the group consisting of Pro (P), Val (V), Ile (I), Leu (L), Ser (S), Thr (T), Cys (C), Met (M), Lys (K), Gly (G), Ala (A), in particular Pro (P), Val (V), Ile (I), Leu (L), Lys (K), Ser (S), Gly (G), Ala (A), preferably Gly (G) and Ala (A). Preferably, the mutation comprises a deletion of one or more segment(s) of the surface loop comprising amino acids Glu1007 and/or Tyr1013 (and preferred options as given above. Also preferably, said deleted segment is between amino acids Leu1004 and Asp1017 or preferably between Leu1004 and Lys1014 of SpCas9 or corresponding amino acids in other Cas9 protein, wherein said amino acids are preferably maintained or preserved, and wherein the length of the deleted segment is thus limited by these amino acids (i.e. at most 12 or preferably 9 in said Cas9 protein, preferably SpCas9 protein).

Preferably, said insertion is selected from the group of peptides and amino acids as defined above, in any of paragraphs i) or ii).

In a highly preferred embodiment the isolated protein further comprises any fidelity-increasing mutation of an increased fidelity variant,

preferably wherein the mutation (in particular the fidelity-increasing mutation) is selected from a group of mutations present in one or more increased fidelity variant(s) selected from the group consisting of SpCas9-HF1, HypaSpCas9, evoSpCas9, HiFi SpCas9, Sniper SpCas9, eSpCas9, Hypa2SpCas9 and HeFSpCas9 or any fidelity increasing mutation in an increased fidelity Cas9.

Preferably, the isolated spCas9 of the invention further comprises one or more mutations that reduce nuclease activity to generate nuclease inactive or nickase variants.

In a preferred embodiment the mutation that reduces nuclease activity is selected from the group consisting of a mutation in D10, E762, D839, H983, or D986, and H840 or N863, preferably from the group consisting of the following mutations (i) D10A or D10N, and (ii) H840A, H840N or H840Y.

In a highly preferred embodiment the nuclease activity of the variant Cas9 protein is abolished.

In a preferred embodiment a fusion protein comprises the isolated Cas9 protein of the invention fused to a heterologous functional domain, optionally by an intervening linker, wherein the linker does not interfere with the activity of the fusion protein.

In an alternative embodiment the crRNA or the sgRNA comprises a binding site, and a heterologous functional domain is provided which is linked to and/or comprises a binding domain capable of binding to the binding site. In an example the binding site in the crRNA or in the sgRNA is an aptamer and the binding domain is an aptamer binding domain.

Preferably the heterologous functional domain is a domain capable functioning on DNA. Preferably said heterologous functional domain is selected from a group consisting of a transcriptional activation domain, transcriptional silencer or a transcriptional repression domain, an enzyme that alters the methylation state of DNA, enzyme that modifies histone subunits, a biological tether, a reverse transcriptase and a deaminase domain.

Preferably, in the fusion protein

the transcription activation domain is selected from VP64 or NF-κB p65;

the transcriptional repression domain is a Kruppel associated box (KRAB) domain, an ERF repressor domain (ERD), or an mSin3A interaction domain (SID),

the transcription silencer is heterochromatin protein 1 (HP1), preferably HP1a or HP1β

the enzyme that alters the methylation state of the DNA is DNA methyltransferase (DNMT) or TET protein, wherein preferably the TET protein is TET1,

the enzyme that modifies histone subunits is histone acetyltransferase (HAT), histone deacetylase (HDAC), histone methyltransferase (HMT), or histone demethylase,

the biological tether is MS2, Csy4 or lambda N protein,

the heterologous functional domain is a deaminase, preferably the deaminase is a ApoBac, AID or TADA,

the heterologous functional domain is a reverse transcriptase and/or

the heterologous functional domain is Fold.

In a preferred embodiment, in the isolated variant (mutant) Cas9 protein the mutation(s) in said surface loop

increases on-target activities of an increased fidelity variant having the same mutation except mutation(s) in said surface loop,

in a target dependent manner with 5′-extended single guide RNAs (sgRNAs).

In a preferred embodiment the isolated mutant (variant) Cas9 protein of the invention, e.g. as defined above (in particular comprising the one or more deletion(s) and insertion as defined herein or above), whereas comprises an amino acid sequence that has at least 50% or at least 60% or at least 70% or preferably at least 80% or at least 90% sequence identity to a wild type sequence or to the following amino acid sequence, wherein Lys1003, Leu1004 and Glu1007 are marked in bold and underlined, respectively and Tyr1013, Lys1014 and Asp1017 are marked in bold, respectively,

| | |
|---|---|
| MDYKDHDGDYKDHDIDYKDDDDK MAPKKKRKVGIHGVPAA | 40 |
| DKKYSIGLDIGTNSVGWAVITDEYKVPSKKFKVLGNTDRHSIKKNLIGALLFDSGETAEA | 100 |
| TRLKRTARRRYTRRKNRICYLQEIFSNEMAKVDDSFFHRLEESFLVEEDKKHERHPIFGN | 160 |
| IVDEVAYHEKYPTIYHLRKKLVDSTDKADLRLIYLALAHMIKFRGHFLIEGDLNPDNSDV | 220 |
| DKLFIQLVQTYNQLFEENPINASGVDAKAILSARLSKSRRLENLIAQLPGEKKNGLFGNL | 280 |
| IALSLGLTPNFKSNFDLAEDAKLQLSKDTYDDDLDNLLAQIGDQYADLFLAAKNLSDAIL | 340 |
| LSDILRVNTEITKAPLSASMIKRYDEHHQDLTLLKALVRQQLPEKYKEIFFDQSKNGYAG | 400 |
| YIDGGASQEEFYKFIKPILEKMDGTEELLVKLNREDLLRKQRTFDNGSIPHQIHLGELHA | 460 |
| ILRRQEDFYPFLKDNREKIEKILTFRIPYYVGPLARGNSRFAWMTRKSEETITPWNFEEV | 520 |
| VDKGASAQSFIERMTNFDKNLPNEKVLPKHSLLYEYFTVYNELTKVKYVTEGMRKPAFLS | 580 |
| GEQKKAIVDLLFKTNRKVTVKQLKEDYFKKIECFDSVEISGVEDRFNASLGTYHDLLKII | 640 |
| KDKDFLDNEENEDILEDIVLTLTLFEDREMIEERLKTYAHLFDDKVMKQLKRRRYTGWGR | 700 |
| LSRKLINGIRDKQSGKTILDFLKSDGFANRNFMQLIHDDSLTFKEDIQKAQVSGQGDSLH | 760 |
| EHIANLAGSPAIKKGILQTVKVVDELVKVMGRHKPENIVIEMARENQTTQKGQKNSRERM | 820 |
| KRIEEGIKELGSQILKEHPVENTQLQNEKLYLYYLQNGRDMYVDQELDINRLSDYDVDHI | 880 |
| VPQSFLKDDSIDNKVLTRSDKNRGKSDNVPSEEVVKKMKNYWRQLLNAKLITQRKFDNLT | 940 |
| KAERGGLSELDKAGFIKRQLVETRQITKHVAQILDSRMNTKYDENDKLIREVKVITLKSK | 1000 |
| LVSDFRKDFQFYKVREINNYHHAHDAYLNAVVGTALIKKYPKLESEFVYGDYKVYDVRKM | 1060 |
| IAKSEQEIGKATAKYFFYSNIMNFFKTEITLANGEIRKRPLIETNGETGEIVWDKGRDFA | 1120 |
| TVRKVLSMPQVNIVKKTEVQTGGFSKESILPKRNSDKLIARKKDWDPKKYGGFDSPTVAY | 1180 |
| SVLVVAKVEKGKSKKLKSVKELLGITIMERSSFEKNPIDFLEAKGYKEVKKDLIIKLPKY | 1240 |
| SLFELENGRKRMLASAGELQKGNELALPSKYVNFLYLASHYEKLKGSPEDNEQKQLFVEQ | 1300 |
| HKHYLDEIIEQISEFSKRVILADANLDKVLSAYNKHRDKPIREQAENIIHLFTLTNLGAP | 1360 |
| AAFKYFDTTIDRKRYTSTKEVLDATLIHQSITGLYETRIDLSQLGGDKRPAATKKAGQAK | 1420 |
| KKK | 1423 |

(SEQ ID NO: 1; wherein N- and C-terminal added peptides are marked
in italics, wherein the first 23 amino acids are a 3XFLAG ® Peptide
sequence and amino acids 24 to 40 as well as amino acids are a
nuclear localization signal (“NLS”;)
or to the following amino acid sequence

| | |
|---|---|
| DKKYSIGLDIGTNSVGWAVITDEYKVPSKKFKVLGNTDRHSIKKNLIGALLFDSGETAEA | 60 |
| TRLKRTARRRYTRRKNRICYLQEIFSNEMAKVDDSFFHRLEESFLVEEDKKHERHPIFGN | 120 |
| IVDEVAYHEKYPTIYHLRKKLVDSTDKADLRLIYLALAHMIKFRGHFLIEGDLNPDNSDV | 180 |
| DKLFIQLVQTYNQLFEENPINASGVDAKAILSARLSKSRRLENLIAQLPGEKKNGLFGNL | 240 |
| IALSLGLTPNFKSNFDLAEDAKLQLSKDTYDDDLDNLLAQIGDQYADLFLAAKNLSDAIL | 300 |
| LSDILRVNTEITKAPLSASMIKRYDEHHQDLTLLKALVRQQLPEKYKEIFFDQSKNGYAG | 360 |
| YIDGGASQEEFYKFIKPILEKMDGTEELLVKLNREDLLRKQRTFDNGSIPHQIHLGELHA | 420 |
| ILRRQEDFYPFLKDNREKIEKILTFRIPYYVGPLARGNSRFAWMTRKSEETITPWNFEEV | 480 |
| VDKGASAQSFIERMTNFDKNLPNEKVLPKHSLLYEYFTVYNELTKVKYVTEGMRKPAFLS | 540 |
| GEQKKAIVDLLFKTNRKVTVKQLKEDYFKKIECFDSVEISGVEDRFNASLGTYHDLLKII | 600 |
| KDKDFLDNEENEDILEDIVLTLTLFEDREMIEERLKTYAHLFDDKVMKQLKRRRYTGWGR | 660 |
| LSRKLINGIRDKQSGKTILDFLKSDGFANRNFMQLIHDDSLTFKEDIQKAQVSGQGDSLH | 720 |
| EHIANLAGSPAIKKGILQTVKVVDELVKVMGRHKPENIVIEMARENQTTQKGQKNSRERM | 780 |
| KRIEEGIKELGSQILKEHPVENTQLQNEKLYLYYLQNGRDMYVDQELDINRLSDYDVDHI | 840 |
| VPQSFLKDDSIDNKVLTRSDKNRGKSDNVPSEEVVKKMKNYWRQLLNAKLITQRKFDNLT | 900 |
| KAERGGLSELDKAGFIKRQLVETRQITKHVAQILDSRMNTKYDENDKLIREVKVITLKSK | 960 |
| LVSDFRKDFQFYKVREINNYHHAHDAYLNAVVGTALIKKYPKLESEFVYGDYKVYDVRKM | 1020 |
| IAKSEQEIGKATAKYFFYSNIMNFFKTEITLANGEIRKRPLIETNGETGEIVWDKGRDFA | 1080 |
| TVRKVLSMPQVNIVKKTEVQTGGFSKESILPKRNSDKLIARKKDWDPKKYGGFDSPTVAY | 1140 |
| SVLVVAKVEKGKSKKLKSVKELLGITIMERSSFEKNPIDFLEAKGYKEVKKDLIIKLPKY | 1200 |
| SLFELENGRKRMLASAGELQKGNELALPSKYVNFLYLASHYEKLKGSPEDNEQKQLFVEQ | 1260 |
| HKHYLDEIIEQISEFSKRVILADANLDKVLSAYNKHRDKPIREQAENIIHLFTLTNLGAP | 1320 |
| AAFKYFDTTIDRKRYTSTKEVLDATLIHQSITGLYETRIDLSQLGGD | 1367 |

(SEQ ID NO: 2) of Uniprot entry no. Q99ZW2; CRISPR-associated
endonuclease Cas9/Csn1 from Streptococcus pyogenes serotype M1,
wherein Lys1003, Leu1004 and Glu1007 are marked in bold and
underligned, respectively and Tyr1013, Lys1014 and Asp1017 are
marked in bold, respectively
or to the following amino acid sequence

| | |
|---|---|
| DKKYSIGLDIGTNSVGWAVITDDYKVPSKKFKVLGNTDRHSIKKNLIGALLFDSGETAEA | 60 |
| TRLKRTARRRYTRRKNRICYLQEIFSNEMAKVDDSFFHRLEESFLVEEDKKHERHPIFGN | 120 |
| IVDEVAYHEKYPTIYHLRKKLADSTDKADLRLIYLALAHMIKFRGHFLIEGDLNPDNSDV | 180 |
| DKLFIQLVQTYNQLFEENPINASRVDAKAILSARLSKSRRLENLIAQLPGEKKNGLFGNL | 240 |
| IALSLGLTPNFKSNFDLAEDAKLQLSKDTYDDDLDNLLAQIGDQYADLFLAAKNLSDATL | 300 |
| LSDILRVNSEITKAPLSASMIKRYDEHHQDLTLLKALVRQQLPEKYKEIFFDQSKNGYAG | 360 |
| YIDGGASQEEFYKFIKPILEKMDGTEELLAKLNREDLLRKQRTFDNGSIPHQIHLGELHA | 420 |
| ILRRQEDFYPFLKDNREKIEKILTFRIPYYVGPLARGNSRFAWMTRKSEETITPWNFEEV | 480 |
| VDKGASAQSFIERMTNFDKNLPNEKVLPKHSLLYEYFTVYNELTKVKYVTEGMRKPAFLS | 540 |
| GEQKKAIVDLLFKTNRKVTVKQLKEDYFKKIECFDSVEISGVEDRFNASLGTYHDLLKII | 600 |
| KDKDFLDNEENEDILEDIVLTLTLFEDKEMIEERLKTYAHLFDDKVMKQLKRRHYTGWGR | 660 |
| LSRKLINGIRDKQSGKTILDFLKSDGFANRNFIQLIHDDSLTFKEAIQKAQVSGQGHSLH | 720 |
| EQIANLAGSPAIKKGILQSVKWDELVKVMGHKPENIVIEMARENQTTQKGQKNSRERMK | 780 |
| RIEEGIKELGSQILKEHPVENTQLQNEKLYLYYLQNGRDMYVDQELDINRLSDYDVDHIV | 840 |
| PQSFIKDDSIDNKVLTRSDKNRGKSDDVPSEEWKKMKNYWRQLLNAKLITQRKFDNLTK | 900 |
| AERGGLSELDKAGFIKRQLVETRQITKHVAQILDSRMNTKYDENDKLIREVKVITLKSKL | 960 |
| VSDFRKDFQFYKVREINNYHHAHDAYLNAVVGTALIKKYPKLESEFVYGDYKVYDVRKMI | 1020 |
| AKSEQEIGKATAKRFFYSNIMNFFKTEITLANGEIRKRPLIETNEETGEIVWDKGRDFAT | 1080 |
| VRKVLSMPQVNIVKKTEVQTGALTNESIYARGSFDKLISRKHRFESSKYGGFGSPTVTYS | 1140 |
| VLWAKSKVQDGKVKKIKTGKELIGITLLDKLVFEKNPLKFIEDKGYGNVQIDKCIKLPK | 1200 |
| YSLFEFENGTRRMLASVMANNNSRGDLQKANEMFLPAKLVTLLYHAHKIESSKELEHEAY | 1260 |
| ILDHYNDLYQLLSYIERFASLYVDVEKNISKVKELFSNIESYSISEICSSVINLLTLTAS | 1320 |
| GAPADFKFLGTTIPRKRYGSPQSILSSTLIHQSITGLYETRIDLSQLGSD | 1370 |

(SEQ ID NO: 3) of Uniprot entry no. Q1J6W2; CRISPR-associated
endonuclease Cas9 from Streptococcus pyogenes serotype M4
(strain MGAS10750), wherein Lysl003, Leul004 and Glu1007 are
marked in bold and underligned, respectively and Tyr1013,
Lys1014 and Asp1017 are marked in bold, respectively;
wherein the variant Cas9 comprises the mutation of the invention
as defined herein.

In a highly preferred embodiment the isolated protein further comprises any fidelity-increasing mutation of an increased fidelity variant, preferably wherein the mutation is selected from a group of mutations present in one or more increased fidelity variant(s) selected from the group consisting of SpCas9-HF1, HypaSpCas9, evoSpCas9, HiFi SpCas9, Sniper SpCas9, eSpCas9, Hypa2SpCas9 and HeFSpCas9 or any fidelity increasing mutation in an increased fidelity Cas9.

In a highly preferred embodiment the variant Cas9 comprises a sequence selected from the following group consisting of SEQ ID NOs 6 to 23 as listed below, wherein said sequence is present in the position of (i.e. replaces) the wild type segment from Lys1003 to Asp1017 of SpCas9 or a corresponding sequence of a wild type Cas9 protein comprising said surface loop:

| 1003-------------1017 | |
|---|---|
| [symbol] ES [symbol] FVYGD [symbol] VY [symbol] | SEQ ID NO: 4 |
| KGKVYD | SEQ ID NO: 6 |
| KD | SEQ ID NO: 7 |
| KGD | SEQ ID NO: 8 |
| KGKD | SEQ ID NO: 9 |
| KLKVYD | SEQ ID NO: 10 |
| KLGKVYD | SEQ ID NO: 11 |
| KLPKVYD | SEQ ID NO: 12 |
| KLGGKVYD | SEQ ID NO: 13 |
| KLGPKVYD | SEQ ID NO: 14 |
| KLGGGKVYD | SEQ ID NO: 15 |
| KLGGGGKVYD | SEQ ID NO: 16 |
| KLGGVYD | SEQ ID NO: 17 |
| KLGGYD | SEQ ID NO: 18 |
| KLGGD | SEQ ID NO: 19 |
| KLSSKVYD | SEQ ID NO: 20 |
| KLSSD | SEQ ID NO: 21 |
| KLESKVYD | SEQ ID NO: 22 |
| KLESGKVYD | SEQ ID NO: 23 |

In a preferred embodiment the isolated mutant (variant) Cas9 protein of the invention as defined above comprises a segment having a sequence selected

from the group consisting of SEQ ID NOs 5 to 23 or preferably
from the group consisting of SEQ ID NOs 6 to 23, or
from the group consisting of SEQ ID NOs 5, and 10 to 16 and 20 and 22 and 23, or
from the group consisting of SEQ ID NOs 6, and 10 to 16 and 20 and 22 and 23, or
from the group consisting of SEQ ID NOs 10 to 16 and 20 and 22 and 23, or
from the group consisting of SEQ ID NOs 10 to 18, or
from the group consisting of SEQ ID NOs 6, 8, 9, 11, 12 and 13 to 21, or
from the group consisting of SEQ ID NOs 13, 14, 17, 18, 19, or
from the group consisting of SEQ ID NOs 6, 8, 9, 11-21 and 23.

In a preferred embodiment the isolated mutant (variant) Cas9 protein of the invention as defined above comprises a segment having a sequence selected

from the group consisting of SEQ ID NOs 6, 11, 13, 14, 17 and 18, or
from the group consisting of SEQ ID NOs 13, 14, 15 and 16, or
from the group consisting of SEQ ID NOs 13.

Preferably the isolated mutant (variant) Cas9 protein of the invention has an amino acid sequence of SEQ ID NO: 1 or a sufficient part thereof to maintain at least DNA binding activity or a part thereof without the added peptide, or an amino acid sequence that has at least 50% or at least 60% or at least 70% or preferably at least 80% or at least 90% sequence identity to SEQ ID NO: 1 except that it comprises, in replacement of the wild type loop sequence, a segment as defined in this preferred embodiment above.

Preferably the isolated mutant (variant) Cas9 protein of the invention has an amino acid sequence of SEQ ID NO: 2 or a sufficient part thereof to maintain at least DNA binding activity, or an amino acid sequence that has at least 50% or at least 60% or at least 70% or preferably at least 80% or at least 90% sequence identity to SEQ ID NO: 2, except that it comprises, in replacement of the wild type loop sequence, a segment as defined in this preferred embodiment above.

Preferably the isolated mutant (variant) Cas9 protein of the invention has an amino acid sequence of SEQ ID NO: 3 or a sufficient part thereof to maintain at least DNA binding activity, or an amino acid sequence that has at least 50% or at least 60% or at least 70% or preferably at least 80% or at least 90% sequence identity to SEQ ID NO: 3, except that it comprises, in replacement of the wild type loop sequence, a segment as defined in this preferred embodiment above.

| | 1003-------------1017 | 1003-------------1017 | |
|---|---|---|---|
| Wild type loop sequence | KLESEFVYGDYKVYD | KLESEFVYGDYKVYD | SEQ ID NO: 4 |
| HF-B1 | KLESGFVYGDGKVYD | KLESGFVYGDGKVYD | SEQ ID NO: 5 |
| HF-B2 | KGKVYD | K-----G----KVYD | SEQ ID NO: 6 |
| HF-B3 | KD | K-------------D | SEQ ID NO: 7 |
| HF-B4 | KGD | K-----G-------D | SEQ ID NO: 8 |
| HF-B5 | KGKD | K-----GK------D | SEQ ID NO: 9 |
| HF-B6 | KLKVYD | KL---------KVYD | SEQ ID NO: 10 |
| HF-B7 | KLGKVYD | KL----G----KVYD | SEQ ID NO: 11 |
| HF-B8 | KLPKVYD | KL----P----KVYD | SEQ ID NO: 12 |
| HF-B9 (B-SpCas9-HF1) | KLGGKVYD | KL----GG---KVYD | SEQ ID NO: 13 |
| HF-B10 | KLGPKVYD | KL----GP---KVYD | SEQ ID NO: 14 |
| HF-B11 | KLGGGKVYD | KL----GGG--KVYD | SEQ ID NO: 15 |
| HF-B12 | KLGGGGKVYD | KL----GGGG-KVYD | SEQ ID NO: 16 |
| HF-B13 | KLGGVYD | KL----GG----VYD | SEQ ID NO: 17 |
| HF-B14 | KLGGYD | KL----GG-----YD | SEQ ID NO: 18 |
| HF-B15 | KLGGD | KL----GG------D | SEQ ID NO: 19 |
| HF-B16 | KLSSKVYD | KL----SS---KVYD | SEQ ID NO: 20 |
| HF-B17 | KLSSD | KL----SS------D | SEQ ID NO: 21 |
| HF-B18 | KLESKVYD | KLES-------KVYD | SEQ ID NO: 22 |
| HF-B19 | KLESGKVYD | KLES--G----KVYD | SEQ ID NO: 23 |

It is to be noted that in case of SEQ ID NO: 7 and 8 in the sequence listing amino acids Xaa have been inserted to indicate deleted amino acids (deletion(s)). However these sequences are equivalent with those without formal addition of deleted amino acids. Such addition was also necessary as WIPO ST. 25 requires at least four amino acids in a sequence listing.

In a highly preferred embodiment the isolated mutant (variant) Cas9 protein of the invention as defined above comprises a segment having a sequence selected from the group consisting of SEQ ID NOs 5 to 23 or preferably 6 to 23 or any of the groups consisting of subsets of these sequences as defined above, wherein said isolated protein further comprises any fidelity-increasing mutation of an increased fidelity variant, preferably wherein the mutation is selected from a group of mutations present in one or more increased fidelity variant(s) selected from the group consisting of SpCas9-HF1, HypaSpCas9, evoSpCas9, HiFi SpCas9, Sniper SpCas9, eSpCas9, Hypa2SpCas9 and HeFSpCas9 or any fidelity increasing mutation in an increased fidelity Cas9.

The invention also relates to a ribonucleoprotein (RNP) complex comprising the isolated mutant (variant) Cas9 protein of the invention, e.g. as defined above, said RNP complex also comprising an RNA having a spacer sequence, preferably a crRNA or a single guide RNA (sgRNA).

Preferably in the RNP complex said spacer sequence or said crRNA or sgRNA is extended with one or two G nucleotide at its 5′ end.

Preferably, in the RNP complex the sgRNA is transcribed intracellularly, in vitro transcribed or custom synthesized and introduced through transfection.

Preferably, in the RNP complex is prepared within a cell. Preferably, in the RNP complex is prepared in an extracellular environment and introduced into the cell.

Also provided herein are nucleic acids, isolated nucleic acids encoding the variant Cas9 proteins described herein, as well as vectors comprising the isolated nucleic acids, optionally operably linked to one or more regulatory domains for expressing the variant Cas9 proteins described herein. Also provided herein are host cells, e.g., bacterial, yeast, insect, or mammalian host cells or transgenic animals (e.g., mice), comprising the nucleic acids described herein, and optionally expressing the variant Cas9 proteins described herein.

In particular, the invention also relates to an isolated nucleic acid encoding a Cas9 protein of the invention, preferably as defined in any of the paragraphs above.

The invention also relates to a nucleic acid, said nucleic acid encoding a Cas9 protein of the invention.

The invention also relates to a nucleic acid, said nucleic acid encoding a Cas9 protein of the invention, said nucleic acid comprising a nucleic acid sequence that has at least 40% or at least 50% or preferably at least 60% or preferably at least 70% or preferably at least 80% or at least 90% sequence identity to the nucleic acid sequence SEQ ID NO: 24 except that it comprises the mutation according to the invention

(SEQ ID NO: 24)

| | |
|---|---|
| | GACAAGAA GTACAGCATC |
| 961 | GGCCTGGACA TCGGCACCAA CTCTGTGGGC TGGGCCGTGA TCACCGACGA GTACAAGGTG CCCAGCAAGA AATTCAAGGT |
| 1041 | GCTGGGCAAC ACCGACCGGC ACAGCATCAA GAAGAACCTG ATCGGAGCCC TGCTGTTCGA CAGCGGCGAA ACAGCCGAGG |
| 1121 | CCACCCGGCT GAAGAGAACC GCCAGAAGAA GATACACCAG ACGGAAGAAC CGGATCTGCT ATCTGCAAGA GATCTTCAGC |
| 1201 | AACGAGATGG CCAAGGTGGA CGACAGCTTC TTCCACAGAC TGGAAGAGTC CTTCCTGGTG GAAGAGGATA AGAAGCACGA |
| 1281 | GCGGCACCCC ATCTTCGGCA ACATCGTGGA CGAGGTGGCC TACCACGAGA AGTACCCCAC CATCTACCAC CTGAGAAAGA |
| 1361 | AACTGGTGGA CAGCACCGAC AAGGCCGACC TGCGGCTGAT CTATCTGGCC CTGGCCCACA TGATCAAGTT CCGGGGCCAC |
| 1441 | TTCCTGATCG AGGGCGACCT GAACCCCGAC AACAGCGACG TGGACAAGCT GTTCATCCAG CTGGTGCAGA CCTACAACCA |
| 1521 | GCTGTTCGAG GAAAACCCCA TCAACGCCAG CGGCGTGGAC GCCAAGGCCA TCCTGTCTGC CAGACTGAGC AAGAGCAGAC |
| 1601 | GGCTGGAAAA TCTGATCGCC CAGCTGCCCG GCGAGAAGAA GAATGGCCTG TTCGGAAACC TGATTGCCCT GAGCCTGGGC |
| 1681 | CTGACCCCCA ACTTCAAGAG CAACTTCGAC CTGGCCGAGG ATGCCAAACT GCAGCTGAGC AAGGACACCT ACGACGACGA |
| 1761 | CCTGGACAAC CTGCTGGCCC AGATCGGCGA CCAGTACGCC GACCTGTTTC TGGCCGCCAA GAACCTGTCC GACGCCATCC |
| 1841 | TGCTGAGCGA CATCCTGAGA GTGAACACCG AGATCACCAA GGCCCCCCTG AGCGCCTCTA TGATCAAGAG ATACGACGAG |
| 1921 | CACCACCAGG ACCTGACCCT GCTGAAAGCT CTCGTGCGGC AGCAGCTGCC TGAGAAGTAC AAAGAGATTT TCTTCGACCA |
| 2001 | GAGCAAGAAC GGCTACGCCG GCTACATTGA CGGCGGAGCC AGCCAGGAAG AGTTCTACAA GTTCATCAAG CCCATCCTGG |
| 2081 | AAAAGATGGA CGGCACCGAG GAACTGCTCG TGAAGCTGAA CAGAGAGGAC CTGCTGCGGA AGCAGCGGAC CTTCGACAAC |
| 2161 | GGCAGCATCC CCCACCAGAT CCACCTGGGA GAGCTGCACG CCATTCTGCG GCGGCAGGAA GATTTTTACC CATTCCTGAA |
| 2241 | GGACAACCGG GAAAAGATCG AGAAGATCCT GACCTTCCGC ATCCCCTACT ACGTGGGCCC TCTGGCCAGG GGAAACAGCA |
| 2321 | GATTCGCCTG GATGACCAGA AAGAGCGAGG AAACCATCAC CCCCTGGAAC TTCGAGGAAG TGGTGGACAA GGGCGCTTCC |
| 2401 | GCCCAGAGCT TCATCGAGCG GATGACCAAC TTCGATAAGA ACCTGCCCAA CGAGAAGGTG CTGCCCAAGC ACAGCCTGCT |
| 2481 | GTACGAGTAC TTCACCGTGT ATAACGAGCT GACCAAAGTG AAATACGTGA CCGAGGGAAT GAGAAAGCCC GCCTTCCTGA |
| 2561 | GCGGCGAGCA GAAAAAGGCC ATCGTGGACC TGCTGTTCAA GACCAACCGG AAAGTGACCG TGAAGCAGCT GAAAGAGGAC |
| 2641 | TACTTCAAGA AAATCGAGTG CTTCGACTCC GTGGAAATCT CCGGCGTGGA AGATCGGTTC AACGCCTCCC TGGGCACATA |
| 2721 | CCACGATCTG CTGAAAATTA TCAAGGACAA GGACTTCCTG GACAATGAGG AAAACGAGGA CATTCTGGAA GATATCGTGC |
| 2801 | TGACCCTGAC ACTGTTTGAG GACAGAGAGA TGATCGAGGA ACGGCTGAAA ACCTATGCCC ACCTGTTCGA CGACAAAGTG |
| 2881 | ATGAAGCAGC TGAAGCGGCG GAGATACACC GGCTGGGGCA GGCTGAGCCG GAAGCTGATC AACGGCATCC GGGACAAGCA |
| 2961 | GTCCGGCAAG ACAATCCTGG ATTTCCTGAA GTCCGACGGC TTCGCCAACA GAAACTTCAT GCAGCTGATC CACGACGACA |
| 3041 | GCCTGACCTT TAAAGAGGAC ATCCAGAAAG CCCAGGTGTC CGGCCAGGGC GATAGCCTGC ACGAGCACAT TGCCAATCTG |
| 3121 | GCCGGCAGCC CCGCCATTAA GAAGGGCATC CTGCAGACAG TGAAGGTGGT GGACGAGCTC GTGAAAGTGA TGGGCCGGCA |
| 3201 | CAAGCCCGAG AACATCGTGA TCGAAATGGC CAGAGAGAAC CAGACCACCC AGAAGGGACA GAAGAACAGC CGCGAGAGAA |
| 3281 | TGAAGCGGAT CGAAGAGGGC ATCAAAGAGC TGGGCAGCCA GATCCTGAAA GAACACCCCG TGGAAAACAC CCAGCTGCAG |
| 3361 | AACGAGAAGC TGTACCTGTA CTACCTGCAG AATGGGCGGG ATATGTACGT GGACCAGGAA CTGGACATCA ACCGGCTGTC |
| 3441 | CGACTACGAT GTGGACCATA TCGTGCCTCA GAGCTTTCTG AAGGACGACT CCATCGACAA CAAGGTGCTG ACCAGAAGCG |
| 3521 | ACAAGAACCG GGGCAAGAGC GACAACGTGC CCTCCGAAGA GGTCGTGAAG AAGATGAAGA ACTACTGGCG GCAGCTGCTG |
| 3601 | AACGCCAAGC TGATTACCCA GAGAAAGTTC GACAATCTGA CCAAGGCCGA GAGAGGCGGC CTGAGCGAAC TGGATAAGGC |
| 3681 | CGGCTTCATC AAGAGACAGC TGGTGGAAAC CCGGCAGATC ACAAAGCACG TGGCACAGAT CCTGGACTCC CGGATGAACA |
| 3761 | CTAAGTACGA CGAGAATGAC AAGCTGATCC GGGAAGTGAA AGTGATCACC CTGAAGTCCA AGCTGGTGTC CGATTTCCGG |
| 3841 | AAGGATTTCC AGTTTTACAA AGTGCGCGAG ATCAACAACT ACCACCACGC CCACGACGCG TACCTGAACG CCGTCGTGGG |
| 3921 | AACCGCCCTG ATCAAAAAGT ACCCTAAGCT GGAAAGCGAG TTCGTGTACG GCGACTACAA GGTGTACGAC GTACGGAAGA |
| 4001 | TGATCGCCAA GAGCGAGCAG GAAATCGGCA AGGCTACCGC CAAGTACTTC TTCTACAGCA ACATCATGAA CTTTTTCAAG |
| 4081 | ACCGAGATTA CCCTGGCCAA CGGCGAGATC CGGAAGCGGC CTCTGATCGA GACAAACGGC GAAACCGGGG AGATCGTGTG |
| 4161 | GGATAAGGGC CGGGATTTTG CCACCGTGCG GAAAGTGCTG AGCATGCCCC AAGTGAATAT CGTGAAAAAG ACCGAGGTGC |
| 4241 | AGACAGGCGG CTTCAGCAAA GAGTCTATCC TGCCCAAGAG GAACAGCGAT AAGCTGATCG CCAGAAAGAA GGACTGGGAC |
| 4321 | CCTAAGAAGT ACGGCGGCTT CGACAGCCCC ACCGTGGCCT ATTCTGTGCT GGTGGTGGCC AAAGTGGAAA AGGGCAAGTC |
| 4401 | CAAGAAACTG AAGAGTGTGA AAGAGCTGCT GGGGATCACC ATCATGGAAA GAAGCAGCTT CGAGAAGAAT CCCATCGACT |
| 4481 | TTCTGGAAGC CAAGGGCTAC AAAGAAGTGA AAAAGGACCT GATCATCAAG CTGCCTAAGT ACTCCCTGTT CGAGCTGGAA |
| 4561 | AACGGCCGGA AGAGAATGCT GGCCTCTGCC GGCGAACTGC AGAAGGGAAA CGAACTGGCC CTGCCCTCCA AATATGTGAA |
| 4641 | CTTCCTGTAC CTGGCCAGCC ACTATGAGAA GCTGAAGGGC TCCCCCGAGG ATAATGAGCA GAAACAGCTG TTTGTGGAAC |
| 4721 | AGCACAAGCA CTACCTGGAC GAGATCATCG AGCAGATCAG CGAGTTCTCC AAGAGAGTGA TCCTGGCCGA CGCTAATCTG |
| 4801 | GACAAAGTGC TGTCCGCCTA CAACAAGCAC CGGGATAAGC CCATCAGAGA GCAGGCCGAG AATATCATCC ACCTGTTTAC |
| 4881 | CCTGACCAAT CTGGGAGCCC CTGCCGCCTT CAAGTACTTT GACACCACCA TCGACCGGAA GAGGTACACC AGCACCAAAG |
| 4961 | AGGTGCTGGA CGCCACCCTG ATCCACCAGA GCATCACCGG CCTGTACGAG ACACGGATCG ACCTGTCTCA GCTGGGAGGC |
| 5041 | GAC. |

The invention also relates to a vector comprising the isolated nucleic acid of the invention wherein said nucleic acid codes for any of the above said variant Cas9s of the invention.

Preferably, in the vector the isolated nucleic acid is operably linked to one or more nucleic acid(s) coding for regulatory domains for expressing the variant Cas9 protein according to the invention, e.g. as defined in any of the paragraphs above.

In one aspect, the invention provides vectors that are used in the engineering and optimization of CRISPR-Cas systems.

The invention also relates to a host cell comprising the nucleic acid of the invention or a vector of the invention. In a preferred embodiment said host cell is a mammalian host cell. Preferably, the cell is a stem cell, preferably an embryonic stem cell, a tissue stem cell, e.g. a mesenchymal stem cell, or an induced pluripotent stem cell (iPSC).

Preferably the host cell is an animal cell, e.g. a mammalian cell, e.g. a human cell.

In an embodiment the host cell is a plant cell.

The invention also relates to a kit comprising the isolated nucleic acid of the invention and/or the vector of the invention and/or an ribonucleoprotein of the invention, and/or a host cell of the invention, and a target specific crRNA or single guide RNA. Preferably, the target specific crRNA or single guide RNA is a library of crRNAs or sgRNAs. In a preferred embodiment the library is a pooled library. A preferred library is a lentiviral library of crRNAs or sgRNAs.

Also provided herein are methods for altering, e.g., selectively altering, the genome of a cell by contacting the cell with, or expressing in the cell, the variant Cas9 protein as described herein, and a crRNA or an sgRNA having a region complementary to a selected portion of the genome of the cell. In some embodiments, the isolated protein or fusion protein comprises one or more of a nuclear localization sequence, cell penetrating peptide sequence, and/or affinity tag.

In particular, the invention also relates to a method of altering the genome or epigenome of a cell, said method comprising

expressing in the cell a Cas9 protein according to the invention, e.g. according to any of the paragraphs above or contacting the cell with said Cas9 protein, preferably a fusion protein as defined above,

providing a crRNA having a target specific spacer sequence, preferably a target-specific single guide RNA (sgRNA) having a region complementary to a selected portion of the genome of the cell,

whereby the genome of the cell is altered.

Preferably, the isolated protein or fusion protein comprises one or more of a nuclear localization sequence, a cell penetrating peptide sequence, and/or an affinity tag.

In a preferred embodiment the invention relates to a method of altering a double-stranded DNA (dsDNA) molecule comprising contacting the dsDNA molecule with a protein according to any of the paragraphs above and a crRNA having a target specific spacer sequence, preferably a target-specific single guide RNA (sgRNA) having a region complementary to a selected portion of the dsDNA molecule i.e. the target sequence. Preferably, the dsDNA molecule is present in vitro.

The invention also relates to a method of altering a double-stranded DNA (dsDNA) molecule, said method comprising

providing a ribonucleoprotein (RNP) complex according to the invention, said RNP complex comprising an isolated mutant (variant) Cas9 protein of the invention and an RNA having a target specific spacer sequence, preferably a target-specific single guide RNA (sgRNA) having a region complementary to a selected portion of the dsDNA molecule i.e. the target sequence,

allowing the RNP complex to act on the dsDNA molecule to have said dsDNA molecule altered.

Preferably the alteration in the genome or in the dsDNA molecule is selected from transcriptional activation, transcriptional silencing, transcriptional repression, alteration of methylation state, modification of histone subunits, deamination.

Preferably, said alteration is effected by an appropriate functional domain as listed above.

In certain aspects of the invention methods are provided for modifying a target polynucleotide in a eukaryotic cell. In some embodiments, the method comprises sampling a cell or population of cells from a human or nonhuman animal or a plant and modifying the cell or cells. Culturing may occur at any stage ex vivo. The cell or cells are then re-introduced into the organism. In a preferred embodiment the cells are stem cells (including tissue stem cells, pluripotent stem cells or iPSCs).

In some embodiments, the method comprises allowing a CRISPR complex to bind to the target polynucleotide to effect cleavage of said target polynucleotide thereby modifying the target polynucleotide, wherein the CRISPR complex comprises a CRISPR enzyme complexed with a guide sequence hybridized to a target sequence within said target polynucleotide, wherein said guide sequence is linked to a tracr mate sequence which in turn hybridizes to a tracr sequence.

In one aspect, the invention provides a method of modifying expression of a polynucleotide in a eukaryotic cell. In one embodiment, this invention provides a method of cleaving a target polynucleotide. In some embodiments, the method comprises allowing a CRISPR complex to bind to the polynucleotide such that said binding results in increased or decreased expression of said polynucleotide; where the CRISPR complex comprises a CRISPR enzyme complexed with a guide sequence hybridized to a target sequence within said polynucleotide, wherein said guide sequence is linked to a tracr mate sequence which in turn hybridizes to a tracr sequence. Similar considerations apply as above for methods of modifying a target polynucleotide. In fact, these sampling, culturing and re-introduction options apply across the aspects of the present invention.

In one aspect, the invention provides kits containing any one or more of the elements disclosed in the above methods and compositions. Elements may be provided individually or in combinations, and may be provided in any suitable container, such as a vial, a bottle, or a tube. In some embodiments, the kit includes instructions in one or more languages, for example in more than one language.

The invention is also described herein by the following numbered paragraphs.

The invention relates to the following:

1. A variant Cas9 protein comprising a mutation in the surface loop proximal to the 5′ end of a target specific spacer sequence in a crRNA or sgRNA when said crRNA or sgRNA is in association with said Cas9 protein, said mutation comprising deletion of a segment of said surface loop to remove amino acids which, in a corresponding surface loop having a wild type sequence, are in contact with the 5′ end of the target specific spacer sequence,
wherein said mutation increases the available target space in the Cas9 protein to accommodate an 5′ extension of the spacer sequence,
whereas the folded three dimensional structure of the Cas9 protein is otherwise maintained and the variant Cas9 protein has Cas9 activity on a target DNA substrate.

1.1 The variant Cas9 protein according to paragraph 1

wherein the mutation also increases the fidelity of said variant Cas9 protein in comparison with a Cas9 protein having the same sequence but not having said mutation (or: in comparison with a reference Cas9 protein not having said mutation),

2. The variant Cas9 protein according to any paragraph 1, which is a variant of a Streptococcus pyogenes Cas9 (SpCas9) protein, having a mutation in the surface loop comprising the following amino acids in the wild type sequence loop: Glu1007 and Tyr1013, wherein said mutation preferably disrupts or removes a capping of the 5′ end of the spacer sequence in the crRNA or sgRNA, said capping being formed by said Glu1007 and Tyr1013 as capping amino acids.

2.1 The variant Cas9 protein according to any of paragraphs 1 and 2 wherein said mutation is between the amino acids Leu1004 and Asp1017, preferably between Leu1004 and Lys1014.

3. The variant SpCas9 protein according to any of paragraphs 2, wherein the surface loop comprises one or more mutations of the following amino acids of the wild type sequence: Glu1007 and Tyr1013, wherein said one or more mutation(s)

disrupt the association between said amino acids and the spacer sequence, preferably the 5′ end of the spacer sequence, and/or

increases the available target space in the Cas9 protein to accommodate an 5′ extension of the spacer sequence, and

increases the fidelity of said Cas9 protein

wherein preferably said mutations are, independently from each other, selected from the group consisting of deletions as well as insertions, preferably said insertions comprising one or more amino acids selected from the group consisting of Pro (P), Val (V), Ile (I), Leu (L), Ser (S), Thr (T), Cys (C), Met (M), Lys (K), Gly (G), Ala (A), in particular substitutions to Leu (L), Thr (T), Cys (C), Lys (K), Ser (S), Gly (G), Ala (A), preferably substitutions to Gly (G) and/or Ala (A), in particular Gly (G).

3.1. The variant SpCas9 protein according to paragraph 3, wherein said mutation comprises substitutions, preferably substitutions to Pro (P), Val (V), Ile (I), Leu (L), Ser (S), Thr (T), Cys (C), Met (M), Lys (K), Gly (G), Ala (A), in particular substitutions to Leu (L), Thr (T), Cys (C), Lys (K), Ser (S), Gly (G), Ala (A), preferably substitutions to Gly (G) and/or Ala (A), in particular Gly (G).

4. The variant Cas9 protein according to any of the previous paragraphs, said mutation comprising a deletion of a segment of said surface loop, preferably wherein the amino acids which are associated with the 5′ end of the spacer sequence are included in the deleted segment; preferably wherein the length of the deleted segment is 6 to 12 amino acids, preferably 7 to 11 amino acids or 8 to 10 amino acids or highly preferably 9 amino acids.

5. The variant Cas9 protein according to paragraph 4, said mutation also comprising an insertion to replace the segment deleted wherein said amino acids being in contact with the 5′ end of the spacer sequence in the wild type loop sequence are replaced or deleted in the variant Cas9, said insertion having a length of 1 to 6 amino acid(s) and comprises amino acids which are different from acidic and aromatic amino acids, and the volume of insertion, and or the space filling or steric effect of the amino acid(s) altogether is smaller than that of the wild type amino acids altogether.

5.1. The variant Cas9 protein according to paragraph 5, wherein the amino acid(s) in the insertion is/are selected from the group consisting of Pro (P), Val (V), Ile (I), Leu (L), Ser (S), Thr (T), Cys (C), Met (M), Lys (K), Gly (G), Ala (A), in particular Pro (P), Val (V), Ile (I), Leu (L), Lys (K), Ser (S), Gly (G), Ala (A), preferably Gly (G) and Ala (A) in particular Gly (G).

6. The variant Cas9 protein according to any of paragraphs 5, wherein said insertion is selected from the following group of peptides and amino acids:

(Gly)ₘ, wherein m is an integer from 1 to 6
(Ala)ₙ, wherein n is an integer from 1 to 6
(Ser)ₒ, wherein o is an integer from 1 to 4
(Gly)ₓ(Pro)_{y}, wherein x and y are, independently from each other, integers from 1 to 4 wherein the sum of x+y is not more than 6,
(Gly)ₓ(Ala)_{y}, wherein x and y are, independently from each other, integers from 1 to 4 wherein the sum of x+y is not more than 6,
(Gly)ₓ(Lys)_{y}, wherein x and y are, independently from each other, integers from 1 to 4 wherein the sum of x+y is not more than 6,
(Gly)ₓ(Ser)_{y}, wherein x and y are, independently from each other, integers from 1 to 4 wherein the sum of x+y is not more than 6,
wherein preferably said insertion is selected from the following group of peptides and amino acids:
(Gly)ₘ, wherein m is an integer from 1 to 4.

7. The variant Cas9 protein according to any of paragraphs 2 to 6,

wherein the wild type Cas9 protein is an SpCas9,
and said mutation comprises a deletion of one or more segment(s) of the surface loop comprising amino acids Glu1007 and/or Tyr1013; preferably wherein the length of the deleted segment is 6 to 12 amino acids, preferably 7 to 11 amino acids or 8 to 10 amino acids or highly preferably 9 amino acids,
and optionally said mutation also comprises an insertion to replace the segment deleted,
so that Glu1007 and/or Tyr1013 are deleted or replaced,
wherein preferably
said insertion has a length of 1 to 6 amino acids and comprises amino acids selected from the group consisting of Pro (P), Val (V), Ile (I), Leu (L), Ser (S), Thr (T), Cys (C), Met (M), Lys (K), Gly (G), Ala (A), in particular Pro (P), Val (V), Ile (I), Leu (L), Lys (K), Ser (S), Gly (G), Ala (A), preferably Gly (G) and Ala (A), in particular Gly (G),
wherein preferably said insertion is selected from the group of peptides and amino acids as defined in par. 6.

8. The protein of any of the previous paragraphs, further comprising any fidelity-increasing mutation of an increased fidelity variant,

preferably wherein the mutation is selected from a group of mutations present in one or more increased fidelity variant(s) selected from the group consisting of SpCas9-HF1, HypaSpCas9, evoSpCas9, HiFi SpCas9, Sniper SpCas9, eSpCas9, Hypa2SpCas9 and HeFSpCas9 or any fidelity increasing mutation in an increased fidelity Cas9; highly preferably the mutation is selected from the group consisting of K848A, K1003A, and R1060A.

9. The variant Cas9 protein of any one of paragraphs 1-8 further comprising one or more mutations that reduce nuclease activity to generate nuclease inactive or nickase variants, wherein preferably the mutation that reduces nuclease activity is selected from the group consisting of a mutation in D10, E762, D839, H983, D986, H840 and N863, preferably from the group consisting of the following mutations D10A or D10N, H840A, H840N and H840Y.

10. A fusion protein comprising the variant Cas9 protein of any one of paragraphs 1-8 fused to a heterologous functional domain,

wherein preferably the heterologous functional domain is selected from a group consisting of a transcriptional activation domain, transcriptional silencer or a transcriptional repression domain, an enzyme that alters the methylation state of DNA, enzyme that modifies histone subunits, a biological tether, a reverse transcriptase and a deaminase domain
wherein optionally the variant Cas9 protein and the heterologous functional domain are connected by an intervening linker, wherein the linker does not interfere with the activity of the fusion protein.

11. The fusion protein according to paragraph 10 wherein

the transcription activation domain is selected from VP64 or NF-κB p65;

the transcriptional repression domain is a Kruppel associated box (KRAB) domain, an ERF repressor domain (ERD), or an mSin3A interaction domain (SID),

the transcription silencer is heterochromatin protein 1 (HP1), preferably HP1a or HP1β,

the enzyme that alters the methylation state of the DNA is DNA methyltransferase (DNMT) or TET protein, wherein preferably the TET protein is TET1,

the enzyme that modifies histone subunits is histone acetyltransferase (HAT), histone deacetylase (HDAC), histone methyltransferase (HMT), or histone demethylase,

the biological tether is MS2, Csy4 or lambda N protein,

the heterologous functional domain is a deaminase, preferably the deaminase is a ApoBac, AID or TADA,

the heterologous functional domain is a reverse transcriptase and/or

the heterologous functional domain is Fold.

12. The variant Cas9 protein of any of paragraphs 1 to 9, or a fusion protein of any of paragraphs 10 to 11, which is an increased fidelity variant Cas9 having mutations other than the mutation in said surface loop, wherein the mutation in the surface loop increases on-target activity of the increased fidelity variant Cas9 having the same mutations except mutation(s) in said surface loop, with an 5′ extended spacer sequence.

13. The mutant (variant) Cas9 protein of any of paragraphs 1 to 12, said protein comprising an amino acid sequence that has at least 80% sequence identity to the following amino acid sequence SEQ ID NO: 1 wherein N- and C-terminal added peptides are marked in italics)

or to the amino acid sequence SEQ ID NO: 2 of Uniprot entry no. Q99ZW2; CRISPR-associated endonuclease Cas9/Csn1 from Streptococcus pyogenes serotype M1
or to the following amino acid sequence SEQ ID NO: 3 of Uniprot entry no. Q1J6W2; CRISPR-associated endonuclease Cas9 from Streptococcus pyogenes serotype M4 (strain MGAS10750).

13.1 The mutant (variant) Cas9 protein of any of paragraphs 1 to 13, preferably of paragraph 13, wherein the isolated mutant (variant) Cas9 protein comprises a segment having a sequence selected

from the group consisting of SEQ ID NOs 5 to 23 or preferably
from the group consisting of SEQ ID NOs 6 to 23, or
from the group consisting of SEQ ID NOs 5, and 10 to 16 and 20 and 22 and 23, or
from the group consisting of SEQ ID NOs 6, 8, 9, 11, 12 and 13 to 21, or
from the group consisting of SEQ ID NOs 13, 14, 17, 18, 19, or
from the group consisting of SEQ ID NOs 6, 8, 9, 11-21 and 23.

In a preferred embodiment the isolated mutant (variant) Cas9 protein of the invention as defined above comprises a segment having a sequence selected

from the group consisting of SEQ ID NOs 6, 11, 13, 14, 17 and 18, or
from the group consisting of SEQ ID NOs 13, 14, 15 and 16, or
from the group consisting of SEQ ID NOs 13.

Preferably the isolated mutant (variant) Cas9 protein of the invention has an amino acid sequence of SEQ ID NO: 1 or a sufficient part thereof to maintain activity, at least DNA binding activity or a part thereof without the added peptide, or an amino acid sequence that has at least 50% or at least 60% or at least 70% or preferably at least 80% or at least 90% sequence identity to SEQ ID NO: 1 except that it comprises, in replacement of the wild type loop sequence, a segment as defined in this preferred embodiment above.

Preferably the isolated mutant (variant) Cas9 protein of the invention has an amino acid sequence of SEQ ID NO: 2 or a sufficient part thereof to maintain activity, at least DNA binding activity, or an amino acid sequence that has at least 50% or at least 60% or at least 70% or preferably at least 80% or at least 90% sequence identity to SEQ ID NO: 2, except that it comprises, in replacement of the wild type loop sequence, a segment as defined in this preferred embodiment above.

Preferably the isolated mutant (variant) Cas9 protein of the invention has an amino acid sequence of SEQ ID NO: 3 or a sufficient part thereof to maintain activity, at least DNA binding activity, or an amino acid sequence that has at least 50% or at least 60% or at least 70% or preferably at least 80% or at least 90% sequence identity to SEQ ID NO: 3, except that it comprises, in replacement of the wild type loop sequence, a segment as defined in this preferred embodiment above.

In a preferred embodiment the variant Cas9 of the invention comprises one or more further fidelity increasing mutation, said mutation(s) is/are selected from a group of mutations present in one or more increased fidelity variant(s) selected from the group consisting of SpCas9-HF1, HypaSpCas9, evoSpCas9, HiFi SpCas9, Sniper SpCas9, eSpCas9, Hypa2SpCas9 and HeFSpCas9 or any fidelity increasing mutation in an increased fidelity Cas9;
highly preferably the mutation is selected from the group consisting of K848A, K1003A, and R1060A.

14. A ribonucleoprotein (RNP) complex comprising the mutant (variant) Cas9 protein of any of paragraphs 1 to 10, said RNP complex also comprising an RNA having a spacer sequence, preferably a crRNA or a single guide RNA (sgRNA).

15. The RNP complex of paragraph 14, said spacer sequence or said crRNA or sgRNA being extended with one or two G nucleotides at its 5′ end.

16. The RNP complex of any of paragraphs 14 to 15, wherein the sgRNA is transcribed intracellularly, in vitro transcribed or custom synthesized and introduced through transfection.

17. An isolated nucleic acid encoding a Cas9 protein as defined in any of paragraphs 1 to 16.

17.1 An isolated nucleic acid according to paragraph 17 comprising a nucleic acid sequence that has at least 40% or at least 50% or preferably at least 60% or preferably at least 70% or preferably at least 80% or at least 90% sequence identity to the following amino acid sequence (SEQ ID NO: 24), said sequence comprising mutation encoding the amino acid mutations as defined in any of paragraphs 1 to 13.

18. A vector comprising the isolated nucleic acid of paragraph 17.

19. The vector of paragraph 18, wherein the isolated nucleic acid is operably linked to one or more nucleic acid(s) coding for regulatory domains for expressing the variant Cas9 protein according to any of paragraphs 1 to 16.

20. A host cell comprising the nucleic acid of paragraph 17 or a vector of any of paragraphs 18 to 19.

21. The host cell according to paragraph 20 wherein said host cell is a mammalian host cell.

22. The host cell according to paragraph 20 or 21, wherein the cell is a stem cell, preferably an embryonic stem cell, a tissue stem cell, e.g. a mesenchymal stem cell, or an induced pluripotent stem cell (iPSC); preferably an animal cell, e.g. a mammalian cell, e.g. a human cell.

23. A kit comprising the isolated nucleic acid of paragraph 17, and/or the vector of any of paragraphs 18 to 19 and/or a host cell of any of paragraphs 20 to 22, and a target specific crRNA or single guide RNA.

24. The kit according to paragraph 23 wherein the target specific crRNA or single guide RNA is a library of crRNAs or sgRNAs.

25. A method of altering the genome or epigenome of a cell, said method comprising

expressing in the cell a Cas9 protein according to any of paragraphs 1 to 10 or contacting the cell with said Cas9 protein, preferably a fusion protein of any of paragraphs 10 to 11,

providing a crRNA having a target specific spacer sequence, preferably a target-specific single guide RNA (sgRNA) having a region complementary to a selected portion of the genome of the cell,

whereby the genome of the cell is altered.

26. The method of paragraph 25 wherein the isolated protein or fusion protein comprises one or more of a nuclear localization sequence, a cell penetrating peptide sequence, and/or an affinity tag.

27. A method of altering a double-stranded DNA (dsDNA) molecule comprising contacting the dsDNA molecule with a protein according to any of paragraphs 1-16 and a crRNA having a target specific spacer sequence, preferably a target-specific single guide RNA (sgRNA) having a region complementary to a selected portion of the dsDNA molecule.

28. The method of paragraph 27, wherein the dsDNA molecule is present in vitro.

Preferably the Cas9 protein of the invention has its activity maintained, preferably the endonuclease activity is maintained on at least one target; in an embodiment at least the DNA binding activity is maintained.

### Definitions

In aspects of the invention the term “single guide RNA” refers to the polynucleotide sequence comprising the guide sequence, the tracr sequence and the tracr mate sequence. The term “guide sequence” refers to the about 20 bp long sequence within the guide RNA that specifies the target site and may be used interchangeably with the terms “guide” or “spacer”. The term “tracr mate sequence” may be used interchangeably with the term “direct repeat(s)”.

As used herein the term “wild type” is a term of the art understood by skilled persons and means the typical form of an organism, strain, gene or characteristic as it occurs in nature as distinguished from mutant or variant forms.

As used herein the term “variant” should be understood as referring to a form having the qualities that have a pattern that deviates from what occurs in nature. In case of a variant protein it has one or more mutations in respect of a wild type sequence, i.e. amino acid deletion(s), insertion(s) or addition(s) and/or substitution(s) in compared to the wild type sequence. In case of a variant nucleic acid it has one or more mutations in respect of a wild type sequence, i.e. nucleotide deletion(s), insertion(s) or addition(s) and/or substitution(s) in compared to the wild type sequence. Preferably said variant is prepared by human interaction.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature. Thus, a variant of a wild type as used herein is necessarily non-naturally occurring.

The term “wild-type” relates to a protein, a nucleic acid or a sequence thereof, including a partial sequence thereof which is the same sequence found in Nature. When any variant of the invention is defined against the wild type background it is to be understood as a comparison to define a sequence or a group or set of sequences deviating form the wild type. Such definition does not exclude further differences in comparison with the wild type which do not interfere with the mutations according to the present invention or allow its effect to be manifested. By such definition, preferably, sequences having the wild type background but comprising the mutation or variation as defined herein, are specifically written and disclosed.

“Complementarity” refers to the ability of a nucleic acid to form hydrogen bonds with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will form hydrogen bonds with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogsteen binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of primer generation reaction (PGR), or the cleavage of a polynucleotide by an enzyme. A sequence capable of hybridizing with a given sequence is referred to as the “complement” of the given sequence.

The terms “polypeptide” and “protein” are used interchangeably herein to refer to polymers of amino acids of any length. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and unnatural or synthetic amino acids. In case of proteins expressed by a living organism the amino acids are preferably protein forming amino acids. As used herein, the one-letter symbols for protein forming amino acid residues are: A (alanine); R (arginine); N (asparagine); D (aspartic acid); C (cysteine); Q (glutamine); E (glutamic acid); G (glycine); H (histidine); I (isoleucine); L (leucine); K (lysine); M (methionine); F (phenylalanine); P (proline); S (serine); T (threonine); V (valine); W (tryptophan); and Y (tyrosine).

As used herein, the term “domain” or “protein domain” refers to a part of a protein that may exist and function separately or independently of the rest of the protein chain. In an embodiment the domain is covalently linked to other parts of the protein and has a well-defined function (functional domain).

“Sequence identity” is related to sequence homology comparisons of sequences which may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. Sequence homologies may be generated by any of a number of computer programs known in the art, for example BLAST or FASTA, etc. Examples of softwares that may perform sequence comparisons include, but are not limited to, the BLAST package (see Ausubel F. M. et al., Short Protocols in Molecular Biology: A Compendium of Methods from Current Protocols in Molecular Biology, John Wiley & Sons, (1999) Short Protocols in Molecular Biology, Chapter 18), FASTA (Altschul et al., 1990) and the GENEWORKS suite of comparison tools. Both BLAST and FASTA are available for offline and online searching (see Ausubel et al., 1999 ibid, pages 7-58 to 7-60). Technology of computing sequence identity is well known in the art.

Sequence homology comparisons of sequences provides a tool to extend the inventive idea to other Cas9 proteins having a loop with the same function as SpCas9 and thus the solution of the invention can be applied thereto. It is contemplated that the invention relates to such variants therefore. The skilled person will understand that selection of such a homologous Cas9 or a variant Cas9 mutated at another site may provide a selection invention and may be non-obvious in view of the present disclosure, even if comprises the solution of the present invention and therefore is covered thereby.

As used herein, a “segment” in a polynucleotide, preferably of a nucleic acid, is a part of the polynucleotide chain consisting of contiguous nucleotide residues, preferably a segment can be considered as an oligonucleotide forming part of a polynucleotide chain. Nucleotide residues of a segment may form a functional unit in a preferred embodiment in a narrower sense.

A segment or a sequence of a Cas9 protein “corresponds” to that of an other Cas9 protein if in a sequence alignment or in a sequence homology comparisons of sequences the two segment or sequence are ordered side by side and found homologous, preferably an N-terminal and a C-terminal part the sequence are ordered side by side irrespective of the sequence between, e.g. which comprises deletion(s) or insertion(s); or the “corresponding” segments or sequences show a level of sequence identity of is at least 50%, 60%, 65%, 70%, 75%, 80%, 85%, 90%, 95%, 97%, 98%, 99%, or 100% over their entire length.

As used herein, a “vector” is a tool that allows or facilitates the transfer of a nucleic acid entity from one environment to another. It is a replicon, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked.

In general, the term “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a spacer sequence (also referred to as a “guide”), or other sequences and transcripts from a CRISPR locus. The crRNA binds the Cas9 protein in a specific manner and comprises the spacer sequence at its 5′ part via which the Cas9 protein, and any molecule bound or linked thereto. Thereby the Cas9 equipped with this spacer sequence or crRNA can target in theory any sequence reflected in this typically 20 or 21 nucleotide length.

A single guide sequence comprises the crRNA and the tracrRNA sequence as well or at least parts thereof which maintain an minimum structure allowing the Cas9 nucleoprotein to work. Typically this is a stem structure formed by a sequence of crRNA origin and a sequence of sgRNA origin being complementary to each other. The two strands of the stem structure is typically or preferably linked by a loop which may have various lengths and is suitable for engineering.

The term “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell.

A “cap” in a Cas9 protein as used herein is a structure formed from amino acids which sterically protrudes and closes the space at the 5′ end of the native spacer sequence thereby hindering the extension of it by one or two nucleotides. In the SpCas9 protein this is formed by a surface loop comprising Glu1007 and Tyr1013 which are most closely situated to the 5′ nucleotide of the spacer sequence so that to be able to form secondary chemical interaction therewith (i.e. are “associated”).

As used herein, a “segment” in a polypeptide, preferably of a protein, is a part of the polypeptide chain consisting of contiguous amino acid residues, preferably a segment can be considered as an oligopeptide forming part of a polypeptide chain. Amino acid residues of a segment may form a functional unit, i.e. the amino acid residues may contribute or several of them may contribute to a function which can be assigned to that segment. For example the segment may form a loop e.g. a surface loop of the polypeptide.

From an other point of view, a loop in the Cas9 protein is a segment of contiguous amino acids which, or at least a part of which is different from a secondary structure, e.g. a structure where the torsion angles are repeated like in an alpha helix and a beta strand.

A loop having a wild type sequence is a segment as defined above which has a sequence which is identical with the corresponding sequence in a wild type Cas9 protein.

A sequence or a segment or a loop in the Cas9 protein is proximal to the space sequence or the 5′ end thereof if there is no other loop structure in the Cas9 structure the closest part of which is closer to one or more 5′ nucleotides of the spacer sequence or, alternatively, wherein one or more amino acids of the proximal loop are in contact with one or more 5′ nucleotide via a secondary bond or steric effect.

A segment between two given (flanking) amino acids or nucleotides in a broader and preferred sense includes said given (flanking) amino acids or nucleotides. In a narrower sense the segment between said (flanking) amino acids or nucleotides does not include said given (flanking) amino acids or nucleotides.

A “composition” is understood herein as a non-naturally occurring composition of matter which comprises at least one biologically active substance as defined herein in an effective amount. Compositions may also comprise further biologically active substances. Furthermore, the compositions may comprise biologically acceptable carriers, formulation agents, excipients etc. which are well known in the art.

“About” and related terms are to be construed herein within the technical context used and in accordance with the usual accuracy and/or tolerance levels of the given field of the art. If not more narrowly understood by a skilled person in a given field, mathematical rules of rounding and the order of magnitude of the given value are to be considered. In case of sequence length about a given value typically refers to a not more than 15% or preferably 10% or in particular 5% variation in the given length of the sequence, for example about 20 is to be understood as a sequence having the length of 17 to 23 monomer units (e.g. amino acids or nucleotides) or preferably 18 to 22 units, in particular 19 to 21 units.

As used herein the singular forms “a”, “an” and if context allows “the” include plural forms as well unless the context dictates otherwise.

The term “comprises” or “comprising” or “including” are to be construed here as having a non-exhaustive meaning and allow the addition or involvement of further features or method steps or components to anything which comprises the listed features or method steps or components.

The expression “consisting essentially of” or “comprising substantially” is to be understood as consisting of mandatory features or method steps or components listed in a list e.g. in a claim whereas allowing to contain additionally other features or method steps or components which do not materially affect the essential characteristics of the use, method, composition or other subject matter. It is to be understood that “comprises” or “comprising” or “including” can be replaced herein by “consisting essentially of” or “comprising substantially” if so required without addition of new matter. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF THE FIGURES

FIG. 1: Structure-guided mutagenesis increases on-target activity of SpCas9-HF1 with 21G-sgRNAs.

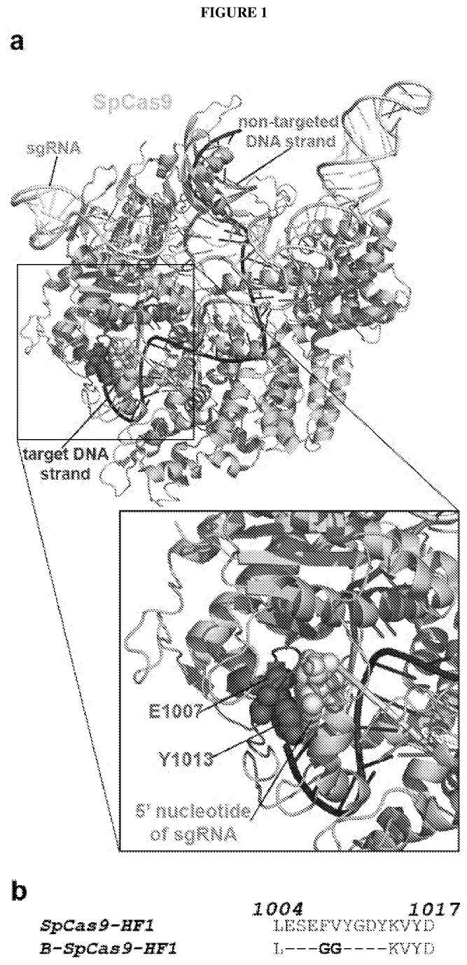

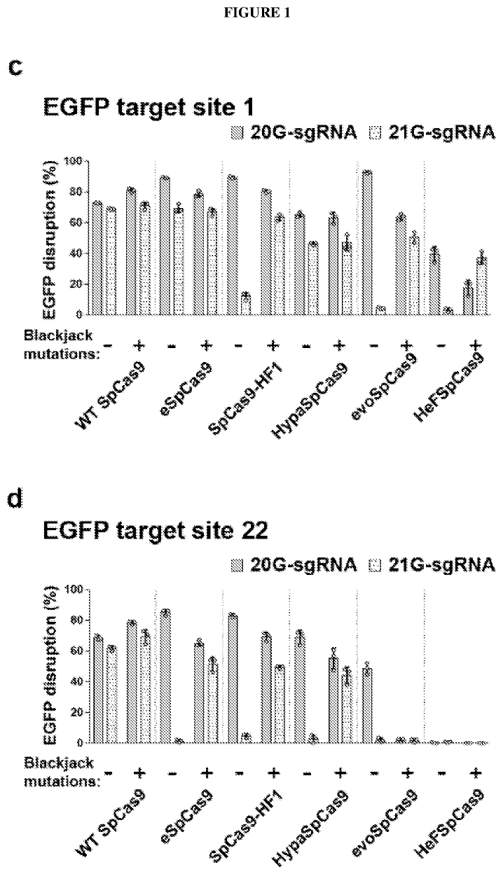

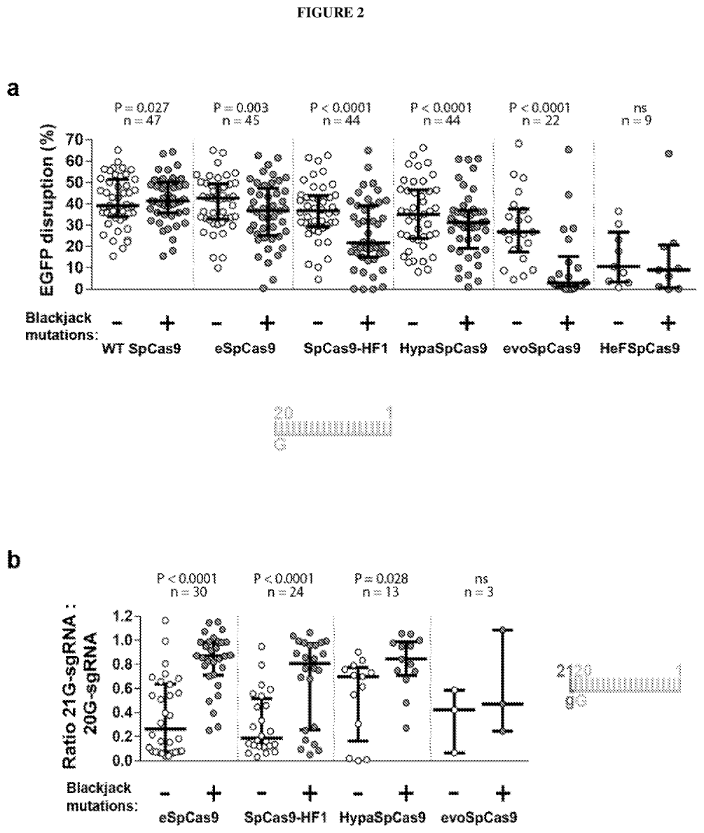

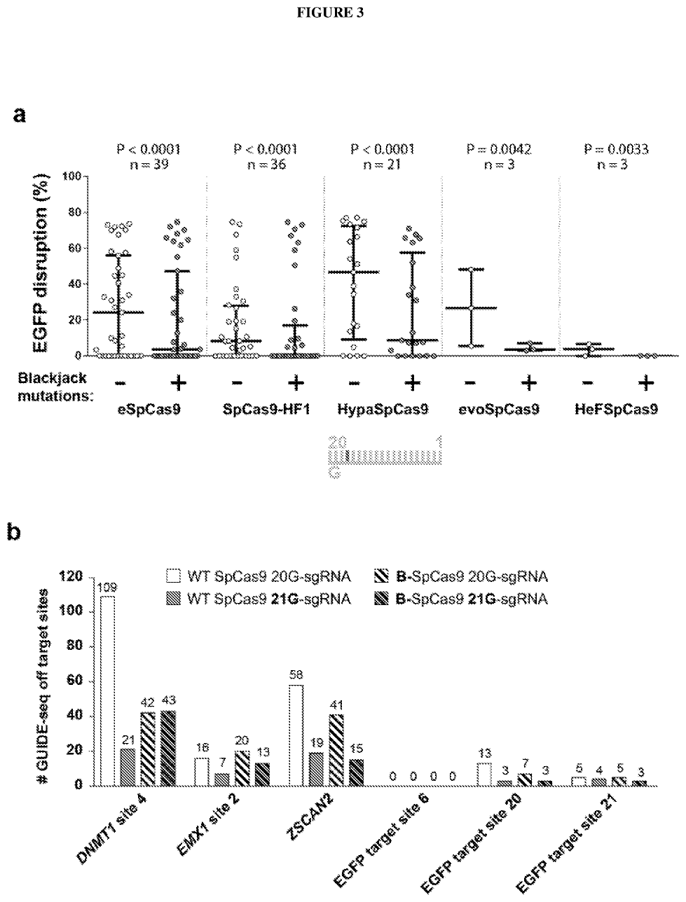

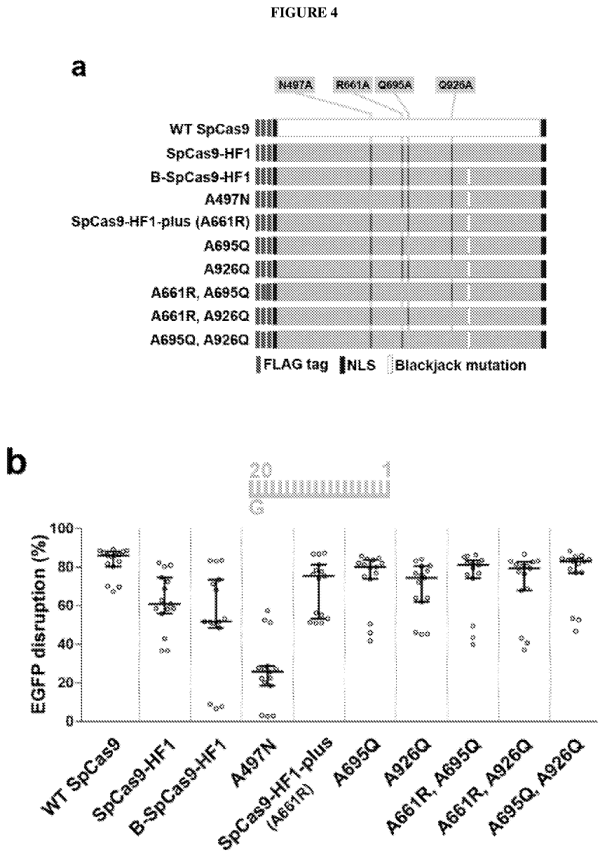

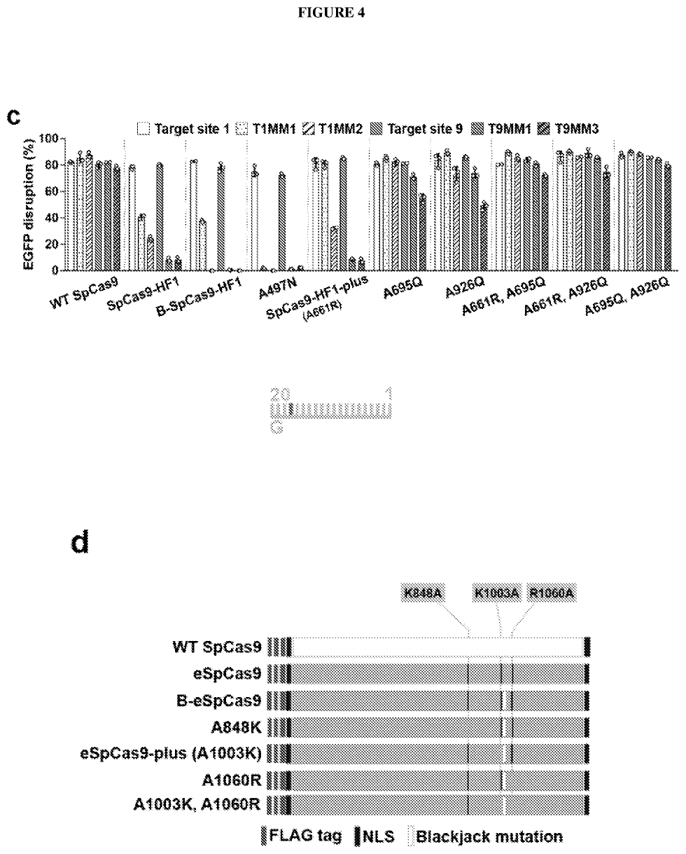

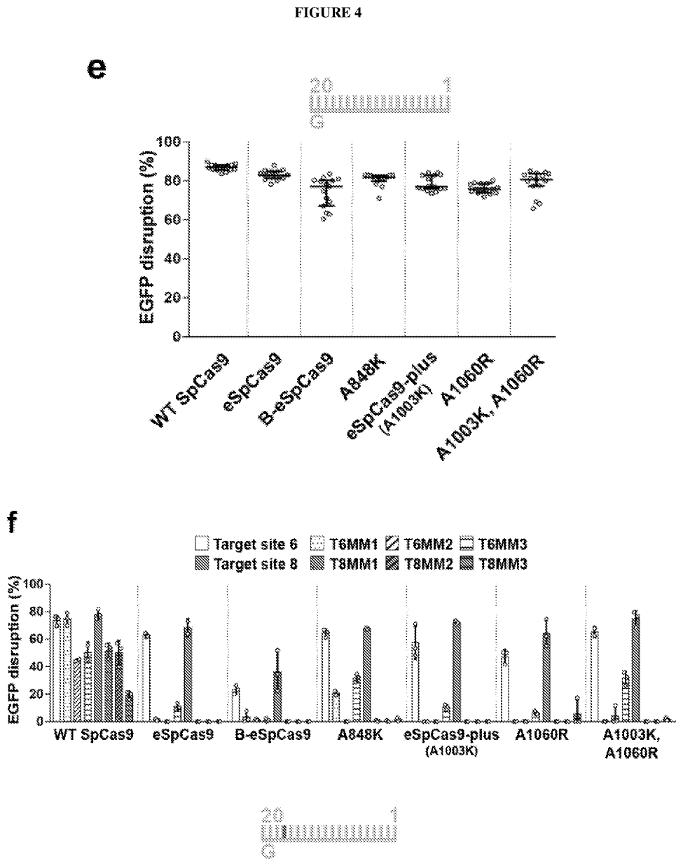

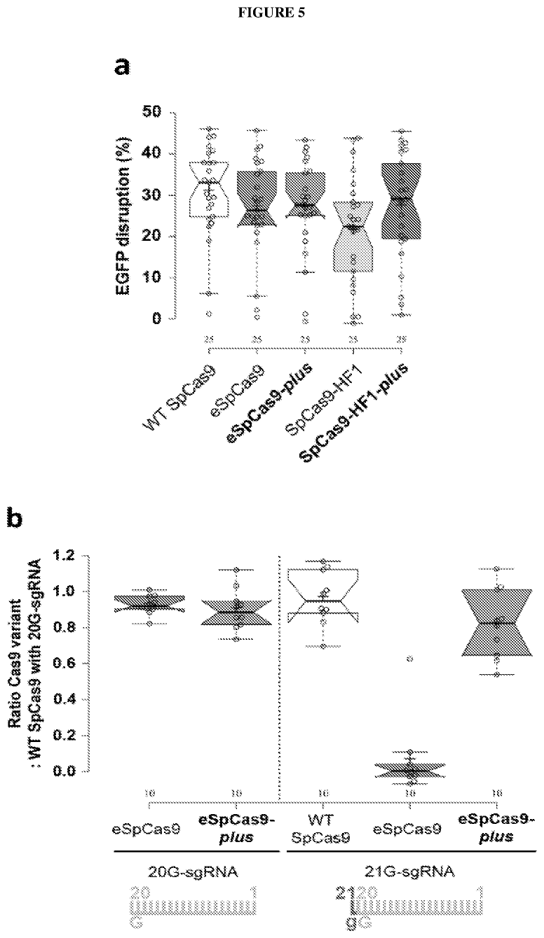

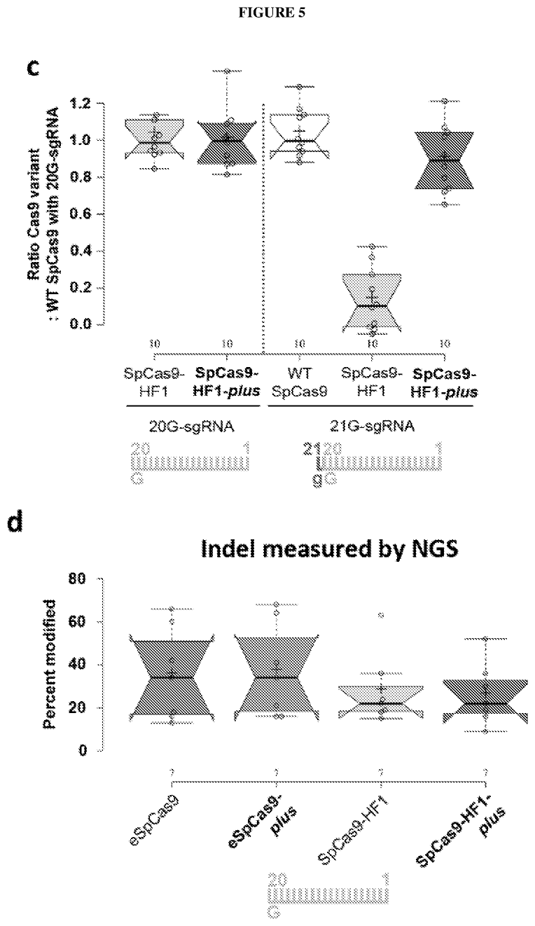

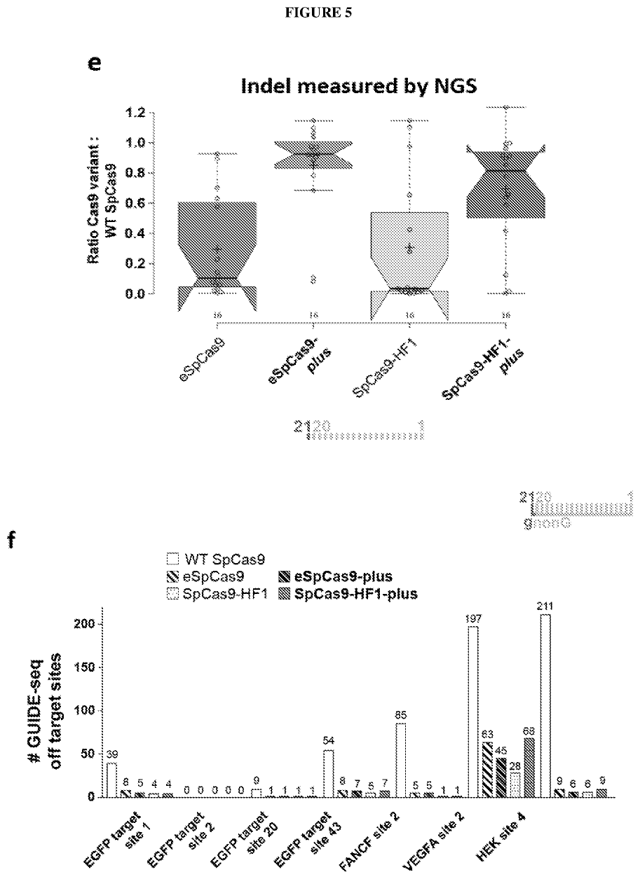

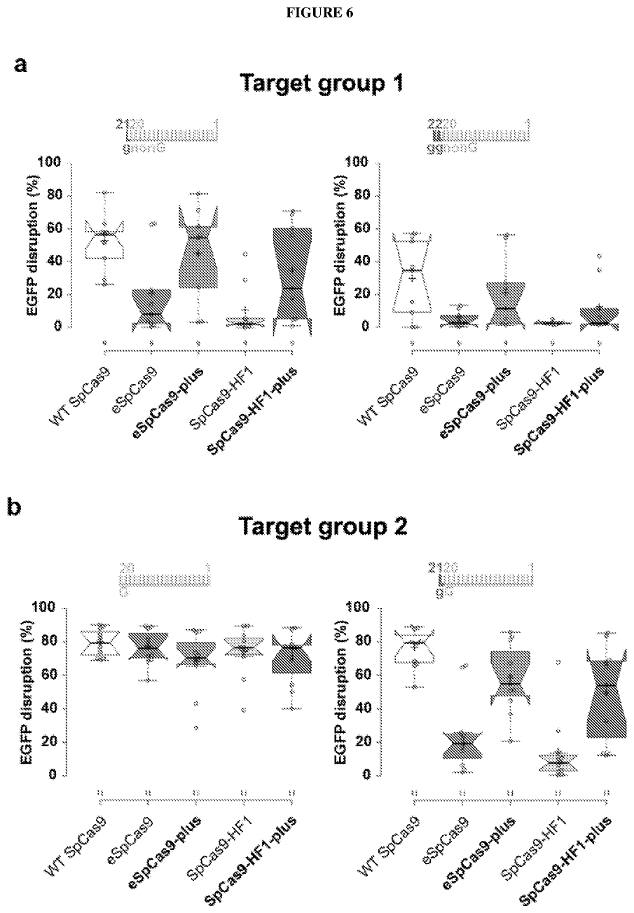

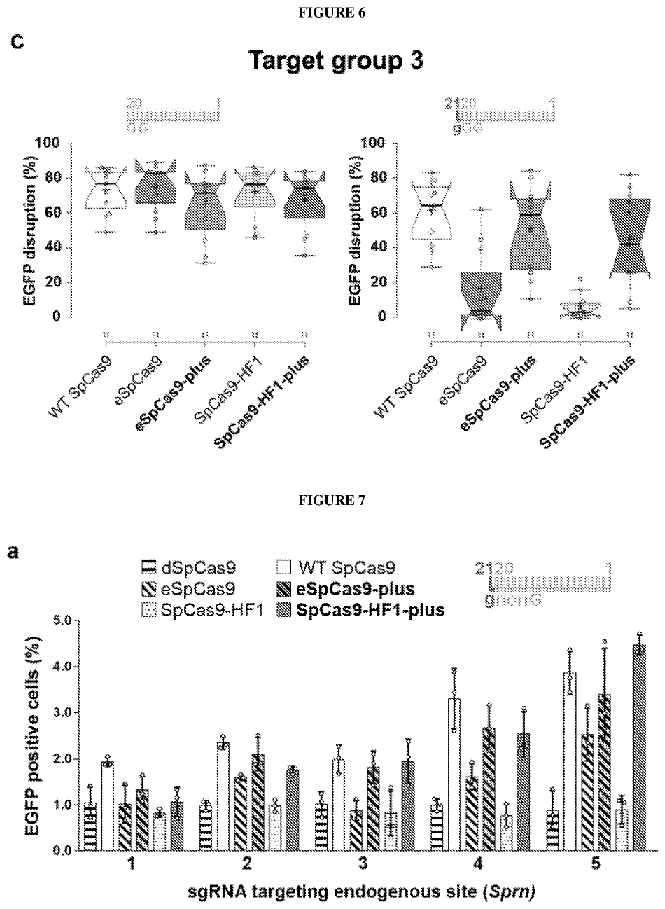

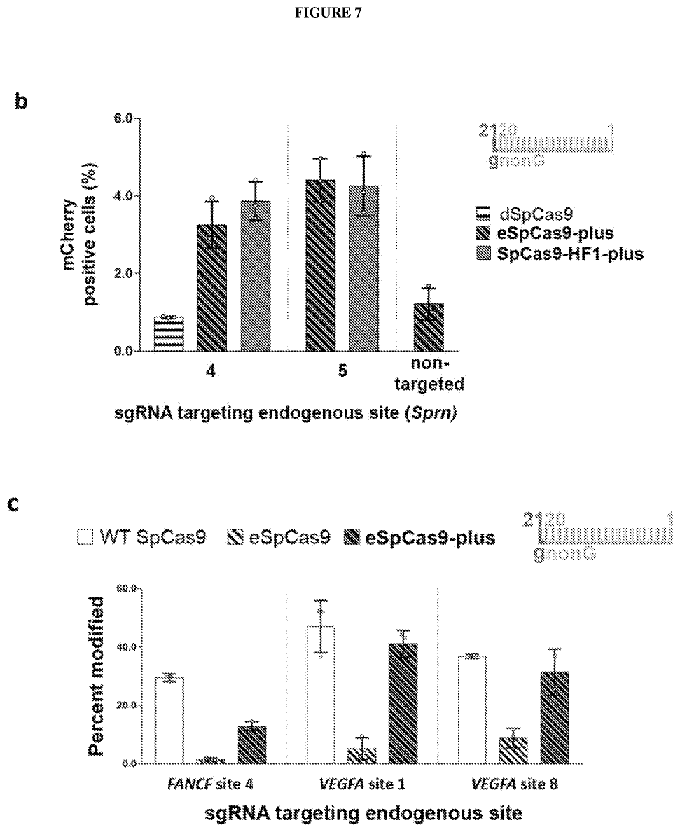

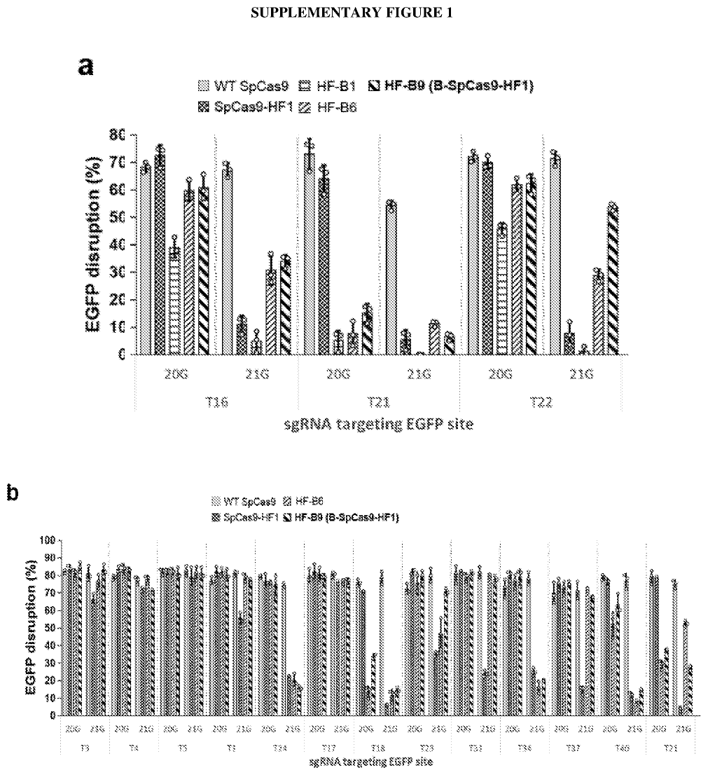

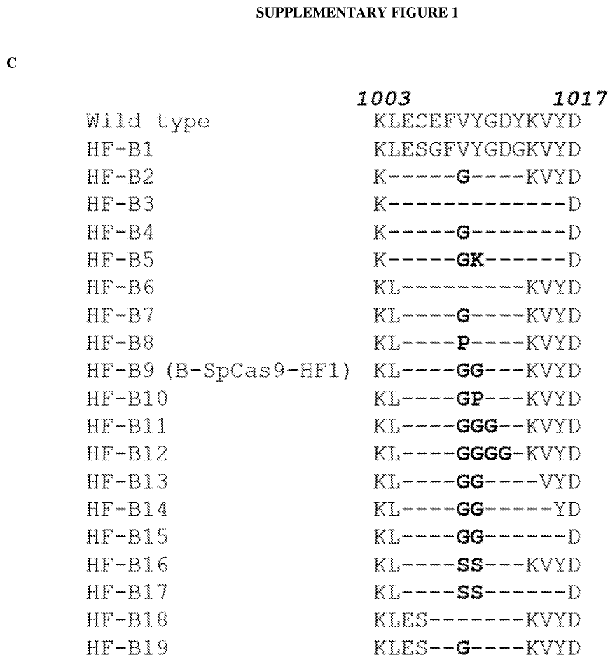

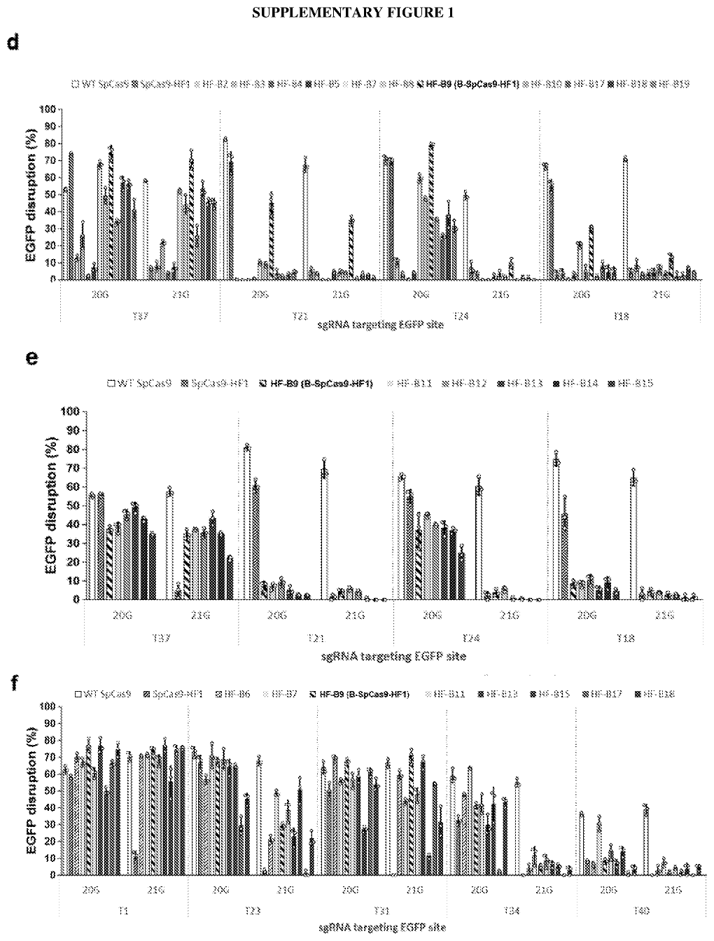

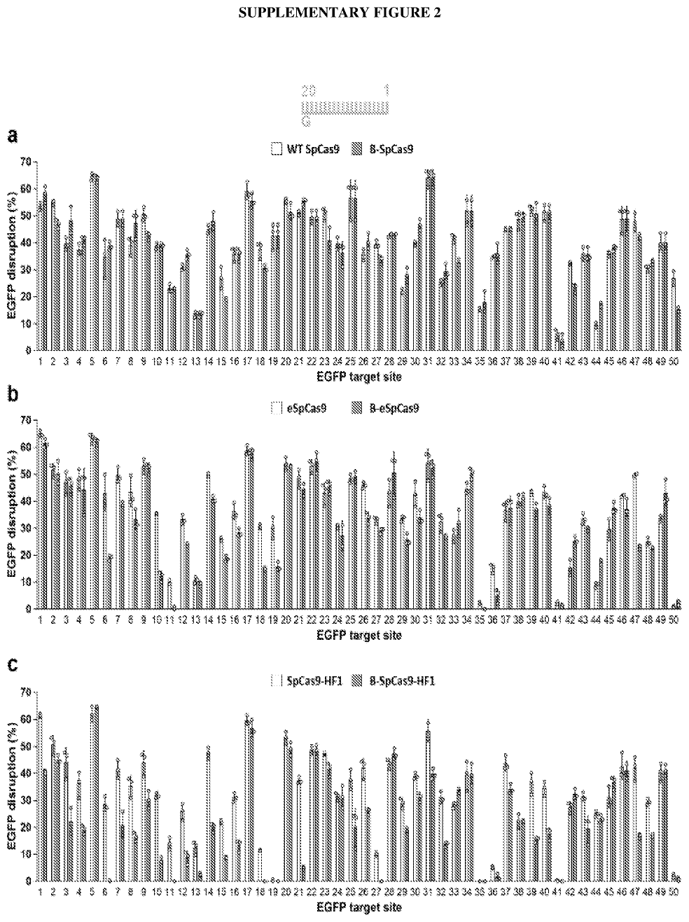

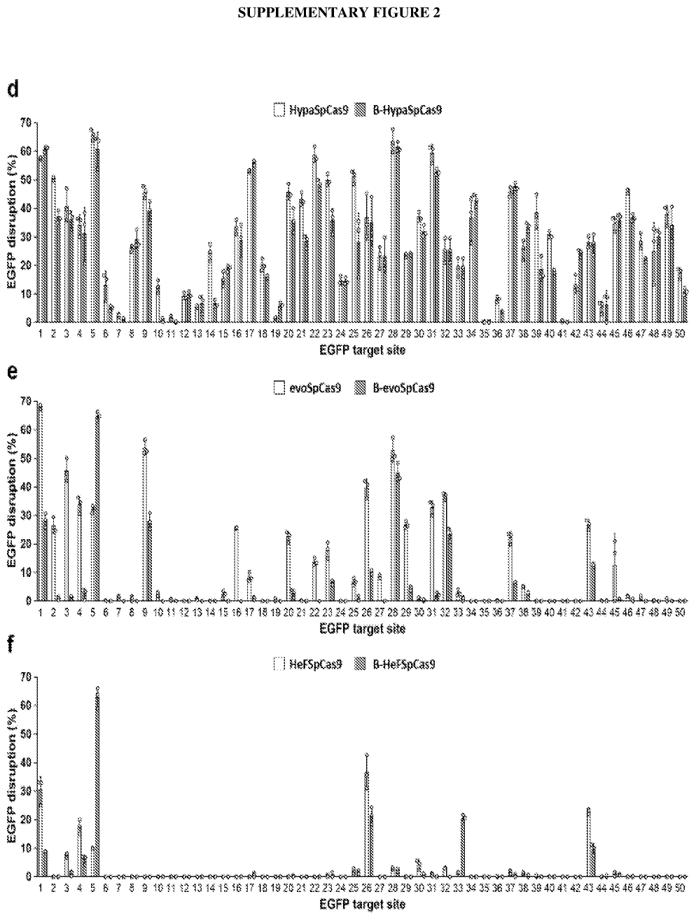

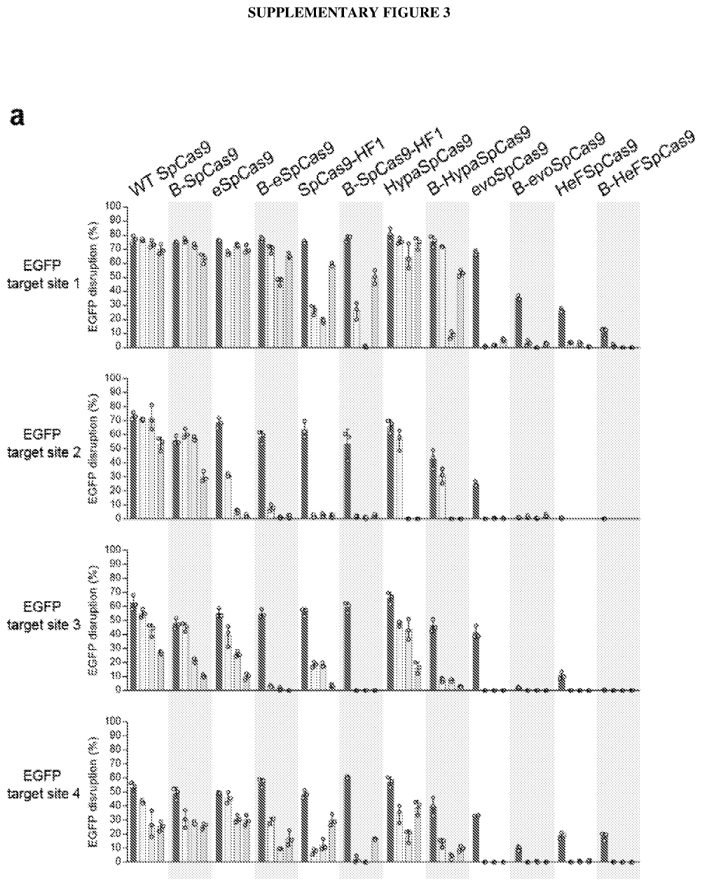

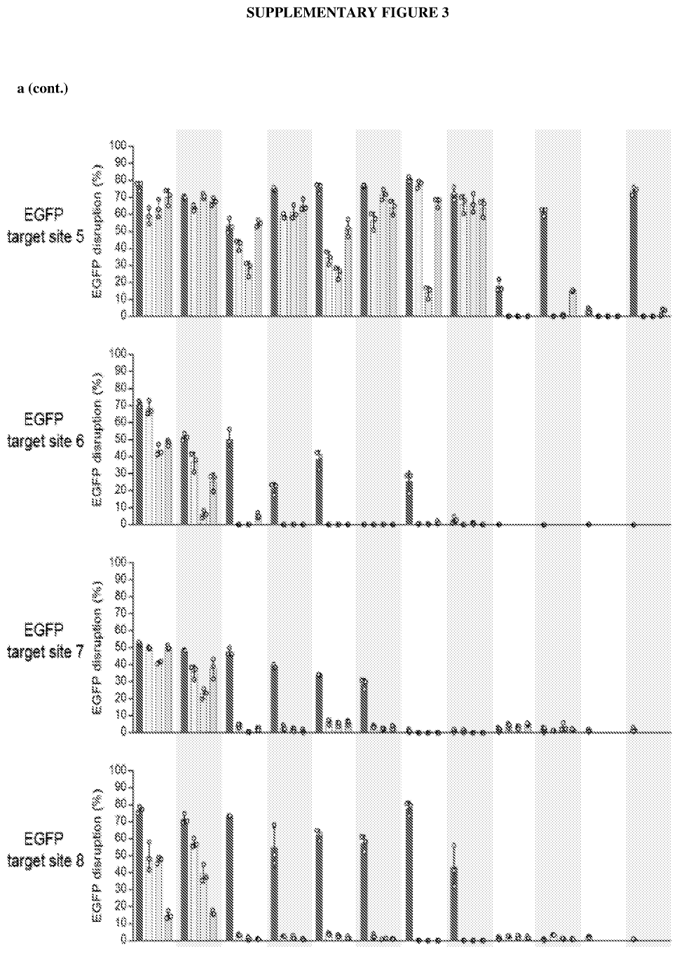

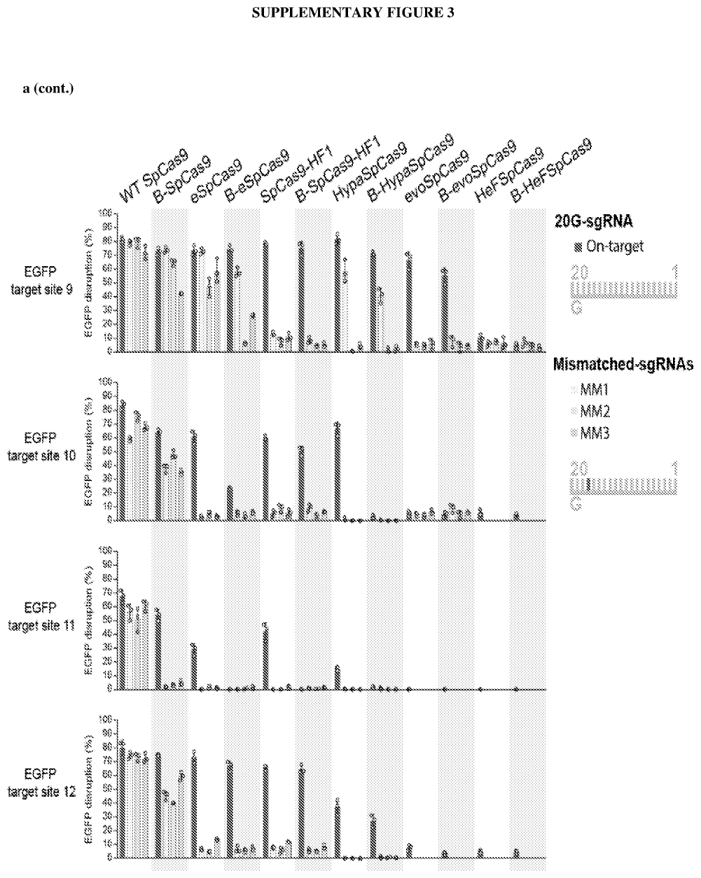

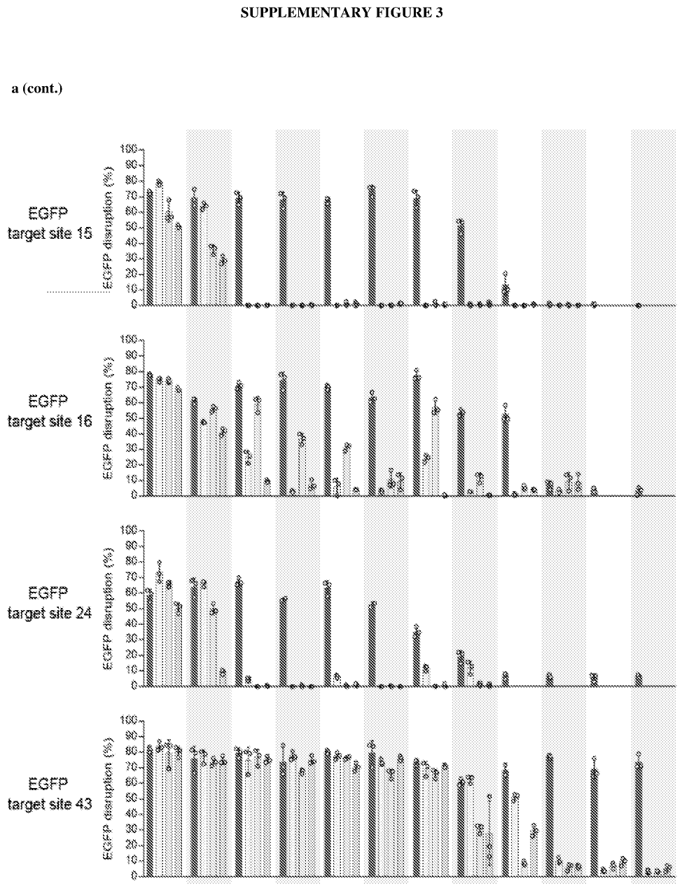

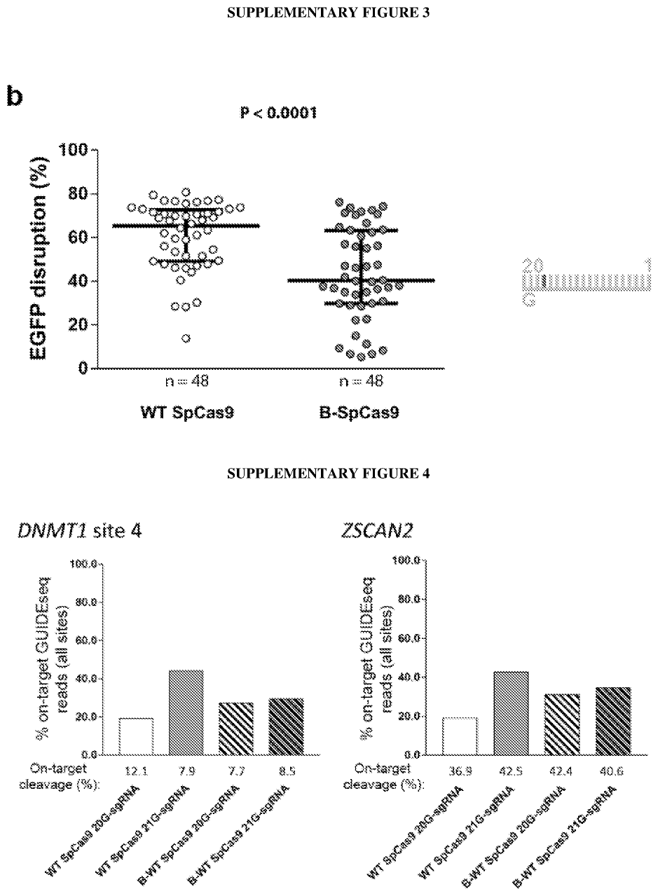

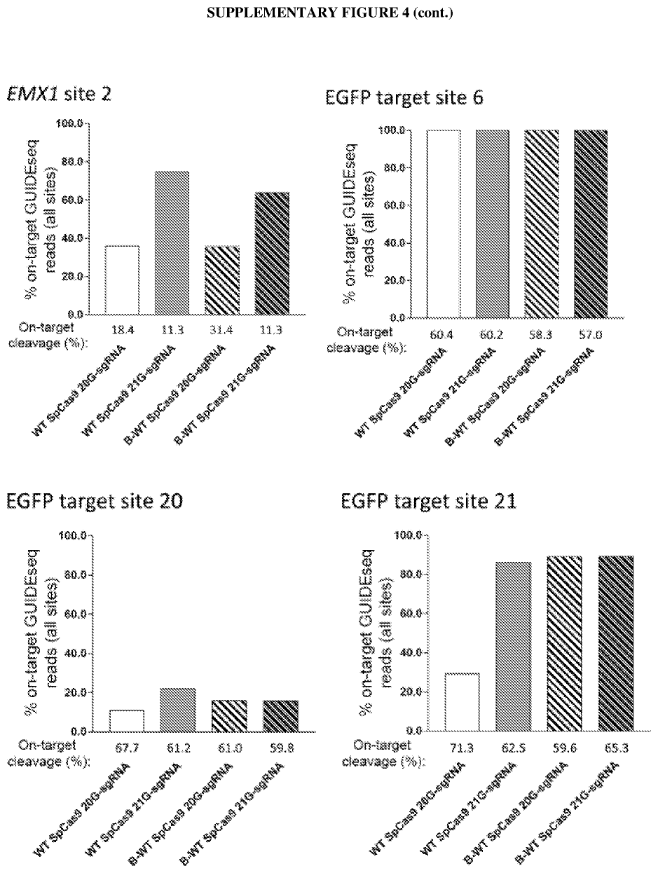

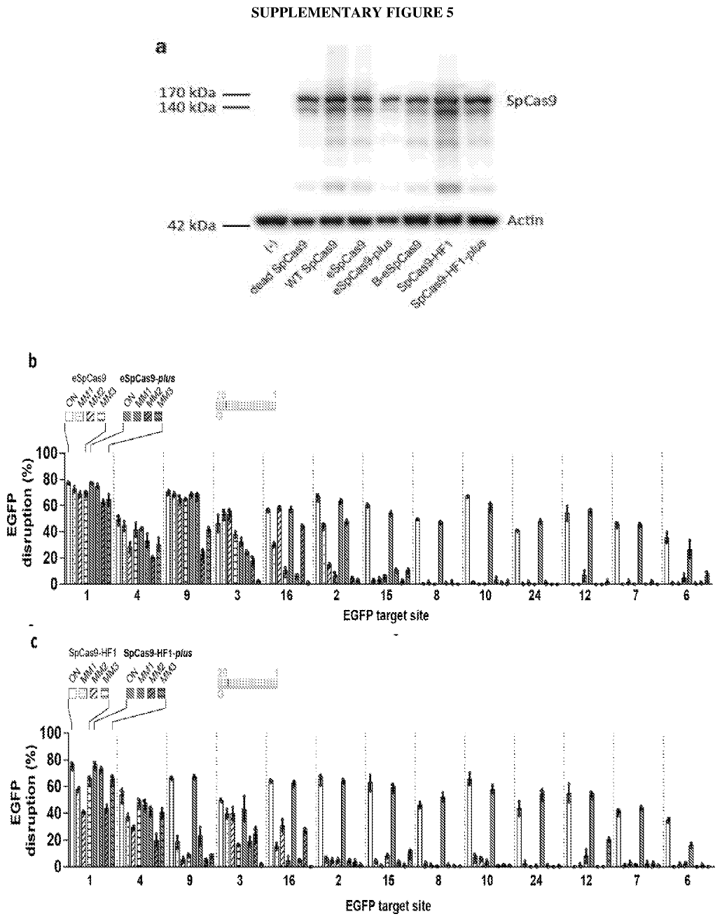

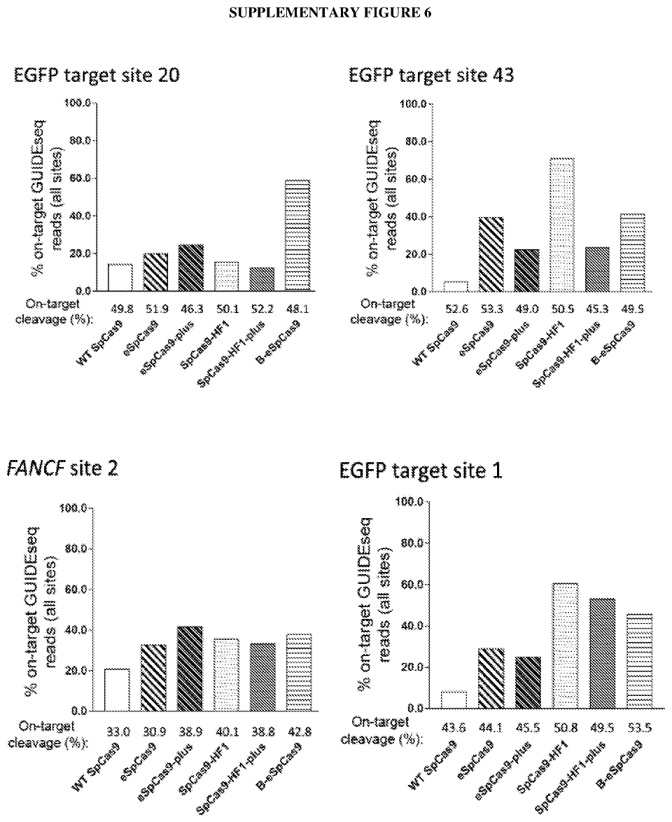

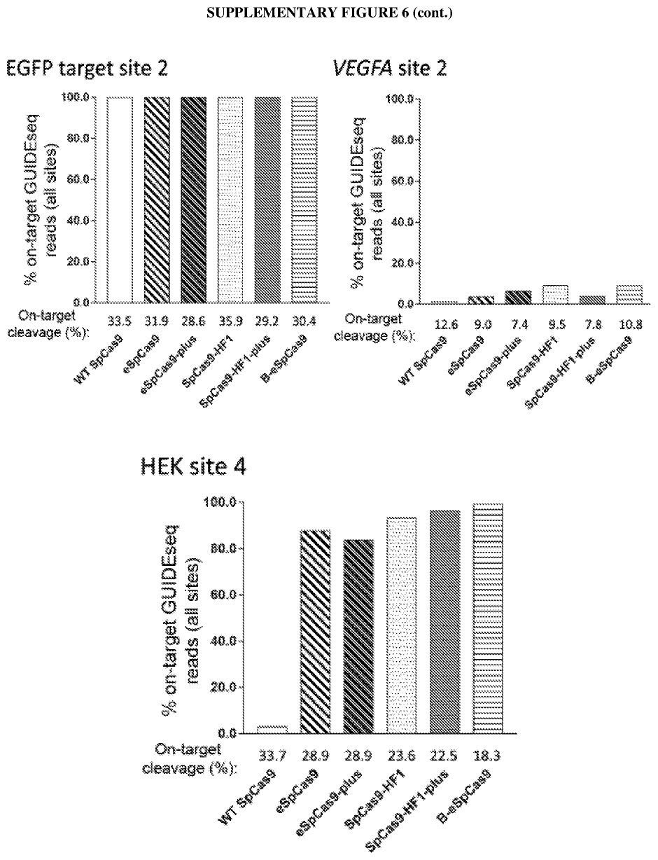

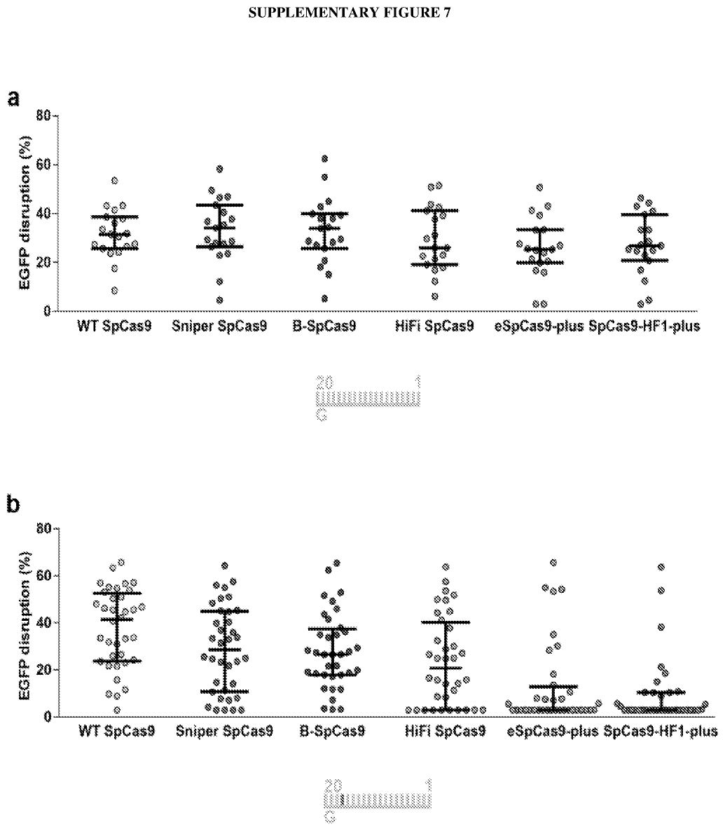

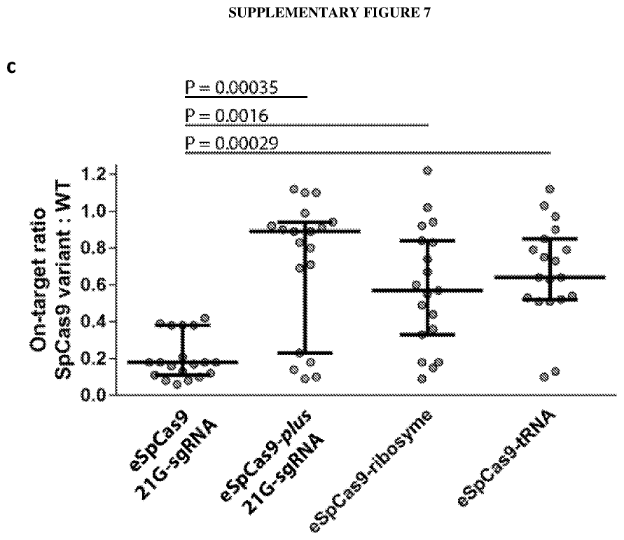

a, X-ray crystallography derived structure of SpCas9-sgRNA-DNA complex in the conformation closest to the cleavage competent state (PDB ID: 5f9r)(Jiang et al., 2016). b, Sequences of SpCas9-HF1 and the selected Blackjack-SpCas9-HF1 at the region affected, between residues L1004 and D1017; deletions (−) and insertions (bold letters) are indicated. See also Supplementary FIG. 1. c, d. Blackjack mutations increase on-target activities of increased fidelity variants with 21G-sgRNAs on different targets. Means are shown, error bars represent the standard deviation (s.d.) for n=3 biologically independent samples (overlaid as white circles).

FIG. 2: The Blackjack mutations increase not only the activity of increased fidelity nucleases charged with 21G-sgRNAs but their target-selectivity in general.

a, Blackjack mutations increase the target-selectivity of their respective parent SpCas9 variants. EGFP disruption activities with perfectly matching 20G-sgRNAs. Results are shown only for those target sites where the SpCas9 variant without Blackjack mutations exhibits higher than background level cleavage. See also Supplementary FIG. 2. b, On-target activities with 21G-sgRNAs on more target sites for which the SpCas9 variant with Blackjack mutations using 20G-sgRNAs exhibits at least 70% on-target activity compared to WT SpCas9. The ratio of the activities for 21G-sgRNA and for 20G-sgRNA are shown for increased fidelity variants eSpCas9, SpCas9-HF1, HypaSpCas9, evoSpCas9 with and without Blackjack mutation. (a-b) The median and the interquartile range are shown; data points are plotted as open circles representing the mean of biologically independent triplicates. Spacers are schematically depicted beside the charts as combs: light grey color teeth indicate matching, while dark grey color tooth indicates the presence of an appended nucleotide within the spacer; numbering of tooth position corresponds to the distance of the nucleotide from the PAM; the starting 20th nucleotide of the spacer is indicated as capital and an appended 21st nucleotide as a dark grey lowercase letter. Statistical significance was assessed using two-sided Paired-samples Student's t-test or two-sided Wilcoxon signed ranks test as appropriate; ns: not significant.

FIG. 3: The Blackjack mutations increase the fidelity of increased fidelity nucleases

a, Blackjack mutations increase the fidelity of their respective parent SpCas9 variants. EGFP disruption activities with partially mismatching 20G-sgRNAs. Results are shown only for those target sites where both the non-Blackjack parent- and Blackjack-SpCas9 variant exhibits at least 70% on-target activity (with perfectly matching 20G-sgRNA) compared to WT SpCas9. Only one target (with three different mismatched positions) matches this condition in the case of evo- or HeFSpCas9. The median and the interquartile range are shown; data points are plotted as open circles representing the mean of biologically independent triplicates. Spacers are schematically depicted beside the charts as combs: light grey color teeth indicate matching, while dark grey color teeth indicates the presence of a mismatching nucleotide (not necessarily the exact position) within the spacer; numbering of the tooth positions corresponds to the distance of the nucleotide from the PAM; the starting 20^{th} nucleotide of the spacer is indicated by an uppercase letter. Statistical significance was assessed using two-sided Paired-samples Student's t-test or two-sided Wilcoxon signed ranks test as appropriate; ns: not significant. See also Supplementary FIG. 3. b, Bar chart of the total number of off-target sites detected by GUIDE-seq for WT and B-SpCas9 variants on six target sites targeted with 20G- or 21G-sgRNAs. See also Supplementary FIG. 4.

FIG. 4: Restoring mutations to wild type amino acids lowers the (on-)target-selectivity and fidelity of B-SpCas9-HF1 and B-eSpCas9.

a, d, Schematic representation of the mutations in each variant of B-SpCas9-HF1 and B-eSpCas9 examined, respectively. b, e, On-target activities using 20G-sgRNAs measured on 5 target sites (n=3 biologically independent samples [overlaid as white circles]), employing EGFP disruption assay, median and interquartile range are shown. c, f, Mismatch screen results from EGFP disruption assay. Target sites and matching (e.g., T1, T6) or mismatching sgRNAs (e.g., T1MM1, T6MM1) are the same as in Supplementary FIG. 3. Schematics highlighting the main features of the spacers used are added to the charts. Means are shown, error bars represent the standard deviation (s.d.) for n=3 biologically independent samples (overlaid as white circles). Spacers are schematically depicted beside the charts as combs: light grey color teeth indicate matching, while a dark grey color tooth indicates the presence of a mismatching nucleotide (not necessarily the exact position) within the spacer; numbering of the tooth positions corresponds to the distance of the nucleotide from the PAM; the starting 20^{th} nucleotide of the spacer is indicated by an uppercase letter. Means are shown, error bars represent the standard deviation (s.d.) for n=3 biologically independent samples (overlaid as white circles).

FIG. 5: eSpCas9-plus and SpCas9-HF1-plus show greatly enhanced on-target activity with 21G-sgRNAs and identical fidelity/target-selectivity compared to eSpCas9 and SpCas9-HF1, respectively, as assessed by EGFP disruption and, indel measured by NGS and GUIDE-seq.

a-c, EGFP disruption activity a, with 20G-sgRNAs targeting 25 sites; b, c, with either 20G- or 21G-sgRNA pairs targeting two alternative sets of 10 different sequences shown as the ratio of variant activity to WT activity. d, e On-target activities of SpCas9 variants across 23 endogenous target sites within the human VEGFA or FANCF loci targeted with d, 20G- or e, 21G-sgRNAs, measured by amplicon sequencing. e, On-target activities of SpCas9 variants across 16 endogenous target sites within the human VEGFA or FANCF loci targeted with 21G-sgRNAs measured by amplicon sequencing. f, Bar chart of the total number of off-target sites detected by GUIDE-seq for SpCas9 variants on seven sites targeted with 20G-sgRNAs. a-e, Tukey-type boxplots by BoxPlotR (Spitzer et al., 2014): center lines show the medians; box limits indicate the 25^{th} and 75^{th} percentiles; whiskers extend to the “minimum” and “maximum” data situated within 1.5 times the interquartile range from the 25^{th} and 75^{th} percentiles, respectively; notches indicate the 95% confidence intervals for the medians; crosses represent sample means; data points are plotted as open circles representing the mean of biologically independent triplicates. Spacers are schematically depicted beside the charts as combs: light grey color teeth indicate matching, while a dark grey color tooth indicates the presence of an appended nucleotide within the spacer; numbering of tooth position corresponds to the distance of the nucleotide from the PAM; the starting 20^{th} nucleotide of the spacer is indicated by an uppercase letter and an appended 21^{st} nucleotide by a dark grey lowercase letter. See also Supplementary FIGS. 5 and 6.

FIG. 6: The plus variants are effective when transfected as preassembled RNP form.

a-c, EGFP disruption assays. Target sequences start with 5′ non-G-, G- or GG-nucleotides. Tukey-type boxplots by BoxPlotR (Spitzer et al., 2014): center lines show the medians; box limits indicate the 25^{th} and 75^{th} percentiles; whiskers extend to the “minimum” and “maximum” data situated within 1.5 times the interquartile range from the 25^{th} and 75^{th} percentiles, respectively; notches indicate the 95% confidence intervals for the medians; crosses represent sample means; data points are plotted as open circles representing the mean of biologically independent triplicates. Spacers are schematically depicted beside the charts as combs: light grey teeth indicate matching, while a dark grey color tooth indicates the presence of an appended nucleotide within the spacer; numbering of tooth position corresponds to the distance of the nucleotide from the PAM; the starting 20^{th} nucleotide or dinucleotide of the spacer is indicated by an uppercase letter and an appended 21^{st} and 22^{nd} nucleotides by dark grey lowercase letters. See also Supplementary FIG. 6.

FIG. 7: Blackjack variants facilitate modification at the 5′ coding region of the endogenous Shadoo (Sprn) gene.

a, Pre-screening targets with increased fidelity nucleases for efficiency by the integration of a donor EGFP cassette. b, Based on (a), the SpCas9-plus variants were selected to generate transgenic lines using the ‘self-cleaving’ EGFP-expression plasmid, which must integrate in-frame for Sprn promoter driven EGFP expression, and downstream from the EGFP coding sequence it also contains a CMV-mCherry cassette; mCherry positive cells were counted. c, Indel formation activity of eSpCas9-plus compared to WT and eSpCas9 with 21G-sgRNAs transcribed from an integrated single copy of lentiviruses measured by TIDE. a-c, Means are shown, error bars represent the standard deviation (s.d.) for n=3 biologically independent samples (overlaid as white circles). In the case of VEGFA site 8 targeted with WT and eSpCas9-plus on (c) one sample point is missing due to sample loss. Spacers are schematically depicted beside the charts as combs: light grey teeth indicate matching, while a dark grey tooth indicates the presence of an appended nucleotide within the spacer; numbering of tooth position corresponds to the distance of the nucleotide from the PAM; the starting 20^{th} nucleotide of the spacer is indicated by an uppercase letter and an appended 21^{st} nucleotide by a dark grey lowercase letter.

Supplementary FIG. 1: Related to FIG. 1

Screening of Candidate Mutant Nucleases to Identify the Optimal “Blackjack” Variant.

a, b, d-h, EGFP disruption activities of WT SpCas9, SpCas9-HF1 and Blackjack mutant SpCas9-HF1 candidates programmed with perfectly matching 20G-sgRNAs or 5′-extended 21G-sgRNAs on various EGFP target sites (labelled as e.g. T37, T1). Means are shown, error bars represent the standard deviation (s.d.) for n=3 biologically independent samples (overlaid as white circles). c, Amino acid sequences between residues 1003 and 1017 of the SpCas9-HF1 and Blackjack SpCas9-HF1 candidates examined. Deletions (−), insertions (bold letters) and substitutions (underlined letters) are indicated.

Supplementary FIG. 2: Related to FIG. 2a

Blackjack Mutations Increase Target-Selectivity of Increased Fidelity SpCas9 Nuclease Variants.

Increased fidelity nucleases and their Blackjack variants on individual targets, when programmed with perfectly matching 20G-sgRNAs. Where the HypaSpCas9 data are derived from different experiments the values are normalized to the corresponding WT data. Spacers are schematically depicted over the charts as combs: light grey color teeth indicate matching nucleotides; numbering of tooth position corresponds to the distance of the nucleotide from the PAM; starting 20^{th} G nucleotide of the spacer is indicated. Means are shown, error bars represent the standard deviation (s.d.) for n=3 biologically independent samples (overlaid as white circles).

Supplementary FIG. 3: Related to FIG. 3a

Blackjack Mutations Further Increase the Fidelity of SpCas9 Nuclease Variants as Well as that of the WT SpCas9.

a, EGFP disruption activities of the SpCas9 nucleases and their Blackjack variants programmed with perfectly matching or partially mismatching 20G-sgRNAs [e.g. MM1 on EGFP target site 43 corresponds to a mixture of three sgRNAs mismatched at the same position (Kulcsar et al., 2017)] on EGFP target sites. Where the HypaSpCas9 data are derived from different experiments the values are normalized to the corresponding WT data. Means are shown, error bars represent the standard deviation (s.d.) for n=3 biologically independent samples (overlaid as white circles). b, Blackjack mutations increase the fidelity of WT SpCas9. The sample points correspond to mismatched positions presented in Supplementary FIG. 3a. The median and the interquartile range are shown; data points are plotted as open circles representing the mean of biologically independent triplicates. Statistical significance was assessed by two-sided Wilcoxon signed ranks test. a-b, Spacers are schematically depicted beside the charts as combs: light grey color teeth indicate matching, while dark grey color teeth indicate the presence of a mismatching nucleotide within the spacer (not necessarily the exact position shown in the comb); numbering of tooth position corresponds to the distance of the nucleotide from the PAM; the starting 20^{th} nucleotide of the spacer is indicated by an uppercase letter.

Supplementary FIG. 4: Related to FIG. 3b

Both 5′ G-Extended sgRNAs and Blackjack Mutations Increase the Fidelity of WT SpCas9 as Assessed by GUIDE-Seq.

Off-target cleavage sites of SpCas9 variants (Blackjack-WT and WT SpCas9) targeted either with 20G- or 21G-sgRNAs identified by GUIDE-seq. Specificity presented as the percentages of on-target reads per all reads captured by GUIDE-seq with the given sgRNAs. On-target cleavage activities were measured either by TIDE (DNMT1 site 4, ZSCAN2, EMX1 site 2) or flow cytometry (EGFP target site 6, 20 and 21) and are shown under the column charts.

Supplementary FIG. 5: Related to FIG. 5

The Plus SpCas9 Variants Exhibit Fidelity Identical to their Respective Non-Blackjack Parent Nuclease Variants eSpCas9 and SpCas9-HF1, but Greatly Increased On-Target Activities with 21G-sgRNA.

a, Immunoblot analysis of the expression levels of SpCas9 nuclease variants (˜160 kDa) in cell lysates of reporter N2a.dd-EGFP cells transfected with the indicated nuclease constructs. β-actin (˜42 kDa) was used as a control for total protein amounts analyzed. b-c, EGFP disruption activities of SpCas9 nuclease variants programmed with 20G-, perfectly matching (ON) or partially mismatching (MM1-3) sgRNAs, targeting EGFP target sites (labelled as e.g. T37, T1). 16 of 23 sites were targeted with 21G-sgRNAs (20^{th} position non-G), 7 of 23 were targeted with perfectly matching 20G-sgRNAs (detailed target information in Table 5). Data are the same as those shown in FIG. 5d, e as a boxplot. b-c, Spacers are schematically depicted beside the charts as combs: light grey teeth indicate matching, while dark grey teeth indicate the presence of an appended nucleotide within the spacer; numbering of tooth position corresponds to the distance of the nucleotide from the PAM; the starting 20^{th} nucleotide of the spacer is indicated by an uppercase letter and an appended 21^{st} nucleotide by a dark grey lowercase letter. Means are shown, error bars represent the standard deviation (s.d.) for n=3 biologically independent samples (overlaid as white circles).

Supplementary FIG. 6: Related to FIG. 5f

The Plus SpCas9 Variants Exhibit Fidelity Identical to their Respective Non-Blackjack Nuclease Variants eSpCas9 and SpCas9-HF1, as Assessed by GUIDE-Seq.

Off-target cleavage sites of SpCas9 variants identified by GUIDE-seq. Seven sgRNAs targeted to either endogenous human genes or EGFP target sites. Specificity presented as the percentages of on-target reads per all reads captured by GUIDE-seq with the given sgRNAs. On-target cleavage activities were measured either by TIDE (FANCF site 2, VEGFA site 2, HEK site 4) or flow cytometry (EGFP target site 1, 2, 20 and 43) and are shown under the column charts.

Supplementary FIG. 7: Related to FIG. 6a-c

Characterization of Sniper- and HiFi-SpCas9, Ribozyme-sgRNA and tRNA-sgRNA Fusion Approaches and Use of Blackjack Variants in RNP Form.

a-b, EGFP disruption activities of WT, Sniper, B-SpCas9, HiFi and eSpCas9-plus and SpCas9-HF1-plus nucleases. a, On-target activities of SpCas9 variants programmed with 20G-sgRNAs. The median and interquartile range are shown; data points are plotted as open circles representing the mean of biologically independent triplicates. b, EGFP disruption activities with partially mismatching 20G-sgRNAs. Results are shown only for those target sites where all SpCas9 variants exhibit at least 70% on-target activity (with perfectly matching 20G-sgRNA) compared to WT SpCas9. The median and the interquartile ranges are shown; data points are plotted as open circles representing the mean of biologically independent triplicates. Where the data are derived from different experiments the values are normalized to the corresponding WT data. a-b, Spacers are schematically depicted beside the charts as combs: light grey color teeth indicate matching, while dark grey color teeth indicate the presence of a mismatching nucleotide within the spacer; numbering of the tooth positions corresponds to the distance of the nucleotide from the PAM; the starting 20^{th} nucleotide of the spacer is indicated as capital. c, EGFP disruption activities of SpCas9 nuclease variants programmed with 5′ extended 21G-sgRNAs, hammerhead ribozyme flanked-sgRNAs (Kim et al., 2017) or rice tRNA flanked-sgRNAs (Dong et al., 2017), targeting 19 EGFP target sites (20^{th} position non-G) shown normalized to WT SpCas9. Two target sequences (EGFP target site 55 and 70) not cleaved to an assessable extent by the WT SpCas9 are not included. The median and the interquartile range are shown; data points are plotted as open circles representing the mean of biologically independent triplicates. Statistical significance was assessed by two-sided Wilcoxon signed rank test.

### DETAILED DESCRIPTION OF THE INVENTION

In an effort to generate an increased fidelity SpCas9 nucleases, the present inventors have found that an 5′ G-extension of sgRNAs affects, i.e. reduces the activity of known increased fidelity variants and have surprisingly discovered that while this limitation or reduction of the activity may result from a capping of the 5′ end of the sgRNA by amino acids which are connected via a surface loop as revealed by some newer X-ray structures of SpCas9 nuclease (FIG. 1a) (Jiang et al., 2016; Nishimasu et al., 2014), appropriate mutations can counter-act this limitation. Such amino acids in the SpCas9 structure are in particular Glu1007 and Tyr1013. Thus, the present inventors variants have prepared variant Cas9 proteins with mutations that alter the interaction of Glu1007 and Tyr1013 with the sgRNA.

Thereby the cap has been removed by the mutations to make space for a 5′ G-extension of the sgRNA without clashing with the polypeptide chain. It has been found that it could be achieved without disrupting the structural features of the folded protein. Such modification allows the increased fidelity nucleases to work with similar efficiency when charged with sgRNAs containing either 20- or 21-nucleotide-long spacers (20G-sgRNA or 21G-sgRNA), thereby extending their target space to non-20G targets without losing fidelity. It has also been surprisingly found that the removal of the cap had another effect as well, namely that it increases the fidelity of the nucleases and transform the WT protein to an increased fidelity nuclease that tolerate a 5′ extension of the sgRNA.

As preferred examples, the most effective mutations were named “Blackjack”, which increase the fidelity of WT SpCas9, hereafter, Blackjack SpCas9 (B-SpCas9) while keeping it effective with 21G-sgRNAs. When combined with various other fidelity-increasing mutations, Blackjack mutations cause essentially the same effect in every case (increase fidelity while making it effective with 21G-sgRNAs). Named according to the other increased fidelity variants, the inventors have generated B-eSpCas9, B-SpCas9-HF1, B-HypaSpCas9, B-evoSpCas9, B-Hypa2SpCas9 and B-HeFSpCas9. Two further “Blackjack” variants, eSpCas9-plus and SpCas9-HF1-plus have also been developed that are further improved variants of eSpCas9 and SpCas9-HF1, respectively, possessing matching on-target activity and fidelity but retaining 20G-level activity with 21G-sgRNAs. These variants of the invention facilitate the use of the existing pooled sgRNA libraries with higher specificity and show similar activities when delivered either as plasmids or as pre-assembled ribonucleoproteins.

Thus, the invention also provides a method to tune increased fidelity Cas9 proteins, or to prepare a tuned fidelity Cas9, by introducing the mutations according to the invention into the Cas9 protein structure wherein a given selected other fidelity increasing mutation is carried out or introduced in order to obtain an engineered variant Cas9 protein having the desired activity and increased fidelity. In another embodiment in the method to tune the increased fidelity Cas9 proteins in an increased fidelity Cas9 protein one or more of the mutation(s) is/are reversed into the wild type amino acid whereas a mutation according to the invention is introduced into said tuned fidelity Cas9 mutant. Examples for such engineered variant Cas9 proteins of the invention are the—plus variants.

Some aspects of the present disclosure provide strategies, systems, reagents, methods and kits that are useful for targeted nucleic acid editing, such as editing a single site within a genome of interest, e.g. within the human genome. In some embodiments, a mutant isolated Cas9 protein or a fusion protein of Cas9 and a nucleic acid editing enzyme or nucleic acid editing enzyme domain, such as a deaminase domain, is provided. In some embodiments, a method for targeted nucleic acid editing is provided. In some embodiments, reagents and kits are provided for generating targeted nucleic acid editing proteins, such as fusion proteins of Cas9 and nucleic acid editing enzymes or nucleic acid editing domains.

The skilled person will understand that this finding serves as a basis for modifications of the corresponding loop in homologous Cas9 nucleases from other species provided that the same loop forms a cap.

Among the exemplary nucleases developed herein, in preferred embodiments three variants have been developed which comprise the Blackjack mutation and are useful to replace the corresponding non-Blackjack nucleases: eSpCas9-plus, SpCas9HF1-plus and B-SpCas9 which are superior variants of eSpCas9, SpCas-HF1 and WT SpCas9, respectively. The Blackjack SpCas9 provides higher fidelity editing than the WT without any detectable decrease in its on-target activity employed with either 20G- or 21G-sgRNAs. Thus, it is worth to use it instead of the WT practically in all applications.

The variants eSpCas9-plus and SpCas9-HF1-plus show even higher fidelity but their activity is slightly decreased on targets in average compared to the WT protein as reported for their original counterparts earlier (Kleinstiver et al., 2016; Kulcsar et al., 2017; Slaymaker et al., 2016). Possessing closely identical fidelity and on-target activity they are excellent substitutes for eSpCas9 and SpCas9-HF1 in all types of applications for which the latter two have been preferentially used but with the added advantage of providing 20G-level editing with 21G-sgRNAs. This advantage is manifested when the sgRNA is transcribed from a DNA template and when finding suitable sequences that are targetable with 20G-sgRNAs is limited such as when a specific position needs to be targeted by exploiting single strand oligos, when using either dCas9-FokI nucleases or base editors or when tagging proteins. One of the most advantageous applications of the plus variants is the usage of pooled sgRNA KO libraries to decrease false positive hits that frequently plague CRISPR screens. SpCas9-HF1-plus offers higher fidelity editing, however, its activity on targets in average is decreased to 80% of that of the WT. Thus, its use may be profitable for those KO libraries where more sgRNAs are targeted to each gene.

Such pooled RNA libraries like crRNA or sgRNA libraries are well known in the art. Preferred libraries are e.g. lentivirus libraries. Pooled sgRNA library screens are exemplified herein. Such libraries are well known n the art (Sanjana et al., 2014). In a preferred embodiment the RNA libraries for use in Cas9 according to the invention comprise 5′ extended target-specific spacer sequences.

A 5′-GG extension of the sgRNA has been reported to increase the fidelity of SpCas9 edition and similar effect is proposed for 21G-sgRNAs that has a 5′-G extension. Here, we showed that 5′-G extension indeed increases the fidelity of WT SpCas9. Since the use of 21G-sgRNAs does not alter the target selectivity/on-target activity of SpCas9 to a detectable extent, 21G-sgRNAs should generally be employed instead of 20G-sgRNAs for all targets to provide higher specificity edition with the WT SpCas9 or better, with B-SpCas9.

Certain Blackjack variants may not be preferred for general use due to their decreased activity on the targets in average. Nevertheless, they have irreplaceable role for modifying specific targets on which only they can provide high specificity editing.

The Blackjack mutations have two effects on the activity of SpCas9 proteins, (i) removing the segments of the protein containing the capping amino acids that would sterically interfere with the extension of the sgRNA potentiates their activity with 21G-sgRNAs and (ii) increasing the fidelity of the protein. An interesting idea is that the disruption of the capping interaction itself is the reason for the increased fidelity observed in the Blackjack variants. Without being bound by theory, assumably this effect is due to using the disruption of an enthalpic interaction of the protein with the 5′ end of the sgRNA, and thus, with the sgRNA-DNA heteroduplex, which is a very similar rationale to that used to design SpCas9-HF1 except that in that case the interactions to be disrupted are mediated via the target DNA strand in the heteroduplex.

An application of the mutations according to the invention, preferably of Blackjack mutations is incorporating Blackjack mutations into PAM-altered variants such as SpCas9.NG (Nishimasu et al., 2018) and xCas9 (Hu et al., 2018). These variants were designed to alter the constraint of the longer, NGG PAM required by SpCas9 nucleases to an NG PAM, and thus to effectively expand the available target space. However, the mutations incorporated to achieve this purpose were found to reduce the activity of xCas9 with 21G-sgRNAs (Lee et al., 2018), limiting its usefulness. The available target sequences are particularly limited when base editors are used to modify nucleotides at specific positions. Combining Blackjack mutations with xCas9 could allow of the realization of the true potential of these PAM-altered variants when used in combination with base editors.

Also provided herein are fusion proteins comprising the isolated Cas9 variant proteins described herein fused to a heterologous functional domain, with an optional intervening linker, wherein the linker does not interfere with activity of the fusion protein. In a preferred embodiment the nuclease activity of the Cas9 variant protein is reduced or inactivated. In preferred embodiments, the heterologous functional domain acts on DNA or protein, e.g., on chromatin. In some embodiments, the heterologous functional domain is a transcriptional activation domain. In some embodiments, the transcriptional activation domain is from VP64 or NF-κB p65. In some embodiments, the heterologous functional domain is a transcriptional silencer or transcriptional repression domain. In some embodiments, the transcriptional repression domain is a Kruppel-associated box (KRAB) domain, ERF repressor domain (ERD), or mSin3A interaction domain (SID). In some embodiments, the transcriptional silencer is Heterochromatin Protein 1 (HP1), e.g., HP1a or HP1β. In some embodiments, the heterologous functional domain is an enzyme that modifies the methylation state of DNA. In some embodiments, the enzyme that modifies the methylation state of DNA is a DNA methyltransferase (DNMT) or the entirety or the dioxygenase domain of a TET protein, e.g., a catalytic module comprising the cysteine-rich extension and the 2OGFeDO domain encoded by 7 highly conserved exons, e.g., the Tet1 catalytic domain comprising amino acids 1580-2052, Tet2 comprising amino acids 1290-1905 and Tet3 comprising amino acids 966-1678. In some embodiments, the TET protein or TET-derived dioxygenase domain is from TET1. In some embodiments, the heterologous functional domain is an enzyme that modifies a histone subunit. In some embodiments, the enzyme that modifies a histone subunit is a histone acetyltransferase (HAT), histone deacetylase (HDAC), histone methyltransferase (HMT), or histone demethylase. In some embodiments, the heterologous functional domain is a biological tether. In some embodiments, the biological tether is MS2, Csy4 or lambda N protein. In some embodiments, the heterologous functional domain is Fold. In some embodiments, the heterologous functional domain is a deaminase, preferably the deaminase is a ApoBac, AID or TADA. In some embodiments, the heterologous functional domain is a reverse transcriptase.

In an other embodiment the functional domains are connected or linked to the Cas9 proteins in a non-covalent binding via a binding interaction or binding molecule or domain.

In a preferred embodiment a binding region is formed on or introduced into the crRNA or sgRNA. The functional domain or protein is linked to a protein which is capable of binding to the binding region on the crRNA or sgRNA. The binding affinity should be sufficiently high to provide an effective direction of the functional domain to the site where the functional effect should be exerted when the Cas9 protein directs the functional domain to the target site.

In a preferred embodiment the binding region is formed by an aptamer and the binding molecule or domain is an aptamer binding molecule or domain.

The crRNA or preferably the sgRNA can be engineered in several ways (Moreno-Mateos M A et al. 2015). In fact, sgRNAs are traditionally derived from the fusion of crRNAs and tracrRNAs, whereas a second part of the crRNA is complementer with the first part of the tracrRNA. These complementer parts form a stem structure which, in the sgRNA is ended in a loop linking the strands of crRNA origin and tracrRNA origin. The stem part may have different length each of which still appropriate to allow Cas9 ribonucleoprotein complex to work. The whole sgRNA without the spacer is about 80 bp long the larger part of which can be modified whereas Cas9 activity is maintained. If the conserved and essential 10 to 20 nucleotides are also changed activity is reduced (while there are mutation which even increase the activity).

This flexibility of the sgRNA is important, and there is with a long segment which may be engineered in various application without impairment of the Cas9 function. Thus, any further functionality may be introduced into this RNA segment having the said loop structure in the sgRNA.

Preparation of Mutations and Vectors

Exemplary wild type Cas9 proteins are those of Uniprot entry no. Q99ZW2 from Streptococcus pyogenes serotype M1 an of Uniprot entry no. Q1J6W2 from Streptococcus pyogenes serotype M4 (strain MGAS10750).

Sequence and data of these proteins are available at

https://www.uniprot.org/uniprot/Q1J6W2; https://www.ncbi.nlm.nih.gov/protein/WP 011528583.1; see also [Haft, D. H., Selengut, J., Mongodin, E. F. and Nelson, K. E. A guild of 45 CRISPR-associated (Cas) protein families and multiple CRISPR/Cas subtypes exist in prokaryotic genomes PLoS Comput. Biol. 1 (6), e60 (2005)]; and

https://www.uniprot.org/uniprot/Q99ZW2; https://www.ncbi.nlm.nih.gov/protein/NP_269215.1;

Nasser, W et al. Evolutionary pathway to increased virulence and epidemic group A Streptococcus disease derived from 3,615 genome sequences Proc. Natl. Acad. Sci. U.S.A. 111 (17), E1768-E1776 (2014)

Also, complete genome of an M1 strain of Streptococcus pyogenes is published by Ferretti et al. [Complete genome sequence of an M1 strain of Streptococcus pyogenes Proc. Natl. Acad. Sci. U.S.A. 98 (8), 4658-4663 (2001)] and is available e.g. at the GenBank database (https://www.ncbi.nlm.nih.gov/nuccore/NC 002737.2 and https://www.ncbi.nlm.nih.gov/nuccore/AE004092).

Alternative Cas9 proteases may come from those of high homology e.g.:

Streptococcus dvsgalactiae subsp. equisimilis RE378

See a review on CRISPR-Cas in Streptococcus pyogenes by Anais Le Rhun et al., RNA Biol. 2019 April; 16(4): 380-389. PMID: 30856357

Vectors useful in the present invention can be constructed using standard molecular biology techniques including the one-pot cloning method (Engler et al., 2008), E. coli DH5α-mediated DNA assembly method (Kostylev et al., 2015), NEBuilder HiFi DNA Assembly and Body Double cloning method (T6th et al., 2014). Plasmids can be transformed into competent bacterial cells, preferably E. coli cells, preferably cells suitable for high efficiency transformation (e.g. NEB Stable competent cells). The skilled person is able to select sgRNA target sites and mismatching sgRNAs sequences; examples are given in Tables 4-7. The sequences of all plasmid constructs advisably should be confirmed by sequencing.

Plasmids useful in the present invention can be acquired from an appropriate plasmid provider, like the non-profit plasmid distribution service Addgene (http://www.addgene.org/). The plasmids used are given in the Examples and have been described in the following publications:

pX330-U6-Chimeric_BB-CBh-hSpCas9 (Addgene #42230) (Cong et al., 2013), eSpCas9 (1.1) (Addgene #71814) (Slaymaker et al., 2016), VP12 (Addgene #72247) (Kleinstiver et al., 2016), sgRNA (MS2) cloning backbone (Plasmid #61424) (Konermann et al., 2015), pMJ806 (#39312) (Jinek et al., 2012), pBMN DHFR (DD)-YFP (#29325) (Iwamoto et al., 2010) and p3s-Sniper-Cas9 (#113912) (Lee et al., 2018), LentiGuide-Puro (#52963) (Sanjana et al., 2014). psPAX2 (#12260) and VSV-G envelope expressing plasmid (#12259) are gifts from Didier Trono.

Based on the methods of the invention the present inventors have developed plasmids and deposited them at Addgene which are available there by the appropriate Addgene nos. given in the examples. The invention is further disclosed and illustrated below by non-limiting illustrative examples.

### EXAMPLES

Blackjack-SpCas9-HF1 Works with 21G-sgRNAs

The present inventors have found that a 5′ G-extension of sgRNAs affects the activity of the increased fidelity variants such as e-, Hypa-, evo-, -HF1, HeF-SpCas9 examined here and came to the idea that in SpCas9 it might result from a capping of the 5′ end of the sgRNA by Glu1007 and Tyr1013, which are connected via a surface loop as revealed by some newer X-ray structures of SpCas9 nuclease (FIG. 1a) (Jiang et al., 2016; Nishimasu et al., 2014). The cap has been removed by mutation to make space for a 5′ G-extension of the sgRNA without clashing with the polypeptide chain. It has been found that it could be achieved without disrupting the structural features of the folded protein. Such modification would allow the increased fidelity nucleases to work with similar efficiency when charged with sgRNAs containing either 20- or 21-nucleotide-long spacers (20G-sgRNA or 21G-sgRNA), thereby extending their target space to non-20G targets without losing fidelity. Recently it has been reported that some 5′-extensions of the sgRNA increase the fidelity (Cho et al., 2014; Kocak et al., 2019) of the WT protein, which we suppose to occur mainly via the perturbation of the cap-interaction. Thus, removing the cap by mutations may also increase the fidelity of the nucleases and transform the WT protein to an increased fidelity nuclease that tolerate a 5′ extension of the sgRNA.

We chose SpCas9-HF1 from among the high-fidelity nucleases as a starting platform and generated a mutant by replacing both Glu1007 and Tyr1013 with glycine to eliminate the presence of sidechains at these positions. In addition, we generated two deletion mutants within the region from aa. 1004 through aa. 1014 (positions where the remaining ends of the polypeptide chain seemed to be connectable without causing major distortions to the protein structure) either by completely removing this segment or by replacing it with two adjacent glycine residues, in order to eliminate the loop. Interestingly, both variants containing the deletions were active with 21G-sgRNAs (Supplementary FIG. 1a) when tested in an EGFP disruption assay, but not the glycine-mutant. In consequence, we decided to proceed further with deletion variants. At first, we screened 13 target sites with the two deletion mutants created, along with the WT and SpCas9-HF1 (Supplementary FIG. 1b) and identified some targets that appeared suitable for easy detection of possibly improved performance of further mutant variants. Based on the same principle, we created 16 further deletion mutant candidates in that region, specifically between aa. 1003 and aa. 1017, by completely removing or exchanging segments of various lengths harboring the loop, with one to up to 4 amino acids with no or small side chains (Supplementary FIG. 1c). The targets selected in the previous step were used to test all mutant candidates in comparison to WT and SpCas9-HF1 (Supplementary FIG. 1d-h). The best candidate was considered to be the one exhibiting the highest on-target activity with 20G-sgRNAs and demonstrating the highest improvement with 21G-sgRNAs. This variant was named Blackjack-SpCas9-HF1 (B-SpCas9-HF1) containing only two glycine residues between the amino acids L1004 and K1014 (FIG. 1b). The Blackjack name, designated by the “B-” prefix refers to its compatibility with 21G-sgRNAs.

Blackjack Mutation Increase the Activity of all Increased Fidelity Variants with 21G-sgRNAs and Increase their Fidelity

Next, we introduced the Blackjack mutations into four increased fidelity variants (e-, -HF, Hypa-, evo-SpCas9) and into the wild type nuclease and compared the on-target activities of these nucleases with 20G- and 21G-sgRNAs to see whether they have increased activity with 21G-sgRNAs. The results obtained with two sequences are presented in FIGS. 1c and d. Blackjack mutations increase the on-target activity of the variants with 21G-sgRNAs up to 17-fold, however, in case of EGFP target 22 the activity of B-evo-SpCas9 decreases even with 20G-sgRNAs suggesting that Blackjack mutations may affect the target selectivity of these nucleases and calling for a more detailed characterization. To assess more comprehensively the effect of Blackjack mutations on the activity of increased fidelity variants with both 20G- and 21G-sgRNAs, we choose 47 EGFP targets. For the 20G experiments, each variant pair is checked on those targets, out of the 47 where the corresponding variants without Blackjack mutations retain their on-target activities with 20G-sgRNAs. The pattern in FIG. 2a and Supplementary FIG. 2 clearly shows that on average the Blackjack mutations increase the target-selectivity (i.e. decrease the activity on the targets in average) of all SpCas9 variants except that of the wild type. For the 21G experiments, to assess specifically, the effect under scrutiny, each variant pair is checked on those targets, out of the 47 where the corresponding variants with Blackjack mutations retain their on-target activities with 20G-sgRNAs. These experiments confirmed that Blackjack variants exhibit greatly increased activities with 21G-sgRNAs (FIG. 3a).

The target selectivity and the fidelity of the increased fidelity variants generally increase in parallel. In line with this expectation, a mismatch screen on the 16 target sequences using the 144 mismatching sgRNAs demonstrated that introduction of the Blackjack mutations increases the fidelity of SpCas9 variants (FIG. 3a and Supplementary FIG. 3a, b).

To validate these conclusions and see whether the disruption of the cap interaction increases the genome-wide fidelity of wild type SpCas9 we applied GUIDE-seq analyses on six targets. Both a 5′ G-extension and the Blackjack mutations were found to decrease the number of off-targets detected and increased the ratio of on-target vs. off-target reads. The effect of the 5′ G-extension compared to the WT protein, however, is reduced in case of the Blackjack variants where these capping interactions are already interrupted (FIG. 3b and Supplementary FIG. 4).

These results show that the Blackjack mutations largely remove the cap from the 5′ end of the sgRNA in the cleavage-competent conformation of the SpCas9-sgRNA-DNA complex, making room for an extension and allowing the effective use of 21G-sgRNAs with increased fidelity variants. However, the mutations also increase the fidelity/target-selectivity of the variants which indicates the fidelity increasing effect of the disruption of cap interactions.

Since the effects of being able to use 21G-sgRNAs and the increased target-selectivity of Blackjack variants are confounded and because the fidelities of the 6 new “up-shifted” Blackjack variants are different from the pre-existing nuclease variants, the higher specificity editing that the Blackjack variants offer is hard to discern.

The Plus Variants of e-SpCas9 and SpCas9-HF1 Match their Fidelity but Effective on a Wider Target Space

To have more complete target coverage and to test our interpretation, we decided to create Blackjack variants that have identical fidelities/target-selectivities to those of eSpCas9 and SpCas9-HF1 based on the following rationale. The Blackjack mutations have two effects: The first is that the deletion potentiates cleavage with a 5′ extended 21G-sgRNA. The second is that it increases the fidelity of SpCas9 when it acts with either 20G- or 21G-sgRNAs. We proposed that by restoring some of the mutations of the Blackjack variants that originate from their corresponding parent increased fidelity nuclease, to their wild type residue (FIG. 4a, d), we can selectively compensate for the second effect. The “parental” eSpCas9 possesses three mutations (K848A, K1003A, R1060A) while SpCas9-HF1 possesses four (N497A, R661A, Q695A, Q926A). After examining the data in the studies describing their development (Kleinstiver et al., 2016; Slaymaker et al., 2016), we constructed four and seven candidates from Blackjack-eSpCas9 and Blackjack-SpCas9-HF1, respectively, lacking one or two “original parental” mutations at a time (FIG. 4a, d). We selected those residues for which we conjectured that their contributions to increasing the fidelity of the respective nuclease would be comparable to those of the Blackjack mutations.

For SpCas9-HF1 we picked 5 targets on which it has considerable activity employing 20G-sgRNAs, on which B-SpCas9-HF1 exhibits strongly decreased activities with 21G-sgRNAs due to its increased target-selectivity, and tested the residue-reverted variants. All new candidates exhibit increased on-target activity with 20G-sgRNAs compared to B-SpCas9-HF1 except that in which A497 was reverted and that, surprisingly, seems to show decreased on-target activities (FIG. 4b). This suggests that the target-selectivity obtained with revertants was successfully reduced and their fidelity correspondingly lowered compared to B-SpCas9-HF1 except for the A497N reversion. To find the variant whose fidelity most closely matches that of SpCas9-HF1 we employed mismatching sgRNAs to two selected targets: one for which SpCas9-HF1 exhibits close to optimal specificity and another for which it demonstrates considerable but decreased off-target activity compared to the wild type nuclease. The reversion of A497 increased the fidelity of B-SpCas9-HF1 consistent with the on-target activity results. Five reversion variants lowered the fidelity of these variants below that of SpCas9-HF1, while the reversion of A661 resulted in a similar fidelity (FIG. 4c). We named this Blackjack variant as SpCas9-HF1-plus and selected it for a more detailed characterization.

For B-eSpCas9, we proceeded as with B-SpCas9-HF1 to create the revertants and picked 5 targets using a similar rationale. Testing the residue-reverted candidate variants, all candidates showed increased on-target activities with 20G-sgRNAs on these targets (FIG. 4e). To find the variant that most closely matches the fidelity of eSpCas9 we selected two targets for which eSpCas9 exhibits close to optimal specificity but on which the B-eSpCas9 demonstrated decreased on-target activity. We employed mismatching sgRNAs to these two targets and tested the variants: the reversion of A1003 resulted in the closest fidelity match to that of eSpCas9 (FIG. 4f). We named this Blackjack variant eSpCas9-plus and selected it for a more detailed characterization.

Western blotting indicated that Blackjack mutations do not alter the expression level of SpCas9 variants and the amounts of the plus variants expressed at steady state are comparable to their parent variants (Supplementary FIG. 5a). We compared the selected plus variants' on-target activities with 20G-sgRNAs on 25 targets with their parental variants. Both eSpCas9-plus and SpCas9-HF1-plus reached the on-target activities of their original counterpart variant on this set of target sequences (FIG. 5a). To challenge the plus variants when checking their activities with 21G-sgRNAs, different sets of ten-targets were assayed with the enhanced and with the high-fidelity variants to exploit targets on which the parent nucleases exhibited strongly decreased on-target activity upon appending a 5′ 21^{st} G. To assess specifically the effect under scrutiny, the same ten sequences were targeted with both 20G- and 21G-sgRNAs. With 21G-sgRNAs both eSpCas9-plus (FIG. 5b) and SpCas9-HF1-plus (FIG. 5c) demonstrated highly increased on-target activities; reaching 90% of that with 20G-sgRNAs on the same targets, in contrast to their parent variants demonstrating only 10% and 16%, respectively. To compare the fidelity of the plus variants with their parents, 13 targets were selected on which both eSpCas9 and SpCas9-HF1 had demonstrated reasonable on-target activities and 117 mismatching sgRNAs were employed. At all of the 39 positions examined the off-target activity of eSpCas9-plus resulted in an identical off-target-cleavage pattern, matching the fidelity of eSpCas9 (Supplementary FIG. 5b). The off-target activity of SpCas9-HF1 and SpCas9-HF1-plus compared in a similar way also gave rise to very similar patterns, closely matching each other's fidelities (Supplementary FIG. 5c). These data demonstrate that the plus variants possess identical fidelity but strongly increased on-target activity with 21G-sgRNAs, and thus can be utilized on a much wider range of targets compared to their parent variants.

Next Generation Sequencing (NGS) Confirmed the Matching Activities with 20- or 21G-sgRNAs of the Plus Variants

We further characterized their on-target activities in HEK293 cells, monitoring their indel-inducing activities by NGS. We selected 23 sequences from the human FANCF and VEGFA loci. There was no prior cleavage information available for them except for one (FANCF site 2). Sixteen of them can be targeted with 21G-sgRNAs and seven with 20G-sgRNAs (Table 5). The on-target activities of the plus variants with 20G-sgRNAs match those of their corresponding original counterparts (FIG. 5d). However, with 21G-sgRNAs they show much higher on-target activity (FIG. 5e). Since their target-selectivity is higher than that of WT SpCas9 (Kleinstiver et al., 2016; Kulcsar et al., 2017; Slaymaker et al., 2016), they are not expected to reach the 21G-sgRNAs WT-level activity. The original counterparts, eSpCas9 and SpCas9-HF1 exhibit slightly decreased on-target activities with 20G-sgRNAs, 93% and 82% on average, respectively, relative to WT SpCas9. This relative activity level has to be reached by the plus variants with 21G-sgRNAs to let us say that they work with identical efficiency using either 20G- or 21G-sgRNAs. What we found was that with 21G-sgRNAs eSpCas9-plus demonstrates 88%, while SpCas9-HF1-plus exhibits 82% of the value of WT SpCas9 with 21G-sgRNA, respectively (FIG. 5e) so we can say they worked with nearly identical efficiency.

We also wanted to compare the fidelity of these nucleases by GUIDE-seq. In HEK293.EGFP cells, we selected 7 target sites that can be targeted by 20G-sgRNAs to make sure that not just the nuclease variants containing Blackjack mutations are able to cleave the on-target sequences. Among these targets, three (VEGFA site 2, HEK site 4, FANCF site 2) have been used to characterize the off-target activities of the increased fidelity variants in earlier studies (Casini et al., 2018; Chen et al., 2017; Kleinstiver et al., 2016; Lee et al., 2018). As expected based on previous results, we found that all four increased fidelity variants demonstrate greatly increased fidelity compared to the wild type protein and that the corresponding parent—plus “variant pairs” behave similarly to each other, eSpCas9-plus cleaving slightly less, while SpCas9-HF1-plus slightly more off-target sites on some targets compared to their parent variants (FIG. 5f and Supplementary FIG. 6). These results are consistent with the contention that the fidelity of these plus variants closely matches that of their parental (non-Blackjack) counterparts.

RNP Form of Blackjack and Plus Variants Increase Specificity

The development of two new increased fidelity variants, Sniper- and HiFi SpCas9 has been reported more recently, claiming they work effectively in RNP form, and Sniper SpCas9 is able to work even with 5′-modified sgRNAs, unlike former increased fidelity variants (Lee et al., 2018; Vakulskas et al., 2018). Sniper SpCas9 being less “attenuated”⁴⁵ has lower target selectivity and fidelity (data not shown) that may offer an explanation for its ability to work with 5′-modified sgRNAs. However, it is not clear why they possess high activity in RNP form, while the former increased fidelity variants have reported to possess a strongly reduced activity in RNP form (Vakulskas et al., 2018). RNPs are the method of choice for prospective clinical applications, and we wondered if Blackjack variants are able to provide optimal high fidelity editing for the majority of the targets on which one of the other increased fidelity nucleases provide better specificity editing, compared to Sniper or HiFi SpCas9. Thus, we selected 31 sequences to assay for EGFP disruption by eSpCas9 and SpCas9-HF1 and by their plus variants delivered in RNP form. Since it seems that there is no consensus about the requirement of the T7 polymerase for the preferred starting sequences of the transcript, we selected sequences that start with non-G, G or GG nucleotides and we targeted them systematically with in vitro transcribed, 5′ G- or GG-extended or fully matching 20G-sgRNAs, as depicted in FIG. 6a-c. Surprisingly, in contrast to that reported by Vakulskas et al. (Vakulskas et al., 2018), all variants show similarly high activities with 20G-sgRNAs as the WT protein (FIG. 6b). Here we used targets on which eSpCas9 and SpCas9-HF1 are active when delivered as plasmids that might account for the discrepancies with the Vakulskas study. By contrast, only the plus variants demonstrate high activities with 21G-sgRNAs in pre-assembled RNP form too, reaching up to 23-fold higher activities than their parental variants (FIG. 6a-c). Thus, we concluded that plus variants are effective in the RNP form, and provide high-fidelity editing with both 20G- and 21G-sgRNAs, and allow the effective use of in vitro transcribed 21G-sgRNAs.

We were also curious to compare the on-target activities of the plus variants (SpCas9HF-1-plus and eSpCas9-plus) with sgRNA processing approaches to see whether the plus variants offers advantages in those applications where the sgRNA processing approach can also be applied (eSpCas9-plus is shown herein). Testing on 19 targets, the plus variants showed higher activities than either the tRNA (Dong et al., 2017) or ribozyme processing approaches (Kim et al., 2017; Lee et al., 2016) (Supplementary FIG. 7c). Some target sequences in case of both approaches were cleaved with reduced efficiency by the WT protein, suggesting that the understanding of the sequence dependencies of the tRNA or ribozyme processing needs a more comprehensive investigation.

Finally, we wanted to test the usefulness of Blackjack variants in a practical application by investigating the expression of the prion protein family-member Shadoo protein after inserting the EGFP sequences downstream of the mouse Shadoo promoter exploiting NHEJ repair (Talas et al., 2017). Five NGG PAM sequences are available at relevant positions but none of them are targetable with 20G-sgRNAs, presenting a good example where Blackjack variants can offer a specific advantage for an endeavor. We pre-screened the available five targets with several increased fidelity nucleases by integrating an EGFP cassette into the targeted site. Pre-screening identified the optimal targets and nuclease variants (FIG. 7a) for the generation of the desired transgenic lines by increased fidelity nucleases (FIG. 7b). The results confirmed the advantage of the Blackjack variants for this practical application and we predict that comparable results will be obtained from a wide range of practical applications.

Finally, to demonstrate that the plus variants are compatible with pooled sgRNA library screens where the sgRNAs are inherently expressed at low levels, we generated three cell lines each expressing one 21G-sgRNA from an integrated lentivirus copy. Each of the three cell lines were interrogated by eSpCas9-plus in parallel to WT and eSpCas9 (FIG. 7c). In contrast to eSpCas9, eSpCas9-plus demonstrated activities approaching nearing that of the WT protein. These results confirm that the activity of eSpCas9-plus renders the nuclease compatible with pooled sgRNA libraries where the sgRNAs are naturally expressed from an integrated single copy of a lentivirus and even when 21G-sgRNAs are used.

Methods

Materials

Restriction enzymes, T4 ligase, Dulbecco's modified Eagle Medium DMEM (Gibco), fetal bovine serum (Gibco), Turbofect, Lipofectamine 2000, TranscriptAid T7 High Yield Tmnscription Kit, Qubit™ dsDNA HS Assay Kit, Shrimp Alkaline Phosphatase (SAP), Taq DNA polymerase (recombinant), Platinum Taq DNA polymerase and penicillin/streptomycin were purchased from Thermo Fischer Scientific, protease inhibitor cocktail was purchased from Roche Diagnostics. DNA oligonucleotides, trimethoprim (TMP) and GenElute HP Plasmid Miniprep kit were acquired from Sigma-Aldrich. ZymoPure Plasmid Midiprep kit and RNA Clean & Concentrator kit were purchased from Zymo Research. NEBuilder HiFi DNA Assembly Master Mix and Q5 High-Fidelity DNA Polymerase were obtained from New England Biolabs Inc. NucleoSpin Gel and PCR Clean-up kit was purchased from Macherey-Nagel. 2 mm electroporation cuvettes were acquired from Cell Projects Ltd, Bioruptor 0.5 ml Microtubes for DNA Shearing from Diagenode. Agencourt AMPure XP beads were purchased from Beckman Coulter. T4 DNA ligase (for GUIDE-seq) and end-repair mix were acquired from Enzymatics. KAPA universal qPCR Master Mix was purchased from KAPA Biosystems.

Plasmid Construction

Vectors were constructed using standard molecular biology techniques including the one-pot cloning method (Engler et al., 2008), E. coli DH5α-mediated DNA assembly method (Kostylev et al., 2015), NEBuilder HiFi DNA Assembly and Body Double cloning method (T6th et al., 2014). Plasmids were transformed into NEB Stable competent cells. sgRNA target sites and mismatching sgRNAs sequences are available in Table 4-7. The sequences of all plasmid constructs were confirmed by Sanger sequencing (Microsynth AG).

Plasmids acquired from the non-profit plasmid distribution service Addgene (http://www.addgene.org/) are the following:

pX330-U6-Chimeric_BB-CBh-hSpCas9 (Addgene #42230) (Cong et al., 2013), eSpCas9 (1.1) (Addgene #71814) (Slaymaker et al., 2016), VP12 (Addgene #72247) (Kleinstiver et al., 2016), sgRNA (MS2) cloning backbone (Plasmid #61424) (Konermann et al., 2015), pMJ806 (#39312) (Jinek et al., 2012), pBMN DHFR (DD)-YFP (#29325) (Iwamoto et al., 2010) and p3s-Sniper-Cas9 (#113912) (Lee et al., 2018), LentiGuide-Puro (#52963) (Sanjana et al., 2014). psPAX2 (#12260) and VSV-G envelope expressing plasmid (#12259) are gifts from Didier Trono.

Plasmids developed by us and deposited at Addgene are the following:

pX330-Flag-dSpCas9 (Addgene #92113), pX330-Flag-WT_SpCas9 (without sgRNA; with silent mutations) (Addgene #126753), pX330-Flag-eSpCas9 (without sgRNA; with silent mutations) (Addgene #126754), pX330-Flag-SpCas9-HF1 (without sgRNA; with silent mutations) (Addgene #126755), pX330-Flag-HypaSpCas9 (without sgRNA; with silent mutations) (Addgene #126756), pX330-Flag-evoSpCas9 (without sgRNA; with silent mutations) (Addgene #126758), pX330-Flag-HeFSpCas9 (without sgRNA; with silent mutations) (Addgene #126759), pX330-Flag-Sniper SpCas9 (without sgRNA; with silent mutations) (Addgene #126777), pX330-Flag-HiFi SpCas9 (without sgRNA; with silent mutations) (Addgene #126778),

B-SpCas9 (Addgene #126760), B-eSpCas9 (Addgene #126761), B-SpCas9-HF1 (Addgene #126762), B-HypaSpCas9 (Addgene #126763), B-evoSpCas9 (Addgene #126765), B-HeFSpCas9 (Addgene #126766)

eSpCas9-plus (Addgene #126767), SpCas9-HF1-plus (Addgene #126768)

pET-FLAG-eSpCas9 (Addgene #126769), pET-FLAG-SpCas9-HF1 (Addgene #126770), pET-FLAG-B-eSpCas9 (Addgene #126772), pET-FLAG-eSpCas9-plus (Addgene #126774), pET-FLAG-SpCas9-HF1-plus (Addgene #126775)

pmCherry_gRNA-ver2 (Addgene #126776), pmCherry_gRNA (Addgene: #80457)

Cell Culturing

Cells employed in the studies are N2a (neuro-2a mouse neuroblastoma cells, ATCC-CCL-131), HEK293 (Gibco 293-H cells), N2a.dd-EGFP (a cell line developed by us containing a single integrated copy of an EGFP-DHFR[DD] [EGFP-folA dihydrofolate reductase destabilization domain] fusion protein coding cassette originating from a donor plasmid with 1,000 bp-long homology arms to the Prnp gene driven by the Prnp promoter (Prnp.HA-EGFP-DHFR[DD])), as well as N2a.EGFP and HEK-293.EGFP (both cell lines containing a single integrated copy of an EGFP cassette driven by the Prnp promoter)(Kulcsar et al., 2017) cells. Cell lines were not authenticated as they were obtained directly from a certified repository or clone from those cell lines. Cells were grown at 37° C. in a humidified atmosphere of 5% CO₂ in high glucose Dulbecco's Modified Eagle medium (DMEM) supplemented with 10% heat inactivated fetal bovine serum, 4 mM L-glutamine (Gibco), 100 units/ml penicillin and 100 μg/ml streptomycin. Cells were passaged up to 20 times (washed with PBS, detached from the plate with 0.05% Trypsin-EDTA and replated). After 20 passages, cells were discarded.

Flow Cytometry

Flow cytometry analyses were carried out on an Attune NxT Acoustic Focusing Cytometer (Applied Biosystems). For data analysis Attune NxT Software v.2.7.0 was used. Viable single cells were gated based on side and forward light-scatter parameters and a total of 5,000-10,000 viable single cell events were acquired in all experiments. The GFP fluorescence signal was detected using the 488 nm diode laser for excitation and the 530/30 nm filter for emission, the mCherry fluorescent signal was detected using the 488 nm diode laser for excitation and a 640LP filter for emission or using the 561 nm diode laser for excitation and a 620/15 nm filter for emission.

EGFP Disruption Assay

All EGFP disruption experiments were conducted on the N2a.dd-EGFP cell line except the on-target screen, which was conducted on N2a.EGFP cells (see details below). Cells were plated one day prior to transfection in 48-well plates at a density of approximately 25,000-30,000 cells/well. Cells were co-transfected with two types of plasmids: SpCas9 variant expression plasmid (137 ng) and sgRNA and mCherry coding plasmid (97 ng) using 1 μl TurboFect reagent per well in 48-well plates. TMP (trimethoprim; 1 μM final concentration) was added to the media ˜48 h before FACS analysis in case of N2a.dd-EGFP cells. Transfected cells were analysed ˜96 h post-transfection by flow cytometry. Transfection efficacy was calculated via mCherry expressing cells. Transfections were performed in triplicate. Replicates not measured due to sample loss are indicated in the raw data (less than 1% in all experiments altogether).

Background EGFP loss for each experiment was determined using co-transfection of dead SpCas9 expression plasmid and different targeting sgRNA and mCherry coding plasmids. EGFP disruption values were calculated as follows: the average EGFP background loss from dead SpCas9 control transfections made in the same experiment was subtracted from each individual treatment in that experiment and the mean values and the standard deviation (s.d.) were calculated from it. In the case of normalization, the results were normalized to the WT SpCas9 data from the same experiment.

On-target activity was measured on N2a.EGFP cell line 4 days post-transfection by flow cytometry. In this cell line the EGFP disruption level is not saturated, this way this assay is a more sensitive reporter of the intrinsic activities of these nucleases compared to N2a.dd-EGFP cell line.

In the case of mismatch screens and 21G-sgRNA screens N2a.dd-EGFP cells were co-transfected with two types of plasmids: with SpCas9 variant expression plasmid (137 ng) and a mix of 3 sgRNAs in which one nucleotide position was mismatched to the target using all 3 possible bases and mCherry coding plasmid (3×˜33.3 ng=97 ng) using 1 μl TurboFect reagent per well in 48-well plates. TMP (trimethoprim; 1 μM final concentration) was added to the media ˜48 h before FACS analysis. Transfected cells were analysed ˜96 h post-transfection by flow cytometry. The 4-day post-transfection results with this cell line show a close to saturated level activity, this way it is a good reporter system for seeing the full spectrum of off-target activities.

Western Blot

N2a.dd-EGFP cells were cultured on 48-well plates and were transfected as described above in the EGFP disruption assay section. Four days post-transfection, 9 parallel samples corresponding to each type of SpCas9 variant transfected were washed with PBS, then trypsinized and mixed, and were analyzed for transfection efficiency via mCherry fluorescence level by using flow cytometry. The cells from the mixtures were centrifuged at 200 rcf for 5 min at 4° C. Pellets were resuspended in ice cold Harlow buffer (50 mM Hepes pH 7.5; 0.2 mM EDTA; 10 mM NaF; 0.5% NP40; 250 mM NaCl; Protease Inhibitor Cocktail 1:100; Calpain inhibitor 1:100; 1 mM DTT) and lysed for 20-30 min on ice. The cell lysates were centrifuged at 19,000 rcf for 10 min. The supernatants were transferred into new tubes and total protein concentrations were measured by the Bradford protein assay. Before SDS gel loading, samples were boiled in Protein Loading Dye for 10 min at 95° C. Proteins were separated by SDS-PAGE using 7.5% polyacrylamide gels and were transferred to a PVDF membrane, using a wet blotting system (Bio-Rad). Membranes were blocked by 5% non-fat milk in Tris buffered saline with Tween20 (TBST) (blocking buffer) for 2 h. Blots were incubated with primary antibodies [anti-FLAG (F1804, Sigma) at 1:1,000 dilution; anti-$-actin (A1978, Sigma) at 1:4,000 dilution in blocking buffer] overnight at 4° C. The next day after washing steps in TBST the membranes were incubated for 1 h with HRP-conjugated secondary anti-mouse antibody 1:20,000 (715-035-151, Jackson ImmunoResearch) in blocking buffer. The signal from detected proteins was visualized by ECL (Pierce ECL Western Blotting Substrate, Thermo Scientific) using a CCD camera (Bio-Rad ChemiDoc MP).

Indel Analysis by Next-Generation Sequencing (NGS)

HEK293 cells were seeded onto 48-well plates a day before transfection at a density of 1.2×10⁴ cells/well. The next day, at around 25% confluence, cells were transfected with plasmid constructs using Jetfect reagent (Biospiral-2006. Ltd.), briefly as follows: 234 ng total plasmid DNA (97 ng sgRNA and mCherry expression plasmid, and 137 ng nuclease expression plasmid) and 1 μl Jetfect reagent were mixed in 50 μl serum free DMEM and the mixture was incubated for 30 min at room temperature prior to adding to cells. Three parallel transfections were made from each sample. Replicates not measured due to sample loss are indicated in the raw data (less than 1%). Transfection efficiency was analysed by flow cytometry five days post-transfection via mCherry fluorescence after which cells were centrifuged at 1,000 rcf for 10 minutes and genomic DNA was purified according to the Puregene DNA Purification protocol (Gentra systems). Amplicons for deep sequencing were generated using two rounds of PCR by Q5 high-fidelity polymerase to attach Illumina handles. The 1^{st} step PCR primers used to amplify target genomic sequences are listed in Table 1. After the 2^{nd} step PCR the samples were quantified with Qubit dsDNA HS Assay kit (Invitrogen) and PCR products were pooled for deep sequencing. Sequencing on an Illumina Miseq instrument was performed by ATGandCo Ltd. Indels were counted computationally among reads that matched at least 75% to the first 20 bp of the reference amplicon. Indels without mismatches were searched at +/−40 bp around the cut site. For each sample, the indel frequency was determined as (number of reads with an indel)/(number of total reads). Average reads per sample was 18801. The following software were used: BBMap 38.08, samtools 1.8, BioPython 1.71, PySam 0.13.

TABLE 1
List of primers used to amplify sequence of interest for deep sequencing

| Name of deep | | |
|---|---|---|
| sequencing | | SEQ |
| first-step PCR | | ID |
| primers | Sequence | NO: |
| i5-FANCF2- | TCGTCGGCAGCGTCAGATGTGTATAAGAGACAGGGTGCTGACGTAGGTAGTGC | 25 |
| ONtarget-FWD | | |
| i7-FANCF2- | GTCTCGTGGGCTCGGAGATGTGTATAAGAGACAGGTCACAGTATGTCTCTGGCGT | 26 |
| ONtarget-REV | | |
| i5-FANCF2- | TCGTCGGCAGCGTCAGATGTGTATAAGAGACAGGGGCCGGGAAAGAGTTGCTG | 27 |
| ONtarget-FWD2 | | |
| i7-FANCF2- | GTCTCGTGGGCTCGGAGATGTGTATAAGAGACAGGCCCTACATCTGCTCTCCCTCC | 28 |
| ONtarget-REV2 | | |
| i5-VEGFA3- | TCGTCGGCAGCGTCAGATGTGTATAAGAGACAGTCCAGATGGCACATTGTCAG | 29 |
| ONtarget-FWD2 | | |
| i7-FVEGFA3- | GTCTCGTGGGCTCGGAGATGTGTATAAGAGACAGAGGGAGCAGGAAAGTGAGGT | 30 |
| ONtarget-REV2 | | |
| i5-VEGFA3- | TCGTCGGCAGCGTCAGATGTGTATAAGAGACAGAGGGAGCAGGAAAGTGAGGT | 31 |
| ONtarget-REV2 | | |
| i7-FVEGFA3- | GTCTCGTGGGCTCGGAGATGTGTATAAGAGACAGTCCAGATGGCACATTGTCAG | 32 |
| ONtarget-FWD2 | | |

In Vitro Transcription

sgRNAs were in vitro transcribed using TranscriptAid T7 High Yield Transcription Kit and PCR-generated double-stranded DNA templates carrying a T7 promoter sequence. Primers used for the preparation of the DNA templates are listed in Table 2. sgRNAs were dephosphorylated with SAP, purified with the RNA Clean & Concentrator kit, and reannealed (95° C. for 5 min, ramp to 4° C. at 0.3° C./s). sgRNAs were quality checked using 10% denaturing polyacrylamide gels and ethidium bromide staining.

TABLE 2
List of primers used to amplify sgRNA sequences for in vitro transcription

| | | | SEQ |
|---|---|---|---|
| | | Template | ID |
| Name of primers | Sequence | prep name | NO: |
| T7-+G-Site51 | AAAAAATAATACGACTCACTATAgCGATGCCCTTCAGCTCGATG | 1550 | 33 |
| T7-+G-Site52 | AAAAAATAATACGACTCACTATAgCAACATCCTGGGGCACAAGC | 1551 | 34 |
| T7-+G-Site53 | AAAAAATAATACGACTCACTATAgCAGCTCGATGCGGTTCACCA | 1552 | 35 |
| T7-+G-Site54 | AAAAAATAATACGACTCACTATAgCAAGGAGGACGGCAACATCC | 1553 | 36 |
| T7-+G-Site55 | AAAAAATAATACGACTCACTATAgTCAGCTCGATGCGGTTCACC | 1554 | 37 |
| T7-+G-Site56 | AAAAAATAATACGACTCACTATAgAAGGAGGACGGCAACATCCT | 1555 | 38 |
| T7-+G-Site57 | AAAAAATAATACGACTCACTATAgAGGAGGACGGCAACATCCTG | 1556 | 39 |
| T7-+G-Site58 | AAAAAATAATACGACTCACTATAgCATGCCCGAAGGCTACGTCC | 1558 | 40 |
| T7-+G-Site59 | AAAAAATAATACGACTCACTATAgCGTGCTGCTTCATGTGGTCG | 1560 | 41 |
| T7-+2G-Site51 | AAAAAATAATACGACTCACTATAggCGATGCCCTTCAGCTCGATG | 1550 | 42 |
| T7-+2G-Site52 | AAAAAATAATACGACTCACTATAggCAACATCCTGGGGCACAAGC | 1551 | 43 |
| T7-+2G-Site53 | AAAAAATAATACGACTCACTATAggCAGCTCGATGCGGTTCACCA | 1552 | 44 |
| T7-+2G-Site54 | AAAAAATAATACGACTCACTATAggCAAGGAGGACGGCAACATCC | 1553 | 45 |
| T7-+2G-Site55 | AAAAAATAATACGACTCACTATAggTCAGCTCGATGCGGTTCACC | 1554 | 46 |
| T7-+2G-Site56 | AAAAAATAATACGACTCACTATAggAAGGAGGACGGCAACATCCT | 1555 | 47 |
| T7-+2G-Site57 | AAAAAATAATACGACTCACTATAggAGGAGGACGGCAACATCCTG | 1556 | 48 |
| T7-+2G-Site58 | AAAAAATAATACGACTCACTATAggCATGCCCGAAGGCTACGTCC | 1558 | 49 |
| T7-+2G-Site59 | AAAAAATAATACGACTCACTATAggCGTGCTGCTTCATGTGGTCG | 1560 | 50 |
| T7-Site-1 | AAAAAATAATACGACTCACTATAgggcacgggcagcttgccgg | 8323 | 51 |
| T7-Site3 | AAAAAATAATACGACTCACTATAGGTGGTGCAGATGAACTTCA | 1582 | 52 |
| T7-Site4 | AAAAAATAATACGACTCACTATAggagcgcaccatcttcttca | 1584 | 53 |
| T7-Site16 | AAAAAATAATACGACTCACTATAggcgagggcgatgccaccta | 13021 | 54 |
| T7-Site21 | AAAAAATAATACGACTCACTATAGGCATCGACTTCAAGGAGGA | 1637 | 55 |
| T7-Site22 | AAAAAATAATACGACTCACTATAGGTGTTCTGCTGGTAGTGGT | 1638 | 56 |
| T7-Site31 | AAAAAATAATACGACTCACTATAGGGCGAGGAGCTGTTCACCG | 8629 | 57 |
| T7-Site33 | AAAAAATAATACGACTCACTATAGGTGCCCATCCTGGTCGAGC | 8631 | 58 |
| T7-Site37 | AAAAAATAATACGACTCACTATAGGTCAGGGTGGTCACGAGGG | 8635 | 59 |
| T7-Site46 | AAAAAATAATACGACTCACTATAGGTTGTCGGGCAGCAGCACG | 8644 | 60 |
| T7-Site48 | AAAAAATAATACGACTCACTATAGGTGCTCAGGTAGTGGTTGT | 8646 | 61 |
| T7-+G-Site1 | AAAAAATAATACGACTCACTATAgGGGCACGGGCAGCTTGCCGG | 8323 | 62 |
| T7-+G-Site3 | AAAAAATAATACGACTCACTATAgGGTGGTGCAGATGAACTTCA | 1582 | 63 |
| T7-+G-Site4 | AAAAAATAATACGACTCACTATAgGGAGCGCACCATCTTCTTCA | 1584 | 64 |
| T7-+G-Site16 | AAAAAATAATACGACTCACTATAgGGCGAGGGCGATGCCACCTA | 13021 | 65 |
| T7-+G-Site21 | AAAAAATAATACGACTCACTATAgGGCATCGACTTCAAGGAGGA | 1637 | 66 |
| T7-+G-Site22 | AAAAAATAATACGACTCACTATAgGGTGTTCTGCTGGTAGTGGT | 1638 | 67 |
| T7-+G-Site31 | AAAAAATAATACGACTCACTATAgGGGCGAGGAGCTGTTCACCG | 8629 | 68 |
| T7-+G-Site33 | AAAAAATAATACGACTCACTATAgGGTGCCCATCCTGGTCGAGC | 8631 | 69 |
| T7-+G-Site37 | AAAAAATAATACGACTCACTATAgGGTCAGGGTGGTCACGAGGG | 8635 | 70 |
| T7-+G-Site46 | AAAAAATAATACGACTCACTATAgGGTTGTCGGGCAGCAGCACG | 8644 | 71 |
| T7-+G-Site48 | AAAAAATAATACGACTCACTATAgGGTGCTCAGGTAGTGGTTGT | 8646 | 72 |
| T7-Site5 | AAAAAATAATACGAGTCACTATAgtcgccctcgaacttcacct | 1585 | 73 |
| T7-Site13 | AAAAAATAATACGACTCACTATAGCTGAAGGGCATCGACTTCA | 1593 | 74 |
| T7-Site17 | AAAAAATAATACGACTCACTATAGCTGAAGCACTGCACGCCGT | 1633 | 75 |
| T7-Site18 | AAAAAATAATACGACTCACTATAGTGAACCGCATCGAGCTGAA | 1634 | 76 |
| T7-Site20 | AAAAAATAATACGACTCACTATAGACGTAGCCTTCGGGCATGG | 1636 | 77 |
| T7-Site23 | AAAAAATAATACGACTCACTATAGTGGTCACGAGGGTGGGCCA | 1639 | 78 |
| T7-Site38 | AAAAAATAATACGACTCACTATAGTAGGTCAGGGTGGTCACGA | 8636 | 79 |
| T7-Site39 | AAAAAATAATACGACTCACTATAGCACTGCACGCCGTAGGTCA | 8637 | 80 |
| T7-Site44 | AAAAAATAATACGACTCACTATAGATGCCGTTCTTCTGCTTGT | 8642 | 81 |
| T7-Site45 | AAAAAATAATACGACTCACTATAGCACGGGGCCGTCGCCGATG | 8643 | 82 |
| T7-Site47 | AAAAAATAATACGACTCACTATAGTGGTTGTCGGGCAGCAGCA | 8645 | 83 |
| T7-+G-Site5 | AAAAAATAATACGACTCACTATAGgtcgccctcgaacttcacct | 1585 | 84 |
| T7-+G-Site13 | AAAAAATAATACGACTCACTATAgGCTGAAGGGCATCGACTTCA | 1593 | 85 |
| T7-+G-Site17 | AAAAAATAATACGACTCACTATAgGCTGAAGCACTGCACGCCGT | 1633 | 86 |
| T7-+G-Site18 | AAAAAATAATACGACTCACTATAgGTGAACCGCATCGAGCTGAA | 1634 | 87 |
| T7-+G-Site20 | AAAAAATAATACGACTCACTATAgGACGTAGCCTTCGGGCATGG | 1636 | 88 |
| T7-+G-Site23 | AAAAAATAATACGACTCACTATAgGTGGTCACGAGGGTGGGCCA | 1639 | 89 |
| T7-+G-Site38 | AAAAAATAATACGACTCACTATAgGTAGGTCAGGGTGGTCACGA | 8636 | 90 |
| T7-+G-Site39 | AAAAAATAATACGACTCACTATAgGCACTGCACGCCGTAGGTCA | 8637 | 91 |
| T7-+G-Site44 | AAAAAATAATACGACTCACTATAgGATGCCGTTCTTCTGCTTGT | 8642 | 92 |
| T7-+G-Site45 | AAAAAATAATACGACTCACTATAgGCACGGGGCCGTCGCCGATG | 8643 | 93 |
| T7-+G-Site47 | AAAAAATAATACGACTCACTATAgGTGGTTGTCGGGCAGCAGCA | 8645 | 94 |
| T7-+G-Site43 | AAAAAATAATACGACTCACTATAgGAAGGGCATCGACTTCAAGG | 8641 | 95 |
| T7-+G- | AAAAAATAATACGACTCACTATAgGACCCCCTCCACCCCGCCTC | 9200 | 96 |
| VEGFAsite2 | | | |
| T7-+G- | AAAAAATAATACGACTCACTATAgGCTGCAGAAGGGATTCCATG | 9199 | 97 |
| FANCFsite2 | | | |
| T7-reverse primer | AAAAAAGCACCGACTCGGTGCC | | 98 |

Protein Purification

All SpCas9 variants were subcloned from pMJ806 (Addgene #39312) (Jinek et al., 2012) (for detailed cloning information see section: SpCas9 variants, bacterial expression plasmids). The resulting fusion constructs contained an N-terminal hexahistidine (His6), a Maltose binding protein (MBP) tag and a Tobacco etch virus (TEV) protease site.

The expression constructs of the SpCas9 variants were transformed into E. coli BL21 Rosetta 2 (DE3) cells, grown in Luria-Bertani (LB) medium at 37° C. for 16 h. 10 ml from this culture was inoculated into 1 l of growth media (12 g/l Tripton, 24 g/l Yeast, 10 g/l NaCl, 883 mg/l NaH₂PO₄H₂O, 4.77 g/l Na₂HPO₄, pH 7.5) and cells were grown at 37° C. to a final cell density of 0.6 OD600, and then were chilled at 18° C. The protein was expressed at 18° C. for 16 h following induction with 0.2 mM IPTG. The protein was purified by a combination of chromatographic steps by NGC Scout Medium-Pressure Chromatography Systems (Bio-Rad). The bacterial cells were centrifuged at 6,000 rcf for 15 min at 4° C. The cells were resuspended in 30 ml of Lysis Buffer (40 mM Tris pH 8.0, 500 mM NaCl, 20 mM imidazole, 1 mM TCEP) supplemented with Protease Inhibitor Cocktail (1 tablet/30 ml; complete, EDTA-free, Roche) and sonicated on ice. Lysate was cleared by centrifugation at 48,000 rcf for 40 min at 4° C. Clarified lysate was bound to a 5 ml Mini Nuvia IMAC Ni-Charged column (Bio-Rad). The resin was washed extensively with a solution of 40 mM Tris pH 8.0, 500 mM NaCl, 20 mM imidazole, and the bound protein was eluted by a solution of 40 mM Tris pH 8.0, 250 mM imidazole, 150 mM NaCl, 1 mM TCEP. 10% glycerol was added to the eluted sample and the His6-MBP fusion protein was cleaved by TEV protease (3 h at 25° C.). The volume of the protein solution was made up to 100 ml with buffer (20 mM HEPES pH 7.5, 100 mM KCl, 1 mM DTT). The cleaved protein was purified on a 5 ml HiTrap SP HP cation exchange column (GE Healthcare) and eluted with 1 M KCl, 20 mM HEPES pH 7.5, 1 mM DTT. The protein was further purified by size exclusion chromatography on a Superdex 200 10/300 GL column (GE Healthcare) in 20 mM HEPES pH 7.5, 200 mM KCl, 1 mM DTT and 10% glycerol. The eluted protein was confirmed by SDS-PAGE and Coomassie brilliant blue R-250 staining. The protein was stored at −20° C.

EGFP Disruption Assay with RNP

N2a.dd-EGFP cells cultured on 48-well plates, were seeded a day before transfection at a density of 3×10⁴ cells/well, in 250 μl complete DMEM. 13.75 pmol SpCas9 and 16.5 pmol sgRNA was complexed in Cas9 storage buffer (20 mM HEPES pH 7.5, 200 mM KCl, 1 mM DTT and 10% glycerol) for 15 minutes at RT. 25 μl serum-free DMEM and 0.8 μl Lipofectamine 2000 was added to the complexed RNP and incubated for 20 minutes prior to adding to the cells. TMP (trimethoprim; 1 μM final concentration) was added to the media ˜48 h before FACS analysis. Transfected cells were analyzed ˜96 h post-transfection by flow cytometry. Transfections were performed in triplicate. Background EGFP loss for each experiment was determined using co-transfection of WT SpCas9 expression plasmid and non-targeted sgRNA and mCherry coding plasmids. EGFP disruption values were calculated as follows: the average EGFP background loss from control transfections made in the same experiment was subtracted from each individual treatment in that experiment and the mean values and the standard deviation (s.d.) were calculated from it.

GUIDE-Seq

GUIDE-seq experiments were performed with WT SpCas9, B-SpCas9, eSpCas9, eSpCas9-plus, SpCas9-HF1, SpCas9-HF1-plus, on thirteen different target sites. Briefly, 2×10⁶ HEK293.EGFP cells were transfected with 3 μg of SpCas9 variant expressing plasmid, 1.5 μg of mCherry and sgRNA coding plasmid. 100 pmol of the dsODN containing phosphorothioate bonds at both ends (according to the original GUIDE-seq protocol (Tsai et al., 2015)) was mixed together with 100 μl home-made nucleofection solution to the plasmid and electroporated as described in Vriend et al. (Vriend et al., 2014) using Nucleofector (Lonza) with A23 program and 2 mm electroporation cuvettes.

Transfected cells were analyzed 3 days post-transfection by flow cytometry. Cells were then centrifuged at 1000 rcf for 10 minutes and genomic DNA was purified according to Puregene DNA Purification protocol (Gentra systems). Genomic DNA was sheared with BioraptorPlus (Diagenode) to 550 bp in average. Sample libraries were assembled as previously described (Tsai et al., 2015) and sequenced on an Illumina MiSeq instrument by ATGandCo Ltd. Data were analyzed using open-source guideseq software (version 1.1)(Tsai et al., 2016). Consolidated reads were mapped to the human reference genome GrCh37 supplemented with the integrated EGFP sequence. Upon identification of the genomic regions integrating double-stranded oligodeoxynucleotide (dsODNs) in aligned data, off-target sites were retained if at most seven mismatches against the target were present and if absent in the background controls. Visualization of aligned off-target sites are provided as a color-coded sequence grid.

TIDE

Tracking of Indels by DEcomposition (TIDE) method (Brinkman et al., 2014) was applied for analyzing mutations and determining their frequency in a cell population using different sgRNAs and SpCas9 proteins. From the isolated genomic DNA PCR was conducted with Q5 High-Fidelity DNA Polymerase in triplicates (for PCR primer details, see Table 3). Genomic PCR products were gel excised via NucleoSpin Gel and PCR Clean-up kit and were Sanger sequenced. Indel efficiencies were analysed by TIDE webtool (https://tide.nki.nl/) by comparing SpCas9 treated and control samples.

TABLE 3
List of primers used to amplify sequence of interest for TIDE

| | | | SEQ ID |
|---|---|---|---|
| Name of primers | Sequence | References | NO: |
| FANCF site 2-for | GGGCCGGGAAAGAGTTGCTG | from Kleinstiver et al., Nature 2016 | 99 |
| FANCF site 2-rev | GCCCTACATCTGCTCTCCCTCC | from Kleinstiver et al., Nature 2016 | 100 |
| VEGFA site 2-for | CGAGGAAGAGAGAGACGGGGTC | from Chen et al., Nature 2017 | 101 |
| VEGFA site 2-rev | CTCCAATGCACCCAAGACAGCAG | from Chen et al., Nature 2017 | 102 |
| HEK site 4-for | AGAGAAGTTGGAGTGAAGGCAGAG | from Casini et al., Nature Biotech. 2018 | 103 |
| HEK site 4-rev | GTCAGACGTCCAAAACCAGACTCC | from Casini et al., Nature Biotech. 2018 | 104 |
| ZSCAN2-for | AGTGTGGGGTGTGTGGGAAG | from Kleinstiver et al., Nature 2016 | 105 |
| ZSCAN2-rev | ACGGGACTTGACTCAGACCACT | from Kleinstiver et al., Nature 2016 | 106 |
| EMXI site 2-for | GGAGCAGCTGGTCAGAGGGG | from Kleinstiver et al., Nature 2016 | 107 |
| EMXI site 2-rev | CCATAGGGAAGGGGGACACTGG | from Kleinstiver et al., Nature 2016 | 108 |
| DNMT1 site 4-for | CCAGAATGCACAAAGTACTGCAC | from Chen et al., Nature 2017 | 109 |
| DNMT1 site 4-rev | GCCAAAGCCCGAGAGAGTGCC | from Chen et al., Nature 2017 | 110 |

Pre-Screening Shadoo (Sprn) Gene Target Sites (HR Mediated Integration Assay)

N2a cells were seeded into 48-well plates a day before transfection at a density of 2.5×10⁴ cells/well. Next day cells were co-transfected with three types of plasmids: an expression plasmid for EGFP flanked by 1,000 bp-long homology arms to the Sprn gene (Sprn.HA-CMV-EGFP plasmid) (166 ng), SpCas9 expressing plasmid (42 ng) and an sgRNA/mCherry coding plasmid (42 ng), giving 250 ng total plasmid DNA, using 1 μl TurboFect reagent per well. Transfected cells were analyzed 4- and 18-days post-transfection by flow cytometry. Transfection efficiency was calculated via mCherry expressing cells measured 4 days post-transfection. EGFP positive cells were counted 18 days post-transfection. Transfections were performed in triplicate.

NHEJ-Mediated Integration Using a ‘Self-Cleaving’ EGFP-Expression Plasmid (Talas et al., 2017)

N2a cells were seeded into 12-well plates a day before transfection at a density of 8×10⁴ cells/well. Next day cells were co-transfected with three types of plasmids: a ‘self-cleaving’ EGFP-expression plasmid (Talas et al., 2017) (which has to integrate in-frame for Sprn promoter driven EGFP expression) (1 μg), SpCas9 expressing plasmid (590 ng) and an sgRNA/mCherry coding plasmid (410 ng), giving 2 μg total plasmid DNA, using 4 μl TurboFect reagent per well. Transfections were performed in triplicate. Transfection efficiency was calculated via mCherry expressing cells measured 4-days post-transfection. EGFP positive cells were counted 14-days post-transfection.

Statistics

Differences between SpCas9 variants were tested by using either Paired-samples Student's t-test (FIG. 2b: evoSpCas9/B-evoSpCas9; FIG. 2a: WT SpCas9/B-SpCas9, eSpCas9/B-eSpCas9; FIG. 3a: evoSpCas9/B-evoSpCas9, HeFSpCas9/B-HeFSpCas9; Supplementary FIG. 7c: eSpCas9-ribosyme/eSpCas9-tRNA) or by using Wilcoxon Signed Ranks test (FIG. 2b: WT SpCas9/B-SpCas9, eSpCas9/B-eSpCas9, SpCas9-HF1/B-SpCas9-HF1, HypaSpCas9/B-HypaSpCas9; FIG. 2a: SpCas9-HF1/B-SpCas9-HF1, HypaSpCas9/B-HypaSpCas9, evoSpCas9/B-evoSpCas9, HeFSpCas9/B-HeFSpCas9; FIG. 3a: eSpCas9/B-eSpCas9, SpCas9-HF1/B-SpCas9-HF1, HypaSpCas9/B-HypaSpCas9; Supplementary FIG. 3b: WT SpCas9/B-SpCas9; Supplementary FIG. 7c: eSpCas9/eSpCas9-plus, eSpCas9/eSpCas9-ribosyme, eSpCas9/eSpCas9-tRNA, eSpCas9-plus/eSpCas9-ribosyme, eSpCas9-plus/eSpCas9-tRNA) in cases where differences did not meet the assumptions of Paired t-test. Normality of data and of differences were tested by Shapiro-Wilk normality test. Statistical tests were performed using IBM SPSS ver. 20 on data including all parallel sample points.

Supplementary Material on Sequences

Supplementary Sequences

For detailed primer, oligonucleotide, Addgene number and SpCas9 construct information see the tables below. The sequences of all plasmid constructs were confirmed by Sanger sequencing.

TABLE 4
List of EGFP target sites and spacers. Modified sgRNAs targeting the identical EGFP sites,
are named with the same number, but with an extension in the name (e.g. B, C, -no 5′ G).

| | | | Spacer | | | |
|---|---|---|---|---|---|---|
| | Prep | | length | Spacer Sequence | Target sequence with PAM | Sense/ |
| | Name | Name | (nt) | SEQ ID NOs 111-219 | SEQ ID NOs 220-329 | Anti-sense |
| | 8323 | EGFP site 1 | 20 | GGGCACGGGCAGCTTGCCGG | GGGCACGGGCAGCTTGCCGGtgg | a |
| | 1581 | EGFP site 2 | 20 | GAGCTGGACGGCGACGTAAA | GAGCTGGACGGCGACGTAAAcgg | s |
| | 1582 | EGFP site 3 | 20 | GGTGGTGCAGATGAACTTCA | GGTGGTGCAGATGAACTTCAggg | s |
| | 1584 | EGFP site 4 | 20 | GGAGCGCACCATCTTCTTCA | GGAGCGCACCATCTTCTTCAagg | s |
| | 1585 | EGFP site 5 | 20 | GTCGCCCTCGAACTTCACCT | GTCGCCCTCGAACTTCACCTcgg | a |
| | 1586 | EGFP site 6 | 20 | GCGCGATCACATGGTCCTGC | GCGCGATCACATGGTCCTGCtgg | s |
| | 1587 | EGFP site 7 | 20 | GTCCATGCCGAGAGTGATCC | GTCCATGCCGAGAGTGATCCcgg | a |
| | 1588 | EGFP site 8 | 20 | GCCGTCGTCCTTGAAGAAGA | GCCGTCGTCCTTGAAGAAGAtgg | a |
| | 1589 | EGFP site 9 | 20 | GAAGTTCGAGGGCGACACCC | GAAGTTCGAGGGCGACACCCtgg | s |
| | 1590 | EGFP site 10 | 20 | GTCTTTGCTCAGGGCGGACT | GTCTTTGCTCAGGGCGGACTggg | a |
| | 1591 | EGFP site 11 | 20 | GGTCTTTGCTCAGGGCGGAC | GGTCTTTGCTCAGGGCGGACtgg | a |
| | 1592 | EGFP site 12 | 20 | GTGCTCAGGTAGTGGTTGTC | GTGCTCAGGTAGTGGTTGTCggg | a |
| | 1593 | EGFP site 13 | 20 | GCTGAAGGGCATCGACTTCA | GCTGAAGGGCATCGACTTCAagg | s |
| | 13012 | EGFP site 14 | 20 | GTACCAGCACTAGCCTCCTG | GTACCAGCACTAGCCTCCTGagg | a |
| | 13020 | EGFP site 15 | 20 | GGCATCGCCCTCGCCCTCGC | GGCATCGCCCTCGCCCTCGCcgg | a |
| | 13021 | EGFP site 16 | 20 | GGCGAGGGCGATGCCACCTA | GGCGAGGGCGATGCCACCTAcgg | s |
| | 1633 | EGFP site 17 | 20 | GCTGAAGCACTGCACGCCGT | GCTGAAGCACTGCACGCCGTagg | a |
| | 1634 | EGFP site 18 | 20 | GTGAACCGCATCGAGCTGAA | GTGAACCGCATCGAGCTGAAggg | s |
| | 1635 | EGFP site 19 | 20 | GTCAGGGTGGTCACGAGGGT | GTCAGGGTGGTCACGAGGGTggg | a |
| | 1636 | EGFP site 20 | 20 | GACGTAGCCTTCGGGCATGG | GACGTAGCCTTCGGGCATGGcgg | a |
| | 1637 | EGFP site 21 | 20 | GGCATCGACTTCAAGGAGGA | GGCATCGACTTCAAGGAGGAcgg | s |
| | 1638 | EGFP site 22 | 20 | GGTGTTCTGCTGGTAGTGGT | GGTGTTCTGCTGGTAGTGGTcgg | a |
| | 1639 | EGFP site 23 | 20 | GTGGTCACGAGGGTGGGCCA | GTGGTCACGAGGGTGGGCCAggg | a |
| | 1557 | EGFP site 24 | 20 | GTCGTGCTGCTTCATGTGGT | GTCGTGCTGCTTCATGTGGTcgg | a |
| | 8623 | EGFP site 25 | 20 | GGTCCGAGAGTCTGTAGCCA | GGTCCGAGAGTCTGTAGCCAtgg | a |
| | 8624 | EGFP site 26 | 20 | GCTGACGGTCAGGAGCCAGG | GCTGACGGTCAGGAGCCAGGagg | a |
| | 8625 | EGFP site 27 | 20 | GGCAGAGCAGGCTGACGGTC | GGCAGAGCAGGCTGACGGTCagg | a |
| | 8626 | EGFP site 28 | 20 | GAGCAGGCAGAGCAGGCTGA | GAGCAGGCAGAGCAGGCTGAcgg | a |
| | 8627 | EGFP site 29 | 20 | GAGGCCAGAGCAGGCAGAGC | GAGGCCAGAGCAGGCAGAGCagg | a |
| | 8628 | EGFP site 30 | 20 | GCTCTGCCTGCTCTGGCCTC | GCTCTGCCTGCTCTGGCCTCagg | s |
| | 8629 | EGFP site 31 | 20 | GGGCGAGGAGCTGTTCACCG | GGGCGAGGAGCTGTTCACCGggg | s |
| | 8630 | EGFP site 32 | 20 | GACCAGGATGGGCACCACCC | GACCAGGATGGGCACCACCCcgg | a |
| | 8631 | EGFP site 33 | 20 | GGTGCCCATCCTGGTCGAGC | GGTGCCCATCCTGGTCGAGCtgg | s |
| | 8632 | EGFP site 34 | 20 | GCCGTCCAGCTCGACCAGGA | GCCGTCCAGCTCGACCAGGAtgg | a |
| | 8633 | EGFP site 35 | 20 | GGCCACAAGTTCAGCGTGTC | GGCCACAAGTTCAGCGTGTCcgg | s |
| | 8634 | EGFP site 36 | 20 | GGTGGTCACGAGGGTGGGCC | GGTGGTCACGAGGGTGGGCCagg | a |
| | 8635 | EGFP site 37 | 20 | GGTCAGGGTGGTCACGAGGG | GGTCAGGGTGGTCACGAGGGtgg | a |
| | 8636 | EGFP site 38 | 20 | GTAGGTCAGGGTGGTCACGA | GTAGGTCAGGGTGGTCACGAggg | a |
| | 8637 | EGFP site 39 | 20 | GCACTGCACGCCGTAGGTCA | GCACTGCACGCCGTAGGTCAggg | a |
| | 8638 | EGFP site 40 | 20 | GCTTCATGTGGTCGGGGTAG | GCTTCATGTGGTCGGGGTAGcgg | a |
| | 8639 | EGFP site 41 | 20 | GCGCTCCTGGACGTAGCCTT | GCGCTCCTGGACGTAGCCTTcgg | a |
| | 8640 | EGFP site 42 | 20 | GGTGAACCGCATCGAGCTGA | GGTGAACCGCATCGAGCTGAagg | s |
| | 8641 | EGFP site 43 | 20 | GAAGGGCATCGACTTCAAGG | GAAGGGCATCGACTTCAAGGagg | s |
| | 8642 | EGFP site 44 | 20 | GATGCCGTTCTTCTGCTTGT | GATGCCGTTCTTCTGCTTGTcgg | a |
| | 8643 | EGFP site 45 | 20 | GCACGGGGCCGTCGCCGATG | GCACGGGGCCGTCGCCGATGggg | a |
| | 8644 | EGFP site 46 | 20 | GGTTGTCGGGCAGCAGCACG | GGTTGTCGGGCAGCAGCACGggg | a |
| | 8645 | EGFP site 47 | 20 | GTGGTTGTCGGGCAGCAGCA | GTGGTTGTCGGGCAGCAGCAcgg | a |
| | 8646 | EGFP site 48 | 20 | GGTGCTCAGGTAGTGGTTGT | GGTGCTCAGGTAGTGGTTGTcgg | a |
| | 8647 | EGFP site 49 | 20 | GTTGGGGTCTTTGCTCAGGG | GTTGGGGTCTTTGCTCAGGGcgg | a |
| | 8648 | EGFP site 50 | 20 | GCCGAGAGTGATCCCGGCGG | GCCGAGAGTGATCCCGGCGGcgg | a |
| [symbol] | 1550 | EGFP site 51- | 20 | CGATGCCCTTCAGCTCGATG | CGATGCCCTTCAGCTCGATGcgg | a |
| not G | | no 5′ G | | | | |
| | 1551 | EGFP site 52- | 20 | CAACATCCTGGGGCACAAGC | CAACATCCTGGGGCACAAGCtgg | s |
| | | no 5′ G | | | | |
| | 1552 | EGFP site 53- | 20 | CAGCTCGATGCGGTTCACCA | CAGCTCGATGCGGTTCACCAggg | a |
| | | no 5′ G | | | | |
| | 1553 | EGFP site 54- | 20 | CAAGGAGGACGGCAACATCC | CAAGGAGGACGGCAACATCCtgg | s |
| | | no 5′ G | | | | |
| | 1554 | EGFP site 55- | 20 | TCAGCTCGATGCGGTTCACC | TCAGCTCGATGCGGTTCACCagg | a |
| | | no 5′ G | | | | |
| | 1555 | EGFP site 56- | 20 | AAGGAGGACGGCAACATCCT | AAGGAGGACGGCAACATCCTggg | s |
| | | no 5′ G | | | | |
| | 1556 | EGFP site 57- | 20 | AGGAGGACGGCAACATCCTG | AGGAGGACGGCAACATCCTGggg | s |
| | | no 5′ G | | | | |
| | 1558 | EGFP site 58- | 20 | CATGCCCGAAGGCTACGTCC | CATGCCCGAAGGCTACGTCCagg | s |
| | | no 5′ G | | | | |
| | 1560 | EGFP site 59- | 20 | CGTGCTGCTTCATGTGGTCG | CGTGCTGCTTCATGTGGTCGggg | a |
| | | no 5′ G | | | | |
| Series B: | 1631 | EGFP site IB | 21 | gGGGCACGGGCAGCTTGCCGG | GGGCACGGGCAGCTTGCCGGtgg | a |
| 21st position | 1621 | EGFP site 2B | 21 | gGAGCTGGACGGCGACGTAAA | GAGCTGGACGGCGACGTAAAcgg | s |
| G mismatches | 1622 | EGFP site 3B | 21 | gGGTGGTGCAGATGAACTTCA | GGTGGTGCAGATGAACTTCAggg | s |
| | 1623 | EGFP site 4B | 21 | gGGAGCGCACCATCTTCTTCA | GGAGCGCACCATCTTCTTCAagg | s |
| | 1624 | EGFP site 5B | 21 | gGTCGCCCTCGAACTTCACCT | GTCGCCCTCGAACTTCACCTcgg | a |
| | 8757 | EGFP site 6B | 21 | gGCGCGATCACATGGTCCTGC | GCGCGATCACATGGTCCTGCtgg | s |
| | 8758 | EGFP site 7B | 21 | gGTCCATGCCGAGAGTGATCC | GTCCATGCCGAGAGTGATCCcgg | a |
| | 1625 | EGFP site 8B | 21 | gGCCGTCGTCCTTGAAGAAGA | GCCGTCGTCCTTGAAGAAGAtgg | a |
| | 1626 | EGFP site 9B | 21 | gGAAGTTCGAGGGCGACACCC | GAAGTTCGAGGGCGACACCCtgg | s |
| | 8760 | EGFP site 13B | 21 | gGCTGAAGGGCATCGACTTCA | GCTGAAGGGCATCGACTTCAagg | s |
| | 1627 | EGFP site 14B | 21 | gGTACCAGCACTAGCCTCCTG | GTACCAGCACTAGCCTCCTGagg | a |
| | 1628 | EGFP site 15B | 21 | gGGCATCGCCCTCGCCCTCGC | GGCATCGCCCTCGCCCTCGCcgg | a |
| | 1632 | EGFP site 24B | 21 | gGTCGTGCTGCTTCATGTGGT | GTCGTGCTGCTTCATGTGGTcgg | a |
| | 8761 | EGFP site 25B | 21 | gGGTCCGAGAGTCTGTAGCCA | GGTCCGAGAGTCTGTAGCCAtgg | a |
| | 8763 | EGFP site 27B | 21 | gGGCAGAGCAGGCTGACGGTC | GGCAGAGCAGGCTGACGGTCagg | a |
| | 8661 | EGFP site 28B | 21 | gGAGCAGGCAGAGCAGGCTGA | GAGCAGGCAGAGCAGGCTGAcgg | a |
| | 8662 | EGFP site 29B | 21 | gGAGGCCAGAGCAGGCAGAGC | GAGGCCAGAGCAGGCAGAGCagg | a |
| | 8663 | EGFP site 30B | 21 | gGCTCTGCCTGCTCTGGCCTC | GCTCTGCCTGCTCTGGCCTCagg | s |
| | 8649 | EGFP site 3IB | 21 | gGGGCGAGGAGCTGTTCACCG | GGGCGAGGAGCTGTTCACCGggg | s |
| | 8650 | EGFP site 32B | 21 | gGACCAGGATGGGCACCACCC | GACCAGGATGGGCACCACCCcgg | a |
| | 8651 | EGFP site 33B | 21 | gGGTGCCCATCCTGGTCGAGC | GGTGCCCATCCTGGTCGAGCtgg | s |
| | 8652 | EGFP site 34B | 21 | gGCCGTCCAGCTCGACCAGGA | GCCGTCCAGCTCGACCAGGAtgg | a |
| | 8764 | EGFP site 35B | 21 | gGGCCACAAGTTCAGCGTGTC | GGCCACAAGTTCAGCGTGTCcgg | s |
| | 8653 | EGFP site 37B | 21 | gGGTCAGGGTGGTCACGAGGG | GGTCAGGGTGGTCACGAGGGtgg | a |
| | 8654 | EGFP site 38B | 21 | gGTAGGTCAGGGTGGTCACGA | GTAGGTCAGGGTGGTCACGAggg | a |
| | 8655 | EGFP site 39B | 21 | gGCACTGCACGCCGTAGGTCA | GCACTGCACGCCGTAGGTCAggg | a |
| | 8656 | EGFP site 40B | 21 | gGCTTCATGTGGTCGGGGTAG | GCTTCATGTGGTCGGGGTAGcgg | a |
| | 8766 | EGFP site 41B | 21 | gGCGCTCCTGGACGTAGCCTT | GCGCTCCTGGACGTAGCCTTcgg | a |
| | 8767 | EGFP site 42B | 21 | gGGTGAACCGCATCGAGCTGA | GGTGAACCGCATCGAGCTGAagg | s |
| | 8664 | EGFP site 43B | 21 | gGAAGGGCATCGACTTCAAGG | GAAGGGCATCGACTTCAAGGagg | s |
| | 8768 | EGFP site 44B | 21 | gGATGCCGTTCTTCTGCTTGT | GATGCCGTTCTTCTGCTTGTcgg | a |
| | 8659 | EGFP site 45B | 21 | gGCACGGGGCCGTCGCCGATG | GCACGGGGCCGTCGCCGATGggg | a |
| | 8665 | EGFP site 46B | 21 | gGGTTGTCGGGCAGCAGCACG | GGTTGTCGGGCAGCAGCACGggg | a |
| | 8666 | EGFP site 47B | 21 | gGTGGTTGTCGGGCAGCAGCA | GTGGTTGTCGGGCAGCAGCAcgg | a |
| | 8660 | EGFP site 49B | 21 | gGTTGGGGTCTTTGCTCAGGG | GTTGGGGTCTTTGCTCAGGGcgg | a |
| | 8770 | EGFP site 50B | 21 | gGCCGAGAGTGATCCCGGCGG | GCCGAGAGTGATCCCGGCGGcgg | a |
| Series C: | 1611 | EGFP site 10C | 21 | gGTCTTTGCTCAGGGCGGACT | gGTCTTTGCTCAGGGCGGACTggg | a |
| 21st position | 8759 | EGFP site 11C | 21 | gGGTCTTTGCTCAGGGCGGAC | GGTCTTTGCTCAGGGCGGACtgg | a |
| G matches | 1612 | EGFP site 12C | 21 | gGTGCTCAGGTAGTGGTTGTC | gGTGCTCAGGTAGTGGTTGTCggg | a |
| | 1609 | EGFP site 16C | 21 | gGGCGAGGGCGATGCCACCTA | gGGCGAGGGCGATGCCACCTAcgg | s |
| | 1613 | EGFP site 17C | 21 | gGCTGAAGCACTGCACGCCGT | gGCTGAAGCACTGCACGCCGTagg | a |
| | 1614 | EGFP site 18C | 21 | gGTGAACCGCATCGAGCTGAA | gGTGAACCGCATCGAGCTGAAggg | s |
| | 1615 | EGFP site 19C | 21 | gGTCAGGGTGGTCACGAGGGT | gGTCAGGGTGGTCACGAGGGTggg | a |
| | 1616 | EGFP site 20C | 21 | gGACGTAGCCTTCGGGCATGG | gGACGTAGCCTTCGGGCATGGcgg | a |
| | 1617 | EGFP site 21C | 21 | gGGCATCGACTTCAAGGAGGA | gGGCATCGACTTCAAGGAGGAcgg | s |
| | 1618 | EGFP site 22C | 21 | gGGTGTTCTGCTGGTAGTGGT | gGGTGTTCTGCTGGTAGTGGTegg | a |
| | 1619 | EGFP site 23C | 21 | gGTGGTCACGAGGGTGGGCCA | gGTGGTCACGAGGGTGGGCCAggg | a |
| | 8762 | EGFP site 26C | 21 | gGCTGACGGTCAGGAGCCAGG | GCTGACGGTCAGGAGCCAGGagg | a |
| | 8765 | EGFP site 36C | 21 | gGGTGGTCACGAGGGTGGGCC | GGTGGTCACGAGGGTGGGCCagg | a |
| | 8769 | EGFP site 48C | 21 | gGGTGCTCAGGTAGTGGTTGT | GGTGCTCAGGTAGTGGTTGTcgg | a |

[symbol]

indicates data missing or illegible when filed

TABLE 5
List of EGFP target sites and 21^{st} G , ribosyme or tRNA flanked spacers.
Modified sgRNAs targeting the identical EGFP sites but flanked either
with 21^{st} G nucleotide, ribozyme or tRNA.

| | | | | tRNA | |
|---|---|---|---|---|---|
| | | 21G-sgRNAs | Ribosyme | flanked- | |
| | Target | (Spacer | flanked-sgRNAs | sgRNAs | |
| | seq. with | sequence) | (Ribosyme-Spacer | (Spacer seq.) | Sense/ |
| | PAM SEQ ID | SEQ ID NOs | sequence) | SEQ ID NOs | Anti- |
| Name | NOs 330-350 | 351-371 | SEQ ID NOs 372-392 | 393-413 | sense |
| EGFP | CGATGCCCTTCAG | gCGATGCCCTT | GCATCGCTGATGAGTCCGTGA | CGATGCCCTT | a |
| site 51 | CTCGATGcgg | CAGCTCGATG | GGACGAAACGAGTAAGCTCGT | CAGCTCGATG | |
| | | | CCGATGCCCTTCAGCTCGATG | | |
| EGFP | CAACATCCTGGGG | gCAACATCCTG | ATGTTGCTGATGAGTCCGTGA | CAACATCCTG | s |
| site 52 | CACAAGCtgg | GGGCACAAGC | GGACGAAACGAGTAAGCTCGT | GGGCACAAGC | |
| | | | CCAACATCCTGGGGCACAAGC | | |
| EGFP | CAGCTCGATGCGG | gCAGCTCGATG | GAGCTGCTGATGAGTCCGTGA | CAGCTCGATG | a |
| site 53 | TTCACCAggg | CGGTTCACCA | GGACGAAACGAGTAAGCTCGT | CGGTTCACCA | |
| | | | CCAGCTCGATGCGGTTCACCA | | |
| EGFP | TCAGCTCGATGCG | gTCAGCTCGAT | AGCTGACTGATGAGTCCGTGA | TCAGCTCGAT | a |
| site 55 | GTTCACCagg | GCGGTTCACC | GGACGAAACGAGTAAGCTCGT | GCGGTTCACC | |
| | | | CTCAGCTCGATGCGGTTCACC | | |
| EGFP | CGTGCTGCTTCAT | gCGTGCTGCTT | AGCACGCTGATGAGTCCGTGA | CGTGCTGCTT | a |
| site 59 | GTGGTCGggg | CATGTGGTCG | GGACGAAACGAGTAAGCTCGT | CATGTGGTCG | |
| | | | CCGTGCTGCTTCATGTGGTCG | | |
| EGFP | CCGTCCAGCTCGA | gCCGTCCAGCT | GGACGGCTGATGAGTCCGTGA | CCGTCCAGCT | a |
| site 60 | CCAGGATggg | GGACCAGGAT | GGACGAAACGAGTAAGCTCGT | GGACCAGGAT | |
| | | | CCCGTCCAGCTCGACCAGGAT | | |
| EGFP | CAACTACAAGACC | gCAACTACAAG | TAGTTGCTGATGAGTCCGTGA | CAACTACAAG | s |
| site 61 | CGCGCCGagg | ACCCGCGCCG | GGACGAAACGAGTAAGCTCGT | ACCCGCGCCG | |
| | | | CCAACTACAAGACCCGCGCCG | | |
| EGFP | AAGGGCGAGGAGC | gAAGGGCGAGG | GCCCTTCTGATGAGTCCGTGA | AAGGGCGAGG | s |
| site 62 | TGTTCACcgg | AGCTGTTCAC | GGACGAAACGAGTAAGCTCGT | AGCTGTTCAC | |
| | | | CAAGGGCGAGGAGCTGTTCAC | | |
| EGFP | AAGTTCAGCGTGT | gAAGTTCAGCG | GAACTTCTGATGAGTCCGTGA | AAGTTCAGCG | s |
| site 63 | CCGGCGAggg | TGTCCGGCGA | GGACGAAACGAGTAAGCTCGT | TGTCCGGCGA | |
| | | | CAAGTTCAGCGTGTCCGGCGA | | |
| EGFP | AGCGTGTCCGGCG | gAGCGTGTCCG | CACGCTCTGATGAGTCCGTGA | AGCGTGTCCG | s |
| site 64 | AGGGCGAggg | GCGAGGGCGA | GGACGAAACGAGTAAGCTCGT | GCGAGGGCGA | |
| | | | CAGCGTGTCCGGCGAGGGCGA | | |
| EGFP | ACGAGGGTGGGCC | gACGAGGGTGG | CCTCGTCTGATGAGTCCGTGA | ACGAGGGTGG | a |
| site 65 | AGGGCACggg | GCCAGGGCAC | GGACGAAACGAGTAAGCTCGT | GCCAGGGCAC | |
| | | | CACGAGGGTGGGCCAGGGCAC | | |
| EGFP | AGAAGTCGTGCTG | gAGAAGTCGTG | ACTTCTCTGATGAGTCCGTGA | AGAAGTCGTG | a |
| site 66 | CTTCATGtgg | CTGCTTCATG | GGACGAAACGAGTAAGCTCGT | CTGCTTCATG | |
| | | | CAGAAGTCGTGCTGCTTCATG | | |
| EGFP | ACCATCTTCTTCA | gACCATCTTCT | GATGGTCTGATGAGTCCGTGA | ACCATCTTCT | s |
| site 67 | AGGACGAcgg | TCAAGGACGA | GGACGAAACGAGTAAGCTCGT | TCAAGGACGA | |
| | | | CACCATCTTCTTCAAGGACGA | | |
| EGFP | AAGGAGGACGGCA | gAAGGAGGACG | CTCCTTCTGATGAGTCCGTGA | AAGGAGGACG | s |
| site 68 | ACATCCTggg | GCAACATCCT | GGACGAAACGAGTAAGCTCGT | GCAACATCCT | |
| | | | CAAGGAGGACGGCAACATCCT | | |
| EGFP | TCAGCTCGATGCG | gTCAGCTCGAT | AGCTGACTGATGAGTCCGTGA | TCAGCTCGAT | a |
| site 69 | GTTCACCagg | GCGGTTCACC | GGACGAAACGAGTAAGCTCGT | GCGGTTCACC | |
| | | | CTCAGCTCGATGCGGTTCACC | | |
| EGFP | TCGTGCTGCTTCA | gTCGTGCTGCT | GCACGACTGATGAGTCCGTGA | TCGTGCTGCT | a |
| site 70 | TGTGGTCggg | TCATGTGGTC | GGACGAAACGAGTAAGCTCGT | TCATGTGGTC | |
| | | | CTCGTGCTGCTTCATGTGGTC | | |
| EGFP | TTCAAGTCCGCCA | gTTCAAGTCCG | CTTGAACTGATGAGTCCGTGA | TTCAAGTCCG | s |
| site 71 | TGCCCGAagg | CCATGCCCGA | GGACGAAACGAGTAAGCTCGT | CCATGCCCGA | |
| | | | CTTCAAGTCCGCCATGCCCGA | | |
| EGFP | TGAAGAAGATGGT | gTGAAGAAGAT | TCTTCACTGATGAGTCCGTGA | TGAAGAAGAT | a |
| site 72 | GCGCTCCtgg | GGTGCGCTCC | GGACGAAACGAGTAAGCTCGT | GGTGCGCTCC | |
| | | | CTGAAGAAGATGGTGCGCTCC | | |
| EGFP | TGTACTCCAGCTT | gTGTACTCCAG | AGTACACTGATGAGTCCGTGA | TGTACTCCAG | a |
| site 73 | GTGCCCCagg | CTTGTGCCCC | GGACGAAACGAGTAAGCTCGT | CTTGTGCCCC | |
| | | | CTGTACTCCAGCTTGTGCCCC | | |
| EGFP | TGCCGTCCTCGAT | gTGCCGTCCTC | ACGGCACTGATGAGTCCGTGA | TGCCGTCCTC | a |
| site 74 | GTTGTGGcgg | GATGTTGTGG | GGACGAAACGAGTAAGCTCGT | GATGTTGTGG | |
| | | | CTGCCGTCCTCGATGTTGTGG | | |
| EGFP | CCGCGCCGAGGTG | gCCGCGCCGAG | GCGCGGCTGATGAGTCCGTGA | CCGCGCCGAG | s |
| site 75 | AAGTTCGagg | GTGAAGTTCG | GGACGAAACGAGTAAGCTCGT | GTGAAGTTCG | |
| | | | CCCGCGCCGAGGTGAAGTTCG | | |

TABLE 6
List of endogenous target sites and spacers. 5′ modified sgRNAs have an
extension in the name (e.g. B, C, 21G, same nomenclature as in App. Table 1).

| | Spacer | | | | |
|---|---|---|---|---|---|
| | length | Spacer Sequence | Sequence with PAM | | Sense/ |
| Name | (nt) | SEQ ID NOs 414-449 | SEQ ID NOs 450-485 | References | Anti-sense |
| FANCF | 20 | GCGATCCAGGTGCTGCAGAA | GCGATCCAGGTGCTGCAGAAggg | | a |
| site 1 | | | | | |
| FANCF | 20 | GCTGCAGAAGGGATTCCATG | GCTGCAGAAGGGATTCCATGagg | Kleinstiver et | a |
| site 2 | | | | al., Nature 2016 | |
| FANCF | 21 | gTAGGGCCTTCGCGCACCTCA | TAGGGCCTTCGCGCACCTCAtgg | | s |
| site 3 | | | | | |
| FANCF | 21 | gCCAAGGTGAAAGCGGAAGTA | CCAAGGTGAAAGCGGAAGTAggg | | s |
| site 4 | | | | | |
| FANCF | 21 | gTCCAAGGTGAAAGCGGAAGT | TCCAAGGTGAAAGCGGAAGTagg | | s |
| site 5 | | | | | |
| FANCF | 21 | gCGCCGTCTCCAAGGTGAAAG | CGCCGTCTCCAAGGTGAAAGcgg | | s |
| site 6 | | | | | |
| FANCF | 21 | gCCTGCTCTCTCTGCGCCTGC | CCTGCTCTCTCTGCGCCTGCtgg | | s |
| site 7 | | | | | |
| FANCF | 21 | gCCAGCAGGCGCAGAGAGAGC | CCAGCAGGCGCAGAGAGAGCagg | | a |
| site 8 | | | | | |
| FANCF | 21 | gCAGGACGTCACAGTGACCGA | CAGGACGTCACAGTGACCGAggg | | a |
| site 9 | | | | | |
| FANCF | 21 | gTTAGCGAACTTCCAGGCCCT | TTAGCGAACTTCCAGGCCCTcgg | | s |
| site 10 | | | | | |
| FANCF | 21 | gCGTCACAGTGACCGAGGGCC | CGTCACAGTGACCGAGGGCCtgg | | a |
| site 11 | | | | | |
| FANCF | 21 | gTCCGGGATTAGCGAACTTCC | TCCGGGATTAGCGAACTTCCagg | | s |
| site 12 | | | | | |
| VEGFA | 21 | gCTTCATGTACAGAGAGCCCA | CTTCATGTACAGAGAGCCCAggg | | a |
| site 1 | | | | | |
| VEGFA | 21 | gAACAGCTACATATTTGGGAC | AACAGCTACATATTTGGGACtgg | | a |
| site 3 | | | | | |
| VEGFA | 20 | GTCCCAAATATGTAGCTGTT | GTCCCAAATATGTAGCTGTTtgg | | s |
| site 4 | | | | | |
| VEGFA | 21 | gCAAATATGTAGCTGTTTGGG | CAAATATGTAGCTGTTTGGGagg | | s |
| site 5 | | | | | |
| VEGFA | 21 | gTTTGGGAGGTCAGAAATAGG | TTTGGGAGGTCAGAAATAGGggg | | s |
| site 6 | | | | | |
| VEGFA | 20 | GGGTGGGGGGAGTTTGCTCC | GGGTGGGGGGAGTTTGCTCCtgg | | a |
| site 7 | | | | | |
| VEGFA | 21 | gCTTTGCTAGGAATATTGAAG | CTTTGCTAGGAATATTGAAGggg | | a |
| site 8 | | | | | |
| VEGFA | 20 | GGCAGGGGAAGGCGGAGAGC | GGCAGGGGAAGGCGGAGAGCcgg | | a |
| site 9 | | | | | |
| VEGFA | 21 | gTCTCCCCACCCGTCCCTGTC | TCTCCCCACCCGTCCCTGTCcgg | | s |
| site 10 | | | | | |
| VEGFA | 20 | GAGAGCCGGACAGGGACGGG | GAGAGCCGGACAGGGACGGGtgg | | a |
| site 11 | | | | | |
| VEGFA | 20 | GGACAGGGACGGGTGGGGAG | GGACAGGGACGGGTGGGGAGagg | | a |
| site 12 | | | | | |
| mouseSprn | 21 | gTTCTGCCCAGTAGGATGAAC | TTCTGCCCAGTAGGATGAACtgg | | s |
| site 1B | | | | | |
| mouseSprn | 21 | gACTGGACTGCTGCCACGTGC | ACTGGACTGCTGCCACGTGCtgg | | s |
| site 2B | | | | | |
| mouseSprn | 21 | gCTGGACTGCTGCCACGTGCT | CTGGACTGCTGCCACGTGCTggg | | s |
| site 3B | | | | | |
| mouseSprn | 21 | gCACGTGCTGGGCTCTGCTGC | CACGTGCTGGGCTCTGCTGCtgg | | s |
| site 4B | | | | | |
| mouseSprn | 21 | gCAGCAGCAGAGCCCAGCACG | CAGCAGCAGAGCCCAGCACGtgg | | a |
| site 5B | | | | | |
| HEK site 4 | 20 | GGCACTGCGGCTGGAGGTGG | GGCACTGCGGCTGGAGGTGGggg | Casini et al., | s |
| | | | | 2018 Nature | |
| | | | | Biotech. | |
| VEGFA | 20 | GACCCCCTCCACCCCGCCTC | GACCCCCTCCACCCCGCCTCcgg | Kleinstiver et | a |
| site 2 | | | | al., Nature 2016 | |
| ZSCAN2 | 20 | GTGCGGCAAGAGCTTCAGCC | GTGCGGCAAGAGCTTCAGCCggg | Kleinstiver et | s |
| | | | | al., Nature 2016 | |
| ZSCAN2- | 21 | gGTGCGGCAAGAGCTTCAGCC | GTGCGGCAAGAGCTTCAGCCggg | | s |
| 21G | | | | | |
| EMX1 site | 20 | GTCACCTCCAATGACTAGGG | GTCACCTCCAATGACTAGGGtgg | Kleinstiver et | s |
| 2 | | | | al., Nature 2016 | |
| EMX1 site | 21 | gGTCACCTCCAATGACTAGGG | GTCACCTCCAATGACTAGGGtgg | | s |
| 2-21G | | | | | |
| DNMT1 | 20 | GGAGTGAGGGAAACGGCCCC | GGAGTGAGGGAAACGGCCCCagg | Chen et al., | s |
| site 4 | | | | Nature 2017 | |
| DNMT1 | 21 | gGGAGTGAGGGAAACGGCCCC | GGAGTGAGGGAAACGGCCCCagg | | s |
| site 4-21G | | | | | |

TABLE 7
List of mismatching sgRNAs. All sgRNAs contain a single mismatched 20 base long spacer
sequence targeting the EGFP coding sequence. The name of the mixed mismatched spacers
indicate the target site (e.g. 1 - EGFP target site 1), the position mismatched
(e.g. 1-G19) and the possible mismatches (e.g. 1-G19H; B: mix of C, G and T;
D: mix of A, G and T; H: mix of A, C and T; V: mix of A, C and G).

| | | | Mixed mis- | Spacer | Mix of spacer |
|---|---|---|---|---|---|
| | | Code of | matched | length | sequences |
| On target site | Prep Names | sgRNAs | spacers | (nt) | SEQ ID NOS 486-533 |
| EGFP site 1 | 8325, 8335-36 | 1MM1 | 1-G19H | 20 | GHGCACGGGCAGCTTGCCGG |
| | 8326, 8337-38 | 1MM2 | 1-C17D | 20 | GGGDACGGGCAGCTTGCCGG |
| | 8344-8346 | 1MM3 | 1-C15D | 20 | GGGCADGGGCAGCTTGCCGG |
| EGFP site 2 | 8371-8373 | 2MM1 | 2-A19B | 20 | GBGCTGGACGGCGACGTAAA |
| | 8374-8376 | 2MM2 | 2-C17D | 20 | GAGDTGGACGGCGACGTAAA |
| | 8377-8379 | 2MM3 | 2-G15H | 20 | GAGCTHGACGGCGACGTAAA |
| EGFP site 3 | 8380-8382 | 3MM1 | 3-T18V | 20 | GGVGGTGCAGATGAACTTGA |
| | 8383-8385 | 3MM2 | 3-G16H | 20 | GGTGHTGCAGATGAACTTCA |
| | 8386-8388 | 3MM3 | 3-G14H | 20 | GGTGGTHCAGATGAACTTCA |
| EGFP site 4 | 8389-8391 | 4MM1 | 4-A18B | 20 | GGBGCGCACCATCTTCTTCA |
| | 8392-8394 | 4MM2 | 4-C16D | 20 | GGAGDGCACCATCTTCTTCA |
| | 8395-8397 | 4MM3 | 4-C14D | 20 | GGAGCGDACCATCTTCTTCA |
| EGFP site 5 | 8398-99, 8430 | 5MM1 | 5-T19V | 20 | GVCGCCCTCGAACTTCACCT |
| | 8440, 50, 60 | 5MM2 | 5-G17H | 20 | GTCHCCCTCGAACTTCACCT |
| | 8470, 80, 90 | 5MM3 | 5-C15D | 20 | GTCGCDCTCGAACTTCACCT |
| EGFP site 6 | 8500-8502 | 6MM1 | 6-C19D | 20 | GDGCGATCACATGGTCCTGC |
| | 8503-8505 | 6MM2 | 6-C17D | 20 | GCGDGATCACATGGTCCTGC |
| | 8506-8508 | 6MM3 | 6-A15B | 20 | GCGCGBTCACATGGTCCTGC |
| EGFP site 7 | 8509-8511 | 7MM1 | 7-T19V | 20 | GVCCATGCCGAGAGTGATCC |
| | 8512-8514 | 7MM2 | 7-C17D | 20 | GTCDATGCCGAGAGTGATCC |
| | 8515-8517 | 7MM3 | 7-T15V | 20 | GTCCAVGCCGAGAGTGATCC |
| EGFP site 8 | 8518-8520 | 8MM1 | 8-C18D | 20 | GCDGTCGTCCTTGAAGAAGA |
| | 8521-8523 | 8MM2 | 8-T16V | 20 | GCCGVCGTCCTTGAAGAAGA |
| | 8524-8526 | 8MM3 | 8-G14H | 20 | GCCGTCHTCCTTGAAGAAGA |
| EGFP site 9 | 8527-8529 | 9MM1 | 9-A18B | 20 | GABGTTCGAGGGCGACACCC |
| | 8530-8532 | 9MM2 | 9-T16V | 20 | GAAGVTCGAGGGCGACACCC |
| | 8533-8535 | 9MM3 | 9-C14D | 20 | GAAGTTDGAGGGCGACACCC |
| EGFP site 10 | 8536-8538 | 10MM1 | 10-T19V | 20 | GVCTTTGCTCAGGGCGGACT |
| | 8539-8541 | 10MM2 | 10-T17V | 20 | GTCVTTGCTCAGGGCGGACT |
| | 8542-8544 | 10MM3 | 10-T15V | 20 | GTCTTVGCTCAGGGCGGACT |
| EGFP site 11 | 8545-8547 | 11MM1 | 11-T18V | 20 | GGVCTTTGCTCAGGGCGGAC |
| | 8548-8550 | 11MM2 | 11-T16V | 20 | GGTCVTTGCTCAGGGCGGAC |
| | 8551-8553 | 11MM3 | 11-T14V | 20 | GGTCTTVGCTCAGGGCGGAC |
| EGFP site 12 | 8554-8556 | 12MM1 | 12-G18H | 20 | GTHCTCAGGTAGTGGTTGTC |
| | 8557-8559 | 12MM2 | 12-T16V | 20 | GTGCVCAGGTAGTGGTTGTC |
| | 8560-8562 | 12MM3 | 12-A14B | 20 | GTGCTCBGGTAGTGGTTGTC |
| EGFP site 15 | 8572-8574 | 15MM1 | 15-C18D | 20 | GGDATCGCCCTCGCCCTCGC |
| | 8575-8577 | 15MM2 | 15-T16V | 20 | GGCAVCGCCCTCGCCCTCGC |
| | 8578-8580 | 15MM3 | 15-G14H | 20 | GGCATCHCCCTCGCCCTCGC |
| EGFP site 16 | 8581-8583 | 16MM1 | 16-C18D | 20 | GGDGAGGGCGATGCCACCTA |
| | 8584-8586 | 16MM2 | 16-A16B | 20 | GGCGBGGGCGATGCCACCTA |
| | 8587-8589 | 16MM3 | 16-G14H | 20 | GGCGAGHGCGATGCCACCTA |
| EGFP site 24 | 8471-8473 | 24MM1 | 24-T19V | 20 | GVCGTGCTGCTTCATGTGGT |
| | 8474-8476 | 24MM2 | 24-G17H | 20 | GTCHTGCTGCTTCATGTGGT |
| | 8477-8479 | 24MM3 | 24-G15H | 20 | GTCGTHCTGCTTCATGTGGT |
| EGFP site 43 | 8676-8678 | 43MM1 | 43-A18B | 20 | GABGGGCATCGACTTCAAGG |
| | 8679-8681 | 43MM2 | 43-G16H | 20 | GAGGHGCATCGACTTCAAGG |
| | 8682-8684 | 43MM3 | 43-C14D | 20 | GAGGGGDATCGACTTCAAGG |

Cas9 Spacer Cloning

sgRNA expression plasmids were constructed by ligating annealed DNA oligonucleotides harboring the spacer sequence with 4 nt-long overhangs into a BbsI restriction enzyme digested pmCherry_gRNA (#80457), or pmCheayKgRNAver2 (Addgene #126776; this plasmid backbone lacks a truncated extra guideRNA scaffold sequence) plasmids. Golden Gate cloning protocol was followed (Engler et al., 2008). The synthetic DNA oligonucleotides were hybridized and the annealed oligonucleotides (2.5 μM) with 50 ng plasmid, 3 units of BbsI restriction enzyme and 1.5 units of T4 DNA ligase were mixed in Green buffer (Thermo Fisher Scientific) containing 500 μM ATP. The mixture was kept at 37° C. for one hour before transforming into chemically competent Stable Competent E. coli cells (NEB). Two single colonies, formed after culturing on an agar plate, were tested by restriction enzyme digestion and appropriate clones were sent for sequencing.

Table 8 shows the list ofSpCas9 variants cloned and examined. In Blackjack mutations column bold indicates insertions, underlined amino acids indicate deletions.
SpCas9 variants, human expression plasmids

TABLE 8
List of SpCas9 variants cloned and examined. In Blackjack mutations column bold indicates insertions,
underlined amino acids indicate deletions.

| | | | | | | | | bacterial expression plasmids | | |
|---|---|---|---|---|---|---|---|---|---|---|
| nuclease | | | | | | | | Addgene | plas-mid | Add- |
| description | Sub #l | Sub #2 | Sub #3 | Sub #4 | Sub #5 | Sub #6 | Sub #7 | ID | # | gene ID |
| dSpCas9 | D10A | H840A | | | | | | #92112 | | |
| WT SpCas9 | | | | | | | | #126753 | 9214 | |
| eSpCas9 | K848A | K1003A | R1060A | | | | | #126754 | 9215 | #126769 |
| SpCas9-HF1 | N497A | R661A | Q695A | Q926A | | | | #126755 | 9216 | #126770 |
| HypaSpCas9 | N692A | M694A | Q695A | H698A | | | | #126756 | | |
| evoSpCas9 | M495V | Y515N | K526E | R661Q | | | | #126758 | | |
| HeFSpCas9 | N497A | R661A | Q695A | K848A | Q926A | K1003A | R1060A | #126759 | 9217 | #126771 |
| HF-B1 | N497A | R661A | Q695A | Q926A | | | | | | |
| HF-B2 | N497A | R661A | Q695A | Q926A | | | | | | |
| HF-B3 | N497A | R661A | Q695A | Q926A | | | | | | |
| HF-B4 | N497A | R661A | Q695A | Q926A | | | | | | |
| HF-B5 | N497A | R661A | Q695A | Q926A | | | | | | |
| HF-B6 | N497A | R661A | Q695A | Q926A | | | | | | |
| HF-B7 | N497A | R661A | Q695A | Q926A | | | | | | |
| HF-B8 | N497A | R661A | Q695A | Q926A | | | | | | |
| B-SpCas9- | N497A | R661A | Q695A | Q926A | | | | #126762 | | |
| HF1 (HF-B9) | | | | | | | | | | |
| HF-B10 | N497A | R661A | Q695A | Q926A | | | | | | |
| HF-B11 | N497A | R661A | Q695A | Q926A | | | | | | |
| HF-B12 | N497A | R661A | Q695A | Q926A | | | | | | |
| HF-B13 | N497A | R661A | Q695A | Q926A | | | | | | |
| HF-B14 | N497A | R661A | Q695A | Q926A | | | | | | |
| HF-B15 | N497A | R661A | Q695A | Q926A | | | | | | |
| HF-B16 | N497A | R661A | Q695A | Q926A | | | | | | |
| HF-B17 | N497A | R661A | Q695A | Q926A | | | | | | |
| HF-B18 | N497A | R661A | Q695A | Q926A | | | | | | |
| HF-B19 | N497A | R661A | Q695A | Q926A | | | | | | |
| B-SpCas9 | | | | | | | | #126760 | | |
| B-eSpCas9 | K848A | K1003A | R1060A | | | | | #126761 | 6181 | #126772 |
| B-HypaSpCas9 | N692A | M694A | Q695A | H698A | | | | #126763 | | |
| B-evoSpCas9 | M495V | Y515N | K526E | R661Q | | | | #126765 | 6182 | #126773 |
| B-HeFSpCas9 | N497A | R661A | Q695A | K848A | Q926A | K1003A | R1060A | #126766 | | |
| e + 1 | | K1003A | R1060A | | | | | #126767 | 6183 | #126774 |
| eSpCas9-plus | K848A | | R1060A | | | | | | | |
| (e + 2) | | | | | | | | | | |
| e + 3 | K848A | K1003A | | | | | | | | |
| e + 4 | K848A | | | | | | | | | |
| HF + 1 | | R661A | Q695A | Q926A | | | | #126768 | 6184 | #126775 |
| SpCas9-HFl- | N497A | | Q695A | Q926A | | | | | | |
| plus (HF + 2) | | | | | | | | | | |
| HF + 3 | N497A | R661A | | Q926A | | | | | | |
| HF + 4 | N497A | R661A | Q695A | | | | | | | |
| HF + 5 | N497A | | | Q926A | | | | | | |
| HF + 6 | N497A | | Q695A | | | | | | | |
| HF + 7 | N497A | R661A | | | | | | | | |
| Sniper | F539S | M763I | K890N | | | | | #126777 | | |
| SpCas9 | | | | | | | | | | |
| HiFiSpCas9 | R691A | | | | | | | #126778 | | |

| | | SEQ | mammalian expression | | bacterial expression | |
|---|---|---|---|---|---|---|
| nuclease | | ID | plasmids | | plasmids | |
| description | Blackjack (B) mutation/deletion/insertion | NO | plasmid # | Addgene ID | plasmid # | Addgene ID |
| dSpCas9 | — | | 1537 | #92112 | | |
| WT SpCas9 | — | | 8731 | #126753 | 9214 | |
| eSpCas9 | — | | 8749 | #126754 | 9215 | #126769 |
| SpCas9-HF1 | — | | 8720 | #126755 | 9216 | #126770 |
| HypaSpCas9 | — | | 8801 | #126756 | | |
| evoSpCas9 | — | | 8735 | #126758 | | |
| HeFSpCas9 | — | | 8750 | #126759 | 9217 | #126771 |
| HF-B1 | E1007G; Y1013G | 5 | 8717 | | | |
| HF-B2 | K1003-G-Δ LESEFVYGDY-K1014 | 6 | 8727 | | | |
| HF-B3 | K1003-Δ LESEFVYGDYKVY-D1017 | 7 | 8724 | | | |
| HF-B4 | K1003-G-Δ LESEFVYGDYKVY-D1017 | 8 | 8725 | | | |
| HF-B5 | K1003-GK-Δ LESEFVYGDYKVY-D1017 | 9 | 8726 | | | |
| HF-B6 | L1004-Δ ESEFVYGDY-K1014 | 10 | 8718 | | | |
| HF-B7 | L1004-G-Δ ESEFVYGDY-K1014 | 11 | 8721 | | | |
| HF-B8 | L1004-P-Δ ESEFVYGDY-K1014 | 12 | 8730 | | | |
| B-SpCas9- | L1004-GG-Δ ESEFVYGDY-K1014 | 13 | 8719 | #126762 | | |
| HF1 (HF-B9) | | | | | | |
| HF-B10 | L1004-GP-Δ ESEFVYGDY-K1014 | 14 | 8729 | | | |
| HF-BH | L1004-GGG-Δ ESEFVYGDY-K1014 | 15 | 8741 | | | |
| HF-B12 | L1004-GGGG-Δ ESEFVYGDY-K1014 | 16 | 8742 | | | |
| HF-B13 | L1004-GG-Δ ESEFVYGDYK-V1015 | 17 | 8738 | | | |
| HF-B14 | L1004-GG-Δ ESEFVYGDYKV-Y1016 | 18 | 8739 | | | |
| HF-B15 | L1004-GG-Δ ESEFVYGDYKVY-D1017 | 19 | 8740 | | | |
| HF-B16 | L1004-GG-Δ EFVYGDY-K1014 | 20 | 8756 | | | |
| HF-B17 | L1004-GG-Δ EFVYGDYKVY-D1017 | 21 | 8728 | | | |
| HF-B18 | S1006-Δ EFVYGDY-K1014 | 22 | 8722 | | | |
| HF-B19 | S1006-G-Δ EFVYGDY-K1014 | 23 | 8723 | | | |
| B-SpCas9 | L1004-GG-Δ ESEFVYGDY-K1014 | 13 | 8751 | #126760 | | |
| B-eSpCas9 | L1004-GG-Δ ESEFVYGDY-K1014 | 13 | 8752 | #126761 | 6181 | #126772 |
| B-HypaSpCas9 | L1004-GG-Δ ESEFVYGDY-K1014 | 13 | 8802 | #126763 | | |
| B-evoSpCas9 | L1004-GG-Δ ESEFVYGDY-K1014 | 13 | 8755 | #126765 | 6182 | #126773 |
| B-HeFSpCas9 | L1004-GG-Δ ESEFVYGDY-K1014 | 13 | 8753 | #126766 | | |
| e + 1 | L1004-GG-Δ ESEFVYGDY-K1014 | 13 | 8771 | | | |
| eSpCas9-plus | L1004-GG-Δ ESEFVYGDY-K1014 | 13 | 8772 | #126767 | 6183 | #126774 |
| (e + 2) | | | | | | |
| e + 3 | L1004-GG-Δ ESEFVYGDY-K1014 | 13 | 8774 | | | |
| e + 4 | L1004-GG-Δ ESEFVYGDY-K1014 | 13 | 8788 | | | |
| HF + 1 | L1004-GG-Δ ESEFVYGDY-K1014 | 13 | 8775 | | | |
| SpCas9-HF1- | L1004-GG-Δ ESEFVYGDY-K1014 | 13 | 8776 | #126768 | 6184 | #126775 |
| plus (HF + 2) | | | | | | |
| HF + 3 | L1004-GG-Δ ESEFVYGDY-K1014 | 13 | 8777 | | | |
| HF + 4 | L1004-GG-Δ ESEFVYGDY-K1014 | 13 | 8778 | | | |
| HF + 5 | L1004-GG-Δ ESEFVYGDY-K1014 | 13 | 8785 | | | |
| HF + 6 | L1004-GG-Δ ESEFVYGDY-K1014 | 13 | 8786 | | | |
| HF + 7 | L1004-GG-Δ ESEFVYGDY-K1014 | 13 | 8787 | | | |
| Sniper SpCas9 | — | | 5243 | #126777 | | |
| HiFiSpCas9 | — | | 8803 | #126778 | | |

pX330-Flag-WT SpCas9 (without sgRNA; with Silent Mutations) (Addgene #126753)
All SpCas9 variant coding plasmids' backbone are identical with the hereunder shown sequence.
Whole DNA sequence of the plasmid:

Human codon optimized S. pyogenes Cas9 (943-5043) in bold, NLS underlined (898-928), 3xFLAG tag
underlined with dashed line (826-891).

| | |
|---|---|
| 1 | CTAGAGGTAC CCGTTACATA ACTTACGGTA AATGGCCCGC CTGGCTGACC GCCCAACGAC CCCCGCCCAT TGACGTCAAT |
| 81 | AGTAACGCCA ATAGGGACTT TCCATTGACG TCAATGGGTG GAGTATTTAC GGTAAACTGC CCACTTGGCA GTACATCAAG |
| 161 | TGTATCATAT GCCAAGTACG CCCCCTATTG ACGTCAATGA CGGTAAATGG CCCGCCTGGC ATTGTGCCCA GTACATGACC |
| 241 | TTATGGGACT TTCCTACTTG GCAGTACATC TACGTATTAG TCATCGCTAT TACCATGGTC GAGGTGAGCC CCACGTTCTG |
| 321 | CTTCACTCTC CCCATCTCCC CCCCCTCCCC ACCCCCAATT TTCTATTTAT TTATTTTTTA ATTATTTTGT CCACCCATCC |
| 401 | GGGCGGGGGG GGGGGGGGGG CGCGCGCCAG GCGGGGCGGG GCGGGGCGAG GGGCGGGGCG GGGCGAGGCG GAGAGGTGCG |
| 481 | GCGGCAGCCA ATCAGAGCGG CGCGCTCCGA AAGTTTCCTT TTATGGCGAG GCGGCGGCGG CGGCGGCCCT ATAAAAAGCG |
| 561 | AAGCGCGCGG CGGGCGGGAG TCGCTGCGAC GCTGCCTTCG CCCCGTGCCC CGCTCCGCCG CCGCCTCGCG CCGCCCGCCC |
| 641 | CGGCTCTGAC TGACCGCGTT ACTCCCACAG GTGAGCGGGC GGGACGGCCC TTCTCCTCCG GGCTGTAATT AGCTGAGCAA |
| 721 | GAGGTAAGGG TTTAAGGGAT GGTTGGTTGG TGGGGTATTA ATGTTTAATT ACCTGGAGCA CCTGCCTGAA ATCACTTTTT |
| 801 | 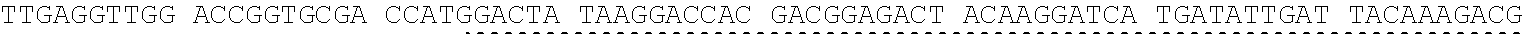 |
| 881 | 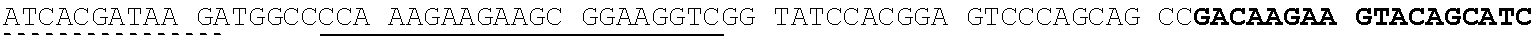 |
| 961 | GGCCTGGACA TCGGCACCAA CTCTGTGGGC TGGGCCGTGA TCACCGACGA GTACAAGGTG CCCAGCAAGA AATTCAAGGT |
| 1041 | GCTGGGCAAC ACCGACCGGC ACAGCATCAA GAAGAACCTG ATCGGAGCCC TGCTGTTCGA CAGCGGCGAA ACAGCCGAGG |
| 1121 | CCACCCGGCT GAAGAGAACC GCCAGAAGAA GATACACCAG ACGGAAGAAC CGGATCTGCT ATCTGCAAGA GATCTTCAGC |
| 1201 | AACGAGATGG CCAAGGTGGA CGACAGCTTC TTCCACAGAC TGGAAGAGTC CTTCCTGGTG GAAGAGGATA AGAAGCACGA |
| 1281 | GCGGCACCCC ATCTTCGGCA ACATCGTGGA CGAGGTGGCC TACCACGAGA AGTACCCCAC CATCTACCAC CTGAGAAAGA |
| 1361 | AACTGGTGGA CAGCACCGAC AAGGCCGACC TGCGGCTGAT CTATCTGGCC CTGGCCCACA TGATCAAGTT CCGGGGCCAC |
| 1441 | TTCCTGATCG AGGGCGACCT GAACCCCGAC AACAGCGACG TGGACAAGCT GTTCATCCAG CTGGTGCAGA CCTACAACCA |
| 1521 | GCTGTTCGAG GAAAACCCCA TCAACGCCAG CGGCGTGGAC GCCAAGGCCA TCCTGTCTGC CAGACTGAGC AAGAGCAGAC |
| 1601 | GGCTGGAAAA TCTGATCGCC CAGCTGCCCG GCGAGAAGAA GAATGGCCTG TTCGGAAACC TGATTGCCCT GAGCCTGGGC |
| 1681 | CTGACCCCCA ACTTCAAGAG CAACTTCGAC CTGGCCGAGG ATGCCAAACT GCAGCTGAGC AAGGACACCT ACGACGACGA |
| 1761 | CCTGGACAAC CTGCTGGCCC AGATCGGCGA CCAGTACGCC GACCTGTTTC TGGCCGCCAA GAACCTGTCC GACGCCATCC |
| 1841 | TGCTGAGCGA CATCCTGAGA GTGAACACCG AGATCACCAA GGCCCCCCTG AGCGCCTCTA TGATCAAGAG ATACGACGAG |
| 1921 | CACCACCAGG ACCTGACCCT GCTGAAAGCT CTCGTGCGGC AGCAGCTGCC TGAGAAGTAC AAAGAGATTT TCTTCGACCA |
| 2001 | GAGCAAGAAC GGCTACGCCG GCTACATTGA CGGCGGAGCC AGCCAGGAAG AGTTCTACAA GTTCATCAAG CCCATCCTGG |
| 2081 | AAAAGATGGA CGGCACCGAG GAACTGCTCG TGAAGCTGAA CAGAGAGGAC CTGCTGCGGA AGCAGCGGAC CTTCGACAAC |
| 2161 | GGCAGCATCC CCCACCAGAT CCACCTGGGA GAGCTGCACG CCATTCTGCG GCGGCAGGAA GATTTTTACC CATTCCTGAA |
| 2241 | GGACAACCGG GAAAAGATCG AGAAGATCCT GACCTTCCGC ATCCCCTACT ACGTGGGCCC TCTGGCCAGG GGAAACAGCA |
| 2321 | GATTCGCCTG GATGACCAGA AAGAGCGAGG AAACCATCAC CCCCTGGAAC TTCGAGGAAG TGGIGGACAA GGGCGCTTCC |
| 2401 | GCCCAGAGCT TCATCGAGCG GATGACCAAC TTCGATAAGA ACCTGCCCAA CGAGAAGGTG CTGCCCAAGC ACAGCCTGCT |
| 2481 | GTACGAGTAC TTCACCGTGT ATAACGAGCT GACCAAAGTG AAATACGTGA CCGAGGGAAT GAGAAAGCCC GCCTTCCTGA |
| 2561 | GCGGCGAGCA GAAAAAGGCC ATCGTGGACC TGCTGTTCAA GACCAACCGG AAAGTGACCG TGAAGCAGCT GAAAGAGGAC |
| 2641 | TACTTCAAGA AAATCGAGTG CTTCGACTCC GTGGAAATCT CCGGCGTGGA AGATCGGTTC AACGCCTCCC TGGGCACATA |
| 2721 | CCACGATCTG CTGAAAATTA TCAAGGACAA GGACTTCCTG GACAATGAGG AAAACGAGGA CATTCTGGAA GATATCGTGC |
| 2801 | TGACCCTGAC ACTGTTTGAG GACAGAGAGA TGATCGAGGA ACGGCTGAAA ACCTATGCCC ACCTGTTCGA CGACAAAGTG |
| 2881 | ATGAAGCAGC TGAAGCGGCG GAGATACACC GGCTGGGGCA GGCTGAGCCG GAAGCTGATC AACGGCATCC GGGACAAGCA |
| 2961 | GTCCGGCAAG ACAATCCTGG ATTTCCTGAA GTCCGACGGC TTCGCCAACA GAAACTTCAT GCAGCTGATC CACGACGACA |
| 3041 | GCCTGACCTT TAAAGAGGAC ATCCAGAAAG CCCAGGTGTC CGGCCAGGGC GATAGCCTGC ACGAGCACAT TGCCAATCTG |
| 3121 | GCCGGCAGCC CCGCCATTAA GAAGGGCATC CTGCAGACAG TGAAGGTGGT GGACGAGCTC GTGAAAGTGA TGGGCCGGCA |
| 3201 | CAAGCCCGAG AACATCGTGA TCGAAATGGC CAGAGAGAAC CAGACCACCC AGAAGGGACA GAAGAACAGC CGCGAGAGAA |
| 3281 | TGAAGCGGAT CGAAGAGGGC ATCAAAGAGC TGGGCAGCCA GATCCTGAAA GAACACCCCG TGGAAAACAC CCAGCTGCAG |
| 3361 | AACGAGAAGC TGTACCTGTA CTACCTGCAG AATGGGCGGG ATATGTACGT GGACCAGGAA CTGGACATCA ACCGGCTGTC |
| 3441 | CGACTACGAT GTGGACCATA TCGTGCCTCA GAGCTTTCTG AAGGACGACT CCATCGACAA CAAGGTGCTG ACCAGAAGCG |
| 3521 | ACAAGAACCG GGGCAAGAGC GACAACGTGC CCTCCGAAGA GGTCGTGAAG AAGATGAAGA ACTACTGGCG GCAGCTGCTG |
| 3601 | AACGCCAAGC TGATTACCCA GAGAAAGTTC GACAATCTGA CCAAGGCCGA GAGAGGCGGC CTGAGCGAAC TGGATAAGGC |
| 3681 | CGGCTTCATC AAGAGACAGC TGGTGGAAAC CCGGCAGATC ACAAAGCACG TGGCACAGAT CCTGGACTCC CGGATGAACA |
| 3761 | CTAAGTACGA CGAGAATGAC AAGCTGATCC GGGAAGTGAA AGTGATCACC CTGAAGTCCA AGCTGGTGTC CGATTTCCGG |
| 3841 | AAGGATTTCC AGTTTTACAA AGTGCGCGAG ATCAACAACT ACCACCACGC CCACGACGCG TACCTGAACG CCGTCGTGGG |
| 3921 | AACCGCCCTG ATCAAAAAGT ACCCTAAGCT GGAAAGCGAG TTCGTGTACG GCGACTACAA GGTGTACGAC GTACGGAAGA |
| 4001 | TGATCGCCAA GAGCGAGCAG GAAATCGGCA AGGCTACCGC CAAGTACTTC TTCTACAGCA ACATCATGAA CTTTTTCAAG |
| 4081 | ACCGAGATTA CCCTGGCCAA CGGCGAGATC CGGAAGCGGC CTCTGATCGA GACAAACGGC GAAACCGGGG AGATCGTGTG |
| 4161 | GGATAAGGGC CGGGATTTTG CCACCGTGCG GAAAGTGCTG AGCATGCCCC AAGTGAATAT CGTGAAAAAG ACCGAGGTGC |
| 4241 | AGACAGGCGG CTTCAGCAAA GAGTCTATCC TGCCCAAGAG GAACAGCGAT AAGCTGATCG CCAGAAAGAA GGACTGGGAC |
| 4321 | CCTAAGAAGT ACGGCGGCTT CGACAGCCCC ACCGTGGCCT ATTCTGTGCT GGTGGTGGCC AAAGTGGAAA AGGGCAAGTC |
| 4401 | CAAGAAACTG AAGAGTGTGA AAGAGCTGCT GGGGATCACC ATCATGGAAA GAAGCAGCTT CGAGAAGAAT CCCATCGACT |
| 4481 | TTCTGGAAGC CAAGGGCTAC AAAGAAGTGA AAAAGGACCT GATCATCAAG CTGCCTAAGT ACTCCCTGTT CGAGCTGGAA |
| 4561 | AACGGCCGGA AGAGAATGCT GGCCTCTGCC GGCGAACTGC AGAAGGGAAA CGAACTGGCC CTGCCCTCCA AATATGTGAA |
| 4641 | CTTCCTGTAC CTGGCCAGCC ACTATGAGAA GCTGAAGGGC TCCCCCGAGG ATAATGAGCA GAAACAGCTG TTTGTGGAAC |
| 4721 | AGCACAAGCA CTACCTGGAC GAGATCATCG AGCAGATCAG CGAGTTCTCC AAGAGAGTGA TCCTGGCCGA CGCTAATCTG |
| 4801 | GACAAAGTGC TGTCCGCCTA CAACAAGCAC CGGGATAAGC CCATCAGAGA GCAGGCCGAG AATATCATCC ACCTGTTTAC |
| 4881 | CCTGACCAAT CTGGGAGCCC CTGCCGCCTT CAAGTACTTT GACACCACCA TCGACCGGAA GAGGTACACC AGCACCAAAG |
| 4961 | AGGTGCTGGA CGCCACCCTG ATCCACCAGA GCATCACCGG CCTGTACGAG ACACGGATCG ACCTGTCTCA GCTGGGAGGC |
| 5041 | GACAAAAGGC CGGCGGCCAC GAAAAAGGCC GGCCAGGCAA AAAAGAAAAA GTAAGAATTC CTAGAGCTCG CTGATCAGCC |
| 5121 | TCGACTGTGC CTTCTAGTTG CCAGCCATCT GTTGTTTGCC CCTCCCCCGT GCCTTCCTTG ACCCTGGAAG GTGCCACTCC |
| 5201 | CACTGTCCTT TCCTAATAAA ATGAGGAAAT TGCATCGCAT TGTCTGAGTA GGTGTCATTC TATTCTGGGG GGTGGGGTGG |
| 5281 | GGCAGGACAG CAAGGGGGAG GATTGGGAAG AGAATAGCAG GCATGCTGGG GAGCGGCCGC AGGAACCCCT AGTGATGGAG |
| 5361 | TTGGCCACTC CCTCTCTGCG CGCTCGCTCG CTCACTGAGG CCGGGCGACC AAAGGTCGCC CGACGCCCGG GCTTTGCCCG |
| 5441 | GGCGGCCTCA GTGAGCGAGC GAGCGCGCAG CTGCCTGCAG GGGCGCCTGA TGCGGTATTT TCTCCTTACG CATCTGTGCG |
| 5521 | GTATTTCACA CCGCATACGT CAAAGCAACC ATAGTACGCG CCCTGTAGCG GCGCATTAAG CGCGGCGGGT GTGGTGGTTA |
| 5601 | CGCGCAGCGT GACCGCTACA CTTGCCAGCG CCCTAGCGCC CGCTCCTTTC GCTTTCTTCC CTTCCTTTCT CGCCACGTTC |
| 5681 | GCCGGCTTTC CCCGTCAAGC TCTAAATCGG GGGCTCCCTT TAGGGTTCCG ATTTAGTGCT TTACGGCACC TCGACCCCAA |
| 5761 | AAAACTTGAT TTGGGTGATG GTTCACGTAG TGGGCCATCG CCCTGATAGA CGGTTTTTCG CCCTTTGACG TTGGAGTCCA |
| 5841 | CGTTCTTTAA TAGTGGACTC TTGTTCCAAA CTGGAACAAC ACTCAACCCT ATCTCGGGCT ATTCTTTTGA TTTATAAGGG |
| 5921 | ATTTTGCCGA TTTCGGCCTA TTGGTTAAAA AATGAGCTGA TTTAACAAAA ATTTAACGCG AATTTTAACA AAATATTAAC |
| 6001 | GTTTACAATT TTATGGTGCA CTCTCAGTAC AATCTGCTCT GATGCCGCAT AGTTAAGCCA GCCCCGACAC CCGCCAACAC |
| 6081 | CCGCTGACGC GCCCTGACGG GCTTGTCTGC TCCCGGCATC CGCTTACAGA CAAGCTGTGA CCGTCTCCGG GAGCTGCATG |
| 6161 | TGTCAGAGGT TTTCACCGTC ATCACCGAAA CGCGCGAGAC GAAAGGGCCT CGTGATACGC CTATTTTTAT AGGTTAATGT |
| 6241 | CATGATAATA ATGGTTTCTT AGACGTCAGG TGGCACTTTT CGGGGAAATG TGCGCGGAAC CCCTATTTGT TTATTTTTCT |
| 6321 | AAATACATTC AAATATGTAT CCGCTCATGA GACAATAACC CTGATAAATG CTTCAATAAT ATTGAAAAAG GAAGAGTATG |
| 6401 | AGTATTCAAC ATTTCCGTGT CGCCCTTATT CCCTTTTTTG CGGCATTTTG CCTTCCTGTT TTTGCTCACC CAGAAACGCT |
| 6481 | GGTGAAAGTA AAAGATGCTG AAGATCAGTT GGGTGCACGA GTGGGTTACA TCGAACTGGA TCTCAACAGC GGTAAGATCC |
| 6561 | TTGAGAGTTT TCGCCCCGAA GAACGTTTTC CAATGATGAG CACTTTTAAA GTTCTGCTAT GTGGCGCGGT ATTATCCCGT |
| 6641 | ATTGACGCCG GGCAAGAGCA ACTCGGTCGC CGCATACACT ATTCTCAGAA TGACTTGGTT GAGTACTCAC CAGTCACAGA |
| 6721 | AAAGCATCTT ACGGATGGCA TGACAGTAAG AGAATTATGC AGTGCTGCCA TAACCATGAG TGATAACACT GCGGCCAACT |
| 6801 | TACTTCTGAC AACGATCGGA GGACCGAAGG AGCTAACCGC TTTTTTGCAC AACATGGGGG ATCATGTAAC TCGCCTTGAT |
| 6881 | CGTTGGGAAC CGGAGCTGAA TGAAGCCATA CCAAACGACG AGCGTGACAC CACGATGCCT GTAGCAATGG CAACAACGTT |
| 6961 | GCGCAAACTA TTAACTGGCG AACTACTTAC TCTAGCTTCC CGGCAACAAT TAATAGACTG GATGGAGGCG GATAAAGTTG |
| 7041 | CAGGACCACT TCTGCGCTCG GCCCTTCCGG CTGGCTGGTT TATTGCTGAT AAATCTGGAG CCGGTGAGCG TGGAAGCCGC |
| 7121 | GGTATCATTG CAGCACTGGG GCCAGATGGT AAGCCCTCCC GTATCGTAGT TATCTACACG ACGGGGAGTC AGGCAACTAT |
| 7201 | GGATGAACGA AATAGACAGA TCGCTGAGAT AGGTGCCTCA CTGATTAAGC ATTGGTAACT GTCAGACCAA GTTTACTCAT |
| 7281 | ATATACTTTA GATTGATTTA AAACTTCATT TTTAATTTAA AAGGATCTAG GTGAAGATCC TTTTTGATAA TCTCATGACC |
| 7361 | AAAATCCCTT AACGTGAGTT TTCGTTCCAC TGAGCGTCAG ACCCCGTAGA AAAGATCAAA GGATCTTCTT GAGATCCTTT |
| 7441 | TTTTCTGCGC GTAATCTGCT GCTTGCAAAC AAAAAAACCA CCGCTACCAG CGGTGGTTTG TTTGCCGGAT CAAGAGCTAC |
| 7521 | CAACTCTTTT TCCGAAGGTA ACTGGCTTCA GCAGAGCGCA GATACCAAAT ACTGTCCTTC TAGTGTAGCC GTAGTTAGGC |
| 7601 | CACCACTTCA AGAACTCTGT AGCACCGCCT ACATACCTCG CTCTGCTAAT CCTGTTACCA GTGGCTGCTG CCAGTGGCGA |
| 7681 | TAAGTCGTGT CTTACCGGGT TGGACTCAAG ACGATAGTTA CCGGATAAGG CGCAGCGGTC GGGCTGAACG GGGGGTTCGT |
| 7761 | GCACACAGCC CAGCTTGGAG CGAACGACCT ACACCGAACT GAGATACCTA CAGCGTGAGC TATGAGAAAG CGCCACGCTT |
| 7841 | CCCGAAGGGA GAAAGGCGGA CAGGTATCCG GTAAGCGGCA GGGTCGGAAC AGGAGAGCGC ACGAGGGAGC TTCCAGGGGG |
| 7921 | AAACGCCTGG TATCTTTATA GTCCTGTCGG GTTTCGCCAC CTCTGACTTG AGCGTCGATT TTTGTGATGC TCGTCAGGGG |
| 8001 | GGCGGAGCCT ATGGAAAAAC GCCAGCAACG CGGCCTTTTT ACGGTTCCTG GCCTTTTGCT GGCCTTTTGC TCACATG |

List of nucleotide differences compared to pX330-Flag-WT SpCas9 (without sgRNA; with silent mutations) (Addgene #126753):

pX330-Flag-eSpCas9 (without sgRNA; with silent mutations) (Addgene #126754)

### A3481G, A3482C, G3483C, A3946G, A3947C, C4117G, G4118C

pX330-Flag-SpCas9-HF1 (without sgRNA; with silent mutations) (Addgene #126755)

### A2428G, A2429C, A2920G, G2921C, G2952C, C3022G, A3023C, G3120C, G3153C, C3180G, C3270T, G3357C, G3387C, G3480T, C3715G, A3716C, C4320T, A4407G, G4410T, C4545G, G4548C

pX330-Flag-HypaSpCas9 (without sgRNA; with silent mutations) (Addgene #126756)

### G2952C, A3013G, A3014C, C3018T, A3019G, T3020C, G3021A, C3022G, A3023C, G3024C, C3031G, A3032C, C3033T

pX330-Flag-evoSpCas9 (without sgRNA: with silent mutations) (Addgene #126758)

### A2422G, T2482A, A2515G, A2920C, G2921A

pX330-Flag-HeFSpCas9 (without sgRNA; with silent mutations) (Addgene #126759)

### A2428G, A2429C, A2920G, G2921C, G2952C, C3022G, A3023C, G3120C, G3153C, C3180G, C3270T, G3357C, G3387C, A3481G, A3482C, C3715G, A3716C, A3946G, A3947C, C4117G, G4118C

pX330-Flag-Sniper SpCas9 (without sgRNA; with silent mutations) (Addgene #126777

### C2553T, T2554A, T2555G, G3228C, C3231G, C3606G, G3609T, C3610T

pX330-Flag-HiFi SpCas9 (without sgRNA; with silent mutations) (Addgene #126778)

### A3010G, G3011C

SpCas9 Variants, Bacterial Expression Plasmids

Bacterial expression plasmids [pET-FLAG-eSpCas9 (Addgene #126769), pET-FLAG-SpCas9-HF1 (Addgene #126770), pET-FLAG-B-eSpCas9 (Addgene #126772), pET-FLAG-eSpCas9-plus (Addgene #126774), pET-FLAG-SpCas9-HF1-plus (Addgene #126775)] were constructed from pMJ806 (#39312)(Jinek et al., 2012) plasmid by digestion with BcuI and NotI restriction enzymes and by ligating a fragment (containing the TEV-3xFLAG-NLS-SpCas9 variant-NLS coding sequence) generated as follows: PCR products were generated from the mammalian expression plasmids [pX330-Flag-wtSpCas9 (without sgRNA) (Addgene #92353), pX330-Flag-eSpCas9 (without sgRNA) (Addgene #92354), pX330-Flag-SpCas9-HF1 (without sgRNA) (Addgene #92102), eSpCas9-plus (Addgene #126767), SpCas9-HF1-plus (Addgene #126768)] using 9214-SpCas9_bact_exp-for (AAAAAAACTAGTGAAAACCTGTATTTCCAGGGAGCAGCCTCGatggactataaggaccacgacg) and 9214-SpCas9_bact_exp-rev (AAAAAAGCGGCCGcaacagatggctggcaacta) primers and after that the PCR products were digested with BcuI and NotI restriction enzymes.

Cloning of the Blackjack (B-) SpCas9-HF1 Candidates

SpCas9-HF1 Blackjack candidates were constructed from pX330-Flag-SpCas9-HF1 (without sgRNA; with silent mutations) (Addgene #126755) plasmid by digestion with MluI and Pfl23II restriction enzymes and the hybridized synthetic DNA oligonucleotides were ligated into the digested vector (for details of the sequences see Table 9).

TABLE 9
List of primers used for the generation of the Blackjack variants. Primer names (coming from prep name
[plasmid #] in Table 8: mammalian expression plasmid names) and SEQ ID NOs are listed in first and second
column, respectively

| Primer | Seq# | Sequence |
|---|---|---|
| 8717for | 537 | CGCgTACCTGAACGCCGTCGTGGGAACCGCCCTGATCAAAAAGTACCCTaaGCTGGAAAGCGgGTTCGTGTACGGCGACg |
| | | gCAAGGTGTACGAt |
| 8717rev | 538 | gtacaTCGTACACCTTGccGTCGCCGTACACGAACcCGCTTTCCAGCttAGGGTACTTTTTGATCAGGGCGGTTCCCACG |
| | | ACGGCGTTCAGGTA |
| 8727for | 539 | CGCgTACCTGAACGCCGTCGTGGGAACCGCCCTGATCAAAAAGTACCCTaaGggtAAGGTGTACGAt |
| 8727rev | 540 | gtacaTCGTACACCTTaccCttAGGGTACTTTTTGATCAGGGCGGTTCCCACGACGGCGTTCAGGTA |
| 8724for | 541 | CGCgTACCTGAACGCCGTCGTGGGAACCGCCCTGATCAAAAAGTACCCTaaGGAt |
| 8724rev | 542 | gtacaTCCttAGGGTACTTTTTGATCAGGGCGGTTCCCACGACGGCGTTCAGGTA |
| 8725for | 543 | CGCgTACCTGAACGCCGTCGTGGGAACCGCCCTGATCAAAAAGTACCCTaaGggtGAt |
| 8725rev | 544 | gtacaTCaccCttAGGGTACTTTTTGATCAGGGCGGTTCCCACGACGGCGTTCAGGTA |
| 8726for | 545 | CGCgTACCTGAACGCCGTCGTGGGAACCGCCCTGATCAAAAAGTACCCTaaGggtaaaGAt |
| 8726rev | 546 | gtacaTCtttaccCttAGGGTACTTTTTGATCAGGGCGGTTCCCACGACGGCGTTCAGGTA |
| 8718for | 547 | CGCgTACCTGAACGCCGTCGTGGGAACCGCCCTGATCAAAAAGTACCCTaaGCTGAAGGTGTACGAt |
| 8718rev | 548 | gtacaTCGTACACCTTCAGCttAGGGTACTTTTTGATCAGGGCGGTTCCCACGACGGCGTTCAGGTA |
| 8721for | 549 | CGCgTACCTGAACGCCGTCGTGGGAACCGCCCTGATCAAAAAGTACCCTaaGCTGggtAAGGTGTACGAt |
| 8721rev | 550 | gtacaTCGTACACCTTaccCAGCttAGGGTACTTTTTGATCAGGGCGGTTCCCACGACGGCGTTCAGGTA |
| 8730for | 551 | CGCgTACCTGAACGCCGTCGTGGGAACCGCCCTGATCAAAAAGTACCCTaaGCTGcctAAGGTGTACGAt |
| 8730rev | 552 | gtacaTCGTACACCTTaggCAGCttAGGGTACTTTTTGATCAGGGCGGTTCCCACGACGGCGTTCAGGTA |
| 8719for | 553 | CGCgTACCTGAACGCCGTCGTGGGAACCGCCCTGATCAAAAAGTACCCTaaGCTGggtggcAAGGTGTACGAt |
| 8719rev | 554 | gtacaTCGTACACCTTgccaccCAGCttAGGGTACTTTTTGATCAGGGCGGTTCCCACGACGGCGTTCAGGTA |
| 8729for | 555 | CGCgTACCTGAACGCCGTCGTGGGAACCGCCCTGATCAAAAAGTACCCTaaGCTGggtcctAAGGTGTACGAt |
| 8729rev | 556 | gtacaTCGTACACCTTaggaccCAGCttAGGGTACTTTTTGATCAGGGCGGTTCCCACGACGGCGTTCAGGTA |
| 8741for | 557 | CGCgTACCTGAACGCCGTCGTGGGAACCGCCCTGATCAAAAAGTACCCTaaGCTGggtggcggaAAGGTGTACGAt |
| 8741rev | 558 | gtacaTCGTACACCTTtccgccaccCAGCttAGGGTACTTTTTGATCAGGGCGGTTCCCACGACGGCGTTCAGGTA |
| 8742for | 559 | CGCgTACCTGAACGCCGTCGTGGGAACCGCCCTGATCAAAAAGTACCCTaaGCTGggtggcggaggtAAGGTGTACGAt |
| 8742rev | 560 | gtacaTCGTACACCTTacctccgccaccCAGCttAGGGTACTTTTTGATCAGGGCGGTTCCCACGACGGCGTTCAGGTA |
| 8738for | 561 | CGCgTACCTGAACGCCGTCGTGGGAACCGCCCTGATCAAAAAGTACCCTaaGCTGggtggcGTGTACGAt |
| 8738rev | 562 | gtacaTCGTACACgccaccCAGCttAGGGTACTTTTTGATCAGGGCGGTTCCCACGACGGCGTTCAGGTA |
| 8739for | 563 | CGCgTACCTGAACGCCGTCGTGGGAACCGCCCTGATCAAAAAGTACCCTaaGCTGggtggcTACGAt |
| 8739rev | 564 | gtacaTCGTAgccaccCAGCttAGGGTACTTTTTGATCAGGGCGGTTCCCACGACGGCGTTCAGGTA |
| 8740for | 565 | CGCgTACCTGAACGCCGTCGTGGGAACCGCCCTGATCAAAAAGTACCCTaaGCTGggtggcGAt |
| 8740rev | 566 | gtacaTCgccaccCAGCttAGGGTACTTTTTGATCAGGGCGGTTCCCACGACGGCGTTCAGGTA |
| 8756for | 567 | CGCgTACCTGAACGCCGTCGTGGGAACCGCCCTGATCAAAAAGTACCCTaaGCTGtctAGCAAGGTGTACGAt |
| 8756rev | 568 | gtacaTCGTACACCTTGCTagaCAGCttAGGGTACTTTTTGATCAGGGCGGTTCCCACGACGGCGTTCAGGTA |
| 8728for | 569 | CGCgTACCTGAACGCCGTCGTGGGAACCGCCCTGATCAAAAAGTACCCTaaGCTGtctAGCGAt |
| 8728rev | 570 | gtacaTCGCTagaCAGCttAGGGTACTTTTTGATCAGGGCGGTTCCCACGACGGCGTTCAGGTA |
| 8722for | 571 | CGCgTACCTGAACGCCGTCGTGGGAACCGCCCTGATCAAAAAGTACCCTaaGCTGGAAAGCAAGGTGTACGAt |
| 8722rev | 572 | gtacaTCGTACACCTTGCTTTCCAGCttAGGGTACTTTTTGATCAGGGCGGTTCCCACGACGGCGTTCAGGTA |
| 8723for | 573 | CGCgTACCTGAACGCCGTCGTGGGAACCGCCCTGATCAAAAAGTACCCTaaGCTGGAAAGCggtAAGGTGTACGAt |
| 8723rev | 574 | gtacaTCGTACACCTTaccGCTTTCCAGCttAGGGTACTTTTTGATCAGGGCGGTTCCCACGACGGCGTTCAGGTA |
| 8752for | 575 | CGCgTACCTGAACGCCGTCGTGGGAACCGCCCTGATCAAAAAGTACCCTgcGCTGggtggcAAGGTGTACGAt |
| 8752rev | 576 | gtacaTCGTACACCTTgccaccCAGCgcAGGGTACTTTTTGATCAGGGCGGTTCCCACGACGGCGTTCAGGTA |

Cloning of all the B-SpCas9 Variants

SpCas9 Blackjack variants were constructed from the parent SpCas9 variants (Addgene numbers: #126754, #126755, #126756, #126758, #126759) by digestion with MluI and Pfl23II restriction enzymes (cleavage sites introduced by silent mutations into the parent SpCas9s plasmid sequence). The synthetic DNA oligonucleotides (in the case of WT, (-HF1), Hypa-, evoSpCas9 8719for and 8719rev; in the case of e- and HeFSpCas9 8752for and 8752rev oligonucleotides were used; for details of the sequences see Table 9) were hybridized and the annealed oligonucleotides were ligated into the digested vectors.

Cloning of the Plus Candidates

B-eSpCas9-plus and B-SpCas9-HF1-plus candidates were constructed from B-eSpCas9 (Addgene #126761), B-SpCas9-HF1 (Addgene #126762), respectively.

e+1: was constructed from the B-eSpCas9 plasmid by digestion with ApaI and MluI restriction enzymes and ligation with a fragment picked from the pX330-Flag-WTSpCas9 (without sgRNA; with silent mutations) (Addgene #126753) plasmid by digestion with ApaI and MluI restriction enzymes.

eSpCas9-plus (e+2) (Addgene #126767): was constructed from the pX330-Flag-eSpCas9 (without sgRNA; with silent mutations)(Addgene #126754) plasmid by digestion with MluI and Pfl23II restriction enzymes and ligation with the 8719for and 8719rev annealed oligonucleotides (for details of the sequences see Table 9).

e+3: was constructed in two steps. Step one: a construct was made from the pX330-Flag-eSpCas9 (without sgRNA; with silent mutations) (Addgene #126754) plasmid by digestion with EcoRI and Pfl23II restriction enzymes and ligation with a fragment picked from the pX330-Flag-WTSpCas9 (without sgRNA; with silent mutations) (Addgene #126753) plasmid by digestion with EcoRI and Pfl23II restriction enzymes. Step two: The plasmid constructed in the first step was digested with MluI and Pfl23II restriction enzymes and ligated with the 8752for and 8752rev annealed oligonucleotides (for details of the sequences see Table 9).

e+4: was constructed from the e+3 first step plasmid by digestion with MluI and Pfl23II restriction enzymes and ligated with the 8719for and 8719rev annealed oligonucleotides (for details of the sequences see Table 9).

HF+1: was constructed from the B-SpCas9-HF1 plasmid by digestion with BgIII and Eco32I restriction enzymes and ligation with a fragment picked from the pX330-Flag-WTSpCas9 (without sgRNA; with silent mutations) (Addgene #126753) plasmid by digestion with BgIII and Eco32I restriction enzymes.

HF+2 (SpCas9-HF1-plus) (Addgene #126768), HF+3 and HF+4: These were constructed from the B-SpCas9-HF1 plasmid (Addgene #126762) by digestion with BamHI and Eco32I restriction enzymes and assembling two fragments from the digest using the NEBuilder HiFi DNA Assembly Master Mix. Fragment one was a PCR product generated from the B-SpCas9-HF1 plasmid using the HF+primer_rev and one of the following primers: A661Rfor (HF+2), A695Qfor (HF+3), A926Qfor (HF+4). Fragment two was a PCR product generated from B-SpCas9-HF1 plasmid using HF+primer_for and one of the following primers: A661Rrev (HF+2), A695Qrev (HF+3), A926Qrev (HF+4) (for details of the sequences see Table 10).

HF+5 and HF+6: These were constructed from the HF+2 plasmid by digestion with BamHI and Eco32I restriction enzymes and assembling two fragments from the digest using the NEBuilder HiFi DNA Assembly Master Mix. Fragment one was a PCR product generated from HF+2 plasmid using HF+primer_rev and one of the following primers: A695Qfor (HF+5), A926Qfor (HF+6). Fragment two was a PCR product generated from HF+2 plasmid using HF+primer_for and one of the following primers: A695Qrev (HF+5), A926Qrev (HF+6) (for details of the sequences see Table 10).

HF+7: was constructed from the HF+3 plasmid by digestion with BamHI and Eco32I restriction enzymes and by assembling two fragments from the digest using the NEBuilder HiFi DNA Assembly Master Mix. Fragment one was a PCR product generated from the HF+3 plasmid using HF+primer_rev and A926Qfor. Fragment two was a PCR product generated from the HF+3 plasmid using HF+primer_for and A926Qrev (for details of the sequences see Table 10).

TABLE 10
List of primers used for cloning SpCas9-HF1-plus candidates.

| Primer | SEQ ID NO. | Sequence |
|---|---|---|
| HF + primer_rev | 577 | gaagCCGCCGTACTTCTTAGGaTC |
| HF + primer_for | 578 | GAGGAAAACGAGGACATTCTGGAAGAT |
| A661Rfor | 579 | CGGCTGGGGCAGGCTGAGCCGGAAGCTGATCAA |
| A661Rrev | 580 | CGGCTCAGCCTGCCCCAGCCGGTGTATC |
| A695Qfor | 581 | aaacttcatgcagCTGATCCACGACGACAGCCTGA |
| A695Qrev | 582 | CGTGGATCAGctgCATGAAGTTTCTGTTGGCGAAGCCGT |
| A926Qrev | 583 | GCTTTGTGATctgCCGGGTTTCCACCAGCTGT |
| A926Qfor | 584 | GGAAACCCGGcaGATCACAAAGCACGTGGCACA |

### REFERENCES

Altschul S F, Gish W, Miller W, Myers E W and Lipman D J (1990) Basic local alignment search tool. J Mol Biol 215:403-410.
Anders C, Bargsten K and Jinek M (2016) Structural Plasticity of PAM Recognition by Engineered Variants of the RNA-Guided Endonuclease Cas9. Molecular cell 61:895-902.
Anders C, Niewoehner O, Duerst A and Jinek M (2014) Structural basis of PAM-dependent target DNA recognition by the Cas9 endonuclease. Nature 513:569-573.
Beckert B and Masquida B (2011) Synthesis of RNA by in vitro transcription. Methods Mol Biol 703:29-41.
Brinkman E K, Chen T, Amendola M and van Steensel B (2014) Easy quantitative assessment of genome editing by sequence trace decomposition. Nucleic Acids Res 42:e168.
Casini A, Olivieri M, Petris G, Montagna C, Reginato G, Maule G, Lorenzin F, Prandi D, Romanel A, Demichelis F, Inga A and Cereseto A (2018) A highly specific SpCas9 variant is identified by in vivo screening in yeast. Nat Biotechnol 36:265-271.
Chen J S, Dagdas Y S, Kleinstiver B P, Welch M M, Sousa A A, Harrington L B, Sternberg S H, Joung J K, Yildiz A and Doudna J A (2017) Enhanced proofreading governs CRISPR-Cas9 targeting accuracy. Nature 550:407-410.
Cho S W, Kim S, Kim J M and Kim J S (2013) Targeted genome engineering in human cells with the Cas9 RNA-guided endonuclease. Nat Biotechnol 31:230-232.
Cho S W, Kim S, Kim Y, Kweon J, Kim H S, Bae S and Kim J S (2014) Analysis of off-target effects of CRISPR/Cas-derived RNA-guided endonucleases and nickases. Genome Res 24:132-141.
Cong L, Ran F A, Cox D, Lin S, Barretto R, Habib N, Hsu P D, Wu X, Jiang W, Marraffini L A and Zhang F (2013) Multiplex genome engineering using CRISPR/Cas systems. Science 339:819-823.
Dong F, Xie K, Chen Y, Yang Y and Mao Y (2017) Polycistronic tRNA and CRISPR guide-RNA enables highly efficient multiplexed genome engineering in human cells. Biochem Biophys Res Commun 482:889-895.
Engler C, Kandzia R and Marillonnet S (2008) A one pot, one step, precision cloning method with high throughput capability. PloS one 3:e3647.
Fu Y, Reyon D and Joung J K (2014a) Targeted genome editing in human cells using CRISPR/Cas nucleases and truncated guide RNAs. Methods Enzymol 546:21-45.
Fu Y, Sander J D, Reyon D, Cascio V M and Joung J K (2014b) Improving CRISPR-Cas nuclease specificity using truncated guide RNAs. Nat Biotechnol 32:279-284.
Gao Y and Zhao Y (2014) Self-processing of ribozyme-flanked RNAs into guide RNAs in vitro and in vivo for CRISPR-mediated genome editing. J Integr Plant Biol 56:343-349.
Garneau J E, Dupuis M E, Villion M, Romero D A, Barrangou R, Boyaval P, Fremaux C, Horvath P, Magadan A H and Moineau S (2010) The CRISPR/Cas bacterial immune system cleaves bacteriophage and plasmid DNA. Nature 468:67-71.
Goomer R S and Kunkel G R (1992) The transcriptional start site for a human U6 small nuclear RNA gene is dictated by a compound promoter element consisting of the PSE and the TATA box. Nucleic Acids Res 20:4903-4912.
Guilinger J P, Thompson D B and Liu D R (2014) Fusion of catalytically inactive Cas9 to FokI nuclease improves the specificity of genome modification. Nat Biotechnol 32:577-582.
Hsu P D, Lander E S and Zhang F (2014) Development and applications of CRISPR-Cas9 for genome engineering. Cell 157:1262-1278.
Hu J H, Miller S M, Geurts M H, Tang W, Chen L, Sun N, Zeina C M, Gao X, Rees H A, Lin Z and Liu D R (2018) Evolved Cas9 variants with broad PAM compatibility and high DNA specificity. Nature 556:57-63.
Iwamoto M, Bjorklund T, Lundberg C, Kirik D and Wandless T J (2010) A general chemical method to regulate protein stability in the mammalian central nervous system. Chem Biol 17:981-988.
Jiang F, Taylor D W, Chen J S, Kornfeld J E, Zhou K, Thompson A J, Nogales E and Doudna J A (2016) Structures of a CRISPR-Cas9R-loop complex primed for DNA cleavage. Science 351:867-871.
Jiang F, Zhou K, Ma L, Gressel S and Doudna J A (2015) STRUCTURAL BIOLOGY. A Cas9-guide RNA complex preorganized for target DNA recognition. Science 348:1477-1481.
Jinek M, Chylinski K, Fonfara I, Hauer M, Doudna J A and Charpentier E (2012) A programmable dual-RNA-guided DNA endonuclease in adaptive bacterial immunity. Science 337:816-821.
Jinek M, East A, Cheng A, Lin S, Ma E and Doudna J (2013) RNA-programmed genome editing in human cells. Elife 2:e00471.
Jinek M, Jiang F, Taylor D W, Sternberg S H, Kaya E, Ma E, Anders C, Hauer M, Zhou K, Lin S, Kaplan M, Iavarone A T, Charpentier E, Nogales E and Doudna J A (2014) Structures of Cas9 endonucleases reveal RNA-mediated conformational activation. Science 343:1247997.
Kim S, Bae T, Hwang J and Kim J S (2017) Rescue of high-specificity Cas9 variants using sgRNAs with matched 5′ nucleotides. Genome Biol 18:218.
Kleinstiver B P, Pattanayak V, Prew M S, Tsai S Q, Nguyen N T, Zheng Z and Joung J K (2016) High-fidelity CRISPR-Cas9 nucleases with no detectable genome-wide off-target effects. Nature 529:490-495.
Kocak D D, Josephs E A, Bhandarkar V, Adkar S S, Kwon J B and Gersbach C A (2019) Increasing the specificity of CRISPR systems with engineered RNA secondary structures. Nat Biotechnol.
Komor A C, Badran A H and Liu D R (2017) CRISPR-Based Technologies for the Manipulation of Eukaryotic Genomes. Cell 169:559.
Konermann S, Brigham M D, Trevino A E, Joung J, Abudayyeh O O, Barcena C, Hsu P D, Habib N, Gootenberg J S, Nishimasu H, Nureki O and Zhang F (2015) Genome-scale transcriptional activation by an engineered CRISPR-Cas9 complex. Nature 517:583-588.
Koonin E V, Makarova K S and Zhang F (2017) Diversity, classification and evolution of CRISPR-Cas systems. Curr Opin Microbiol 37:67-78.
Kostylev M, Otwell A E, Richardson R E and Suzuki Y (2015) Cloning Should Be Simple: Escherichia coli DH5 alpha-Mediated Assembly of Multiple DNA Fragments with Short End Homologies. Plos One 10.
Kulcsar P I, Talas A, Huszar K, Ligeti Z, Toth E, Weinhardt N, Fodor E and Welker E (2017) Crossing enhanced and high fidelity SpCas9 nucleases to optimize specificity and cleavage. Genome Biol 18:190.
Lee J K, Jeong E, Lee J, Jung M, Shin E, Kim Y H, Lee K, Jung I, Kim D, Kim S and Kim J S (2018) Directed evolution of CRISPR-Cas9 to increase its specificity. Nat Commun 9:3048.
Lee R T, Ng A S and Ingham P W (2016) Ribozyme Mediated gRNA Generation for In Vitro and In Vivo CRISPR/Cas9 Mutagenesis. PLoS One 11:e0166020.
Makarova K S, Wolf Y I, Alkhnbashi O S, Costa F, Shah S A, Saunders S J, Barrangou R, Brouns S J, Charpentier E, Haft D H, Horvath P, Moineau S, Mojica F J, Terns R M, Terns M P, White M F, Yakunin A F, Garrett R A, van der Oost J, Backofen R and Koonin E V (2015) An updated evolutionary classification of CRISPR-Cas systems. Nat Rev Microbiol 13:722-736.
Makarova K S, Wolf Y I and Koonin E V (2018) Classification and Nomenclature of CRISPR-Cas Systems: Where from Here? CRISPR J 1:325-336.
Mali P, Aach J, Stranges P B, Esvelt K M, Moosburner M, Kosuri S, Yang L and Church G M (2013a) CAS9 transcriptional activators for target specificity screening and paired nickases for cooperative genome engineering. Nat Biotechnol 31:833-838.
Mali P, Esvelt K M and Church G M (2013b) Cas9 as a versatile tool for engineering biology. Nat Methods 10:957-963.
Mali P, Yang L, Esvelt K M, Aach J, Guell M, DiCarlo J E, Norville J E and Church G M (2013c) RNA-guided human genome engineering via Cas9. Science 339:823-826.
Milligan J F, Groebe D R, Witherell G W and Uhlenbeck O C (1987) Oligoribonucleotide synthesis using T7 RNA polymerase and synthetic DNA templates. Nucleic Acids Res 15:8783-8798.
Mojica F J, Diez-Villasenor C, Garcia-Martinez J and Almendros C (2009) Short motif sequences determine the targets of the prokaryotic CRISPR defence system. Microbiology 155:733-740.
Moreno-Mateos M A, Vejnar C E, Beaudoin J D, Fernandez J P, Mis E K, Khokha M K and Giraldez A J (2015) CRISPRscan: designing highly efficient sgRNAs for CRISPR-Cas9 targeting in vivo. Nat Methods 12:982-988.
Nishimasu H, Ran F A, Hsu P D, Konermann S, Shehata S I, Dohmae N, Ishitani R, Zhang F and Nureki O (2014) Crystal structure of Cas9 in complex with guide RNA and target DNA. Cell 156:935-949.
Nishimasu H, Shi X, Ishiguro S, Gao L, Hirano S, Okazaki S, Noda T, Abudayyeh O O, Gootenberg J S, Mori H, Oura S, Holmes B, Tanaka M, Seki M, Hirano H, Aburatani H, Ishitani R, Ikawa M, Yachie N, Zhang F and Nureki O (2018) Engineered CRISPR-Cas9 nuclease with expanded targeting space. Science.
Nissim L, Perli S D, Fridkin A, Perez-Pinera P and Lu T K (2014) Multiplexed and programmable regulation of gene networks with an integrated RNA and CRISPR/Cas toolkit in human cells. Mol Cell 54:698-710.
Nowak C M, Lawson S, Zerez M and Bleris L (2016) Guide RNA engineering for versatile Cas9 functionality. Nucleic Acids Res 44:9555-9564.
Ran F A, Hsu P D, Lin C Y, Gootenberg J S, Konermann S, Trevino A E, Scott D A, Inoue A, Matoba S, Zhang Y and Zhang F (2013) Double nicking by RNA-guided CRISPR Cas9 for enhanced genome editing specificity. Cell 154:1380-1389.
Sander J D and Joung J K (2014) CRISPR-Cas systems for editing, regulating and targeting genomes. Nat Biotechnol 32:347-355.
Sanjana N E, Shalem O and Zhang F (2014) Improved vectors and genome-wide libraries for CRISPR screening. Nat Methods 11:783-784.
Slaymaker I M, Gao L, Zetsche B, Scott D A, Yan W X and Zhang F (2016) Rationally engineered Cas9 nucleases with improved specificity. Science 351:84-88.
Spitzer M, Wildenhain J, Rappsilber J and Tyers M (2014) BoxPlotR: a web tool for generation of box plots. Nat Methods 11:121-122.
Talas A, Kulcsar P I, Weinhardt N, Borsy A, Toth E, Szebenyi K, Krausz S L, Huszar K, Vida I, Sturm A, Gordos B, Hoffmann O I, Bencsura P, Nyeste A, Ligeti Z, Fodor E and Welker E (2017) A convenient method to pre-screen candidate guide RNAs for CRISPR/Cas9 gene editing by NHEJ-mediated integration of a ‘self-cleaving’ GFP-expression plasmid. DNA Res 24:609-621.
Tanenbaum M E, Gilbert L A, Qi L S, Weissman J S and Vale R D (2014) A protein-tagging system for signal amplification in gene expression and fluorescence imaging. Cell 159:635-646.
Tóth E, Huszir K, Bencsura P, Kulcsir P I, Vodicska B, Nyeste A, Welker Z, T6th S and Welker E (2014) Restriction enzyme body doubles and PCR cloning: on the general use of type IIs restriction enzymes for cloning. PloS one 9.
Tsai S Q, Topkar V V, Joung J K and Aryee M J (2016) Open-source guideseq software for analysis of GUIDE-seq data. Nat Biotechnol 34:483.
Tsai S Q, Wyvekens N, Khayter C, Foden J A, Thapar V, Reyon D, Goodwin M J, Aryee M J and Joung J K (2014) Dimeric CRISPR RNA-guided FokI nucleases for highly specific genome editing. Nat Biotechnol 32:569-576.
Tsai S Q, Zheng Z, Nguyen N T, Liebers M, Topkar V V, Thapar V, Wyvekens N, Khayter C, Iafrate A J, Le L P, Aryee M J and Joung J K (2015) GUIDE-seq enables genome-wide profiling of off-target cleavage by CRISPR-Cas nucleases. Nat Biotechnol 33:187-197.
Vakulskas C A, Dever D P, Rettig G R, Turk R, Jacobi A M, Collingwood M A, Bode N M, McNeill M S, Yan S, Camarena J, Lee C M, Park S H, Wiebking V, Bak R O, Gomez-Ospina N, Pavel-Dinu M, Sun W, Bao G, Porteus M H and Behlke M A (2018) A high-fidelity Cas9 mutant delivered as a ribonucleoprotein complex enables efficient gene editing in human hematopoietic stem and progenitor cells. Nat Med 24:1216-1224.
Vora S, Tuttle M, Cheng J and Church G (2016) Next stop for the CRISPR revolution: RNA-guided epigenetic regulators. FEBS J 283:3181-3193.
Vriend L E, Jasin M and Krawczyk P M (2014) Assaying break and nick-induced homologous recombination in mammalian cells using the D R-GFP reporter and Cas9 nucleases. Methods Enzymol 546:175-191.
Wu W Y, Lebbink J H G, Kanaar R, Geijsen N and van der Oost J (2018) Genome editing by natural and engineered CRISPR-associated nucleases. Nat Chem Biol 14:642-651.
Wyvekens N, Topkar V V, Khayter C, Joung J K and Tsai S Q (2015) Dimeric CRISPR RNA-Guided FokI-dCas9 Nucleases Directed by Truncated gRNAs for Highly Specific Genome Editing. Hum Gene Ther 26:425-431.
Xie K, Minkenberg B and Yang Y (2015) Boosting CRISPR/Cas9 multiplex editing capability with the endogenous tRNA-processing system. Proc Natl Acad Sci USA 112:3570-3575.
Zhang D, Zhang H, Li T, Chen K, Qiu J L and Gao C (2017) Perfectly matched 20-nucleotide guide RNA sequences enable robust genome editing using high-fidelity SpCas9 nucleases. Genome Biol 18:191.
Zuris J A, Thompson D B, Shu Y, Guilinger J P, Bessen J L, Hu J H, Maeder M L, Joung J K, Chen Z Y and Liu D R (2015) Cationic lipid-mediated delivery of proteins enables efficient protein-based genome editing in vitro and in vivo. Nat Biotechnol 33:73-80.

1. A variant Cas9 protein comprising a mutation in the surface loop proximal to the 5′ end of a target specific spacer sequence in a crRNA or sgRNA when said crRNA or sgRNA is in association with said Cas9 protein, said mutation comprising deletion of a segment of said surface loop to remove amino acids which, in a corresponding surface loop having a wild type sequence, are in contact with the 5′ end of the target specific spacer sequence, wherein the amino acids which are in contact with the 5′ end of the spacer sequence are included in the deleted segment; and wherein the length of the deleted segment is 6 to 12 amino acids,
wherein said mutation increases the available target space in the Cas9 protein to accommodate an 5′ extension of the spacer sequence, whereas the folded three dimensional structure of the Cas9 protein is otherwise maintained and the variant Cas9 protein has Cas9 activity on a target DNA substrate, wherein preferably the mutation also increases the fidelity of said variant Cas9 protein in comparison with a Cas9 protein having the same sequence but not having said mutation.

2. The variant Cas9 protein according to claim 1, which is a variant of a Streptococcus pyogenes Cas9 (SpCas9) protein, having a mutation in the surface loop comprising the following amino acids in a loop having a wild type sequence: Glu1007 and Tyr1013, wherein said mutation disrupts or removes a capping of the 5′ end of the spacer sequence in the crRNA or sgRNA, said capping being formed by said Glu1007 and Tyr1013 as capping amino acids and wherein said mutation is between the amino acids Leu1004 and Asp1017, preferably between Leu1004 and Lys1014.

3. The variant Cas9 protein according to claim 1, wherein the variant Cas9 comprises a sequence selected from the following group consisting of SEQ ID NOs 6 to 23 as listed below, wherein said sequence is present in the position of (i.e. replaces) the wild type segment from Lys1003 to Asp1017 of SpCas9 or a corresponding sequence of a wild type Cas9 protein comprising said surface loop:

| 1003-------------1017 | |
|---|---|
| KLESEFVYGDYKVYD | SEQ ID NO: 4 |
| KGKVYD | SEQ ID NO: 6 |
| KD | SEQ ID NO: 7 |
| KGD | SEQ ID NO: 8 |
| KGKD | SEQ ID NO: 9 |
| KLKVYD | SEQ ID NO: 10 |
| KLGKVYD | SEQ ID NO: 11 |
| KLPKVYD | SEQ ID NO: 12 |
| KLGGKVYD | SEQ ID NO: 13 |
| KLGPKVYD | SEQ ID NO: 14 |
| KLGGGKVYD | SEQ ID NO: 15 |
| KLGGGGKVYD | SEQ ID NO: 16 |
| KLGGVYD | SEQ ID NO: 17 |
| KLGGYD | SEQ ID NO: 18 |
| KLGGD | SEQ ID NO: 19 |
| KLSSKVYD | SEQ ID NO: 20 |
| KLSSD | SEQ ID NO: 21 |
| KLESKVYD | SEQ ID NO: 22 |
| KLESGKVYD | SEQ ID NO: 23 |

4. The variant Cas9 protein according to claim 1, said variant Cas9 comprising SEQ ID NO. 13, wherein said sequence replaces the wild type segment from Lys1003 to Asp1017 of SpCas9 or a corresponding sequence of a wild type Cas9 protein comprising said surface loop.

5. The variant Cas9 protein according to claim 1, said mutation comprising a deletion of a segment of said surface loop, wherein the length of the deleted segment is 7 to 11 amino acids or 8 to 10 amino acids or highly preferably 9 amino acids.

6. The variant Cas9 protein according to claim 1, said mutation also comprising an insertion to replace the segment deleted wherein said amino acids being in contact with the 5′ end of the spacer sequence in the wild type loop sequence are replaced or deleted in the variant Cas9,
said insertion having a length of 1 to 6 amino acid(s) and comprises amino acids which are different from acidic and aromatic amino acids, and the volume of insertion, and or the space filling or steric effect of the amino acid(s) altogether is smaller than that of the wild type amino acids altogether.

7. The variant Cas9 protein according to claim 6, wherein said insertion is selected from the following group of peptides and amino acids:
(Gly)ₘ, wherein m is an integer from 1 to 6
(Ala)ₙ, wherein n is an integer from 1 to 6
(Ser)ₒ, wherein o is an integer from 1 to 4
(Gly)ₓ(Pro)_{y}, wherein x and y are, independently from each other, integers from 1 to 4 wherein the sum of x+y is not more than 6,
(Gly)ₓ(Ala)_{y}, wherein x and y are, independently from each other, integers from 1 to 4 wherein the sum of x+y is not more than 6,
(Gly)ₓ(Lys)_{y}, wherein x and y are, independently from each other, integers from 1 to 4 wherein the sum of x+y is not more than 6,
(Gly)ₓ(Ser)_{y}, wherein x and y are, independently from each other, integers from 1 to 4 wherein the sum of x+y is not more than 6,
wherein preferably said insertion is selected from the following group of peptides and amino acids:
(Gly)ₘ, wherein m is an integer from 1 to 4.

8. The variant Cas9 protein of claim 1, further comprising any fidelity-increasing mutation of an increased fidelity variant,
preferably wherein the mutation is selected from a group of mutations present in one or more increased fidelity variant(s) selected from the group consisting of SpCas9-HF1, HypaSpCas9, evoSpCas9, HiFi SpCas9, Sniper SpCas9, eSpCas9, Hypa2SpCas9 and HeFSpCas9 or any fidelity increasing mutation in an increased fidelity Cas9,
preferably said mutation is selected from the group consisting of K848A, K1003A, R1060A.

9. The variant Cas9 protein of claim 1, said protein comprising an amino acid sequence that has at least 80% sequence identity to the following amino acid sequence,
wherein Lys 1003, Leu1004 and Glu1007 are marked in bold and underlined, respectively and Tyr1013, Leu1004 and Asp1017 are marked in bold, respectively,

| | |
|---|---|
| MDYKDHDGDYKDHDIDYKDDDDK MAPKKKRKVGIHGVPAA | 40 |
| DKKYSIGLDIGTNSVGWAVITDEYKVPSKKFKVLGNTDRHSIKKNLIGALLFDSGETAEA | 100 |
| TRLKRTARRRYTRRKNRICYLQEIFSNEMAKVDDSFFHRLEESFLVEEDKKHERHPIFGN | 160 |
| IVDEVAYHEKYPTIYHLRKKLVDSTDKADLRLIYLALAHMIKFRGHFLIEGDLNPDNSDV | 220 |
| DKLFIQLVQTYNQLFEENPINASGVDAKAILSARLSKSRRLENLIAQLPGEKKNGLFGNL | 280 |
| IALSLGLTPNFKSNFDLAEDAKLQLSKDTYDDDLDNLLAQIGDQYADLFLAAKNLSDAIL | 340 |
| LSDILRVNTEITKAPLSASMIKRYDEHHQDLTLLKALVRQQLPEKYKEIFFDQSKNGYAG | 400 |
| YIDGGASQEEFYKFIKPILEKMDGTEELLVKLNREDLLRKQRTFDNGSIPHQIHLGELHA | 460 |
| ILRRQEDFYPFLKDNREKIEKILTFRIPYYVGPLARGNSRFAWMTRKSEETITPWNFEEV | 520 |
| VDKGASAQSFIERMTNFDKNLPNEKVLPKHSLLYEYFTVYNELTKVKYVTEGMRKPAFLS | 580 |
| GEQKKAIVDLLFKTNRKVTVKQLKEDYFKKIECFDSVEISGVEDRFNASLGTYHDLLKII | 640 |
| KDKDFLDNEENEDILEDIVLTLTLFEDREMIEERLKTYAHLFDDKVMKQLKRRRYTGWGR | 700 |
| LSRKLINGIRDKQSGKTILDFLKSDGFANRNFMQLIHDDSLTFKEDIQKAQVSGQGDSLH | 760 |
| EHIANLAGSPAIKKGILQTVKVVDELVKVMGRHKPENIVIEMARENQTTQKGQKNSRERM | 820 |
| KRIEEGIKELGSQILKEHPVENTQLQNEKLYLYYLQNGRDMYVDQELDINRLSDYDVDHI | 880 |
| VPQSFLKDDSIDNKVLTRSDKNRGKSDNVPSEEVVKKMKNYWRQLLNAKLITQRKFDNLT | 940 |
| KAERGGLSELDKAGFIKRQLVETRQITKHVAQILDSRMNTKYDENDKLIREVKVITLKSK | 1000 |
| LVSDFRKDFQFYKVREINNYHHAHDAYLNAVVGTALIKKYPKLESEFVYGDYKVYDVRKM | 1060 |
| IAKSEQEIGKATAKYFFYSNIMNFFKTEITLANGEIRKRPLIETNGETGEIVWDKGRDFA | 1120 |
| TVRKVLSMPQVNIVKKTEVQTGGFSKESILPKRNSDKLIARKKDWDPKKYGGFDSPTVAY | 1180 |
| SVLVVAKVEKGKSKKLKSVKELLGITIMERSSFEKNPIDFLEAKGYKEVKKDLIIKLPKY | 1240 |
| SLFELENGRKRMLASAGELQKGNELALPSKYVNFLYLASHYEKLKGSPEDNEQKQLFVEQ | 1300 |
| HKHYLDEIIEQISEFSKRVILADANLDKVLSAYNKHRDKPIREQAENIIHLFTLTNLGAP | 1360 |
| AAFKYFDTTIDRKRYTSTKEVLDATLIHQSITGLYETRIDLSQLGGDKRPAATKKAGQAK | 1420 |
| KKK | 1423 |

(SEQ ID NO: 1) wherein N- and C-terminal added peptides are
marked in intalics) or to the following amino acid sequence

| | |
|---|---|
| DKKYSIGLDIGTNSVGWAVITDEYKVPSKKFKVLGNTDRHSIKKNLIGALLFDSGETAEA | 60 |
| TRLKRTARRRYTRRKNRICYLQEIFSNEMAKVDDSFFHRLEESFLVEEDKKHERHPIFGN | 120 |
| IVDEVAYHEKYPTIYHLRKKLVDSTDKADLRLIYLALAHMIKFRGHFLIEGDLNPDNSDV | 180 |
| DKLFIQLVQTYNQLFEENPINASGVDAKAILSARLSKSRRLENLIAQLPGEKKNGLFGNL | 240 |
| IALSLGLTPNFKSNFDLAEDAKLQLSKDTYDDDLDNLLAQIGDQYADLFLAAKNLSDAIL | 300 |
| LSDILRVNTEITKAPLSASMIKRYDEHHQDLTLLKALVRQQLPEKYKEIFFDQSKNGYAG | 360 |
| YIDGGASQEEFYKFIKPILEKMDGTEELLVKLNREDLLRKQRTFDNGSIPHQIHLGELHA | 420 |
| ILRRQEDFYPFLKDNREKIEKILTFRIPYYVGPLARGNSRFAWMTRKSEETITPWNFEEV | 480 |
| VDKGASAQSFIERMTNFDKNLPNEKVLPKHSLLYEYFTVYNELTKVKYVTEGMRKPAFLS | 540 |
| GEQKKAIVDLLFKTNRKVTVKQLKEDYFKKIECFDSVEISGVEDRFNASLGTYHDLLKII | 600 |
| KDKDFLDNEENEDILEDIVLTLTLFEDREMIEERLKTYAHLFDDKVMKQLKRRRYTGWGR | 660 |
| LSRKLINGIRDKQSGKTILDFLKSDGFANRNFMQLIHDDSLTFKEDIQKAQVSGQGDSLH | 720 |
| EHIANLAGSPAIKKGILQTVKVVDELVKVMGRHKPENIVIEMARENQTTQKGQKNSRERM | 780 |
| KRIEEGIKELGSQILKEHPVENTQLQNEKLYLYYLQNGRDMYVDQELDINRLSDYDVDHI | 840 |
| VPQSFLKDDSIDNKVLTRSDKNRGKSDNVPSEEVVKKMKNYWRQLLNAKLITQRKFDNLT | 900 |
| KAERGGLSELDKAGFIKRQLVETRQITKHVAQILDSRMNTKYDENDKLIREVKVITLKSK | 960 |
| LVSDFRKDFQFYKVREINNYHHAHDAYLNAVVGTALIKKYPKLESEFVYGDYKVYDVRKM | 1020 |
| IAKSEQEIGKATAKYFFYSNIMNFFKTEITLANGEIRKRPLIETNGETGEIVWDKGRDFA | 1080 |
| TVRKVLSMPQVNIVKKTEVQTGGFSKESILPKRNSDKLIARKKDWDPKKYGGFDSPTVAY | 1140 |
| SVLVVAKVEKGKSKKLKSVKELLGITIMERSSFEKNPIDFLEAKGYKEVKKDLIIKLPKY | 1200 |
| SLFELENGRKRMLASAGELQKGNELALPSKYVNFLYLASHYEKLKGSPEDNEQKQLFVEQ | 1260 |
| HKHYLDEIIEQISEFSKRVILADANLDKVLSAYNKHRDKPIREQAENIIHLFTLTNLGAP | 1320 |
| AAFKYFDTTIDRKRYTSTKEVLDATLIHQSITGLYETRIDLSQLGGD | 1367 |

(SEQ ID NO: 2) of Uniprot entry no. Q99ZW2; CRISPR-associated
endonuclease Cas9/Csn1 from Streptococcus pyogenes serotype M1
or to the following amino acid sequence

| | |
|---|---|
| DKKYSIGLDIGTNSVGWAVITDDYKVPSKKFKVLGNTDRHSIKKNLIGALLFDSGETAEA | 60 |
| TRLKRTARRRYTRRKNRICYLQEIFSNEMAKVDDSFFHRLEESFLVEEDKKHERHPIFGN | 120 |
| IVDEVAYHEKYPTIYHLRKKLADSTDKADLRLIYLALAHMIKFRGHFLIEGDLNPDNSDV | 180 |
| DKLFIQLVQTYNQLFEENPINASRVDAKAILSARLSKSRRLENLIAQLPGEKKNGLFGNL | 240 |
| IALSLGLTPNFKSNFDLAEDAKLQLSKDTYDDDLDNLLAQIGDQYADLFLAAKNLSDATL | 300 |
| LSDILRVNSEITKAPLSASMIKRYDEHHQDLTLLKALVRQQLPEKYKEIFFDQSKNGYAG | 360 |
| YIDGGASQEEFYKFIKPILEKMDGTEELLAKLNREDLLRKQRTFDNGSIPHQIHLGELHA | 420 |
| ILRRQEDFYPFLKDNREKIEKILTFRIPYYVGPLARGNSRFAWMTRKSEETITPWNFEEV | 480 |
| VDKGASAQSFIERMTNFDKNLPNEKVLPKHSLLYEYFTVYNELTKVKYVTEGMRKPAFLS | 540 |
| GEQKKAIVDLLFKTNRKVTVKQLKEDYFKKIECFDSVEISGVEDRFNASLGTYHDLLKII | 600 |
| KDKDFLDNEENEDILEDIVLTLTLFEDKEMIEERLKTYAHLFDDKVMKQLKRRHYTGWGR | 660 |
| LSRKLINGIRDKQSGKTILDFLKSDGFANRNFIQLIHDDSLTFKEAIQKAQVSGQGHSLH | 720 |
| EQIANLAGSPAIKKGILQSVKVVDELVKVMGHKPENIVIEMARENQTTQKGQKNSRERMK | 780 |
| RIEEGIKELGSQILKEHPVENTQLQNEKLYLYYLQNGRDMYVDQELDINRLSDYDVDHIV | 840 |
| PQSFTKDDSIDNKVLTRSDKNRGKSDDVPSEEVVKKMKNYWRQLLNAKLITQRKFDNLTK | 900 |
| AERGGLSELDKAGFIKRQLVETRQITKHVAQILDSRMNTKYDENDKLIREVKVITLKSKL | 960 |
| VSDFRKDFQFYKVREINNYHHAHDAYLNAVVGTALIKKYPKLESEFVYGDYKVYDVRKMI | 1020 |
| AKSEQEIGKATAKRFFYSNIMNFFKTEITLANGEIRKRPLIETNEETGEIVWDKGRDFAT | 1080 |
| VRKVLSMPQVNIVKKTEVQTGALTNESIYARGSFDKLISRKHRFESSKYGGFGSPTVTYS | 1140 |
| VLVVAKSKVQDGKVKKIKTGKELIGITLLDKLVFEKNPLKFIEDKGYGNVQIDKCIKLPK | 1200 |
| YSLFEFENGTRRMLASVMANNNSRGDLQKANEMFLPAKLVTLLYHAHKIESSKELEHEAY | 1260 |
| ILDHYNDLYQLLSYIERFASLYVDVEKNISKVKELFSNIESYSISEICSSVINLLTLTAS | 1320 |
| GAPADFKFLGTTIPRKRYGSPQSILSSTLIHQSITGLYETRIDLSQLGSD | 1370 |

(SEQ ID NO: 3) of Uniprot entry no. Q1J6W2; CRISPR-associated
endonuclease Cas9 from Streptococcus pyogenes serotype M4
(strain MGAS10750),
wherein preferably the variant Cas9 comprises a sequence selected from the group consisting of SEQ ID NOs 5 to 23, preferably SEQ ID NOs 6 to 23, more preferably SEQ ID NOs 11 to 21, highly preferably SEQ ID NO 13.

10. The variant Cas9 protein of claim 9 said protein comprising an amino acid sequence that has at least 80% sequence identity to the amino acid sequence of (SEQ ID NO: 2),
wherein said variant Cas9 comprises a sequence selected from the following group consisting of SEQ ID NOs 6 to 23 as listed below, wherein said sequence is present in the position of (i.e. replaces) the wild type segment from Lys1003 to Asp1017 of SpCas9 or a corresponding sequence of a wild type Cas9 protein comprising said surface loop.

11. A fusion protein comprising the variant Cas9 protein of claim 1 fused to a heterologous functional domain or attached to a heterologous functional domain via a non-covalent bond.

12. A ribonucleoprotein (RNP) complex comprising the variant Cas9 protein of claim 1, said RNP complex also comprising an RNA having a spacer sequence, preferably a crRNA or a single guide RNA (sgRNA).

13. An isolated nucleic acid encoding a Cas9 protein as defined in claim 1.

14. A vector comprising the isolated nucleic acid of claim 13.

15. A host cell comprising the nucleic acid of claim 13 or a vector comprising said nucleic acid wherein preferably said host cell is a mammalian host cell.

16. A kit comprising the isolated nucleic acid of claim 13, and/or a vector comprising said nucleic acid and/or a host cell comprising said nucleic acid, and a target specific crRNA or single guide RNA,
wherein preferably the target specific crRNA or single guide RNA is a library of crRNAs or sgRNAs.

17. A method of altering the genome or epigenome of a cell, said method comprising
expressing in the cell a Cas9 protein according to claim 1 or contacting the cell with said Cas9 protein,
providing a crRNA having a target specific spacer sequence, preferably a target-specific single guide RNA (sgRNA) having a region complementary to a selected portion of the genome of the cell,
whereby the genome of the cell is altered.
